(19) United States
(12) Patent Application Publication
(10) Pub. No.: US 2012/0149661 A1
(43) Pub. Date: Jun. 14, 2012

(54) TETRA-SUBSTITUTED HETEROARYL COMPOUNDS AND THEIR USE AS MDM2 AND/OR MDM4 MODULATORS

(75) Inventors: Guido Bold, Gipf-Oberfrick (CH); Pascal Furet, Thann (FR); Francois Gessier, Altkirch (FR); Joerg Kallen, Basel (CH); Joanna Hergovich Lisztwan, Walton-on-Thames (GB); Keiichi Masuya, Basel (CH); Andrea Vaupel, Riehen (CH)

(73) Assignee: NOVARTIS AG, Basel (CH)

(21) Appl. No.: 13/392,231

(22) PCT Filed: Aug. 24, 2010
(86) PCT No.: PCT/EP10/62300

§ 371 Date: Feb. 24, 2012

Related U.S. Application Data
(60) Provisional application No. 61/237,107, filed on Aug. 26, 2009.

(51) Int. Cl. A61K 31/4164; C07D 403/04; A61K 31/4178; C07D 413/12; A61P 35/00; C07F 9/40; A61K 31/675; C07F 7/18; A61K 31/695; C07D 233/90; A61K 31/5377

(52) U.S. Cl. 514/63; 548/333.5; 514/400; 548/254; 514/381; 544/139; 514/235.8; 514/236.2; 548/111; 514/94; 548/110

(57) ABSTRACT

The invention relates to tetra-substituted heteroarylic compounds of the formula (I)

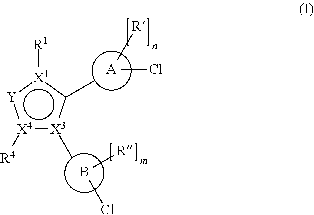

wherein X¹, X³ and X⁴ are independently C or N, Y is C—H, N—H or N, wherein the total number of nitrogen atoms represented by X¹, X³, X⁴ and Y is 1 or 2; rings A and B are independently selected from phenyl or pyridyl; R1, R4, R′, R″, n and m are as defined herein. Such compounds are suitable for the treatment of a disorder or disease which is mediated by the activity of MDM2 and/or MDM4, or variants thereof.

### INTRODUCTION

The present invention relates to tetra-substituted 5-membered heteroaryl compounds, capable of inhibiting the interaction between p53, or variants thereof, and MDM2 and/or MDM4, or variants thereof, respectively, especially binding to MDM2 and/or MDM4, or variants thereof, a process for the preparation of such compounds, pharmaceutical preparations comprising such compounds, uses and methods of use for such compounds in the treatment (including therapy and/or prophylaxis), and/or related subject matter as specified below. p53 refers to all genes and/or proteins encoded thereof with the names TP53, p53, TP73, p73, TP63, TP73L, p63. MDM2 refers to all genes and/or proteins encoded thereof with the names MDM2, Mdm2, HDM2, Hdm2. MDM4 refers to all genes and/or proteins encoded thereof with the names MDM4, Mdm4, HDM4, Hdm4, MDMX, MdmX, HDMX, HdmX.

Protein p53 is known as a tumor suppressor protein which helps to control cellular integrity and prevents the proliferation of permanently damaged cells by initiating, among other responses, growth arrest or apoptosis (controlled cell death). p53 mediates its effects in that it is a transcription factor capable of regulating a number of genes that regulate e.g. cell cycle and apoptosis. Thus, p53 is an important cell cycle inhibitor. These activities are tightly controlled by MDM2, an important negative regulator of the p53 tumor supressor. “MDM2” (originally from the oncogene “murine double minute 2”) refers both to the name of the gene as well as the protein encoded by that gene. MDM2 protein functions both as a E3 ubiquitin ligase that recognizes the N-terminal trans-activation domain (TAD) of the p53 tumor suppressor and thus mediates the ubiquitin-dependent degradation of p53, and as an inhibitor of p53 transcriptional activation.

The original mouse oncogene, which codes for the MDM2 protein, was originally cloned from a transformed mouse cell line. The human homologue of this protein was later identified and is sometimes also called HDM2 (for “human double minute 2”). Further supporting the role of MDM2 as an oncogene, several human tumor and proliferative disease types have been shown to have increased levels of MDM2, including inter alia soft tissue sarcomas, bone cancer, e.g. osteosarcomas, breast tumors, bladder cancer, Li-Fraumeni syndrome, brain tumor, rhabdomyosarcoma and adrenocortical carcinoma and the like. Another protein belonging to the MDM2 family is MDM4, also known as MDMX.

Dysregulation of the MDM2/p53 ratio, e.g. due to mutations, polymorphisms or molecular defects in the affected cells, can thus be found in many proliferative diseases. MDM2, in view of its mentioned effects, is capable to inhibit the activity of the tumor suppressor protein p53, thus leading to loss of p53's tumor suppressor activity and inhibiting regulatory mechanisms that impede cells from uncontrolled proliferation. As a consequence, uncontrolled proliferation can take place, leading to tumors, leukemias or other proliferative diseases.

Thus there is a need for new drugs that are capable to interfere with the interaction between p53 and MDM2 or especially oncogenic variants thereof and that thus allow p53 to exert its beneficial effect against uncontrolled tumor growth, allowing it e.g. to accumulate, to arrest the cell cycle and/or to cause apoptosis of affected cells.

### SUMMARY OF THE INVENTION

It has now been found that a novel class of 5-membered, substituted aromatic heterocycles shows potent inhibition of the MDM2/p53 interaction (this term including MDM2/p53 interaction and/or MDM4/p53 interaction herein, in particular Hdm21p53 and/or Hdm41p53 interaction) and the corresponding compounds thus represent a novel type of compounds that are useful in the treatment of a number of disorders, such as proliferative diseases. The invention relates therefore to these compounds as drugs as well as to the other inventive embodiments indicated above and below.

### DETAILED DESCRIPTION OF THE INVENTION

In a first and preferred embodiment, the invention relates to heteroarylic compounds of the formula (I), containing between 1 to 2 nitrogen atoms, and/or tautomers and/or N-oxides and/or pharmaceutically acceptable salts and/or solvates thereof,

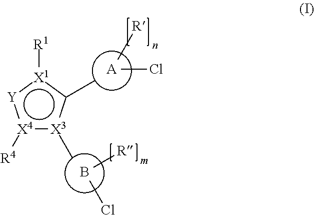

wherein
X¹, X³ and X⁴ are independently C or N,

### Y is C—H, N—H or N,

wherein the total number of nitrogen atoms represented by X¹, X³, X⁴ and Y is 1 or 2;
   rings A and B are independently selected from phenyl or pyridyl, wherein the Chlorine substituents are independently in the 3 or 4 position;
   R¹ is selected from the group consisting of
   cyano-
   cyano-methyl-
   carboxy-C₁-C₂-alkyl-
   carboxyl-
   C₁-C₇-alkoxy-carbonyl-
   amino-carbonyl-
   N—C₁-C₇-alkyl-amino-carbonyl-
   N,N-di-C₁-C₇-alkyl-amino-carbonyl-

### N-hydroxyl-amino-carbonyl-

N-hydroxyl-N—C₁-C₇-alkyl-amino-carbonyl-
N—C₁-C₇-alkoxy-amino-carbonyl-
N—C₁-C₇-alkoxy-N—C₁-C₇-alkyl-amino-carbonyl-
C₁-C₇-alkyl-carbonyl-amino-C₁-C₂-alkyl-
heterocyclyl-
C₁-C₇-alkyl-carbonyl-
formyl-
hydroxy-C₁-C₂-alkyl-
heterocyclyl-carbonyl-
S—C₁-C₇-alkyl-sulfonimidoyl-
S—C₁-C₇-alkyl-N—C₁-C₇-alkyl-sulfonimidoyl-
C₁-C₇-alkyl-sulfonyl-
amino-
S—C₁-C₇-alkyl-sulfoximino-
N—C₁-C₇-alkyl-amino-
N,N-di-C₁-C₇-alkyl-amino-
C₁-C₇-alkoxy-carbonyl-amino-
N—(C₁-C₇-alkoxy-carbonyl)-N—C₁-C₇-alkyl-amino-
C₁-C₇-alkyl-carbonyl-amino-
N—(C₁-C₇-alkyl-carbonyl)-N—C₁-C₇-alkyl-amino-
amino-sulfonyl-
N—C₁-C₇-alkyl-amino-sulfonyl-
N,N-di-C₁-C₇-alkyl-amino-sulfonyl-
hydrazino-carbonyl-
N—C₁-C₇-alkyl-hydrazino-carbonyl-
N,N-di-C₁-C₇-alkyl-hydrazino-carbonyl-
N—C₁-C₇-alkyl-hydrazino-carbonyl-
N,N-di-C₁-C₇-alkyl-hydrazino-carbonyl-
N,N-di-C₁-C₇-alkyl-N′—C₁-C₇-alkyl-hydrazino-carbonyl-
C₁-C₇-alkyl-carbonyl-hydrazino-carbonyl-
C₁-C₇-alkyl-carbonyl-N—C₁-C₇-alkyl-N′—C₁-C₇-alkyl-hydrazino-carbonyl-
phosphonyl-
C₁-C₇-alkyl-phosphonyl-
di-C₁-C₇-alkyl-phosphonyl-,
wherein C₁-C₇-alkyl or C₁-C₇-alkoxy groups are unsubstituted or substituted by 1-4 substituents selected from:
amino-
N—C₁-C₇-alkyl-amino-
N,N-di-C₁-C₇-alkyl-amino-
N-aryl-amino-
N-aryl-N—C₁-C₇-alkyl-amino-
heterocyclyl-
heterocyclyl-carbonyl-
C₃-C₁₀-cycloalkyl-
hydroxy-
cyano-
halogen-
halo-C₁-C₇-alkyl-
C₁-C₇-alkoxy-
C₁-C₇-alkyl-carbonyl-amino-
N—(C₁-C₇-alkyl-carbonyl)-N—C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
amino-carbonyl-amino-
N—C₁-C₇-alkyl-amino-carbonyl-amino-
N,N-di-C₁-C₇-alkyl-amino-carbonyl-amino-
amino-carbonyl-N′—(C₁-C₇-alkyl)-amino-
N—C₁-C₇-alkyl-amino-carbonyl-N′—(C₁-C₇-alkyl)-amino-
N,N-di-C₁-C₇-alkyl-amino-carbonyl-N′—(C₁-C₇-alkyl)-amino-
carboxyl-
C₁-C₇-alkoxy-carbonyl-
aryl- and
and wherein C₁-C₇-alkyl and C₁-C₇-alkoxy groups as part of these substituents can be further substituted as described above for C₁-C₇-alkyl and C₁-C₇-alkoxy;
and wherein heterocyclic groups are unsubstituted or substituted by 1-4 substituents selected from:
amino-
N—C₁-C₇-alkyl-amino-
N,N-di-C₁-C₇-alkyl-amino-
heterocyclyl-
C₃-C₁₀-cycloalkyl-
cyano-
halogen-
halo-C₁-C₇-alkyl-
C₁-C₇-alkoxy-
C₁-C₇-alkoxy-C₁-C₇-alkyl-
protected hydroxy-C₁-C₇-alkyl-
C₁-C₇-alkyl-carbonyl-amino-
N—(C₁-C₇-alkyl-carbonyl)-N—C₁-C₇-alkyl-amino-
N,N-di-C₁-C₇-alkyl-amino-C₁-C₇-alkyl-amino-
C₁-C₇-alkoxy-C₁-C₇-alkyl-amino-
aryl-C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
carboxyl-
C₁-C₇-alkoxy-carbonyl-
C₁-C₇-alkyl-
oxo (O═)
thiono (S═)
wherein C₁-C₇-alkyl and C₁-C₇-alkoxy as part of these substituents can be further substituted as described above for C₁-C₇-alkyl and C₁-C₇-alkoxy.
   R⁴ is selected from the group consisting of
   substituted C₁-alkyl-
   C₂-C₇-alkyl-
   aryl-
   heteroaryl-
   heterocyclyl-
   C₃-C₁₀-cycloalkyl-
   aryl-C₁-C₇-alkyl-
   heteroaryl-C₁-C₇-alkyl-
   heterocyclyl-C₁-C₇-alkyl-
   C₃-C₁₀-cycloalkyl-C₁-C₇-alkyl-
wherein substituents R⁴ are unsubstituted or substituted by 1-3 substituents selected from
hydroxy-
C₁-C₇-alkoxy-
C₁-C₇-alkoxy-carbonyl-
halogen-
halo-C₁-C₇-alkyl-
nitro-
C₁-C₇-alkyl-carbonyl-
formyl-
amino-
N—C₁-C₇-alkyl-amino-
N,N-di-C₁-C₇-alkyl-amino-
C₁-C₇-alkyl-carbonyl-amino-
N—(C₁-C₇-alkyl-carbonyl)-N—C₁-C₇-alkyl-amino-
hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N,N-di-C₁-C₇-alkyl-N′—C₁-C₇-alkyl-hydrazino-carbonyl-C₁-C₇-alkyl-amino-
C₁-C₇-alkyl-carbonyl-hydrazino-carbonyl-C₁-C₇-alkyl-amino-
C₁-C₇-alkyl-carbonyl-N—C₁-C₇-alkyl-N′—C₁-C₇-alkyl-hydrazino-carbonyl-C₁-C₇-alkyl-amino-
hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N,N-di-C₁-C₇-alkyl-N′—C₁-C₇-alkyl-hydrazino-carbonyl-C₁-C₇-alkyl-N—(C₁-C₇-alkyl)-amino-
C₁-C₇-alkyl-carbonyl-hydrazino-carbonyl-C₁-C₇-alkyl-N—(C₁-C₇-alkyl)-amino-
C₁-C₇-alkyl-carbonyl-N—C₁-C₇-alkyl-N′—C₁-C₇-alkyl-hydrazino-carbonyl-C₁-C₇-alkyl-
N—(C₁-C₇-alkyl)-amino-
tert-butyl-diphenyl-silanyloxy-
heterocyclyl-
protected hydroxy-
   wherein C₁-C₇-alkyl or C₁-C₇-alkoxy groups as part of substituents for R⁴ as defined above are unsubstituted or substituted by 1-4 groups, independently selected from:
   amino-
   N—C₁-C₇-alkyl-amino-
   N,N-di-C₁-C₇-alkyl-amino-
   N-aryl-amino-
   N-aryl-N—C₁-C₇-alkyl-amino-
   heterocyclyl-
   heterocyclyl-carbonyl-
   C₃-C₁₀-cycloalkyl-
   hydroxy-
   cyano-
   halogen-
   halo-C₁-C₇-alkyl-
   C₁-C₇-alkoxy-
   C₁-C₇-alkyl-carbonyl-amino-
   N—(C₁-C₇-alkyl-carbonyl)-N—C₁-C₇-alkyl-amino-
   C₁-C₇-alkyl-carbonyl-
   formyl-
   amino-carbonyl-
   N—C₁-C₇-alkyl-amino-carbonyl-
   N,N-di-C₁-C₇-alkyl-amino-carbonyl-
   amino-carbonyl-amino-
   N—C₁-C₇-alkyl-amino-carbonyl-amino-
   N,N-di-C₁-C₇-alkyl-amino-carbonyl-amino-
   amino-carbonyl-N′—(C₁-C₇-alkyl)-amino-
   N—C₁-C₇-alkyl-amino-carbonyl-N′—(C₁-C₇-alkyl)-amino-
   N,N-di-C₁-C₇-alkyl-amino-carbonyl-N′—(C₁-C₇-alkyl)-amino-
   carboxyl-
   C₁-C₇-alkoxy-carbonyl-
   aryl- and
   and wherein C₁-C₇-alkyl and C₁-C₇-alkoxy groups as part of these substituents can be further substituted as described above for C₁-C₇-alkyl and C₁-C₇-alkoxy;
   and wherein heterocyclyl as part of substituents for R⁴ as defined above is unsubstituted or substituted by 1-4 groups, independently selected from:
   amino-
   N—C₁-C₇-alkyl-amino-
   heterocyclyl-
   C₃-C₁₀-cycloalkyl-
   cyano-
   halogen-
   halo-C₁-C₇-alkyl-
   C₁-C₇-alkoxy-
   C₁-C₇-alkoxy-C₁-C₇-alkyl-
   protected hydroxy-C₁-C₇-alkyl-
   C₁-C₇-alkyl-carbonyl-amino-
   N—(C₁-C₇-alkyl-carbonyl)-N—C₁-C₇-alkyl-amino-
   N,N-di-C₁-C₇-alkyl-amino-C₁-C₇-alkyl-amino-
   C₁-C₇-alkoxy-C₁-C₇-alkyl-amino-
   aryl-C₁-C₇-alkyl-amino-
   C₁-C₇-alkyl-carbonyl-
   formyl-
   amino-carbonyl-
   N—C₁-C₇-alkyl-amino-carbonyl-
   N,N-di-C₁-C₇-alkyl-amino-carbonyl-
   carboxyl-
   C₁-C₇-alkoxy-carbonyl-
   C₁-C₇-alkyl-
   oxo (O═)
   thiono (S═)
   and wherein C₁-C₇-alkyl and C₁-C₇-alkoxy groups as part of these substituents can be further substituted as described above for C₁-C₇-alkyl and C₁-C₇-alkoxy;
      R′ and R″ are independently selected from the group consisting of:
      hydroxy-
      C₁-C₇-alkoxy-
      halogen-
      halo-C₁-C₇-alkyl-
      cyano-
      C₁-C₇-alkyl-carbonyl-
      formyl-
      C₁-C₇-alkyl-
      amino-carbonyl-
      N—C₁-C₇-alkyl-amino-carbonyl-
      N,N-di-C₁-C₇-alkyl-amino-carbonyl-
      heterocyclyl-
      N-(hydroxy-C₁-C₇-alkyl)-amino-carbonyl-C₁-C₇-alkyl-
      N—(C₁-C₇-alkoxy-C₁-C₇-alkyl)-amino-carbonyl-C₁-C₇-alkyl-
      N-(hydroxy-C₁-C₇-alkyl)-N—C₁-C₇-alkyl-amino-carbonyl-C₁-C₇-alkyl-
      N—(C₁-C₇-alkoxy-C₁-C₇-alkyl)-N—C₁-C₇-alkyl-amino-carbonyl-C₁-C₇-alkyl-
      C₁-C₇-alkoxy-carbonyl-
      C₁-C₇-alkyl-carbonyl-amino-
      carboxyl- and
      where A and B, or A or B are pyridyl, R′ and R″ may also be independently selected from ═O, to form the group

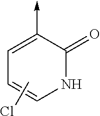

which may be further substituted with R′ and R″ as described above;
wherein C₁-C₇-alkyl or C₁-C₇-alkoxy groups as part of substituents on R′ or R″ as defined above are unsubstituted or substituted by 1-4 groups, independently selected from:
amino-
N—C₁-C₇-alkyl-amino-
N,N-di-C₁-C₇-alkyl-amino-
N-aryl-amino-
N-aryl-N—C₁-C₇-alkyl-amino-
heterocyclyl-
heterocyclyl-carbonyl-
C₃-C₁₀-cycloalkyl-
hydroxy-
cyano-
halogen-
halo-C₁-C₇-alkyl-
C₁-C₇-alkoxy-
C₁-C₇-alkyl-carbonyl-amino-
N—(C₁-C₇-alkyl-carbonyl)-N—C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
amino-carbonyl-amino-
N—C₁-C₇-alkyl-amino-carbonyl-amino-
N,N-di-C₁-C₇-alkyl-amino-carbonyl-amino-
amino-carbonyl-N—(C₁-C₇-alkyl)-amino-
N—C₁-C₇-alkyl-amino-carbonyl-N′—(C₁-C₇-alkyl)-amino-
N,N-di-C₁-C₇-alkyl-amino-carbonyl-N′—(C₁-C₇-alkyl)-amino-
carboxyl-
C₁-C₇-alkoxy-carbonyl-
C₁-C₇-alkoxy-carbonyl-amino-
aryl-
aryl-amino-carbonyl-, wherein said aryl is optionally substituted as described herein,
C₃-C₁₀-cycloalkyl-amino-carbonyl-
heterocyclyl-amino-carbonyl- and
and wherein C₁-C₇-alkyl and C₁-C₇-alkoxy groups as part of these substituents can be further substituted as described above for C₁-C₇-alkyl and C₁-C₇-alkoxy;
and wherein heterocyclyl as part of substituents on R′ or R″ as defined above is unsubstituted or substituted by 1-4 groups, independently selected from:
amino-
N—C₁-C₇-alkyl-amino-
N,N-di-C₁-C₇-alkyl-amino-
heterocyclyl-
C₃-C₁₀-cycloalkyl-
cyano-
halogen-
halo-C₁-C₇-alkyl-
C₁-C₇-alkoxy-
C₁-C₇-alkoxy-C₁-C₇-alkyl-
protected hydroxy-C₁-C₇-alkyl-
C₁-C₇-alkyl-carbonyl-amino-
N—(C₁-C₇-alkyl-carbonyl)-N—C₁-C₇-alkyl-amino-
N,N-di-C₁-C₇-alkyl-amino-C₁-C₇-alkyl-amino-
C₁-C₇-alkoxy-C₁-C₇-alkyl-amino-
aryl-C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
carboxyl-
C₁-C₇-alkoxy-carbonyl-
C₁-C₇-alkyl-
oxo (O═)
thiono (S═)
and wherein C₁-C₇-alkyl and C₁-C₇-alkoxy groups as part of these substituents can
be further substituted as described above for C₁-C₇-alkyl and C₁-C₇-alkoxy;
   and
   n and m are independently 0 to 2.

Wherever a compound or compounds of the formula (I) are mentioned, this is further also intended to include N-oxides of such compounds, tautomers thereof, and/or a (preferably pharmaceutically acceptable) salt thereof.

For purposes of interpreting this specification, the following definitions will apply and whenever appropriate, terms used in the singular will also include the plural and vice versa.

As used herein, the term “alkyl” refers to a fully saturated branched, including single or multiple branching, or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethyl pentyl, n-heptyl, n-octyl, n-nonyl, n-decyl and the like. Typically, alkyl groups have 1-7, more preferably 1-4 carbons.

As used herein, the term “halo-alkyl” refers to an alkyl as defined herein, that is substituted by one or more halo groups as defined herein. The halo-alkyl can be mono-halo-alkyl, di-halo-alkyl or poly-halo-alkyl including per-halo-alkyl. A mono-halo-alkyl can have one iodo, bromo, chloro or fluoro within the alkyl group. Di-halo-alky and poly-halo-alkyl groups can have two or more of the same halo atoms or a combination of different halo groups within the alkyl. Typically the poly-halo-alkyl contains up to 12, or 10, or 8, or 6, or 4, or 3, or 2 halo groups. Non-limiting examples of halo-alkyl include fluoro-methyl, di-fluoro-methyl, tri-fluoro-methyl, chloro-methyl, di-chloro-methyl, tri-chloro-methyl, penta-fluoro-ethyl, hepta-fluoro-propyl, di-fluoro-chloro-methyl, di-chloro-fluoro-methyl, di-fluoro-ethyl, di-fluoro-propyl, di-chloro-ethyl and dichloro-propyl. A per-halo-alkyl refers to an alkyl having all hydrogen atoms replaced with halo atoms.

As used herein, the term “alkylene” refers to divalent alkyl group as defined herein above having 1 to 20 carbon atoms. It comprises 1 to 20 carbon atoms, Unless otherwise provided, alkylene refers to moieties having 1 to 16 carbon atoms, 1 to 10 carbon atoms, 1 to 7 carbon atoms, or 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have 1-7, more preferably 1-4 carbons.

The term “aryl” refers to an aromatic hydrocarbon group having 6-20 carbon atoms in the ring portion. Typically, aryl is monocyclic, bicyclic or tricyclic aryl having 6-20 carbon atoms.

Furthermore, the term “aryl” as used herein, refers to an aromatic substituent which can be a single aromatic ring, or multiple aromatic rings that are fused together. Non-limiting examples include phenyl or naphthyl. Aryl may be unsubstituted or substituted by 1-4 substituents, selected from the group selected from

C₁-C₇-alkyl-
halo-C₁-C₇-alkyl-
hydroxy-C₁-C₇-alkyl-
C₃-C₁₀-cycloalkyl-
halogen-
hydroxy-
protected hydroxy-
C₁-C₇-alkoxy-
C₁-C₇-alkoxy-carbonyl-
C₁-C₇-alkyl-carbonyl-oxy-
aryl-carbonyl-oxy-
aryl-oxy-
heterocyclyl-oxy-
amino-
N—C₁-C₇-alkyl-amino-
N—C₁-C₇-alkyl-amino-N—C₁-C₇-alkyl-amino-
N—C₁-C₇-alkyl-amino-N,N-di-C₁-C₇-alkyl-amino-
N,N-di-C₁-C₇-alkyl-amino
N,N-di-C₁-C₇-alkyl-amino-N—C₁-C₇-alkyl-amino
N,N-di-C₁-C₇-alkyl-amino-N,N-di-C₁-C₇-alkyl-amino
C₁-C₇-alkoxy-N—C₁-C₇-alkyl-amino-
C₁-C₇-alkoxy-N,N-di-C₁-C₇-alkyl-amino-
aryl-C₁-C₇-alkyl-amino-
thio-
C₁-C₇-alkyl-thio-
aryl-thio-
aryl-C₁-C₇-alkyl-
nitro-
cyano-
carboxy-
C₁-C₇-alkoxy-carbonyl-
C₁-C₇-alkyl-carbonyl-
formyl-
amino-carbonyl-
C₁-C₇-alkyl-carbonyl-amino-
N—(C₁-C₇-alkyl-carbonyl)-N—C₁-C₇-alkyl-amino-
N—C₁-C₇-alkyl-amino-carbonyl-
N,N-di-C₁-C₇-alkyl-amino-carbonyl-
C₁-C₇-alkyl-sulfinyl-
C₁-C₇-alkyl-sulfonyl-
amino-sulfonyl-
N—C₁-C₇-alkyl-amino-sulfonyl-
N,N-di-C₁-C₇-alkyl-amino-sulfonyl-
aryl-
trimethylsilanyl-ethoxymethyl
heterocyclyl-.

In one embodiment, where aryl substituents are or contain C₁-C₇-alkyl, said alkyl is preferably C₁-C₄-alkyl, and in another embodiment C₁-C₂-alkyl.

As used herein, the term “aryl” preferably refers to unsubstituted phenyl or substituted phenyl, wherein the substituents for substituted phenyl are those as described above for “aryl”.

As used herein, the term “heterocyclyl”, “heterocyclic” or “heterocyclo” refers to an unsaturated (carrying the highest possible number of conjugated double bonds in the ring(s), then also called heteroaryl), saturated (then also called saturated heterocyclyl) or partially saturated ring or ring system, for example a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from N, O and S, where the N and S can also optionally be oxidized to various oxidation states. The heterocyclic group can be attached at a heteroatom or a carbon atom. The heterocyclyl can include fused or bridged rings as well as spirocyclic rings.

Examples of heterocycles include oxiranyl, azirinyl, aziridinyl, 1,2-oxathiolanyl, thienyl, furanyl, tetrahydrofuryl, pyranyl, tetrahydropyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, oxadiazolyl, isoxazolyl, pyridyl, pyrazinyl, pyrimidinyl, piperidinyl, piperazinyl, pyridazinyl, morpholinyl, thiomorpholinyl, indolizinyl, azepanyl, diazepanyl, especially 1,4-diazepanyl, isoindolyl, 3H-indolyl, indolyl, isoindolyl, indazolyl, benzimidazolyl, cumaryl, triazolyl, tetrazolyl, purinyl, 4H-quinolizinyl, isoquinolyl, isoquinolyl, tetrahydroquinolyl, tetrahydroisoquinolyl, decahydroquinolyl, octahydroisoquinolyl, benzofuranyl, thiophenyl, isobenzofuranyl, dibenzofuranyl, benzothiophenyl, dibenzothiophenyl, phthalazinyl, naphthyridinyl, quinoxalyl (=quinoxalinyl), quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromenyl, chromanyl, benzo[1,3]dioxol-5-yl, 2,3-dihydro-benzo[1,4]dioxin-6-yl, thiochromenyl and isothiochromenyl; each of which may be unsubstituted or substituted by 1-4 substituents, selected from the group of substituents described for “aryl” and/or from oxo (O═) or thiono (S═).

As R¹, the term “heterocyclyl-” refers preferably to 5-membered monocyclic unsaturated or partially saturated ring systems. Examples include, but are not limited to pyrrolyl, pyrazolyl, imidazolyl, oxazolyl, oxadiazolyl, isoxazolyl, triazolyl, tetrazolyl. As a group in a substituent for R¹, the term “heterocyclyk” refers preferably to 5- to 6-membered monocyclic saturated or partially saturated ring systems. Examples include, but are not limited to pyrrolidinyl, piperazinyl, piperidinyl and morpholinyl.

As R⁴, the term “heterocyclyk” (also in “heterocyclyl-C₁-C₇-alkyl-”) refers preferably to 5- to 6-membered monocyclic saturated or partially saturated ring systems. Examples include, but are not limited to pyrrolinyl, piperidinyl. As R⁴, the term “heteroaryl-” (also in “heteroaryl-C₁-C₇-alkyl-”) refers preferably to 5- to 12-membered mono- or bicyclic unsaturated ring systems. Examples include, but are not limited to pyridyl, benzothiophenyl, thiophenyl, indolyl. As group in a substituent for R⁴, the term “heterocyclyl-” refers preferrably to 5- to 6-membered monocyclic unsaturated or partially saturated ring systems. Examples include, but are not limited oxadiazolyl, dihydroimidazolyl, pyridyl.

As R′ and R″, the term “heterocyclyk” refers preferably to 5-membered monocyclic unsaturated ring systems. Examples include, but are not limited to oxatriazolyl. As group in a substituent for R′ and R″, the term “heterocyclyl-” refers preferrably to 5- to 6-membered monocyclic saturated, unsaturated or partially saturated ring systems. Examples include, but are not limited to pyrrolidinyl, piperazinyl, piperidinyl, morpholinyl, pyridyl.

As used herein, the term “cycloalkyl” refers to saturated or partially unsaturated monocyclic, bicyclic or tricyclic hydrocarbon groups of 3-12 carbon atoms. Unless otherwise provided, cycloalkyl refers to cyclic hydrocarbon groups having between 3 and 10 ring carbon atoms or between 3 and 7 ring carbon atoms, each of which are unsubstituted or substituted by one, or two, or three, or more substituents independently selected from the group of substituents described for “aryl”. Exemplary monocyclic hydrocarbon groups include, but are not limited to, cyclopropyl, cyclobutyl, cyclopentyl, cyclopentenyl, cyclohexyl and cyclohexenyl. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octy. Exemplary tricyclic hydrocarbon groups include adamantyl.

As used herein, the term “cycloalkyl” preferably refers to cyclopropyl, cyclopentyl, cycloheptyl or cyclohexyl.

As used herein, the term “oxy” refers to an —O— linking group.

As used herein, the term “carboxy” or “carboxyl” is —COOH.

As used herein, all substituents are written in a way to show the order of functional groups (groups) they are composed of. The functional groups are defined herein above. The point of their attachment is indicated with a hyphen (-) or an equal sign (=), as appropriate.

As used herein, the term “protected hydroxy” refers to a hydroxy functionality bearing a “protecting group”. Within the scope of this text, only a readily removable group that is not a constituent of the particular desired end product of the compounds of the present invention is designated a “protecting group”, unless the context indicates otherwise; e.g. a protecting group can be part of a compound of the formula (I), if specifically mentioned. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/1, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

The term “and/or an N-oxide thereof, a tautomer thereof and/or a (preferably pharmaceutically acceptable) salt thereof” especially means that a compound of the formula (I) may be present as such or in mixture with its N-oxide, as tautomer (e.g. due to keto-enol, lactam-lactim, amide-imidic acid or enamine-imine tautomerism) or in (e.g. equivalency reaction caused) mixture with its tautomer, or as a salt of the compound of the formula (I) and/or any of these forms or mixtures of two or more of such forms.

Various embodiments of the invention are described herein. It will be recognized that features specified in each embodiment may be combined with other specified features to provide further embodiments.

In a preferred embodiment the invention provides a compound of the formula (I), wherein the total number of nitrogen atoms represented by X¹, X³, X⁴ and Y is 2.

In a preferred embodiment the invention provides a compound of the formula (I) according to the formula (Ia)

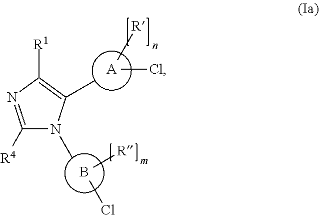

and/or a tautomer and/or an N-oxide and/or a pharmaceutically acceptable salt and/or a solvate thereof.

In another embodiment the invention provides a compound of the formula (I) according to the formula (Ib)

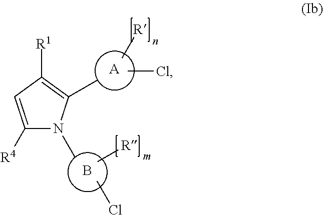

and/or a tautomer and/or an N-oxide and/or a pharmaceutically acceptable salt and/or a solvate thereof.

In another embodiment the invention provides a compound of the formula (I) according to the formula (Ic)

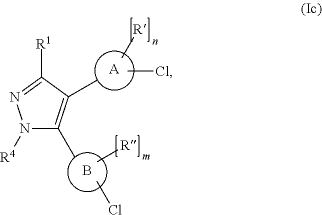

and/or a tautomer and/or an N-oxide and/or a pharmaceutically acceptable salt and/or a solvate thereof.

In another embodiment the invention provides a compound of the formula (I) according to the formula (Id)

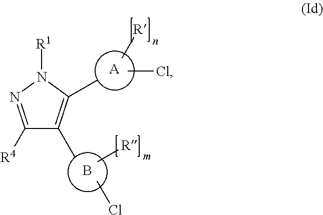

and/or a tautomer and/or an N-oxide and/or a pharmaceutically acceptable salt and/or a solvate thereof.

In another embodiment the invention provides a compound of the formula (I) according to the formula (Ie)

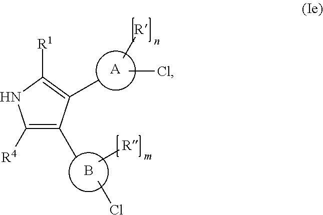

and/or a tautomer and/or an N-oxide and/or a pharmaceutically acceptable salt and/or a solvate thereof.

In a one embodiment the invention provides a compound of the formula (I) and/or a tautomer and/or an N-oxide and/or a pharmaceutically acceptable salt and/or a solvate thereof, wherein

both rings A and B are phenyl, wherein the Chlorine substituents are independently in the 3 or 4 position.

In another embodiment the invention provides a compound of the formula (I) and/or a tautomer and/or an N-oxide and/or a pharmaceutically acceptable salt and/or a solvate thereof, wherein

ring A is pyridyl and ring B is phenyl, wherein the chlorine substituents are independently in the 3 or 4 position.

In another embodiment the invention provides a compound of the formula (I) and/or a tautomer and/or an N-oxide and/or a pharmaceutically acceptable salt and/or a solvate thereof, wherein

ring A is phenyl and ring B is pyridyl, wherein the chlorine substituents are independently in the 3 or 4 position.

In a another embodiment the invention provides a compound of the formula (I) and/or a tautomer and/or an N-oxide and/or a pharmaceutically acceptable salt and/or a solvate thereof, wherein

ring A is phenyl, wherein the Chlorine substituent is in the 3 position and ring B is phenyl, wherein the Chlorine substituent is in the 4 position.

In a another embodiment the invention provides a compound of the formula (I) and/or a tautomer and/or an N-oxide and/or a pharmaceutically acceptable salt and/or a solvate thereof, wherein

ring A is phenyl, wherein the Chlorine substituent is in the 4 position and ring B is phenyl, wherein the Chlorine substituent is in the 3 position.

In a preferred embodiment the invention provides a compound of the formula (I) and/or a tautomer and/or an N-oxide and/or a pharmaceutically acceptable salt and/or a solvate thereof, wherein

ring A is phenyl, wherein the Chlorine substituent is in the 3 position and ring B is phenyl, wherein the Chlorine substituent is in the 3 position.

In another embodiment the invention provides a compound of the formula (I) and/or a tautomer and/or an N-oxide and/or a pharmaceutically acceptable salt and/or a solvate thereof, wherein

both rings A and B are pyridyl, wherein the Chlorine substituents are independently in the 3 or 4 position.

In a preferred embodiment the invention provides a compound of the formula (I) and/or a tautomer and/or an N-oxide and/or a pharmaceutically acceptable salt and/or a solvate thereof, wherein rings A are selected from a group as shown for R² in Table 1.

In another preferred embodiment the invention provides a compound of the formula (I) and/or a tautomer and/or an N-oxide and/or a pharmaceutically acceptable salt and/or a solvate thereof, wherein rings B are selected from a group as shown for R³ in Table 1.

In one embodiment the invention provides a compound of the formula (I) and/or a tautomer and/or an N-oxide and/or a pharmaceutically acceptable salt and/or a solvate thereof, wherein R¹ is selected from the group consisting of
cyano-
carboxyl-
C₁-C₇-alkoxy-carbonyl-
amino-carbonyl-
N—C₁-C₇-alkyl-amino-carbonyl-
N,N-di-C₁-C₇-alkyl-amino-carbonyl-
N,N-di-C₁-C₇-alkyl-amino-C₁-C₇-alkyl-amino-carbonyl-
N,N-di-C₁-C₇-alkyl-amino-C₁-C₇-alkyl-N′—C₁-C₇-alkyl-amino-carbonyl-
amino-C₁-C₇-alkyl-amino-carbonyl-
amino-C₁-C₇-alkyl-N′—C₁-C₇-alkyl-amino-carbonyl-
N—C₁-C₇-alkyl-amino-C₁-C₇-alkyl-amino-carbonyl-
N—C₁-C₇-alkyl-amino-C₁-C₇-alkyl-N′—C₁-C₇-alkyl-amino-carbonyl-
N,N-di-C₁-C₇-alkyl-amino-C₁-C₇-alkyl-amino-carbonyl-
N,N-di-C₁-C₇-alkyl-amino-C₁-C₇-alkyl-N′—C₁-C₇-alkyl-amino-carbonyl-
N-(heterocyclyl-C₁-C₇-alkyl)-amino-carbonyl-
N-(cycloalkyl-C₁-C₇-alkyl)-amino-carbon yl-
N-hydroxyl-amino-carbonyl-
N-hydroxyl-N—C₁-C₇-alkyl-amino-carbonyl-
N—C₁-C₇-alkoxy-amino-carbonyl-
N—C₁-C₇-alkoxy-N—C₁-C₇-alkyl-amino-carbonyl-
N-benzyloxy-amino-carbonyl-
heterocyclyl-
heterocyclyl-C₁-C₇-alkyl-
hydroxy-C₁-C₇-alkyl-
C₁-C₇-alkyl-carbonyl-
formyl-
cyano-C₁-C₇-alkyl-
carboxyl-C₁-C₇-alkyl-
C₁-C₇-alkoxy-C₁-C₇-alkyl-
heterocyclyl-carbonyl-
C₁-C₇-alkyl-carbonyl-amino-C₁-C₇-alkyl-
C₁-C₇-alkyl-carbonyl-N—C₁-C₇-alkyl-amino-C₁-C₇-alkyl-
hydroxy-C₁-C₇-alkyl-amino-carbonyl-
C₁-C₇-alkoxy-C₁-C₇-alkyl-amino-carbonyl-
hydroxy-C₁-C₇-alkyl-N—C₁-C₇-alkyl-amino-carbonyl-
C₁-C₇-alkoxy-C₁-C₇-alkyl-N—C₁-C₇-alkyl-amino-carbonyl-
C₁-C₇-alkyl-carbonyl-C₁-C₇-alkyl-amino-carbonyl-
C₁-C₇-alkyl-carbonyl-C₁-C₇-alkyl-N—C₁-C₇-alky-amino-carbonyl-
N—(C₁-C₇-alkoxy-C₁-C₇-alkyl)-amino-sulfonyl-
N—(C₁-C₇-alkoxy-C₁-C₇-alkyl)-N—C₁-C₇-alkyl-amino-sulfonyl-
S—C₁-C₇-alkyl-sulfonimidoyl-
S—C₁-C₇-alkyl-N—C₁-C₇-alkyl-sulfonimidoyl-
N,N-di-C₁-C₇-alkyl-amino-C₁-C₇-alkyl-amino-carbonyl-C₁-C₇-alkyl-amino-carbonyl-
N,N-di-C₁-C₇-alkyl-amino-C₁-C₇-alkyl-N′—C₁-C₇-alkyl-amino-carbonyl-C₁-C₇-alkyl-N″—C₁-C₇-alkyl-amino-carbonyl-
N,N-di-C₁-C₇-alkyl-amino-C₁-C₇-alkyl-amino-carbonyl-C₁-C₇-alkyl-N″—C₁-C₇-alkyl-amino-carbonyl-
N,N-di-C₁-C₇-alkyl-amino-C₁-C₇-alkyl-N′—C₁-C₇-alkyl-amino-carbonyl-C₁-C₇-alkyl-amino-carbonyl-
N—C₁-C₇-alkyl-amino-C₁-C₇-alkyl-amino-carbonyl-C₁-C₇-alkyl-amino-carbonyl-
N—C₁-C₇-alkyl-amino-C₁-C₇-alkyl-N′—C₁-C₇-alkyl-amino-carbonyl-C₁-C₇-alkyl-N″—C₁-C₇-alkyl-amino-carbonyl-
N—C₁-C₇-alkyl-amino-C₁-C₇-alkyl-amino-carbonyl-C₁-C₇-alkyl-N″—C₁-C₇-alkyl-amino-carbonyl-
N—C₁-C₇-alkyl-amino-C₁-C₇-alkyl-N′—C₁-C₇-alkyl-amino-carbonyl-C₁-C₇-alkyl-amino-carbonyl-
amino-C₁-C₇-alkyl-amino-carbonyl-C₁-C₇-alkyl-amino-carbonyl-
amino-C₁-C₇-alkyl-N′—C₁-C₇-alkyl-amino-carbonyl-C₁-C₇-alkyl-N″—C₁-C₇-alkyl-amino-carbonyl-
amino-C₁-C₇-alkyl-amino-carbonyl-C₁-C₇-alkyl-N″—C₁-C₇-alkyl-amino-carbonyl-
amino-C₁-C₇-alkyl-N′—C₁-C₇-alkyl-amino-carbonyl-C₁-C₇-alkyl-amino-carbonyl-
C₁-C₇-alkoxy-carbonyl-C₁-C₇-alkyl-amino-carbonyl-
C₁-C₇-alkoxy-carbonyl-C₁-C₇-alkyl-N—C₁-C₇-alkyl-amino-carbonyl-
carboxyl-C₁-C₇-alkyl-amino-carbonyl-
C₁-C₇-alkyl-sulfonyl-
amino-
N—C₁-C₇-alkyl-amino-
N,N-di-C₁-C₇-alkyl-amino-
C₁-C₇-alkoxy-carbonyl-amino-
C₁-C₇-alkoxy-carbonyl-N—C₁-C₇-alkyl-amino-
C₁-C₇-alkyl-carbonyl-amino-
C₁-C₇-alkyl-carbonyl-N—C₁-C₇-alkyl-amino-
amino-sulfonyl-
N—C₁-C₇-alkyl-amino-sulfonyl-
N,N-di-C₁-C₇-alkyl-amino-sulfonyl-
hydroxy-C₁-C₇-alkyl-amino-sulfonyl-
C₁-C₇-alkoxy-C₁-C₇-alkyl-amino-sulfonyl-
hydroxy-C₁-C₇-alkyl-N—C₁-C₇-alkyl-amino-sulfonyl-
C₁-C₇-alkoxy-C₁-C₇-alkyl-N—C₁-C₇-alkyl-amino-sulfonyl-
hydrazino-carbonyl-
N—C₁-C₇-alkyl-hydrazino-carbonyl-
N,N-di-C₁-C₇-alkyl-hydrazino-carbonyl-
N—C₁-C₇-alkyl-hydrazino-carbonyl-
N,N-di-C₁-C₇-alkyl-hydrazino-carbonyl-
N,N-di-C₁-C₇-alkyl-N′—C₁-C₇-alkyl-hydrazino-carbonyl-
C₁-C₇-alkyl-carbonyl-hydrazino-carbonyl-
C₁-C₇-alkyl-carbonyl-N—C₁-C₇-alkyl-N′—C₁-C₇-alkyl-hydrazino-carbonyl-
phosphonyl-
C₁-C₇-alkyl-phosphonyl-
di-C₁-C₇-alkyl-phosphonyl-
benzyloxycarbonyl
hydroxy-C₁-C₇-alkyl-carbonyl-, and
C₁-C₇-alkoxy-C₁-C₇-alkyl-amino-sulfonyl-

In another embodiment the invention provides a compound of the formula (I) and/or a tautomer and/or an N-oxide and/or a pharmaceutically acceptable salt and/or a solvate thereof, wherein R¹ is selected from the group consisting of

cyano-
carboxyl-
C₁-C₇-alkoxy-carbonyl-
amino-carbonyl-
N—C₁-C₇-alkyl-amino-carbonyl-
N,N-di-C₁-C₇-alkyl-amino-carbonyl-
N,N-di-C₁-C₇-alkyl-amino-C₁-C₇-alkyl-amino-carbonyl-
N,N-di-C₁-C₇-alkyl-amino-C₁-C₇-alkyl-N′—C₁-C₇-alkyl-amino-carbonyl-
N-(heterocyclyl-C₁-C₇-alkyl)-amino-carbonyl-
N-(cycloalkyl-C₁-C₇-alkyl)-amino-carbonyl-

### N-hydroxyl-amino-carbonyl-

N—C₁-C₇-alkoxy-amino-carbonyl-

### N-benzyloxy-amino-carbonyl-

benzyloxycarbonyl
heterocyclyl-
heterocyclyl-C₁-C₇-alkyl-
hydroxy-C₁-C₇-alkyl-
hydroxy-C₁-C₇-alkyl-carbonyl-
C₁-C₇-alkyl-carbonyl-
cyano-C₁-C₇-alkyl-
carboxyl-C₁-C₇-alkyl-
C₁-C₇-alkoxy-C₁-C₇-alkyl-
heterocyclyl-carbonyl-
C₁-C₇-alkyl-carbonyl-amino-C₁-C₇-alkyl-
hydroxy-C₁-C₇-alkyl-amino-carbonyl-
C₁-C₇-alkyl-carbonyl-C₁-C₇-alkyl-amino-carbonyl-
N—(C₁-C₇-alkoxy-C₁-C₇-alkyl)-amino-sulfonyl-
S—C₁-C₇-alkyl-sulfonimidoyl-
N,N-di-C₁-C₇-alkyl-amino-C₁-C₇-alkyl-amino-carbonyl-C₁-C₇-alkyl-amino-carbonyl-
C₁-C₇-alkoxy-carbonyl-C₁-C₇-alkyl-amino-carbonyl-
carboxyl-C₁-C₇-alkyl-amino-carbonyl-
C₁-C₇-alkyl-sulfonyl-
amino-
N,N-di-C₁-C₇-alkyl-amino-
C₁-C₇-alkoxy-carbonyl-amino-
C₁-C₇-alkyl-carbonyl-amino-
amino-sulfonyl-
N—C₁-C₇-alkyl-amino-sulfonyl-
C₁-C₇-alkoxy-C₁-C₇-alkyl-amino-sulfonyl-
hydroxy-C₁-C₇-alkyl-amino-sulfonyl-
hydrazino-carbonyl-
C₁-C₇-alkyl-carbonyl-hydrazino-carbonyl-
phosphonyl-
C₁-C₇-alkyl-phosphonyl-
di-C₁-C₇-alkyl-phosphonyl-.

In another embodiment the invention provides a compound of the formula (I) and/or a tautomer and/or an N-oxide and/or a pharmaceutically acceptable salt and/or a solvate thereof, wherein R¹ is selected from the group consisting of

cyano-
carboxyl-
C₁-C₇-alkoxy-carbonyl-
amino-carbonyl-
N—C₁-C₇-alkyl-amino-carbonyl-
N,N-di-C₁-C₇-alkyl-amino-carbonyl-
heterocyclyl-
heterocyclyl-C₁-C₇-alkyl-
hydroxy-C₁-C₇-alkyl-
C₁-C₇-alkyl-carbonyl-
cyano-C₁-C₇-alkyl-
carboxyl-C₁-C₇-alkyl-
C₁-C₇-alkoxy-C₁-C₇-alkyl-
heterocyclyl-carbonyl-
amino-
N,N-di-C₁-C₇-alkyl-amino-
C₁-C₇-alkoxy-carbonyl-amino-
C₁-C₇-alkyl-carbonyl-amino-
phosphonyl-
C₁-C₇-alkyl-phosphonyl-
di-C₁-C₇-alkyl-phosphonyl-
benzyloxycarbonyl
hydroxy-C₁-C₇-alkyl-carbonyl-, and
C₁-C₇-alkoxy-C₁-C₇-alkyl-amino-sulfonyl-

In another embodiment the invention provides a compound of the formula (I) and/or a tautomer and/or an N-oxide and/or a pharmaceutically acceptable salt and/or a solvate thereof, wherein R¹ is selected from the group consisting of

cyano-
carboxyl-
C₁-C₃-alkoxy-carbonyl-
amino-carbonyl-
N—C₁-C₃-alkyl-amino-carbonyl-
N,N-di-C₁-C₃-alkyl-amino-carbonyl-
N,N-di-C₁-C₃-alkyl-amino-C₁-C₃-alkyl-amino-carbonyl-
N,N-di-C₁-C₃-alkyl-amino-C₁-C₇-alkyl-N′—C₁-C₃-alkyl-amino-carbonyl-
N-(heterocyclyl-C₁-C₃-alkyl)-amino-carbonyl-
N-(cycloalkyl-C₁-C₃-alkyl)-amino-carbonyl-

### N-hydroxyl-amino-carbonyl-

N—C₁-C₄-alkoxy-amino-carbonyl-

### N-benzyloxy-amino-carbonyl-

benzyloxycarbonyl
heterocyclyl-
heterocyclyl-C₁-C₃-alkyl-
hydroxy-C₁-C₃-alkyl-
hydroxy-C₁-C₃-alkyl-carbonyl-
C₁-C₃-alkyl-carbonyl-
cyano-C₁-C₃-alkyl-
carboxyl-C₁-C₃-alkyl-
C₁-C₃-alkoxy-C₁-C₃-alkyl-
heterocyclyl-carbonyl-
C₁-C₃-alkyl-carbonyl-amino-C₁-C₃-alkyl-
hydroxy-C₁-C₃-alkyl-amino-carbonyl-
C₁-C₃-alkyl-carbonyl-C₁-C₃-alkyl-amino-carbonyl-
N—(C₁-C₃-alkoxy-C₁-C₃-alkyl)-amino-sulfonyl-
S—C₁-C₃-alkyl-sulfonimidoyl-
N,N-di-C₁-C₃-alkyl-amino-C₁-C₃-alkyl-amino-carbonyl-C₁-C₃-alkyl-amino-carbonyl-
C₁-C₃-alkoxy-carbonyl-C₁-C₃-alkyl-amino-carbonyl-
carboxyl-C₁-C₃-alkyl-amino-carbonyl-
C₁-C₃-alkyl-sulfonyl-
amino-
N,N-di-C₁-C₃-alkyl-amino-
C₁-C₄-alkoxy-carbonyl-amino-
C₁-C₇-alkyl-carbonyl-amino-
amino-sulfonyl-
N—C₁-C₄-alkyl-amino-sulfonyl-
C₁-C₃-alkoxy-C₁-C₃-alkyl-amino-sulfonyl-
hydroxy-C₁-C₃-alkyl-amino-sulfonyl-
hydrazino-carbonyl-
C₁-C₃-alkyl-carbonyl-hydrazino-carbonyl-
phosphonyl-
C₁-C₃-alkyl-phosphonyl-
di-C₁-C₃-alkyl-phosphonyl-.

In a preferred embodiment, when R¹ is heterocyclyl-, said heterocycle is selected from tetrazolyl, oxadiazolyl, imidazolyl, oxadiazolonyl, oxazolyl, pyrrolyl, pyrazoiyi or dihydrotriazolethionyl, wherein said heterocyclyl is optionally substituted by 1 or 2 groups independently selected from methyl, ethyl, amino and methylamino, and when the heterocyclyl group is pyrazolyl or oxadiazolyl, said heterocyclyl may also be optionally substituted by N—C₁-C₃-alkylamino-carbonyl, C₁-C₃-alkyl-carbonyl, carboxy, C₁-C₃-alkoxy-C₁-C₃-alkyl-amino, trimethylsilyl-ethoxy-methyl or benzylamino.

In another preferred embodiment, R¹ aminooxadiazolyl.

In a preferred embodiment the invention provides a compound of the formula (I) and/or a tautomer and/or an N-oxide and/or a pharmaceutically acceptable salt and/or a solvate thereof, wherein R¹ selected from a group as shown in Table 1.

In one embodiment the invention provides a compound of the formula (I) and/or a tautomer and/or an N-oxide and/or a pharmaceutically acceptable salt and/or a solvate thereof, wherein R⁴ is selected from the group consisting of

substituted C₁-alkyl-
C₂-C₇-alkyl-
aryl-
heteroaryl-
heterocyclyl-
C₃-C₁₀-cycloalkyl-
aryl-C₁-C₇-alkyl-
heteroaryl-C₁-C₇-alkyl-
heterocyclyl-C₁-C₇-alkyl-
C₃-C₁₀-cycloalkyl-C₁-C₇-alkyl-
unsubstituted or substituted by 1-3 substituents selected from
hydroxy-
C₁-C₇-alkoxy-
halogen-
hydroxy-C₁-C₇-alkyl-
N,N-di-C₁-C₇-alkyl-aminocarbonyl C₁-C₇-alkyl-amino-
amino-heterocyclyl-C₁-C₇-alkyl-amino-phenyl-
formyl-
carboxy-C₁-C₇-alkyl-amino
halo-C₁-C₇-alkyl-
nitro-
C₁-C₇-alkyl-carbonyl-
formyl-
C₁-C₇-alkyl-
amino-
N—C₁-C₇-alkyl-amino-
N,N-di-C₁-C₇-alkyl-amino-
amino-C₁-C₇-alkyl-amino-
amino-C₁-C₇-alkyl-N′—C₁-C₇-alkyl-amino-
N—C₁-C₇-alkyl-amino-C₁-C₇-alkyl-amino-
N—C₁-C₇-alkyl-amino-C₁-C₇-alkyl-N′—C₁-C₇-alkyl-amino-
N,N-di-C₁-C₇-alkyl-amino-C₁-C₇-alkyl-amino-
N,N-di-C₁-C₇-alkyl-amino-C₁-C₇-alkyl-N′—C₁-C₇-alkyl-amino-
N,N-di-C₁-C₇-alkyl-amino-carbonyl-C₁-C₇-alkyl-N′—C₁-C₇-alkyl-amino-
N,N-di-C₁-C₇-alkyl-amino-carbonyl-C₁-C₇-alkyl-amino-
N—C₁-C₇-alkyl-amino-carbonyl-C₁-C₇-alkyl-N′—C₁-C₇-alkyl-amino-
N—C₁-C₇-alkyl-amino-carbonyl-C₁-C₇-alkyl-amino-
amino-carbonyl-C₁-C₇-alkyl-N′—C₁-C₇-alkyl-amino-
amino-carbonyl-C₁-C₇-alkyl-amino-
hydroxy-alkyl-
C₁-C₇-alkyl-carbonyl-amino-
hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N,N-di-C₁-C₇-alkyl-N′—C₁-C₇-alkyl-hydrazino-carbonyl-C₁-C₇-alkyl-amino-
C₁-C₇-alkyl-carbonyl-hydrazino-carbonyl-C₁-C₇-alkyl-amino-
C₁-C₇-alkyl-carbonyl-N—C₁-C₇-alkyl-N′—C₁-C₇-alkyl-hydrazino-carbonyl-C₁-C₇-alkyl-amino-
hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N,N-di-C₁-C₇-alkyl-N′—C₁-C₇-alkyl-hydrazino-carbonyl-C₁-C₇-alkyl-N—(C₁-C₇-alkyl)-amino-
C₁-C₇-alkyl-carbonyl-hydrazino-carbonyl-C₁-C₇-alkyl-N—(C₁-C₇-alkyl)-amino-
C₁-C₇-alkyl-carbonyl-N—C₁-C₇-alkyl-N′—C₁-C₇-alkyl-hydrazino-carbonyl-C₁-C₇-alkyl-
N—(C₁-C₇-alkyl)-amino-
heterocyclyl-C₁-C₇-alkyl-N—C₁-C₇-alkyl-amino-carbonyl-C₁-C₇-alkyl-amino-
heterocyclyl-C₁-C₇-alkyl-amino-carbonyl-C₁-C₇-alkyl-amino-
heterocyclyl-C₁-C₇-alkyl-N—C₁-C₇-alkyl-amino-carbonyl-C₁-C₇-alkyl-N′—C₁-C₇-alkyl-amino-
heterocyclyl-C₁-C₇-alkyl-amino-carbonyl-C₁-C₇-alkyl-N′—C₁-C₇-alkyl-amino-
heterocyclyl-C₁-C₇-alkyl-N—C₁-C₇-alkyl-amino-
heterocyclyl-C₁-C₇-alkyl-amino-
C₃-C₁₀-cycloalkyl-C₁-C₇-alkyl-N—C₁-C₇-alkyl-amino-carbonyl-C₁-C₇-alkyl-amino-
C₃-C₁₀-cycloalkyl-C₁-C₇-alkyl-amino-carbonyl-C₁-C₇-alkyl-amino-
C₃-C₁₀-cycloalkyl-C₁-C₇-alkyl-N—C₁-C₇-alkyl-amino-carbonyl-C₁-C₇-alkyl-N′—C₁-C₇-alkyl-amino-
C₃-C₁₀-cycloalkyl-C₁-C₇-alkyl-amino-carbonyl-C₁-C₇-alkyl-N′—C₁-C₇-alkyl-amino-alkyl-amino-
di-C₁-C₇-alkoxy-C₁-C₇-alkyl-N—C₁-C₇-alkyl-amino-carbonyl-C₁-C₇-alkyl-amino-
di-C₁-C₇-alkoxy-C₁-C₇-alkyl-amino-carbonyl-C₁-C₇-alkyl-N′—C₁-C₇-alkyl-amino-
di-C₁-C₇-alkoxy-C₁-C₇-alkyl-amino-carbonyl-C₁-C₇-alkyl-amino-
C₁-C₇-alkoxy-carbonyl-C₁-C₇-alkyl-amino-
C₁-C₇-alkoxy-carbonyl-C₁-C₇-alkyl-N—C₁-C₇-alkyl-amino-
hydroxy-carbonyl-C₁-C₇-alkyl-amino-
hydroxy-carbonyl-C₁-C₇-alkyl-N—C₁-C₇-alkyl-amino-
C₁-C₇-alkyl-carbonyl-C₁-C₇-alkyl-amino-
C₁-C₇-alkyl-carbonyl-C₁-C₇-alkyl-N—C₁-C₇-alkyl-amino-
C₁-C₇-alkyl-carbonyl-amino-C₁-C₇-alkyl-amino-
C₁-C₇-alkyl-carbonyl-amino-C₁-C₇-alkyl-N′—C₁-C₇-alkyl-amino-
C₁-C₇-alkyl-carbonyl-N—C₁-C₇-alkyl-amino-C₁-C₇-alkyl-amino-
C₁-C₇-alkyl-carbonyl-N—C₁-C₇-alkyl-amino-C₁-C₇-alkyl-N′—C₁-C₇-alkyl-amino-
C₁-C₇-alkyl-amino-carbonyl-amino-C₁-C₇-alkyl-amino-
C₁-C₇-alkyl-amino-carbonyl-amino-C₁-C₇-alkyl-N″—C₁-C₇-alkyl-amino-
C₁-C₇-alkyl-amino-carbonyl-N′—C₁-C₇-alkyl-amino-C₁-C₇-alkyl-amino-
C₁-C₇-alkyl-amino-carbonyl-N′—C₁-C₇-alkyl-amino-C₁-C₇-alkyl-N″—C₁-C₇-alkyl-amino-
C₁-C₇-alkyl-N—C₁-C₇-alkyl-amino-carbonyl-amino-C₁-C₇-alkyl-amino-
C₁-C₇-alkyl-N—C₁-C₇-alkyl-amino-carbonyl-amino-C₁-C₇-alkyl-N″—C₁-C₇-alkyl-amino-
C₁-C₇-alkyl-N—C₁-C₇-alkyl-amino-carbonyl-N′—C₁-C₇-alkyl-amino-C₁-C₇-alkyl-N″-benzyloxy-carbonyl-
C₁-C₇-alkyl-carbonyl-C₁-C₇-alkyl-amino-carbonyl-
heterocyclyl-
protected hydroxy-.

In another embodiment the invention provides a compound of the formula (I) and/or a tautomer and/or an N-oxide and/or a pharmaceutically acceptable salt and/or a solvate thereof, wherein R⁴ is selected from the group consisting of

substituted C₁-alkyl-
C₂-C₇-alkyl-
aryl-
heteroaryl-
heterocyclyl-
C₃-C₁₀-cycloalkyl-
aryl-C₁-C₇-alkyl-
heterocyclyl-C₁-C₇-alkyl-
C₃-C₁₀-cycloalkyl-C₁-C₇-alkyl-
unsubstituted or substituted by 1-2 substituents selected from
hydroxy-
C₁-C₇-alkoxy-
halogen-
hydroxy-C₁-C₇-alkyl-
N,N-di-C₁-C₇-alkyl-aminocarbonyl C₁-C₇-alkyl-amino-
amino-heterocyclyl-C₁-C₇-alkyl-amino-phenyl-
formyl-
carboxy-C₁-C₇-alkyl-amino
halo-C₁-C₇-alkyl-
nitro-
C₁-C₇-alkyl-carbonyl-
formyl-
C₁-C₇-alkyl-
amino-
N—C₁-C₇-alkyl-amino-
N,N-di-C₁-C₇-alkyl-amino-
amino-C₁-C₇-alkyl-amino-
amino-C₁-C₇-alkyl-N′—C₁-C₇-alkyl-amino-
N—C₁-C₇-alkyl-amino-C₁-C₇-alkyl-amino-
N—C₁-C₇-alkyl-amino-C₁-C₇-alkyl-N′—C₁-C₇-alkyl-amino-
N,N-di-C₁-C₇-alkyl-amino-C₁-C₇-alkyl-amino-
N,N-di-C₁-C₇-alkyl-amino-C₁-C₇-alkyl-N′—C₁-C₇-alkyl-amino-
N,N-di-C₁-C₇-alkyl-amino-carbonyl-C₁-C₇-alkyl-N′—C₁-C₇-alkyl-amino-
N,N-di-C₁-C₇-alkyl-amino-carbonyl-C₁-C₇-alkyl-amino-
N—C₁-C₇-alkyl-amino-carbonyl-C₁-C₇-alkyl-N′—C₁-C₇-alkyl-amino-
N—C₁-C₇-alkyl-amino-carbonyl-C₁-C₇-alkyl-amino-
amino-carbonyl-C₁-C₇-alkyl-N′—C₁-C₇-alkyl-amino-
amino-carbonyl-C₁-C₇-alkyl-amino-
hydroxy-alkyl-
C₁-C₇-alkyl-carbonyl-amino-
hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N,N-di-C₁-C₇-alkyl-N′—C₁-C₇-alkyl-hydrazino-carbonyl-C₁-C₇-alkyl-amino-
C₁-C₇-alkyl-carbonyl-hydrazino-carbonyl-C₁-C₇-alkyl-amino-
C₁-C₇-alkyl-carbonyl-N—C₁-C₇-alkyl-N′—C₁-C₇-alkyl-hydrazino-carbonyl-C₁-C₇-alkyl-amino-
hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N,N-di-C₁-C₇-alkyl-N′—C₁-C₇-alkyl-hydrazino-carbonyl-C₁-C₇-alkyl-N—(C₁-C₇-alkyl)-amino-
C₁-C₇-alkyl-carbonyl-hydrazino-carbonyl-C₁-C₇-alkyl-N—(C₁-C₇-alkyl)-amino-
C₁-C₇-alkyl-carbonyl-N—C₁-C₇-alkyl-N′—C₁-C₇-alkyl-hydrazino-carbonyl-C₁-C₇-alkyl-N—(C₁-C₇-alkyl)-amino-
heterocyclyl-C₁-C₇-alkyl-N—C₁-C₇-alkyl-amino-carbonyl-C₁-C₇-alkyl-amino-
heterocyclyl-C₁-C₇-alkyl-amino-carbonyl-C₁-C₇-alkyl-amino-
heterocyclyl-C₁-C₇-alkyl-N—C₁-C₇-alkyl-amino-carbonyl-C₁-C₇-alkyl-N′—C₁-C₇-alkyl-amino-
heterocyclyl-C₁-C₇-alkyl-amino-carbonyl-C₁-C₇-alkyl-N′—C₁-C₇-alkyl-amino-
heterocyclyl-C₁-C₇-alkyl-N—C₁-C₇-alkyl-amino-
heterocyclyl-C₁-C₇-alkyl-amino-
C₃-C₁₀-cycloalkyl-C₁-C₇-alkyl-N—C₁-C₇-alkyl-amino-carbonyl-C₁-C₇-alkyl-amino-
C₃-C₁₀-cycloalkyl-C₁-C₇-alkyl-amino-carbonyl-C₁-C₇-alkyl-amino-
C₃-C₁₀-cycloalkyl-C₁-C₇-alkyl-N—C₁-C₇-alkyl-amino-carbonyl-C₁-C₇-alkyl-N′—C₁-C₇-alkyl-amino-
C₃-C₁₀-cycloalkyl-C₁-C₇-alkyl-amino-carbonyl-C₁-C₇-alkyl-N′—C₁-C₇-alkyl-amino-
di-C₁-C₇-alkoxy-C₁-C₇-alkyl-N—C₁-C₇-alkyl-amino-carbonyl-C₁-C₇-alkyl-N′—C₁-C₇-alkyl-amino-
di-C₁-C₇-alkoxy-C₁-C₇-alkyl-N—C₁-C₇-alkyl-amino-carbonyl-C₁-C₇-alkyl-amino-
di-C₁-C₇-alkoxy-C₁-C₇-alkyl-amino-carbonyl-C₁-C₇-alkyl-N′—C₁-C₇-alkyl-amino-
di-C₁-C₇-alkoxy-C₁-C₇-alkyl-amino-carbonyl-C₁-C₇-alkyl-amino-
C₁-C₇-alkoxy-carbonyl-C₁-C₇-alkyl-amino-
C₁-C₇-alkoxy-carbonyl-C₁-C₇-alkyl-N—C₁-C₇-alkyl-amino-
hydroxy-carbonyl-C₁-C₇-alkyl-amino-
hydroxy-carbonyl-C₁-C₇-alkyl-N—C₁-C₇-alkyl-amino-
C₁-C₇-alkyl-carbonyl-C₁-C₇-alkyl-amino-
C₁-C₇-alkyl-carbonyl-C₁-C₇-alkyl-N—C₁-C₇-alkyl-amino-
C₁-C₇-alkyl-carbonyl-amino-C₁-C₇-alkyl-amino-
C₁-C₇-alkyl-carbonyl-amino-C₁-C₇-alkyl-N′—C₁-C₇-alkyl-amino-
C₁-C₇-alkyl-carbonyl-N—C₁-C₇-alkyl-amino-C₁-C₇-alkyl-amino-
C₁-C₇-alkyl-carbonyl-N—C₁-C₇-alkyl-amino-C₁-C₇-alkyl-N′—C₁-C₇-alkyl-amino-
C₁-C₇-alkyl-amino-carbonyl-amino-C₁-C₇-alkyl-amino-
C₁-C₇-alkyl-amino-carbonyl-amino-C₁-C₇-alkyl-N″—C₁-C₇-alkyl-amino-
C₁-C₇-alkyl-amino-carbonyl-N′—C₁-C₇-alkyl-amino-C₁-C₇-alkyl-amino-
C₁-C₇-alkyl-amino-carbonyl-N′—C₁-C₇-alkyl-amino-C₁-C₇-alkyl-N″—C₁-C₇-alkyl-amino-
C₁-C₇-alkyl-N—C₁-C₇-alkyl-amino-carbonyl-amino-C₁-C₇-alkyl-amino-
C₁-C₇-alkyl-N—C₁-C₇-alkyl-amino-carbonyl-amino-C₁-C₇-alkyl-N″—C₁-C₇-alkyl-amino-
C₁-C₇-alkyl-N—C₁-C₇-alkyl-amino-carbonyl-N′—C₁-C₇-alkyl-amino-C₁-C₇-alkyl-amino-
C₁-C₇-alkyl-N—C₁-C₇-alkyl-amino-carbonyl-N′—C₁-C₇-alkyl-amino-C₁-C₇-alkyl-N″—C₁-C₇-alkyl-amino-
benzyloxy-carbonyl-
C₁-C₇-alkyl-carbonyl-C₁-C₇-alkyl-amino-carbonyl-
heterocyclyl-
protected hydroxy-.

In another embodiment the invention provides a compound of the formula (I) and/or a tautomer and/or an N-oxide and/or a pharmaceutically acceptable salt and/or a solvate thereof, wherein R⁴ is selected from the group consisting of

substituted C₁-alkyl-
C₂-C₇-alkyl-
aryl-
heteroaryl-
heterocyclyl-
C₃-C₁₀-cycloalkyl-
aryl-C₁-C₇-alkyl-
heterocyclyl-C₁-C₇-alkyl-
C₃-C₁₀-cycloalkyl-C₁-C₇-alkyl-
unsubstituted or substituted by 1-2 substituents selected from
hydroxy-
C₁-C₇-alkoxy-
halogen-
hydroxy-C₁-C₇-alkyl-
N,N-di-C₁-C₇-alkyl-aminocarbonyl C₁-C₇-alkyl-amino-
amino-heterocyclyl-C₁-C₇-alkyl-amino-phenyl-
formyl-
carboxy-C₁-C₇-alkyl-amino
halo-C₁-C₇-alkyl-
nitro-
C₁-C₇-alkyl-carbonyl-
C₁-C₇-alkyl-
amino-
N,N-di-C₁-C₇-alkyl-amino-
amino-C₁-C₇-alkyl-amino-
N,N-di-C₁-C₇-alkyl-amino-carbonyl-C₁-C₇-alkyl-amino-
amino-carbonyl-C₁-C₇-alkyl-amino-
hydroxy-alkyl-
C₁-C₇-alkyl-carbonyl-amino-
hydrazino-carbonyl-C₁-C₇-alkyl-amino-
heterocyclyl-C₁-C₇-alkyl-N—C₁-C₇-alkyl-amino-carbonyl-C₁-C₇-alkyl-amino-
heterocyclyl-C₁-C₇-alkyl-amino-
C₃-C₁₀-cycloalkyl-C₁-C₇-alkyl-N—C₁-C₇-alkyl-amino-carbonyl-C₁-C₇-alkyl-amino-
di-C₁-C₇-alkoxy-C₁-C₇-alkyl-amino-carbonyl-C₁-C₇-alkyl-amino-
C₁-C₇-alkoxy-carbonyl-C₁-C₇-alkyl-amino-
hydroxy-carbonyl-C₁-C₇-alkyl-amino-
C₁-C₇-alkyl-carbonyl-C₁-C₇-alkyl-amino-
C₁-C₇-alkyl-carbonyl-amino-C₁-C₇-alkyl-amino-
C₁-C₇-alkyl-amino carbonyl-amino-C₁-C₇-alkyl-amino-
benzyloxy-carbonyl-
C₁-C₇-alkyl-carbonyl-C₁-C₇-alkyl-amino-carbonyl-
heterocyclyl-
protected hydroxy-.

In another embodiment, when R⁴ is heteroaryl, said heteroaryl is selected from thiophenyl, indolyl, benzothiophenyl, pyridyl, piperidinyl and pyrrolidinyl.

In another embodiment, when R⁴ is C₃-C₁₀-cycloalkyl-, said C₃-C₁₀-cycloalkyl- is cyclohexyl-.

In another embodiment, when R⁴ is C₃-C₁₀-cycloalkyl-C₁-C₇-alkyl-, said C₃-C₁₀-cycloalkyl-C₁-C₇-alkyl- is cyclopropylmethyl-.

In another embodiment, R⁴ is selected from

phenyl, said phenyl being optionally substituted by one or two substituents independently selected from methyl, amino and halo,
cyclohexyl,
cyclopropylmethyl,
benzyl,
pyridyl, said pyridyl being optionally substituted by methyl,
C₁-C₅-alkyl,
thiophenyl,
cyclopentylmethyl,
indolyl, said indolyl being optionally substituted by methyl and cycloheptyl.

In another embodiment, R⁴ is phenyl or cyclohexyl.

In another embodiment, R⁴ is as disclosed herein with the proviso that R⁴ is not phenyl directly substituted with at least one alkoxy substituent.

In a preferred embodiment the invention provides a compound of the formula (I) and/or a tautomer and/or an N-oxide and/or a pharmaceutically acceptable salt and/or a solvate thereof, wherein R⁴ selected from a group as shown in Table 1.

In one embodiment the invention provides a compound of the formula (I) and/or a tautomer and/or an N-oxide and/or a pharmaceutically acceptable salt and/or a solvate thereof, wherein R′ and R″ are independently selected from the group consisting of

heterocyclyl-heterocyclyl-carbonyl-
hydroxy-
C₁-C₇-alkoxy-
halogen-
halo-C₁-C₇-alkyl-
cyano-
C₁-C₇-alkyl-carbonyl-
formyl-
C₁-C₇-alkyl-
amino-carbonyl-
N—C₁-C₇-alkyl-amino-carbonyl-
N,N-di-C₁-C₇-alkyl-amino-carbonyl-
amino-C₁-C₇-alkyl-
heterocyclyl-C₁-C₇-alkyl-
N—C₁-C₇-alkyl-amino-C₁-C₇-alkyl-
N,N-di-C₁-C₇-alkyl-amino-C₁-C₇-alkyl-
heterocyclyl-carbonyl-C₁-C₇-alkyl-
heterocyclyl-heterocyclyl-carbonyl-C₁-C₇-alkyl-heterocyclyl-C₁-C₇-alkyl-amino-carbonyl-
heterocyclyl-C₁-C₇-alkyl-aminocarbonyl
heterocyclyl-C₁-C₇-alkyl-N—C₁-C₇-alkyl-amino-carbonyl-
amino-carbonyl-C₁-C₇-alkyl-
N—C₁-C₇-alkyl-amino-carbonyl-C₁-C₇-alkyl-
N,N-di-C₁-C₇-alkyl-amino-carbonyl-C₁-C₇-alkyl-
amino-C₁-C₇-alkyl-amino-C₁-C₇-alkyl-
amino-C₁-C₇-alkyl-N′—C₁-C₇-alkyl-amino-C₁-C₇-alkyl-
N—C₁-C₇-alkyl-amino-C₁-C₇-alkyl-amino-C₁-C₇-alkyl-
N—C₁-C₇-alkyl-amino-C₁-C₇-alkyl-N′—C₁-C₇-alkyl-amino-C₁-C₇-alkyl-
N,N-di-C₁-C₇-alkyl-amino-C₁-C₇-alkyl-amino-C₁-C₇-alkyl-
N,N-di-C₁-C₇-alkyl-amino-C₁-C₇-alkyl-N′—C₁-C₇-alkyl-amino-C₁-C₇-alkyl-
C₁-C₇-alkoxy-C₁-C₇-alkyl-amino-carbonyl-C₁-C₇-alkyl-
C₁-C₇-alkoxy-C₁-C₇-alkyl-N—C₁-C₇-alkyl-amino-carbonyl-C₁-C₇-alkyl-
C₁-C₇-alkoxy-carbonyl-C₁-C₇-alkyl-
C₃-C₁₀-cycloalkyl-C₁-C₇-alkyl-amino-carbonyl-C₁-C₇-alkyl-
C₃-C₁₀-cycloalkyl-C₁-C₇-alkyl-N—C₁-C₇-alkyl-amino-carbonyl-C₁-C₇-alkyl-
C₃-C₁₀-cycloalkyl-amino-carbonyl-C₁-C₇-alkyl-
heterocyclyl-C₁-C₇-alkyl-amino-carbonyl-C₁-C₇-alkyl-
heterocyclyl-amino-carbonyl-C₁-C₇-alkyl-
heterocyclyl-C₁-C₇-alkyl-N—C₁-C₇-alkyl-amino-carbonyl-C₁-C₇-alkyl-
aryl-C₁-C₇-alkyl-amino-carbonyl-C₁-C₇-alkyl-
aryl-C₁-C₇-alkyl-N—C₁-C₇-alkyl-amino-carbonyl-C₁-C₇-alkyl-
aryl-amino-carbonyl-C₁-C₇-alkyl-
aryl-amino-C₁-C₇-alkyl-amino-carbonyl-C₁-C₇-alkyl-
aryl-amino-C₁-C₇-alkyl-N—C₁-C₇-alkyl-amino-carbonyl-C₁-C₇-alkyl-
aryl-N—C₁-C₇-alkyl-amino-C₁-C₇-alkyl-amino-carbonyl-C₁-C₇-alkyl-
aryl-N—C₁-C₇-alkyl-amino-C₁-C₇-alkyl-N′—C₁-C₇-alkyl-amino-carbonyl-C₁-C₇-alkyl-
amino-C₁-C₇-alkyl-amino-carbonyl-
amino-C₁-C₇-alkyl-N′—C₁-C₇-alkyl-amino-carbonyl-
N—C₁-C₇-alkyl-amino-C₁-C₇-alkyl-amino-carbonyl-
N—C₁-C₇-alkyl-amino-C₁-C₇-alkyl-N′—C₁-C₇-alkyl-amino-carbonyl-
N,N-di-C₁-C₇-alkyl-amino-C₁-C₇-alkyl-amino-carbonyl-
N,N-di-C₁-C₇-alkyl-amino-C₁-C₇-alkyl-N′—C₁-C₇-alkyl-amino-carbonyl-
amino-C₁-C₇-alkyl-amino-carbonyl-C₁-C₇-alkyl-
amino-C₁-C₇-alkyl-N′—C₁-C₇-alkyl-amino-carbonyl-C₁-C₇-alkyl-
N—C₁-C₇-alkyl-amino-C₁-C₇-alkyl-amino-carbonyl-C₁-C₇-alkyl-
N—C₁-C₇-alkyl-amino-C₁-C₇-alkyl-N′—C₁-C₇-alkyl-amino-carbonyl-C₁-C₇-alkyl-
N,N-di-C₁-C₇-alkyl-amino-C₁-C₇-alkyl-amino-carbonyl-C₁-C₇-alkyl-
N,N-di-C₁-C₇-alkyl-amino-C₁-C₇-alkyl-N′—C₁-C₇-alkyl-amino-carbonyl-C₁-C₇-alkyl-
di-C₁-C₇-alkoxy-C₁-C₇-alkyl-amino-carbonyl-C₁-C₇-alkyl-
carboxyl-C₁-C₇-alkyl-
hydroxy-C₁-C₇-alkyl-
heterocyclyl-
N-(hydroxy-C₁-C₇-alkyl)-amino-carbonyl-C₁-C₇-alkyl-
N—(C₁-C₇-alkoxy-C₁-C₇-alkyl)-amino-carbonyl-C₁-C₇-alkyl-
N-(hydroxy-C₁-C₇-alkyl)-N—C₁-C₇-alkyl-amino-carbonyl-C₁-C₇-alkyl-
N—(C₁-C₇-alkoxy-C₁-C₇-alkyl)-N—C₁-C₇-alkyl-amino-carbonyl-C₁-C₇-alkyl-
C₁-C₇-alkyl-carbonyl-amino-C₁-C₇-alkyl-
C₁-C₇-alkyl-carbonyl-N—C₁-C₇-alkyl-amino-C₁-C₇-alkyl-
C₁-C₇-alkoxy-carbonyl-
C₁-C₇-alkyl-carbonyl-amino-
carboxyl-
hydroxy-C₁-C₇-alkyl-cyclopropyl-amino-carbonyl-methyl-, and
C₁-C₇-alkoxy-carbonyl-amino-C₁-C₇-alkyl-aminocarbonyl-alkyl-.

In another embodiment the invention provides a compound of the formula (I) and/or a tautomer and/or an N-oxide and/or a pharmaceutically acceptable salt and/or a solvate thereof, wherein R′ and R″ are independently selected from the group consisting of

hydroxy-
C₁-C₇-alkoxy-
halogen-
halo-C₁-C₇-alkyl-
cyano-
formyl-
C₁-C₇-alkyl-
amino-carbonyl-
N—C₁-C₇-alkyl-amino-carbonyl-
amino-carbonyl-
amino-C₁-C₇-alkyl-
heterocyclyl-C₁-C₇-alkyl-
N,N-di-C₁-C₇-alkyl-amino-C₁-C₇-alkyl-
heterocyclyl-carbonyl-C₁-C₇-alkyl-
heterocyclyl-heterocyclyl-carbonyl-C₁-C₇-alkyl-
heterocyclyl-C₁-C₇-alkyl-amino-carbonyl-
N—C₁-C₇-alkyl-amino-carbonyl-C₁-C₇-alkyl-
N,N-di-C₁-C₇-alkyl-amino-C₁-C₇-alkyl-N′—C₁-C₇-alkyl-amino-C₁-C₇-alkyl-
C₁-C₇-alkoxy-C₁-C₇-alkyl-amino-carbonyl-C₁-C₇-alkyl-
C₁-C₇-alkoxy-carbonyl-C₁-C₇-alkyl-
C₃-C₁₀-cycloalkyl-C₁-C₇-alkyl-amino-carbonyl-C₁-C₇-alkyl-
heterocyclyl-C₁-C₇-alkyl-amino-carbonyl-C₁-C₇-alkyl-
heterocyclyl-C₁-C₇-alkyl-N—C₁-C₇-alkyl-amino-carbonyl-C₁-C₇-alkyl-
aryl-C₁-C₇-alkyl-amino-carbonyl-C₁-C₇-alkyl-
aryl-C₁-C₇-alkyl-N—C₁-C₇-alkyl-amino-carbonyl-C₁-C₇-alkyl-
aryl-amino-carbonyl-C₁-C₇-alkyl-
aryl-amino-C₁-C₇-alkyl-amino-carbonyl-C₁-C₇-alkyl-
aryl-N—C₁-C₇-alkyl-amino-C₁-C₇-alkyl-amino-carbonyl-C₁-C₇-alkyl-
N,N-di-C₁-C₇-alkyl-amino-C₁-C₇-alkyl-amino-carbonyl-
N,N-di-C₁-C₇-alkyl-amino-C₁-C₇-alkyl-amino-carbonyl-C₁-C₇-alkyl-
di-C₁-C₇-alkoxy-C₁-C₇-alkyl-amino-carbonyl-C₁-C₇-alkyl-
carboxyl-C₁-C₇-alkyl-
hydroxy-C₁-C₇-alkyl-
heterocyclyl-
N-(hydroxy-C₁-C₇-alkyl)-amino-carbonyl-C₁-C₇-alkyl-
N—(C₁-C₇-alkoxy-C₁-C₇-alkyl)-amino-carbonyl-C₁-C₇-alkyl-
C₁-C₇-alkyl-carbonyl-amino-C₁-C₇-alkyl-
C₁-C₇-alkoxy-carbonyl-
C₁-C₇-alkyl-carbonyl-amino-.
carboxyl-.

Where R′ and/or R″ are or contain a C₁-C₇-alkyl or C₁-C₇-alkoxy group, said C₁-C₇-alkyl and/or C₁-C₇-alkoxy groups are preferably a C₁-C₄-alkyl or a C₁-C₄-alkoxy group.

In another embodiment, where R′ and/or R″ are heterocyclyl-heterocyclyl-carbonyl-C₁-C₇-alkyl-, said heterocyclyl-heterocyclyl-carbonyl-C₁-C₇-alkyl- is piperazinyl-piperidinyl-carbonyl-methyl-, wherein said piperazinyl is optionally methyl substituted.

In another embodiment, where R′ and/or R″ are heterocyclyl-C₁-C₇-alkyl-, said heterocyclyl-C₁-C₇-alkyl- is piperazinyl-C₁-C₇-alkyl-, wherein said piperazinyl is optionally methyl substituted.

In another embodiment, where R′ and/or R″ are heterocyclyl-carbonyl-C₁-C₇-alkyl-, said heterocyclyl-carbonyl-C₁-C₇-alkyl- is azetidinyl-carbonyl-methyl, wherein said azetidinyl is optionally substituted by one or two methyl substituents.

In another embodiment, where R′ and/or R″ are heterocyclyl-C₁-C₇-alkyl-aminocarbonyl, said heterocyclyl is morpholinyl or piperazinyl, wherein said piperazinyl is optionally methyl substituted.

In another embodiment, where R′ and/or R″ are heterocyclyl-C₁-C₇-alkyl-amino-carbonyl-C₁-C₇-alkyl-, said heterocyclyl-C₁-C₇-alkyl-amino-carbonyl-C₁-C₇-alkyl- is pyridyl-methyl-amino-carbonyl-methyl-.

In another embodiment, where R′ and/or R″ are aryl-C₁-C₇-alkyl-amino-carbonyl-C₁-C₇-alkyl-, said aryl-C₁-C₇-alkyl-amino-carbonyl-C₁-C₇-alkyl- is phenyl-methyl-aminocarbonyl-methyl-, wherein said phenyl is optionally substituted with a methyl or methoxy group.

In another embodiment, where R′ and/or R″ are heterocyclyl, said heterocyclyl is oxatriazolyl.

In another embodiment, where R′ and/or R″ are C₁-C₇-alkyl-carbonyl-amino-C₁-C₇-alkyl-, said C₁-C₇-alkyl-carbonyl-amino-C₁-C₇-alkyl- is methyl-carbonyl-amino-methyl-.

In another preferred embodiment the invention provides a compound of the formula (I) and/or a tautomer and/or an N-oxide and/or a pharmaceutically acceptable salt and/or a solvate thereof, wherein R′ and R″ are independently selected from the group consisting of

hydrogen,
chloro,
fluoro,
methoxy
hydroxy
amino-carbonyl-C₁-C₇-alkyl-
N—C₁-C₇-alkyl-amino-carbonyl-C₁-C₇-alkyl-, or
N,N-di-C₁-C₇-alkyl-amino-carbonyl-C₁-C₇-alkyl-
C₁-C₇-alkoxy-C₁-C₇-alkyl-amino-carbonyl-C₁-C₇-alkyl-
C₁-C₇-alkoxy-C₁-C₇-alkyl-N—C₁-C₇-alkyl-amino-carbonyl-C₁-C₇-alkyl-
C₃-C₁₀-cycloalkyl-C₁-C₇-alkyl-amino-carbonyl-C₁-C₇-alkyl-
C₃-C₁₀-cycloalkyl-C₁-C₇-alkyl-N—C₁-C₇-alkyl-amino-carbonyl-C₁-C₇-alkyl-
C₃-C₁₀-cycloalkyl-amino-carbonyl-C₁-C₇-alkyl-
heterocyclyl-C₁-C₇-alkyl-amino-carbonyl-C₁-C₇-alkyl-
heterocyclyl-amino-carbonyl-C₁-C₇-alkyl-
heterocyclyl-C₁-C₇-alkyl-N—C₁-C₇-alkyl-amino-carbonyl-C₁-C₇-alkyl-
aryl-C₁-C₇-alkyl-amino-carbonyl-C₁-C₇-alkyl-
aryl-C₁-C₇-alkyl-N—C₁-C₇-alkyl-amino-carbonyl-C₁-C₇-alkyl-
aryl-amino-carbonyl-C₁-C₇-alkyl-
aryl-amino-C₁-C₇-alkyl-amino-carbonyl-C₁-C₇-alkyl-
aryl-amino-C₁-C₇-alkyl-N—C₁-C₇-alkyl-amino-carbonyl-C₁-C₇-alkyl-
aryl-N—C₁-C₇-alkyl-amino-C₁-C₇-alkyl-amino-carbonyl-C₁-C₇-alkyl-
aryl-N—C₁-C₇-alkyl-amino-C₁-C₇-alkyl-N′—C₁-C₇-alkyl-amino-carbonyl-C₁-C₇-alkyl-
amino-C₁-C₇-alkyl-amino-carbonyl-C₁-C₇-alkyl-
amino-C₁-C₇-alkyl-N′—C₁-C₇-alkyl-amino-carbonyl-C₁-C₇-alkyl-
N—C₁-C₇-alkyl-amino-C₁-C₇-alkyl-amino-carbonyl-C₁-C₇-alkyl-
N—C₁-C₇-alkyl-amino-C₁-C₇-alkyl-N′—C₁-C₇-alkyl-amino-carbonyl-C₁-C₇-alkyl-
N,N-di-C₁-C₇-alkyl-amino-C₁-C₇-alkyl-amino-carbonyl-C₁-C₇-alkyl-
N,N-di-C₁-C₇-alkyl-amino-C₁-C₇-alkyl-N′—C₁-C₇-alkyl-amino-carbonyl-C₁-C₇-alkyl-
di-C₁-C₇-alkoxy-C₁-C₇-alkyl-amino-carbonyl-C₁-C₇-alkyl-
N-(hydroxy-C₁-C₇-alkyl)-amino-carbonyl-C₁-C₇-alkyl-
N—(C₁-C₇-alkoxy-C₁-C₇-alkyl)-amino-carbonyl-C₁-C₇-alkyl-
N-(hydroxy-C₁-C₇-alkyl)-N—C₁-C₇-alkyl-amino-carbonyl-C₁-C₇-alkyl-
N—(C₁-C₇-alkoxy-C₁-C₇-alkyl)-N—C₁-C₇-alkyl-amino-carbonyl-C₁-C₇-alkyl-
hydroxy-C₁-C₇-alkyl-cyclopropyl-amino-carbonyl-methyl-, and
C₁-C₇-alkoxy-carbonyl-amino-C₁-C₇-alkyl-aminocarbonyl-alkyl-.

In one preferred embodiment, R′ and/or R″ are selected from at least one of the group consisting of

hydrogen,
chloro,
fluoro,
methoxy and
hydroxy.

In another preferred embodiment the invention provides a compound of the formula (I) and/or a tautomer and/or an N-oxide and/or a pharmaceutically acceptable salt and/or a solvate thereof, wherein R′ and/or R″ are fluoro, and optionally another R′ and/or R″ substituent is present as defined herein.

In a preferred embodiment the invention provides a compound of the formula (I) and/or a tautomer and/or an N-oxide and/or a pharmaceutically acceptable salt and/or a solvate thereof, wherein R″ is selected from the group consisting of

chloro-
fluoro-.

In another preferred embodiment, ring A is 3-chloro-4-fluoro-phenyl.

Compounds of the formula (I) and/or tautomers and/or N-oxides and/or pharmaceutically acceptable salts and/or solvates thereof, which are particularly preferred embodiments of the invention, are:

Example 1: 1-(5-Chloro-2-methyl-phenyl)-5-(3-chloro-phenyl)-2-(3,4-dimethyl-phenyl)-1H-imidazole-4-carboxylic acid ethylamide
Example 2: 5-[1-(5-Chloro-2-methyl-phenyl)-5-(3-chloro-phenyl)-2-(3,4-dimethyl-phenyl)-1H-imidazol-4-yl]-tetrazole
Example 3: 1-(5-chloro-2-methyl-phenyl)-5-(3-chloro-phenyl)-2-(3,4-dimethyl-phenyl)-1H-imidazole-4-nitrile
Example 4: 1-(5-Chloro-2-methyl-phenyl)-5-(3-chloro-phenyl)-2-(3,4-dimethyl-phenyl)-1H-imidazole-4-carboxylic acid amide
Example 5: 1-(5-Chloro-2-methyl-phenyl)-5-(3-chloro-phenyl)-2-(3,4-dimethyl-phenyl)-1H-imidazole-4-carboxylic acid
Example 6: 1-(5-Chloro-2-methyl-phenyl)-5-(3-chloro-phenyl)-2-(3,4-dimethyl-phenyl)-1H-imidazole-4-carboxylic acid ethyl ester
Example 7: 5-(3-Chloro-4-fluoro-phenyl)-1-(3-chloro-2-fluoro-phenyl)-2-thiophen-3-yl-1H-imidazole-4-carboxylic acid ethyl ester
Example 8: 1-(3-Chloro-2-fluoro-phenyl)-2-(3-chloro-phenyl)-5-phenyl-1H-pyrrole-3-carboxylic acid
Example 9: 1-(3-Chloro-2-fluoro-phenyl)-2-(3-chloro-phenyl)-5-phenyl-1H-pyrrole-3-carboxylic acid ethyl ester
Example 10: 4,5-Bis-(3-chloro-phenyl)-1-phenyl-1H-pyrazole-3-carboxylic acid
Example 11: 1,5-Bis-(3-chloro-phenyl)-2-phenyl-1H-imidazole-4-carboxylic acid ethyl ester
Example 12: 1,5-Bis-(3-chloro-phenyl)-2-phenyl-1H-imidazole-4-carboxylic acid
Example 13: 1,5-Bis-(3-chloro-phenyl)-2-phenyl-1H-imidazole-4-carboxylic acid (2-morpholin-4-yl-ethyl)-amide
Example 14: 1,5-Bis-(3-chloro-phenyl)-2-phenyl-1H-imidazole-4-carboxylic acid methylamide
Example 15: 1,5-Bis-(3-chloro-phenyl)-2-phenyl-1H-imidazole-4-carboxylic acid (3-dimethylamino-propyl)-methyl-amide
Example 16: 5-[5-(3-Chloro-4-fluoro-phenyl)-1-(3-chloro-2-fluoro-phenyl)-2-(3-chloro-phenyl)-1H-imidazol-4-yl]-2H-tetrazole
Example 17: 6-[5-(3-Chloro-4-fluoro-phenyl)-1-(3-chloro-2-fluoro-phenyl)-4-(2H-tetrazol-5-yl)-1H-imidazol-2-yl]-1H-indole
Example 18: 6-[5-(3-Chloro-4-fluoro-phenyl)-1-(3-chloro-2-fluoro-phenyl)-4-(2H-tetrazol-5-yl)-1H-imidazol-2-yl]-1-methyl-1H-indole
Example 19: 5-[2-Benzo[b]thiophen-5-yl-5-(3-chloro-4-fluoro-phenyl)-1-(3-chloro-2-fluoro-phenyl)-1H-imidazol-4-yl]-2H-tetrazole
Example 20: 5-[1-(5-Chloro-2-methyl-phenyl)-5-(3-chloro-phenyl)-2-(3,4-dimethyl-phenyl)-1H-imidazol-4-yl]-2-methyl-2H-tetrazole
Example 21: 5-[1-(5-Chloro-2-methyl-phenyl)-5-(3-chloro-phenyl)-2-(3,4-dimethyl-phenyl)-1H-imidazol-4-yl]-1-methyl-1H-tetrazole
Example 22: 3-Chloro-5-[3-(3-chloro-2-fluoro-phenyl)-5-cyano-2-cyclohexyl-3H-imidazol-4-yl]-N,N-dimethyl-benzamide
Example 23: 3-Chloro-5-[3-(3-chloro-2-fluoro-phenyl)-5-cyano-2-cyclohexyl-3H-imidazol-4-yl]-N-(2-dimethylamino-ethyl)-benzamide
Example 24: 3-Chloro-5-[3-(3-chloro-2-fluoro-phenyl)-5-cyano-2-cyclohexyl-3H-imidazol-4-yl]-N-(2-morpholin-4-yl-ethyl)-benzamide
Example 25: 3-Chloro-5-[3-(3-chloro-2-fluoro-phenyl)-2-cyclohexyl-5-(1H-tetrazol-5-yl)-3H-imidazol-4-yl]-N,N-dimethyl-benzamide
Example 26: 5-(3-Chloro-phenyl)-1-(4-chloro-phenyl)-2-cyclopropylmethyl-1H-imidazole-4-carboxylic acid ethyl ester
Example 27: 5-(3-Chloro-phenyl)-1-(4-chloro-phenyl)-2-cyclopropylmethyl-1H-imidazole-4-carboxylic acid
Example 28: 5-(3-Chloro-phenyl)-1-(4-chloro-phenyl)-2-cyclopropylmethyl-1H-imidazole-4-carboxylic acid methylamide
Example 29: 5-(3-Chloro-phenyl)-1-(4-chloro-phenyl)-2-cyclopropyl methyl-1H-imidazole-4-carboxylic acid (2-morpholin-4-yl-ethyl)-amide
Example 30: 3-Chloro-5-[3-(3-chloro-2-fluoro-phenyl)-2-cyclohexyl-5-(1H-tetrazol-5-yl)-3H-imidazol-4-yl]-N-(2-dimethylamino-ethyl)-benzamide
Example 31: 3-Chloro-5-[3-(3-chloro-2-fluoro-phenyl)-2-cyclohexyl-5-(1H-tetrazol-5-yl)-3H-imidazol-4-yl]-N-(2-morpholin-4-yl-ethyl)-benzamide
Example 32: 5-(3-Chloro-4-fluoro-phenyl)-1-(3-chloro-2-fluoro-phenyl)-2-phenyl-1H-imidazole-4-carboxylic acid (2-hydroxy-ethyl)-amide
Example 33: 3-Chloro-5-[3-(3-chloro-2-fluoro-phenyl)-5-cyano-2-cyclohexyl-3H-imidazol-4-yl]-N,N-dimethyl-benzamide
Example 34: 3-Chloro-5-[3-(3-chloro-2-fluoro-phenyl)-5-cyano-2-cyclohexyl-3H-imidazol-4-yl]-N-(2-dimethylamino-ethyl)-benzamide
Example 35: 3-Chloro-5-[3-(3-chloro-2-fluoro-phenyl)-5-cyano-2-cyclohexyl-3H-imidazol-4-yl]-N-(2-morpholin-4-yl-ethyl)-benzamide
Example 36: 1-(5-Chloro-2-methyl-phenyl)-5-(3-chloro-phenyl)-2-phenyl-1H-imidazole-4-carboxylic acid ethyl ester
Example 37: 1-(5-Chloro-2-methyl-phenyl)-5-(3-chloro-phenyl)-2-phenyl-1H-imidazole-4-carboxylic acid
Example 38: 1-(5-Chloro-2-methyl-phenyl)-5-(3-chloro-phenyl)-2-phenyl-1H-imidazole-4-carboxylic acid amide
Example 39: 1-(5-Chloro-2-methyl-phenyl)-5-(3-chloro-phenyl)-2-phenyl-1H-imidazole-4-carboxylic acid methylamide
Example 40: 1-(5-Chloro-2-methyl-phenyl)-5-(3-chloro-phenyl)-2-phenyl-1H-imidazole-4-carboxylic acid hydroxyamide
Example 41: 1-(5-Chloro-2-methyl-phenyl)-5-(3-chloro-phenyl)-2-phenyl-1H-imidazole-4-carboxylic acid dimethylamide
Example 45: 5-(3-Chloro-4-fluoro-phenyl)-1-(3-chloro-2-fluoro-phenyl)-2-phenyl-1H-imidazole-4-carboxylic acid (2-hydroxy-ethyl)-amide
Example 46: 2-{4-Chloro-2-[5-(3-chloro-4-fluoro-phenyl)-2-cyclohexyl-4-(1H-tetrazol-5-yl)-imidazol-1-yl]-phenyl}-N-methyl-acetamide
Example 47: 1-(5-Chloro-2-methyl-phenyl)-5-(3-chloro-phenyl)-2-m-tolyl-1H-imidazole-4-carboxylic acid ethyl ester
Example 48: 1-(5-Chloro-2-methyl-phenyl)-5-(3-chloro-phenyl)-2-m-tolyl-1H-imidazole-4-carboxylic acid
Example 49: 1-(5-Chloro-2-methyl-phenyl)-5-(3-chloro-phenyl)-2-m-tolyl-1H-imidazole-4-carboxylic acid methylamide
Example 50: 1-(5-Chloro-2-methyl-phenyl)-5-(3-chloro-phenyl)-2-m-tolyl-1H-imidazole-4-carboxylic acid amide
Example 51: 1-(5-Chloro-2-methyl-phenyl)-5-(3-chloro-phenyl)-2-m-tolyl-1H-imidazole-4-carboxylic acid ethoxy-amide
Example 52: 1-(5-Chloro-2-methyl-phenyl)-5-(3-chloro-phenyl)-2-m-tolyl-1H-imidazole-4-carboxylic acid isobutoxy-amide
Example 53: 1-(5-Chloro-2-methyl-phenyl)-5-(3-chloro-phenyl)-2-m-tolyl-1H-imidazole-4-carboxylic acid benzyloxy-amide
Example 54: 1-(5-Chloro-2-methyl-phenyl)-5-(3-chloro-phenyl)-2-m-tolyl-1H-imidazole-4-carboxylic acid hydroxyamide
Example 55: 5-(5-Chloro-2-methoxy-phenyl)-1-(5-chloro-2-methyl-phenyl)-2-p-tolyl-1H-imidazole-4-carboxylic acid ethyl ester
Example 56: 5-(5-Chloro-2-methoxy-phenyl)-1-(5-chloro-2-methyl-phenyl)-2-p-tolyl-1H-imidazole-4-carboxylic acid
Example 57: 5-(5-Chloro-2-hydroxy-phenyl)-1-(5-chloro-2-methyl-phenyl)-2-p-tolyl-1H-imidazole-4-carboxylic acid
Example 58: 1-(5-Chloro-2-methyl-phenyl)-5-(3-chloro-phenyl)-2-p-tolyl-1H-imidazole-4-carboxylic acid ethyl ester
Example 59: 1-(5-Chloro-2-methyl-phenyl)-5-(3-chloro-phenyl)-2-p-tolyl-1H-imidazole-4-carboxylic acid
Example 60: 1-(5-Chloro-2-methyl-phenyl)-5-(2,5-dichloro-phenyl)-2-m-tolyl-1H-imidazole-4-carboxylic acid ethyl ester
Example 61: 1-(5-Chloro-2-methyl-phenyl)-5-(2,5-dichloro-phenyl)-2-m-tolyl-1H-imidazole-4-carboxylic acid
Example 62: 5-(3-Chloro-2-fluoro-phenyl)-1-(5-chloro-2-methyl-phenyl)-2-m-tolyl-1H-imidazole-4-carboxylic acid ethyl ester
Example 63: 5-(3-Chloro-2-fluoro-phenyl)-1-(5-chloro-2-methyl-phenyl)-2-m-tolyl-1H-imidazole-4-carboxylic acid
Example 64: 5-(5-Chloro-2-methoxy-phenyl)-1-(5-chloro-2-methyl-phenyl)-2-m-tolyl-1H-imidazole-4-carboxylic acid ethyl ester
Example 65: 5-(5-Chloro-2-methoxy-phenyl)-1-(5-chloro-2-methyl-phenyl)-2-m-tolyl-1H-imidazole-4-carboxylic acid
Example 66: 5-(5-Chloro-2-hydroxy-phenyl)-1-(5-chloro-2-methyl-phenyl)-2-m-tolyl-1H-imidazole-4-carboxylic acid
Example 67: 5-(5-Chloro-2-methoxy-phenyl)-1-(5-chloro-2-methyl-phenyl)-2-m-tolyl-1H-imidazole-4-carboxylic acid amide
Example 68: 5-(5-Chloro-2-methoxy-phenyl)-1-(5-chloro-2-methyl-phenyl)-2-m-tolyl-1H-imidazole-4-carbonitrile
Example 69: 5-[5-(5-Chloro-2-methoxy-phenyl)-1-(5-chloro-2-methyl-phenyl)-2-m-tolyl-1H-imidazol-4-yl]-2H-tetrazole
Example 70: N-[5-(5-Chloro-2-methoxy-phenyl)-1-(5-chloro-2-methyl-phenyl)-2-m-tolyl-1H-imidazol-4-ylmethyl]-acetamide
Example 71: 1-(5-Chloro-2-methyl-phenyl)-5-(3,4-dichloro-phenyl)-2-m-tolyl-1H-imidazole-4-carboxylic acid ethyl ester
Example 72: 1-(5-Chloro-2-methyl-phenyl)-5-(3,4-dichloro-phenyl)-2-m-tolyl-1H-imidazole-4-carboxylic acid
Example 73: 2-{4-Chloro-2-[5-(3-chloro-4-fluoro-phenyl)-2-cyclohexyl-4-(2H-tetrazol-5-yl)-imidazol-1-yl]-phenyl}-N-(2-morpholin-4-yl-ethyl)-acetamide
Example 74: 2-{4-Chloro-2-[5-(3-chloro-4-fluoro-phenyl)-2-cyclohexyl-4-(2H-tetrazol-5-yl)-imidazol-1-yl]-phenyl}-N-[2-(4-methyl-piperazin-1-yl)-ethyl]-acetamide
Example 75: 5-(3-Chloro-4-fluoro-phenyl)-1-(3-chloro-2-fluoro-phenyl)-2-m-tolyl-1H-imidazole-4-carboxylic acid ethyl ester
Example 76: 5-(3-Chloro-4-fluoro-phenyl)-1-(3-chloro-2-fluoro-phenyl)-2-m-tolyl-1H-imidazole-4-carboxylic acid
Example 77: 1-(3-Chloro-2-fluoro-phenyl)-5-(3,4-dichloro-phenyl)-2-m-tolyl-1H-imidazole-4-carboxylic acid ethyl ester
Example 78: 1-(3-Chloro-2-fluoro-phenyl)-5-(3,4-dichloro-phenyl)-2-m-tolyl-1H-imidazole-4-carboxylic acid
Example 79: 1-(3-Chloro-phenyl)-5-(3,5-dichloro-phenyl)-2-phenyl-1H-imidazole-4-carboxylic acid ethyl ester
Example 80: 1-(3-Chloro-phenyl)-5-(3,5-dichloro-phenyl)-2-phenyl-1H-imidazole-4-carboxylic acid
Example 81: 1-(3-Chloro-phenyl)-5-(3,5-dichloro-phenyl)-2-phenyl-1H-imidazole-4-carboxylic acid methylamide
Example 82: 1-(3-Chloro-2-fluoro-phenyl)-5-(5-chloro-2-methoxy-phenyl)-2-m-tolyl-1H-imidazole-4-carboxylic acid ethyl ester
Example 83: 1-(3-Chloro-2-fluoro-phenyl)-5-(5-chloro-2-methoxy-phenyl)-2-m-tolyl-1H-imidazole-4-carboxylic acid
Example 84: 1-(3-Chloro-2-fluoro-phenyl)-5-(5-chloro-2-methoxy-phenyl)-2-m-tolyl-1H-imidazole-4-carboxylic acid amide
Example 85: 1-(3-Chloro-2-fluoro-phenyl)-5-(5-chloro-2-methoxy-phenyl)-2-m-tolyl-1H-imidazole-4-carbonitrile
Example 86: 5-[1-(3-Chloro-2-fluoro-phenyl)-5-(5-chloro-2-methoxy-phenyl)-2-m-tolyl-1H-imidazol-4-yl]-2H-tetrazole
Example 87: 1-(3-Chloro-2-fluoro-phenyl)-5-(3-chloro-phenyl)-2-m-tolyl-1H-imidazole-4-carboxylic acid ethyl ester
Example 88: 1-(3-Chloro-2-fluoro-phenyl)-5-(3-chloro-phenyl)-2-m-tolyl-1H-imidazole-4-carboxylic acid
Example 89: 5-(3-Chloro-2-fluoro-phenyl)-1-(4-chloro-phenyl)-2-cyclopropylmethyl-1H-imidazole-4-carboxylic acid ethyl ester
Example 90: 5-(3-Chloro-2-fluoro-phenyl)-1-(4-chloro-phenyl)-2-cyclopropylmethyl-1H-imidazole-4-carboxylic acid
Example 91: 5-(3-Chloro-2-fluoro-phenyl)-1-(4-chloro-phenyl)-2-cyclopropylmethyl-1H-imidazole-4-carboxylic acid methylamide
Example 92: 1,5-Bis-(3-chloro-phenyl)-2-isobutyl-1H-imidazole-4-carboxylic acid ethyl ester
Example 93: 1,5-Bis-(3-chloro-phenyl)-2-isobutyl-1H-imidazole-4-carboxylic acid
Example 94: 1,5-Bis-(3-chloro-phenyl)-2-isobutyl-1H-imidazole-4-carboxylic acid methylamide
Example 95: 1,5-Bis-(3-chloro-phenyl)-2-isobutyl-1H-imidazole-4-carboxylic acid ethylamide
Example 96: 1,5-Bis-(3-chloro-phenyl)-2-isobutyl-1H-imidazole-4-carboxylic acid (2-morpholin-4-yl-ethyl)-amide
Example 97: 5-(3-Chloro-2-fluoro-phenyl)-1-(3-chloro-phenyl)-2-isobutyl-1H-imidazole-4-carboxylic acid ethyl ester
Example 98: 5-(3-Chloro-2-fluoro-phenyl)-1-(3-chloro-phenyl)-2-isobutyl-1H-imidazole-4-carboxylic acid
Example 99: 5-(3-Chloro-2-fluoro-phenyl)-1-(3-chloro-phenyl)-2-isobutyl-1H-imidazole-4-carboxylic acid methylamide
Example 100: 5-(3-Chloro-2-fluoro-phenyl)-1-(3-chloro-phenyl)-2-isobutyl-1H-imidazole-4-carboxylic acid ethylamide
Example 101: N-Benzyl-2-{4-chloro-2-[5-(3-chloro-4-fluoro-phenyl)-2-cyclohexyl-4-(2H-tetrazol-5-yl)-imidazol-1-yl]-phenyl}-acetamide
Example 102: N-tert-Butyl-2-{4-chloro-2-[5-(3-chloro-4-fluoro-phenyl)-2-cyclohexyl-4-(2H-tetrazol-5-yl)-imidazol-1-yl]-phenyl}-acetamide
Example 103: 2-{4-Chloro-2-[5-(3-chloro-4-fluoro-phenyl)-2-cyclohexyl-4-(2H-tetrazol-5-yl)-imidazol-1-yl]-phenyl}-N-pyridin-3-ylmethyl-acetamide
Example 104: 2-{4-Chloro-2-[5-(3-chloro-4-fluoro-phenyl)-2-cyclohexyl-4-(2H-tetrazol-5-yl)-imidazol-1-yl]-phenyl}-N-pyridin-2-ylmethyl-acetamide
Example 105: 3-{2-[4-Carbamoyl-5-(3-chloro-4-fluoro-phenyl)-1-(3-chloro-2-fluoro-phenyl)-1H-imidazol-2-yl]-phenylamino}-propionic acid tert-butyl ester
Example 106: 5-(3-Chloro-2-fluoro-phenyl)-1-(3-chloro-phenyl)-2-phenyl-1H-imidazole-4-carboxylic acid ethyl ester
Example 107: 5-(3-Chloro-2-fluoro-phenyl)-1-(3-chloro-phenyl)-2-phenyl-1H-imidazole-4-carboxylic acid
Example 108: 5-(3-Chloro-2-fluoro-phenyl)-1-(3-chloro-phenyl)-2-phenyl-1H-imidazole-4-carboxylic acid isopropylamide
Example 109: 5-(3-Chloro-2-fluoro-phenyl)-1-(3-chloro-phenyl)-2-phenyl-1H-imidazole-4-carboxylic acid methylamide
Example 110: 1,5-Bis-(3-chloro-phenyl)-2-(2-fluoro-phenyl)-1H-imidazole-4-carboxylic acid ethyl ester
Example 111: 1,5-Bis-(3-chloro-phenyl)-2-(2-fluoro-phenyl)-1H-imidazole-4-carboxylic acid
Example 112: 1,5-Bis-(3-chloro-phenyl)-2-(2-fluoro-phenyl)-1H-imidazole-4-carboxylic acid amide
Example 113: 1,5-Bis-(3-chloro-phenyl)-2-(2-fluoro-phenyl)-1H-imidazole-4-carboxylic acid methylamide
Example 114: 3-{2-[4-Carbamoyl-5-(3-chloro-4-fluoro-phenyl)-1-(3-chloro-2-fluoro-phenyl)-1H-imidazol-2-yl]-phenylamino}-propionic acid
Example 115: 5-(3-Chloro-4-fluoro-phenyl)-1-(3-chloro-2-fluoro-phenyl)-2-{2-[2-(isobutyl-methyl-carbamoyl)-ethylamino]-phenyl}-1H-imidazole-4-carboxylic acid amide
Example 116: 5-(3-Chloro-4-fluoro-phenyl)-1-(3-chloro-2-fluoro-phenyl)-2-{2-[2-(2,2-dimethoxy-ethylcarbamoyl)-ethylamino]-phenyl}-1H-imidazole-4-carboxylic acid amide
Example 117: 5-(3-Chloro-4-fluoro-phenyl)-1-(3-chloro-2-fluoro-phenyl)-2-{(2-[2-(methyl-pyridin-3-ylmethyl-carbamoyl)-ethylamino]-phenyl}-1H-imidazole-4-carboxylic acid amide
Example 118: 2-Benzyl-1,5-bis-(3-chloro-phenyl)-1H-imidazole-4-carboxylic acid ethyl ester
Example 119: 2-Benzyl-1,5-bis-(3-chloro-phenyl)-1H-imidazole-4-carboxylic acid
Example 120: 2-Benzyl-1,5-bis-(3-chloro-phenyl)-1H-imidazole-4-carboxylic acid methylamide
Example 121: 2-(2-Chloro-phenyl)-1,5-bis-(3-chloro-phenyl)-1H-imidazole-4-carboxylic acid ethyl ester
Example 122: 2-(2-Chloro-phenyl)-1,5-bis-(3-chloro-phenyl)-1H-imidazole-4-carboxylic acid
Example 123: 2-(2-Chloro-phenyl)-1,5-bis-(3-chloro-phenyl)-1H-imidazole-4-carboxylic acid methylamide
Example 124: 5-(3-Chloro-2-fluoro-phenyl)-2-(2-chloro-phenyl)-1-(3-chloro-phenyl)-1H-imidazole-4-carboxylic acid ethyl ester
Example 125: 5-(3-Chloro-2-fluoro-phenyl)-2-(2-chloro-phenyl)-1-(3-chloro-phenyl)-1H-imidazole-4-carboxylic acid
Example 126: 5-(5-Chloro-2-methoxy-phenyl)-1-(3-chloro-phenyl)-2-phenyl-1H-imidazole-4-carboxylic acid ethyl ester
Example 127: 5-(5-Chloro-2-methoxy-phenyl)-1-(3-chloro-phenyl)-2-phenyl-1H-imidazole-4-carboxylic acid
Example 128: 5-(5-Chloro-2-methoxy-phenyl)-1-(3-chloro-phenyl)-2-phenyl-1H-imidazole-4-carboxylic acid methylamide
Example 129: 1-(5-Chloro-2-methyl-phenyl)-5-(3-chloro-phenyl)-2-pyridin-3-yl-1H-imidazole-4-carboxylic acid ethyl ester
Example 130: 1-(5-Chloro-2-methyl-phenyl)-5-(3-chloro-phenyl)-2-pyridin-3-yl-1H-imidazole-4-carboxylic acid
Example 131: 5-(5-Chloro-2-methoxy-phenyl)-1-(5-chloro-2-methyl-phenyl)-2-phenyl-1H-imidazole-4-carboxylic acid ethyl ester
Example 132: 5-(5-Chloro-2-methoxy-phenyl)-1-(5-chloro-2-methyl-phenyl)-2-phenyl-1H-imidazole-4-carboxylic acid
Example 133: 5-(5-Chloro-2-hydroxy-phenyl)-1-(5-chloro-2-methyl-phenyl)-2-phenyl-1H-imidazole-4-carboxylic acid
Example 134: 1-(5-Chloro-2-methyl-phenyl)-5-(3-chloro-phenyl)-2-pyridin-2-yl-1H-imidazole-4-carboxylic acid ethyl ester
Example 135: 1-(5-Chloro-2-methyl-phenyl)-5-(3-chloro-phenyl)-2-pyridin-2-yl-1H-imidazole-4-carboxylic acid
Example 136: 1-(3-Chloro-4-fluoro-phenyl)-5-(3-chloro-phenyl)-2-phenyl-1H-imidazole-4-carboxylic acid ethyl ester
Example 137: 1-(3-Chloro-4-fluoro-phenyl)-5-(3-chloro-phenyl)-2-phenyl-1H-imidazole-4-carboxylic acid
Example 138: 1-(3-Chloro-4-fluoro-phenyl)-5-(3-chloro-phenyl)-2-phenyl-1H-imidazole-4-carboxylic acid methylamide
Example 139: 1-(3-Chloro-4-fluoro-phenyl)-5-(3-chloro-phenyl)-2-phenyl-1H-imidazole-4-carboxylic acid amide
Example 140: 1-(3-Chloro-2-fluoro-phenyl)-5-(5-chloro-2-methoxy-phenyl)-2-phenyl-1H-imidazole-4-carboxylic acid ethyl ester
Example 141: 1-(3-Chloro-2-fluoro-phenyl)-5-(5-chloro-2-methoxy-phenyl)-2-phenyl-1H-imidazole-4-carboxylic acid
Example 142: 1-(3-Chloro-2-fluoro-phenyl)-5-(5-chloro-2-hydroxy-phenyl)-2-phenyl-1H-imidazole-4-carboxylic acid
Example 143: 1-(3-Chloro-2-fluoro-phenyl)-5-(5-chloro-2-methoxy-phenyl)-2-phenyl-1H-imidazole-4-carboxylic acid amide
Example 144: 1-(3-Chloro-2-fluoro-phenyl)-5-(5-chloro-2-methoxy-phenyl)-2-phenyl-1H-imidazole-4-carbonitrile
Example 145: 1-(3-Chloro-2-fluoro-phenyl)-5-(5-chloro-2-hydroxy-phenyl)-2-phenyl-1H-imidazole-4-carbonitrile
Example 146: 5-[1-(3-Chloro-2-fluoro-phenyl)-5-(5-chloro-2-methoxy-phenyl)-2-phenyl-1H-imidazol-4-yl]-2H-tetrazole
Example 147: 5-(5-Chloro-2-methoxy-phenyl)-1-(5-chloro-2-methyl-phenyl)-2-(6-methyl-pyridin-2-yl)-1H-imidazole-4-carboxylic acid ethyl ester
Example 148: 5-(5-Chloro-2-methoxy-phenyl)-1-(5-chloro-2-methyl-phenyl)-2-(6-methyl-pyridin-2-yl)-1H-imidazole-4-carboxylic acid amide
Example 149: 5-(5-Chloro-2-methoxy-phenyl)-1-(5-chloro-2-methyl-phenyl)-2-(6-methyl-pyridin-2-yl)-1H-imidazole-4-carboxylic acid
Example 150: 5-(5-Chloro-2-hydroxy-phenyl)-1-(5-chloro-2-methyl-phenyl)-2-(6-methyl-pyridin-2-yl)-1H-imidazole-4-carboxylic acid
Example 151: 5-(5-Chloro-2-methoxy-phenyl)-1-(5-chloro-2-methyl-phenyl)-2-(6-methyl-pyridin-2-yl)-1H-imidazole-4-carbonitrile
Example 152: 5-(3-Chloro-4-fluoro-phenyl)-1-(3-chloro-2-fluoro-phenyl)-2-{2-[2-(cyclohexylmethyl-methyl-carbamoyl)-ethylamino]-phenyl}-1H-imidazole-4-carboxylic acid amide
Example 153: 2-[2-(2-Carbamoyl-ethylamino)-phenyl]-5-(3-chloro-4-fluoro-phenyl)-1-(3-chloro-2-fluoro-phenyl)-1H-imidazole-4-carboxylic acid amide
Example 154: 5-(3-Chloro-4-fluoro-phenyl)-1-(3-chloro-2-fluoro-phenyl)-2-[2-(2-hydrazinocarbonyl-ethylamino)-phenyl]-1H-imidazole-4-carboxylic acid amide
Example 155: 1-(5-Chloro-2-methyl-phenyl)-5-(3-chloro-phenyl)-2-(6-methyl-pyridin-2-yl)-1H-imidazole-4-carboxylic acid ethyl ester
Example 156: 1-(5-Chloro-2-methyl-phenyl)-5-(3-chloro-phenyl)-2-(6-methyl-pyridin-2-yl)-1H-imidazole-4-carboxylic acid
Example 157: 1-(5-Chloro-2-methyl-phenyl)-5-(3,4-dichloro-phenyl)-2-(6-methyl-pyridin-2-yl)-1H-imidazole-4-carboxylic acid ethyl ester
Example 158: 1-(5-Chloro-2-methyl-phenyl)-5-(3,4-dichloro-phenyl)-2-(6-methyl-pyridin-2-yl)-1H-imidazole-4-carboxylic acid
Example 159: 1-(5-Chloro-2-fluoro-phenyl)-5-(3-chloro-phenyl)-2-m-tolyl-1H-imidazole-4-carboxylic acid ethyl ester
Example 160: 1-(5-Chloro-2-fluoro-phenyl)-5-(3-chloro-phenyl)-2-m-tolyl-1H-imidazole-4-carboxylic acid
Example 161: 1-(5-Chloro-2-fluoro-phenyl)-5-(5-chloro-2-methoxy-phenyl)-2-m-tolyl-1H-imidazole-4-carboxylic acid ethyl ester
Example 162: 1-(5-Chloro-2-fluoro-phenyl)-5-(5-chloro-2-methoxy-phenyl)-2-m-tolyl-1H-imidazole-4-carboxylic acid
Example 163: 1-(5-Chloro-2-fluoro-phenyl)-5-(5-chloro-2-methoxy-phenyl)-2-m-tolyl-1H-imidazole-4-carboxylic acid amide
Example 164: 1-(5-Chloro-2-fluoro-phenyl)-5-(5-chloro-2-methoxy-phenyl)-2-m-tolyl-1H-imidazole-4-carbonitrile
Example 165: 1-(5-Chloro-2-fluoro-phenyl)-5-(5-chloro-2-hydroxy-phenyl)-2-m-tolyl-1H-imidazole-4-carbonitrile
Example 166: 5-(3-Chloro-4-fluoro-phenyl)-1-(5-chloro-2-fluoro-phenyl)-2-m-tolyl-1H-imidazole-4-carboxylic acid ethyl ester
Example 167: 5-(3-Chloro-4-fluoro-phenyl)-1-(5-chloro-2-fluoro-phenyl)-2-m-tolyl-1H-imidazole-4-carboxylic acid
Example 168: 1-(3-Chloro-phenyl)-5-(3,4-dichloro-phenyl)-2-phenyl-1H-imidazole-4-carboxylic acid ethyl ester
Example 169: 1-(3-Chloro-phenyl)-5-(3,4-dichloro-phenyl)-2-phenyl-1H-imidazole-4-carboxylic acid
Example 170: 1-(3-Chloro-phenyl)-5-(3,4-dichloro-phenyl)-2-phenyl-1H-imidazole-4-carboxylic acid methylamide
Example 171: 1-(3-Chloro-2-fluoro-phenyl)-5-(3-chloro-phenyl)-2-phenyl-1H-imidazole-4-carboxylic acid ethyl ester
Example 172: 1-(3-Chloro-2-fluoro-phenyl)-5-(3-chloro-phenyl)-2-phenyl-1H-imidazole-4-carboxylic acid
Example 173: 1-(3-Chloro-2-fluoro-phenyl)-5-(3-chloro-phenyl)-2-phenyl-1H-imidazole-4-carboxylic acid methylamide
Example 174: 1-(3-Chloro-2-fluoro-phenyl)-5-(3-chloro-phenyl)-2-phenyl-1H-imidazole-4-carboxylic acid amide
Example 175: 1-(3-Chloro-2-fluoro-phenyl)-5-(3-chloro-phenyl)-2-phenyl-1H-imidazole-4-carboxylic acid hydroxyamide
Example 176: 1-(3-Chloro-2-fluoro-phenyl)-5-(3-chloro-phenyl)-2-phenyl-1H-imidazole-4-carbonitrile
Example 177: 5-[1-(3-Chloro-2-fluoro-phenyl)-5-(3-chloro-phenyl)-2-phenyl-1H-imidazol-4-yl]-2H-tetrazole
Example 178: 5-(5-Chloro-2-methoxy-phenyl)-1-(5-chloro-2-methyl-phenyl)-2-pyridin-2-yl-1H-imidazole-4-carboxylic acid ethyl ester
Example 179: 5-(5-Chloro-2-methoxy-phenyl)-1-(5-chloro-2-methyl-phenyl)-2-pyridin-2-yl-1H-imidazole-4-carboxylic acid
Example 180: 5-(5-Chloro-2-methoxy-phenyl)-1-(5-chloro-2-methyl-phenyl)-2-(3,4-dimethyl-phenyl)-1H-imidazole-4-carboxylic acid ethyl ester
Example 181: 5-(5-Chloro-2-methoxy-phenyl)-1-(5-chloro-2-methyl-phenyl)-2-(3,4-dimethyl-phenyl)-1H-imidazole-4-carboxylic acid
Example 182: 5-(5-Chloro-2-methoxy-phenyl)-1-(5-chloro-2-methyl-phenyl)-2-(3,4-dimethyl-phenyl)-1H-imidazole-4-carboxylic acid amide
Example 183: 5-(5-Chloro-2-methoxy-phenyl)-1-(5-chloro-2-methyl-phenyl)-2-(3,4-dimethyl-phenyl)-1H-imidazole-4-carbonitrile
Example 184: 5-[5-(5-Chloro-2-methoxy-phenyl)-1-(5-chloro-2-methyl-phenyl)-2-(3,4-dimethyl-phenyl)-1H-imidazol-4-yl]-2H-tetrazole
Example 185: 1-(5-Chloro-2-methyl-phenyl)-5-(3,4-dichloro-phenyl)-2-(3,4-dimethyl-phenyl)-1H-imidazole-4-carboxylic acid ethyl ester
Example 186: 1-(5-Chloro-2-methyl-phenyl)-5-(3,4-dichloro-phenyl)-2-(3,4-dimethyl-phenyl)-1H-imidazole-4-carboxylic acid
Example 187: 5-(3-Chloro-4-fluoro-phenyl)-1-(5-chloro-2-methyl-phenyl)-2-(3,4-dimethyl-phenyl)-1H-imidazole-4-carboxylic acid ethyl ester
Example 188: 5-(3-Chloro-4-fluoro-phenyl)-1-(5-chloro-2-methyl-phenyl)-2-(3,4-dimethyl-phenyl)-1H-imidazole-4-carboxylic acid
Example 189: 5-(3-Chloro-4-fluoro-phenyl)-1-(3-chloro-2-fluoro-phenyl)-2-phenyl-1H-imidazole-4-carboxylic acid ethyl ester
Example 190: 5-(3-Chloro-4-fluoro-phenyl)-1-(3-chloro-2-fluoro-phenyl)-2-phenyl-1H-imidazole-4-carboxylic acid
Example 191: 1-(3-Chloro-2-fluoro-phenyl)-5-(5-chloro-2-methoxy-phenyl)-2-(6-methyl-pyridin-2-yl)-1H-imidazole-4-carboxylic acid ethyl ester
Example 192: 1-(3-Chloro-2-fluoro-phenyl)-5-(5-chloro-2-methoxy-phenyl)-2-(6-methyl-pyridin-2-yl)-1H-imidazole-4-carboxylic acid
Example 193: 1-(3-Chloro-2-fluoro-phenyl)-5-(5-chloro-2-methoxy-phenyl)-2-(6-methyl-pyridin-2-yl)-1H-imidazole-4-carboxylic acid amide
Example 194: 1-(3-Chloro-2-fluoro-phenyl)-5-(5-chloro-2-methoxy-phenyl)-2-(6-methyl-pyridin-2-yl)-1H-imidazole-4-carbonitrile
Example 195: 2-[1-(3-Chloro-2-fluoro-phenyl)-5-(5-chloro-2-methoxy-phenyl)-4-(2H-tetrazol-5-yl)-1H-imidazol-2-yl]-6-methyl-pyridine
Example 196: 1-(3-Chloro-2-fluoro-phenyl)-5-(3-chloro-phenyl)-2-(6-methyl-pyridin-2-yl)-1H-imidazole-4-carboxylic acid ethyl ester
Example 197: 1-(3-Chloro-2-fluoro-phenyl)-5-(3-chloro-phenyl)-2-(6-methyl-pyridin-2-yl)-1H-imidazole-4-carboxylic acid
Example 198: 1-(3-Chloro-2-fluoro-phenyl)-5-(3-chloro-phenyl)-2-(6-methyl-pyridin-2-yl)-1H-imidazole-4-carboxylic acid amide
Example 199: 1-(3-Chloro-2-fluoro-phenyl)-5-(3-chloro-phenyl)-2-(6-methyl-pyridin-2-yl)-1H-imidazole-4-carbonitrile
Example 200: 2-[1-(3-Chloro-2-fluoro-phenyl)-5-(3-chloro-phenyl)-4-(2H-tetrazol-5-yl)-1H-imidazol-2-yl]-6-methyl-pyridine
Example 201: 1-(5-Chloro-2-methyl-phenyl)-5-(3-chloro-phenyl)-2-cyclohexyl-1H-imidazole-4-carboxylic acid ethyl ester
Example 202: 1-(5-Chloro-2-methyl-phenyl)-5-(3-chloro-phenyl)-2-cyclohexyl-1H-imidazole-4-carboxylic acid lithium salt
Example 203: 1-(5-Chloro-2-methyl-phenyl)-5-(3-chloro-phenyl)-2-cyclohexyl-1H-imidazole-4-carboxylic acid amide
Example 204: 1-(5-Chloro-2-methyl-phenyl)-5-(3-chloro-phenyl)-2-cyclohexyl-1H-imidazole-4-carboxylic acid ethylamide
Example 205: 2-{2-[2-(5-Amino-[1,3,4]oxadiazol-2-yl)-ethylamino]-phenyl}-5-(3-chloro-4-fluoro-phenyl)-1-(3-chloro-2-fluoro-phenyl)-1H-imidazole-4-carboxylic acid amide
Example 206: 5-(3-Chloro-4-fluoro-phenyl)-1-(3-chloro-2-fluoro-phenyl)-2-[2-(2-methyl-4,5-dihydro-imidazol-1-yl)-phenyl]-1H-imidazole-4-carboxylic acid N′-acetyl-hydrazide
Example 207: 2-{5-(3-Chloro-4-fluoro-phenyl)-1-(3-chloro-2-fluoro-phenyl)-2-[2-(2-methyl-4,5-dihydro-imidazol-1-yl)-phenyl]-1H-imidazol-4-yl}-5-methyl-[1,3,4]oxadiazole
Example 208: 5-(3-Chloro-4-fluoro-phenyl)-1-(3-chloro-2-fluoro-phenyl)-2-thiophen-3-yl-1H-imidazole-4-carboxylic acid N′-acetyl-hydrazide
Example 209: 1-(3-Chloro-2-fluoro-phenyl)-5-(5-chloro-2-methoxy-phenyl)-2-cyclohexyl-1H-imidazole-4-carboxylic acid ethyl ester
Example 210: 1-(3-Chloro-2-fluoro-phenyl)-5-(5-chloro-2-methoxy-phenyl)-2-cyclohexyl-1H-imidazole-4-carboxylic acid
Example 211: 5-(5-Chloro-2,4-difluoro-phenyl)-1-(3-chloro-2-fluoro-phenyl)-2-cyclohexyl-1H-imidazole-4-carboxylic acid ethyl ester
Example 212: 5-(5-Chloro-2,4-difluoro-phenyl)-1-(3-chloro-2-fluoro-phenyl)-2-cyclohexyl-1H-imidazole-4-carboxylic acid
Example 213: 5-(3-Chloro-4-fluoro-phenyl)-1-(5-chloro-2-methyl-phenyl)-2-cyclohexyl-1H-imidazole-4-carboxylic acid ethyl ester
Example 214: 5-(3-Chloro-4-fluoro-phenyl)-1-(5-chloro-2-methyl-phenyl)-2-cyclohexyl-1H-imidazole-4-carboxylic acid lithium salt
Example 215: 5-(3-Chloro-4-fluoro-phenyl)-1-(5-chloro-2-methyl-phenyl)-2-cyclohexyl-1H-imidazole-4-carboxylic acid amide
Example 216: 5-(3-Chloro-4-fluoro-phenyl)-1-(5-chloro-2-methyl-phenyl)-2-cyclohexyl-1H-imidazole-4-carboxylic acid ethylamide
Example 217: 5-(3-Chloro-4-fluoro-phenyl)-1-(5-chloro-2-methyl-phenyl)-2-cyclohexyl-1H-imidazole-4-carbonitrile
Example 218: 5-[5-(3-Chloro-4-fluoro-phenyl)-1-(5-chloro-2-methyl-phenyl)-2-cyclohexyl-1H-imidazol-4-yl]-2H-tetrazole
Example 219: 5-(3-Chloro-4-fluoro-phenyl)-1-(3-chloro-2-fluoro-phenyl)-2-cyclohexyl-1H-imidazole-4-carboxylic acid ethyl ester
Example 220: 5-(3-Chloro-4-fluoro-phenyl)-1-(3-chloro-2-fluoro-phenyl)-2-cyclohexyl-1H-imidazole-4-carboxylic acid
Example 221: 5-(3-Chloro-4-fluoro-phenyl)-1-(3-chloro-2-fluoro-phenyl)-2-cyclohexyl-1H-imidazole-4-carboxylic acid amide
Example 222: 5-(3-Chloro-4-fluoro-phenyl)-1-(3-chloro-2-fluoro-phenyl)-2-cyclohexyl-1H-imidazole-4-carbonitrile
Example 223: 5-[5-(3-Chloro-4-fluoro-phenyl)-1-(3-chloro-2-fluoro-phenyl)-2-cyclohexyl-1H-imidazol-4-yl]-2H-tetrazole
Example 224: 2-[5-(3-Chloro-4-fluoro-phenyl)-1-(3-chloro-2-fluoro-phenyl)-2-thiophen-3-yl-1H-imidazol-4-yl]-5-methyl-[1,3,4]oxadiazole
Example 225: 5-(3-Chloro-4-fluoro-phenyl)-1-(3-chloro-2-fluoro-phenyl)-2-thiophen-3-yl-1H-imidazole-4-carboxylic acid hydrazide
Example 226: 5-[5-(3-Chloro-4-fluoro-phenyl)-1-(3-chloro-2-fluoro-phenyl)-2-thiophen-3-yl-1H-imidazol-4-yl]-[1,3,4]oxadiazol-2-ylamine
Example 227: 2-(2-Amino-phenyl)-5-(3-chloro-4-fluoro-phenyl)-1-(3-chloro-2-fluoro-phenyl)-1H-imidazole-4-carboxylic acid amide
Example 228: 5-(3-Chloro-phenyl)-1-(4-chloro-phenyl)-2-(2,2-dimethyl-propyl)-1H-imidazole-4-carboxylic acid ethyl ester
Example 229: 5-(3-Chloro-phenyl)-1-(4-chloro-phenyl)-2-(2,2-dimethyl-propyl)-1H-imidazole-4-carboxylic acid
Example 230: 5-(3-Chloro-phenyl)-1-(4-chloro-phenyl)-2-(2,2-dimethyl-propyl)-1H-imidazole-4-carboxylic acid ethylamide
Example 231: 5-(3-Chloro-phenyl)-1-(4-chloro-phenyl)-2-(2,2-dimethyl-propyl)-1H-imidazole-4-carboxylic acid (2-morpholin-4-yl-ethyl)-amide
Example 232: 2-[2-(2-Amino-ethylamino)-phenyl]-5-(3-chloro-4-fluoro-phenyl)-1-(3-chloro-2-fluoro-phenyl)-1H-imidazole-4-carboxylic acid amide
Example 233: 5-(3-Chloro-4-fluoro-phenyl)-1-(3-chloro-2-fluoro-phenyl)-2-{2-[2-(3-methyl-ureido)-ethylamino]-phenyl}-1H-imidazole-4-carboxylic acid amide
Example 234: N-(2-{2-[4-(N′-Acetyl-hydrazinocarbonyl)-5-(3-chloro-4-fluoro-phenyl)-1-(3-chloro-2-fluoro-phenyl)-1H-imidazol-2-yl]-phenylamino}-ethyl)-acetamide
Example 235: N-(2-{2-[5-(3-Chloro-4-fluoro-phenyl)-1-(3-chloro-2-fluoro-phenyl)-4-hydrazinocarbonyl-1H-imidazol-2-yl]-phenylamino}-ethyl)-acetamide
Example 236: 1,5-Bis-(3-chloro-phenyl)-2-(2,2-dimethyl-propyl)-1H-imidazole-4-carboxylic acid ethyl ester
Example 237: 1,5-Bis-(3-chloro-phenyl)-2-(2,2-dimethyl-propyl)-1H-imidazole-4-carboxylic acid lithium salt
Example 238: 1,5-Bis-(3-chloro-phenyl)-2-(2,2-dimethyl-propyl)-1H-imidazole-4-carboxylic acid ethylamide
Example 239: 1,5-Bis-(3-chloro-phenyl)-2-(2,2-dimethyl-propyl)-1H-imidazole-4-carboxylic acid amide
Example 240: 1-(5-Chloro-2-methyl-phenyl)-5-(3-chloro-phenyl)-2-(2,2-dimethyl-propyl)-1H-imidazole-4-carboxylic acid ethyl ester
Example 241: 1-(5-Chloro-2-methyl-phenyl)-5-(3-chloro-phenyl)-2-(2,2-dimethyl-propyl)-1H-imidazole-4-carboxylic acid
Example 242: 1-(5-Chloro-2-methyl-phenyl)-5-(3-chloro-phenyl)-2-(2,2-dimethyl-propyl)-1H-imidazole-4-carboxylic acid ethylamide
Example 243: 1-(5-Chloro-2-methyl-phenyl)-5-(3-chloro-phenyl)-2-(2,2-dimethyl-propyl)-1H-imidazole-4-carboxylic acid amide
Example 244: 5-(5-Chloro-2-methoxy-phenyl)-1-(5-chloro-2-methyl-phenyl)-2-(2,2-dimethyl-propyl)-1H-imidazole-4-carboxylic acid ethyl ester
Example 245: 5-(5-Chloro-2-methoxy-phenyl)-1-(5-chloro-2-methyl-phenyl)-2-(2,2-dimethyl-propyl)-1H-imidazole-4-carboxylic acid
Example 246: 5-(5-Chloro-2-methoxy-phenyl)-1-(5-chloro-2-methyl-phenyl)-2-(2,2-dimethyl-propyl)-1H-imidazole-4-carboxylic acid ethylamide
Example 247: 5-(5-Chloro-2-methoxy-phenyl)-1-(5-chloro-2-methyl-phenyl)-2-(2,2-dimethyl-propyl)-1H-imidazole-4-carboxylic acid amide
Example 248: 5-(3-Chloro-4-fluoro-phenyl)-1-(5-chloro-2-methyl-phenyl)-2-(2,2-dimethyl-propyl)-1H-imidazole-4-carboxylic acid ethyl ester
Example 249: 5-(3-Chloro-4-fluoro-phenyl)-1-(5-chloro-2-methyl-phenyl)-2-(2,2-dimethyl-propyl)-1H-imidazole-4-carboxylic acid
Example 250: 5-(3-Chloro-4-fluoro-phenyl)-1-(5-chloro-2-methyl-phenyl)-2-(2,2-dimethyl-propyl)-1H-imidazole-4-carboxylic acid amide
Example 251: 5-(3-Chloro-4-fluoro-phenyl)-1-(5-chloro-2-methyl-phenyl)-2-(2,2-dimethyl-propyl)-1H-imidazole-4-carbonitrile
Example 252: 5-[5-(3-Chloro-4-fluoro-phenyl)-1-(5-chloro-2-methyl-phenyl)-2-(2,2-dimethyl-propyl)-1H-imidazol-4-yl]-2H-tetrazole
Example 253: 5-(3-Chloro-4-fluoro-phenyl)-1-(3-chloro-2-fluoro-phenyl)-2-(2,2-dimethyl-propyl)-1H-imidazole-4-carboxylic acid ethyl ester
Example 254: 5-(3-Chloro-4-fluoro-phenyl)-1-(3-chloro-2-fluoro-phenyl)-2-(2,2-dimethyl-propyl)-1H-imidazole-4-carboxylic acid
Example 255: 5-(3-Chloro-4-fluoro-phenyl)-1-(3-chloro-2-fluoro-phenyl)-2-(2,2-dimethyl-propyl)-1H-imidazole-4-carboxylic acid amide
Example 256: 5-(3-Chloro-4-fluoro-phenyl)-1-(3-chloro-2-fluoro-phenyl)-2-(2,2-dimethyl-propyl)-1H-imidazole-4-carbonitrile
Example 257: 5-[5-(3-Chloro-4-fluoro-phenyl)-1-(3-chloro-2-fluoro-phenyl)-2-(2,2-dimethyl-propyl)-1H-imidazol-4-yl]-2H-tetrazole
Example 258: 5-(3-Chloro-4-fluoro-phenyl)-1-(3-chloro-2-fluoro-phenyl)-2-cyclopentylmethyl-1H-imidazole-4-carboxylic acid ethyl ester
Example 259: 5-(3-Chloro-4-fluoro-phenyl)-1-(3-chloro-2-fluoro-phenyl)-2-cyclopentylmethyl-1H-imidazole-4-carboxylic acid
Example 260: 5-(3-Chloro-4-fluoro-phenyl)-1-(3-chloro-2-fluoro-phenyl)-2-cyclopentylmethyl-1H-imidazole-4-carboxylic acid amide
Example 261: 5-(3-Chloro-4-fluoro-phenyl)-1-(3-chloro-2-fluoro-phenyl)-2-cyclopentylmethyl-1H-imidazole-4-carbonitrile
Example 262: 5-[5-(3-Chloro-4-fluoro-phenyl)-1-(3-chloro-2-fluoro-phenyl)-2-cyclopentylmethyl-1H-imidazol-4-yl]-2H-tetrazole
Example 263: 5-(3-Chloro-4-fluoro-phenyl)-1-(3-chloro-2-fluoro-phenyl)-2-cyclohexylmethyl-1H-imidazole-4-carboxylic acid ethyl ester
Example 264: [5-(3-Chloro-4-fluoro-phenyl)-1-(3-chloro-2-fluoro-phenyl)-2-cyclohexylmethyl-1H-imidazol-4-yl]-methanol
Example 265: 5-(3-Chloro-4-fluoro-phenyl)-1-(3-chloro-2-fluoro-phenyl)-2-cyclohexylmethyl-1H-imidazole-4-carboxylic acid
Example 266: 5-(3-Chloro-4-fluoro-phenyl)-1-(3-chloro-2-fluoro-phenyl)-2-cyclohexylmethyl-1H-imidazole-4-carboxylic acid amide
Example 267: 5-(3-Chloro-4-fluoro-phenyl)-1-(3-chloro-2-fluoro-phenyl)-2-cyclohexylmethyl-1H-imidazole-4-carbonitrile
Example 268: 5-[5-(3-Chloro-4-fluoro-phenyl)-1-(3-chloro-2-fluoro-phenyl)-2-cyclohexylmethyl-1H-imidazol-4-yl]-2H-tetrazole
Example 269: 5-(3-Chloro-4-fluoro-phenyl)-1-(3-chloro-2-fluoro-phenyl)-2-thiophen-3-yl-1H-imidazole-4-carboxylic acid
Example 270: 5-(3-Chloro-4-fluoro-phenyl)-1-(3-chloro-2-fluoro-phenyl)-2-thiophen-3-yl-1H-imidazole-4-carboxylic acid amide
Example 271: 5-(3-Chloro-4-fluoro-phenyl)-1-(3-chloro-2-fluoro-phenyl)-2-thiophen-3-yl-1H-imidazole-4-carbonitrile
Example 272: 5-[5-(3-Chloro-4-fluoro-phenyl)-1-(3-chloro-2-fluoro-phenyl)-2-thiophen-3-yl-1H-imidazol-4-yl]-2H-tetrazole
Example 273: 5-(3-Chloro-4-fluoro-phenyl)-1-(3-chloro-2-fluoro-phenyl)-2-(3-hydroxy-phenyl)-1H-imidazole-4-carboxylic acid ethyl ester
Example 274: 5-(3-Chloro-4-fluoro-phenyl)-1-(3-chloro-2-fluoro-phenyl)-2-(3-hydroxy-phenyl)-1H-imidazole-4-carboxylic acid
Example 275: 5-(3-Chloro-4-fluoro-phenyl)-1-(3-chloro-2-fluoro-phenyl)-2-(1H-indol-6-yl)-1H-imidazole-4-carboxylic acid ethyl ester
Example 276: 5-(3-Chloro-4-fluoro-phenyl)-1-(3-chloro-2-fluoro-phenyl)-2-(1H-indol-6-yl)-1H-imidazole-4-carboxylic acid
Example 277: 5-(3-Chloro-4-fluoro-phenyl)-1-(3-chloro-2-fluoro-phenyl)-2-(1H-indol-6-yl)-1H-imidazole-4-carboxylic acid amide
Example 278: 5-(3-Chloro-4-fluoro-phenyl)-1-(3-chloro-2-fluoro-phenyl)-2-(1-methyl-1H-indol-5-yl)-1H-imidazole-4-carboxylic acid ethyl ester
Example 279: 5-(3-Chloro-4-fluoro-phenyl)-1-(3-chloro-2-fluoro-phenyl)-2-(1-methyl-1H-indol-5-yl)-1H-imidazole-4-carboxylic acid
Example 280: 2-Benzo[b]thiophen-5-yl-5-(3-chloro-4-fluoro-phenyl)-1-(3-chloro-2-fluoro-phenyl)-1H-imidazole-4-carboxylic acid ethyl ester
Example 281: 2-Benzo[b]thiophen-5-yl-5-(3-chloro-4-fluoro-phenyl)-1-(3-chloro-2-fluoro-phenyl)-1H-imidazole-4-carboxylic acid
Example 282: 2-Benzo[b]thiophen-5-yl-5-(3-chloro-4-fluoro-phenyl)-1-(3-chloro-2-fluoro-phenyl)-1H-imidazole-4-carboxylic acid amide
Example 283: 5-(3-Chloro-4-fluoro-phenyl)-1-(3-chloro-2-fluoro-phenyl)-2-(1H-indol-3-yl)-1H-imidazole-4-carboxylic acid ethyl ester
Example 284: 5-(3-Chloro-4-fluoro-phenyl)-1-(3-chloro-2-fluoro-phenyl)-2-(1H-indol-3-yl)-1H-imidazole-4-carboxylic acid
Example 285: 5-(3-Chloro-4-fluoro-phenyl)-1-(3-chloro-2-fluoro-phenyl)-2-(1-methyl-1H-indol-6-yl)-1H-imidazole-4-carboxylic acid ethyl ester
Example 286: 5-(3-Chloro-4-fluoro-phenyl)-1-(3-chloro-2-fluoro-phenyl)-2-(1-methyl-1H-indol-6-yl)-1H-imidazole-4-carboxylic acid
Example 287: 5-(3-Chloro-4-fluoro-phenyl)-1-(3-chloro-2-fluoro-phenyl)-2-(1-methyl-1H-indol-6-yl)-1H-imidazole-4-carboxylic acid amide
Example 288: [5-(3-Chloro-4-fluoro-phenyl)-1-(3-chloro-2-fluoro-phenyl)-2-(1-methyl-1H-indol-6-yl)-1H-imidazol-4-yl]-methanol
Example 289: 5-(3-Chloro-4-fluoro-phenyl)-1-(3-chloro-2-fluoro-phenyl)-2-(3-chloro-phenyl)-1H-imidazole-4-carboxylic acid ethyl ester
Example 290: 5-(3-Chloro-4-fluoro-phenyl)-1-(3-chloro-2-fluoro-phenyl)-2-(3-chloro-phenyl)-1H-imidazole-4-carboxylic acid
Example 291: 5-(3-Chloro-4-fluoro-phenyl)-1-(3-chloro-2-fluoro-phenyl)-2-(3-chloro-phenyl)-1H-imidazole-4-carboxylic acid amide
Example 292: 5-(3-Chloro-4-fluoro-phenyl)-1-(3-chloro-2-fluoro-phenyl)-2-(5-formyl-thiophen-3-yl)-1H-imidazole-4-carboxylic acid ethyl ester
Example 293: 5-(3-Chloro-4-fluoro-phenyl)-1-(3-chloro-2-fluoro-phenyl)-2-(5-formyl-thiophen-3-yl)-1H-imidazole-4-carboxylic acid
Example 294: 2-(2-Amino-phenyl)-5-(3-chloro-4-fluoro-phenyl)-1-(3-chloro-2-fluoro-phenyl)-1H-imidazole-4-carboxylic acid ethyl ester
Example 295: 2-(2-Amino-phenyl)-5-(3-chloro-4-fluoro-phenyl)-1-(3-chloro-2-fluoro-phenyl)-1H-imidazole-4-carboxylic acid
Example 296: 2-[2-(2-Acetylamino-ethylamino)-phenyl]-5-(3-chloro-4-fluoro-phenyl)-1-(3-chloro-2-fluoro-phenyl)-1H-imidazole-4-carboxylic acid ethyl ester
Example 297: 2-[2-(2-Acetylamino-ethylamino)-phenyl]-5-(3-chloro-4-fluoro-phenyl)-1-(3-chloro-2-fluoro-phenyl)-1H-imidazole-4-carboxylic acid
Example 298: 2-[2-(2-Acetylamino-ethylamino)-phenyl]-5-(3-chloro-4-fluoro-phenyl)-1-(3-chloro-2-fluoro-phenyl)-1H-imidazole-4-carboxylic acid amide
Example 299: 5-(3-Chloro-4-fluoro-phenyl)-1-(3-chloro-2-fluoro-phenyl)-2-(5-hydroxymethyl-thiophen-3-yl)-1H-imidazole-4-carboxylic acid ethyl ester
Example 300: 5-(3-Chloro-4-fluoro-phenyl)-1-(3-chloro-2-fluoro-phenyl)-2-(5-hydroxymethyl-thiophen-3-yl)-1H-imidazole-4-carboxylic acid
Example 301: 5-(3-Chloro-4-fluoro-phenyl)-1-(5-chloro-2-methyl-phenyl)-2-m-tolyl-1H-imidazole-4-carboxylic acid ethyl ester
Example 302: 5-(3-Chloro-4-fluoro-phenyl)-1-(5-chloro-2-methyl-phenyl)-2-m-tolyl-1H-imidazole-4-carboxylic acid
Example 303: 5-(3-Chloro-4-fluoro-phenyl)-1-(5-chloro-2-methyl-phenyl)-2-thiophen-3-yl-1H-imidazole-4-carboxylic acid ethyl ester
Example 304: 5-(3-Chloro-4-fluoro-phenyl)-1-(5-chloro-2-methyl-phenyl)-2-thiophen-3-yl-1H-imidazole-4-carboxylic acid
Example 305: 5-(3-Chloro-4-fluoro-phenyl)-1-(5-chloro-2-methyl-phenyl)-2-(6-methoxy-pyridin-2-yl)-1H-imidazole-4-carboxylic acid ethyl ester
Example 306: 5-(3-Chloro-4-fluoro-phenyl)-1-(5-chloro-2-methyl-phenyl)-2-(6-methoxy-pyridin-2-yl)-1H-imidazole-4-carboxylic acid
Example 307: 1-(3-Chloro-2-fluoro-phenyl)-5-(5-chloro-2-hydroxy-phenyl)-2-phenyl-1H-imidazole-4-carboxylic acid ethyl ester
Example 308: N-(2-{2-[4-(5-Amino-[1,3,4]oxadiazol-2-yl)-5-(3-chloro-4-fluoro-phenyl)-1-(3-chloro-2-fluoro-phenyl)-1H-imidazol-2-yl]-phenylaminoyethyl}-acetamide
Example 309: 5′-(3-Chloro-4-fluoro-phenyl)-1′-(3-chloro-2-fluoro-phenyl)-2′-phenyl-1′H-[1,4]biimidazolyl
Example 310: [5-(3-Chloro-4-fluoro-phenyl)-1-(3-chloro-2-fluoro-phenyl)-2-cyclohexyl-1H-imidazol-4-yl]-phosphonic acid diethyl ester
Example 311: [5-(3-Chloro-4-fluoro-phenyl)-1-(3-chloro-2-fluoro-phenyl)-2-cyclohexyl-1H-imidazol-4-yl]-phosphonic acid monoethyl ester
Example 312: [5-(3-Chloro-4-fluoro-phenyl)-1-(3-chloro-2-fluoro-phenyl)-2-cyclohexyl-1H-imidazol-4-yl]-phosphonic acid
Example 313: 2-(3-Chloro-benzyl)-1-(5-chloro-2-methyl-phenyl)-5-(3-chloro-phenyl)-1H-imidazole-4-carboxylic acid ethyl ester
Example 314: 2-(3-Chloro-benzyl)-1-(5-chloro-2-methyl-phenyl)-5-(3-chloro-phenyl)-1H-imidazole-4-carboxylic acid
Example 315: [5-(3-Chloro-4-fluoro-phenyl)-1-(3-chloro-2-fluoro-phenyl)-2-phenyl-1H-imidazol-4-yl]-phosphonic acid diethyl ester
Example 316: 5-(3-Chloro-4-fluoro-phenyl)-1-(3-chloro-2-fluoro-phenyl)-2-m-tolyl-1H-imidazole-4-NH-methyl-sulfoximine
Example 317: [5-(3-Chloro-4-fluoro-phenyl)-1-(3-chloro-2-fluoro-phenyl)-2-phenyl-1H-imidazol-4-yl]-phosphonic acid monoethyl ester
Example 318: [5-(3-Chloro-4-fluoro-phenyl)-1-(3-chloro-2-fluoro-phenyl)-2-phenyl-1H-imidazol-4-yl]-phosphonic acid
Example 319: 5-[5-(3-Chloro-4-fluoro-phenyl)-1-(3-chloro-2-fluoro-phenyl)-2-phenyl-1H-imidazol-4-yl]-3H-[1,3,4]oxadiazol-2-one
Example 320: 5-(3-Chloro-4-fluoro-phenyl)-1-(3-chloro-2-fluoro-phenyl)-2-phenyl-1H-imidazole-4-carboxylic acid hydrazide
Example 321: 5-[5-(3-Chloro-4-fluoro-phenyl)-1-(3-chloro-2-fluoro-phenyl)-2-phenyl-1H-imidazol-4-yl]-[1,3,4]oxadiazol-2-ylamine
Example 322: 2-[5-(3-Chloro-4-fluoro-phenyl)-1-(3-chloro-2-fluoro-phenyl)-2-phenyl-1H-imidazol-4-yl]-5-methyl-[1,3,4]oxadiazole
Example 323: 5-[5-(3-Chloro-4-fluoro-phenyl)-1-(3-chloro-2-fluoro-phenyl)-2-phenyl-1H-imidazol-4-yl]-4-methyl-2,4-dihydro-[1,2,4]triazole-3-thione
Example 324: 5-(3-Chloro-4-fluoro-phenyl)-1-(3-chloro-2-fluoro-phenyl)-4-methanesulfonyl-2-m-tolyl-1H-imidazole
Example 325: 5-(3-Chloro-phenyl)-1-(4-chloro-phenyl)-2-phenyl-1H-imidazole-4-carboxylic acid ethyl ester
Example 326: 5-(3-Chloro-phenyl)-1-(4-chloro-phenyl)-2-phenyl-1H-imidazole-4-carboxylic acid
Example 327: 5-(3-Chloro-phenyl)-1-(4-chloro-phenyl)-2-phenyl-1H-imidazole-4-carboxylic acid methylamide
Example 328: 5-(3-Chloro-phenyl)-1-(4-chloro-phenyl)-2-phenyl-1H-imidazole-4-carboxylic acid (2-morpholin-4-yl-ethyl)-amide
Example 329: 5-(3-Chloro-phenyl)-1-(4-chloro-phenyl)-2-phenyl-1H-imidazole-4-carboxylic acid (3-dimethylamino-propyl)-methyl-amide
Example 330: 5-(3-Chloro-phenyl)-1-(4-chloro-phenyl)-2-phenyl-1H-imidazole-4-carboxylic acid cyclopropylmethyl-amide
Example 331: 5-[5-(3-Chloro-4-fluoro-phenyl)-1-(3-chloro-2-fluoro-phenyl)-2-phenyl-1H-imidazol-4-yl]-3-methyl-[1,2,4]oxadiazole
Example 332: 1-[2-(Acetylamino-methyl)-5-chloro-phenyl]-5-(3-chloro-4-fluoro-phenyl)-2-cyclohexyl-1H-imidazole-4-carboxylic acid
Example 333: 5-(3-Chloro-4-fluoro-phenyl)-1-(3-chloro-2-fluoro-phenyl)-2-(6-methyl-pyridin-2-yl)-1H-imidazole-4-carboxylic acid ethyl ester
Example 334: 5-(3-Chloro-4-fluoro-phenyl)-1-(3-chloro-2-fluoro-phenyl)-2-(6-methyl-pyridin-2-yl)-1H-imidazole-4-carboxylic acid
Example 335: 5-(3-Chloro-4-fluoro-phenyl)-1-(3-chloro-2-fluoro-phenyl)-2-(6-methyl-pyridin-2-yl)-1H-imidazole-4-carboxylic acid amide
Example 336: 5-(3-Chloro-4-fluoro-phenyl)-1-(3-chloro-2-fluoro-phenyl)-2-(6-methyl-pyridin-2-yl)-1H-imidazole-4-carbonitrile
Example 337: 2-[5-(3-Chloro-4-fluoro-phenyl)-1-(3-chloro-2-fluoro-phenyl)-4-(2H-tetrazol-5-yl)-1H-imidazol-2-yl]-6-methyl-pyridine
Example 338: 3-[1-(5-Chloro-2-methyl-phenyl)-5-(3-chloro-phenyl)-4-ethoxycarbonyl-1H-imidazol-2-yl]-piperidine-1-carboxylic acid benzyl ester
Example 339: 1-(5-Chloro-2-methyl-phenyl)-5-(3-chloro-phenyl)-2-piperidin-3-yl-1H-imidazole-4-carboxylic acid ethyl ester
Example 340: 1-(5-Chloro-2-methyl-phenyl)-5-(3-chloro-phenyl)-2-piperidin-3-yl-1H-imidazole-4-carboxylic acid
Example 341: 1-(5-Chloro-2-methyl-phenyl)-5-(3-chloro-phenyl)-2-(1-methyl-piperidin-3-yl)-1H-imidazole-4-carboxylic acid ethyl ester
Example 342: 1-(5-Chloro-2-methyl-phenyl)-5-(3-chloro-phenyl)-2-(1-methyl-piperidin-3-yl)-1H-imidazole-4-carboxylic acid
Example 343: 1-[2-(Acetylamino-methyl)-5-chloro-phenyl]-5-(3-chloro-4-fluoro-phenyl)-2-cyclohexyl-1H-imidazole-4-carboxylic acid amide
Example 344: {5-[5-(3-Chloro-4-fluoro-phenyl)-1-(3-chloro-2-fluoro-phenyl)-2-cyclohexyl-1H-imidazol-4-yl]-[1,3,4]oxadiazol-2-yl}-methyl-amine
Example 345: 3-[5-(3-Chloro-4-fluoro-phenyl)-1-(3-chloro-2-fluoro-phenyl)-2-phenyl-1H-imidazol-4-yl]-1H-pyrazole-4-carboxylic acid ethylamide
Example 346: 2-[3-(tert-Butyl-diphenyl-silanyloxy)-cyclohexyl]-5-(3-chloro-4-fluoro-phenyl)-1-(5-chloro-2-methyl-phenyl)-1H-imidazole-4-carboxylic acid ethyl ester
Example 347: 2-[3-(tert-Butyl-diphenyl-silanyloxy)-cyclohexyl]-5-(3-chloro-4-fluoro-phenyl)-1-(5-chloro-2-methyl-phenyl)-1H-imidazole-4-carboxylic acid
Example 348: 5-(3-Chloro-4-fluoro-phenyl)-1-(5-chloro-2-methyl-phenyl)-2-(3-hydroxy-cyclohexyl)-1H-imidazole-4-carboxylic acid
Example 349: 2-[3-(tert-Butyl-diphenyl-silanyloxy)-cyclohexyl]-5-(3-chloro-4-fluoro-phenyl)-1-(3-chloro-2-fluoro-phenyl)-1H-imidazole-4-carboxylic acid ethyl ester
Example 350: 2-[3-(tert-Butyl-diphenyl-silanyloxy)-cyclohexyl]-5-(3-chloro-4-fluoro-phenyl)-1-(3-chloro-2-fluoro-phenyl)-1H-imidazole-4-carboxylic acid
Example 351: 2-[3-(tert-Butyl-diphenyl-silanyloxy)-cyclohexyl]-5-(3-chloro-4-fluoro-phenyl)-1-(3-chloro-2-fluoro-phenyl)-1H-imidazole-4-carboxylic acid amide
Example 352: 5-(3-Chloro-4-fluoro-phenyl)-1-(3-chloro-2-fluoro-phenyl)-2-(3-hydroxy-cyclohexyl)-1H-imidazole-4-carboxylic acid
Example 353: 3-[5-(3-Chloro-4-fluoro-phenyl)-1-(3-chloro-2-fluoro-phenyl)-4-(2H-tetrazol-5-yl)-1H-imidazol-2-yl]-cyclohexanol
Example 354: 1-(3-Chloro-2-fluoro-phenyl)-5-(5-chloro-2-formyl-phenyl)-2-phenyl-1H-imidazole-4-carboxylic acid ethyl ester
Example 355: 5-(5-Chloro-2-{[(3-dimethylamino-propyl)-methyl-amino]-methyl}-phenyl)-1-(3-chloro-2-fluoro-phenyl)-2-phenyl-1H-imidazole-4-carboxylic acid ethyl ester
Example 356: 5-(5-Chloro-2-{[(3-dimethylamino-propyl)-methyl-amino]-methyl}-phenyl)-1-(3-chloro-2-fluoro-phenyl)-2-phenyl-1H-imidazole-4-carboxylic acid
Example 357: 5-(5-Chloro-2-dimethylaminomethyl-phenyl)-1-(3-chloro-2-fluoro-phenyl)-2-phenyl-1H-imidazole-4-carboxylic acid ethyl ester
Example 358: 5-(5-Chloro-2-dimethylaminomethyl-phenyl)-1-(3-chloro-2-fluoro-phenyl)-2-phenyl-1H-imidazole-4-carboxylic acid
Example 359: 1-(3-Chloro-2-fluoro-phenyl)-5-[5-chloro-2-(4-methyl-piperazin-1-ylmethyl)-phenyl]-2-phenyl-1H-imidazole-4-carboxylic acid ethyl ester
Example 360: 1-(3-Chloro-2-fluoro-phenyl)-5-[5-chloro-2-(4-methyl-piperazin-1-ylmethyl)-phenyl]-2-phenyl-1H-imidazole-4-carboxylic acid
Example 361: 5-(5-Chloro-2-dimethylaminomethyl-phenyl)-1-(3-chloro-2-fluoro-phenyl)-2-phenyl-1H-imidazole-4-carboxylic acid amide
Example 362: 1-(3-Chloro-2-fluoro-phenyl)-5-[5-chloro-2-(4-methyl-piperazin-1-ylmethyl)-phenyl]-2-phenyl-1H-imidazole-4-carboxylic acid amide
Example 363: 1-(3-Chloro-2-fluoro-phenyl)-5-[5-chloro-2-(4-methyl-piperazin-1-ylmethyl)-phenyl]-2-phenyl-1H-imidazole-4-carbonitrile
Example 364: 1-{4-Chloro-2-[3-(3-chloro-2-fluoro-phenyl)-2-phenyl-5-(2H-tetrazol-5-yl)-3H-imidazol-4-yl]-benzyl}-4-methyl-piperazine
Example 365: 1-(3-Chloro-2-fluoro-phenyl)-5-(5-chloro-2-formyl-phenyl)-2-cyclohexyl-1H-imidazole-4-carboxylic acid ethyl ester
Example 366: 5-(5-Chloro-2-dimethylaminomethyl-phenyl)-1-(3-chloro-2-fluoro-phenyl)-2-cyclohexyl-1H-imidazole-4-carboxylic acid ethyl ester
Example 367: 5-(5-Chloro-2-dimethylaminomethyl-phenyl)-1-(3-chloro-2-fluoro-phenyl)-2-cyclohexyl-1H-imidazole-4-carboxylic acid
Example 368: 1-(3-Chloro-2-fluoro-phenyl)-5-[5-chloro-2-(4-methyl-piperazin-1-ylmethyl)-phenyl]-2-cyclohexyl-1H-imidazole-4-carboxylic acid ethyl ester
Example 369: 1-(3-Chloro-2-fluoro-phenyl)-5-[5-chloro-2-(4-methyl-piperazin-1-ylmethyl)-phenyl]-2-cyclohexyl-1H-imidazole-4-carboxylic acid
Example 370: 1-(3-Chloro-2-fluoro-phenyl)-5-[5-chloro-2-(4-methyl-piperazin-1-ylmethyl)-phenyl]-2-cyclohexyl-1H-imidazole-4-carboxylic acid amide
Example 371: 1-(2-Carboxymethyl-5-chloro-phenyl)-5-(3-chloro-4-fluoro-phenyl)-2-cyclohexyl-1H-imidazole-4-carboxylic acid ethyl ester
Example 372: 1-(2-tert-Butoxycarbonylmethyl-5-chloro-phenyl)-5-(3-chloro-4-fluoro-phenyl)-2-cyclohexyl-1H-imidazole-4-carboxylic acid ethyl ester
Example 373: 1-(2-Carboxymethyl-5-chloro-phenyl)-5-(3-chloro-4-fluoro-phenyl)-2-cyclohexyl-1H-imidazole-4-carboxylic acid
Example 374: 1-(2-Carbamoylmethyl-5-chloro-phenyl)-5-(3-chloro-4-fluoro-phenyl)-2-cyclohexyl-1H-imidazole-4-carboxylic acid ethyl ester
Example 375: 5-(3-Chloro-4-fluoro-phenyl)-1-(5-chloro-2-methylcarbamoylmethyl-phenyl)-2-cyclohexyl-1H-imidazole-4-carboxylic acid ethyl ester
Example 376: 1-(2-Carbamoylmethyl-5-chloro-phenyl)-5-(3-chloro-4-fluoro-phenyl)-2-cyclohexyl-1H-imidazole-4-carboxylic acid
Example 377: 5-(3-Chloro-4-fluoro-phenyl)-1-(5-chloro-2-methylcarbamoylmethyl-phenyl)-2-cyclohexyl-1H-imidazole-4-carboxylic acid
Example 378: 3-Chloro-5-[3-(3-chloro-2-fluoro-phenyl)-5-cyano-2-cyclohexyl-3H-imidazol-4-yl]-benzoic acid
Example 379: 3-Chloro-5-[3-(3-chloro-2-fluoro-phenyl)-5-cyano-2-cyclohexyl-3H-imidazol-4-yl]-N-methyl-benzamide
Example 380: 3-Chloro-5-[3-(3-chloro-2-fluoro-phenyl)-2-phenyl-5-(2H-tetrazol-5-yl)-3H-imidazol-4-yl]-N-methyl-benzamide
Example 381: 3-[5-(3-Chloro-4-fluoro-phenyl)-1-(3-chloro-2-fluoro-phenyl)-2-phenyl-1H-imidazol-4-yl]-1-(2-trimethylsilanyl-ethoxymethyl)-1H-pyrazole-4-carboxylic acid
Example 382: 3-[5-(3-Chloro-4-fluoro-phenyl)-1-(3-chloro-2-fluoro-phenyl)-2-phenyl-1H-imidazol-4-yl]-1-(2-trimethylsilanyl-ethoxymethyl)-1H-pyrazole-4-carboxylic acid methyl ester
Example 383: 5-(3-Chloro-4-fluoro-phenyl)-1-(3-chloro-2-fluoro-phenyl)-2-piperidin-1-ylmethyl-1H-imidazole-4-carboxylic acid
Example 384: 5-(3-Chloro-4-fluoro-phenyl)-1-(3-chloro-2-fluoro-phenyl)-2-pyrrolidin-1-ylmethyl-1H-imidazole-4-carboxylic acid ethyl ester
Example 385: 5-(3-Chloro-4-fluoro-phenyl)-1-(3-chloro-2-fluoro-phenyl)-2-(R)-pyrrolidin-2-yl-1H-imidazole-4-carboxylic acid ethyl ester
Example 386: 3-Chloro-5-[3-(3-chloro-2-fluoro-phenyl)-5-cyano-2-phenyl-3H-imidazol-4-yl]-benzoic acid methyl ester
Example 387: 5-{3-Chloro-5-[3-(3-chloro-2-fluoro-phenyl)-2-phenyl-5-(2H-tetrazol-5-yl)-3H-imidazol-4-yl]-phenyl}-[1,2,3,4]oxatriazole
Example 388: 5-(3-Chloro-4-fluoro-phenyl)-1-(5-chloro-2-methyl-phenyl)-2-(3-chloro-phenyl)-1H-imidazole-4-carboxylic acid ethyl ester
Example 389: 5-(3-Chloro-4-fluoro-phenyl)-1-(5-chloro-2-methyl-phenyl)-2-(3-chloro-phenyl)-1H-imidazole-4-carboxylic acid
Example 390: 1-(5-Chloro-2-methoxy-pyridin-3-yl)-5-(3-chloro-phenyl)-2-phenyl-1H-imidazole-4-carboxylic acid ethyl ester
Example 391: 1-(5-Chloro-2-methoxy-pyridin-3-yl)-5-(3-chloro-phenyl)-2-phenyl-1H-imidazole-4-carboxylic acid
Example 392: 1-(5-Chloro-2-methoxy-pyridin-3-yl)-5-(3-chloro-phenyl)-2-phenyl-1H-imidazole-4-carboxylic acid amide
Example 393: 1-(5-Chloro-2-methoxy-pyridin-3-yl)-5-(3-chloro-phenyl)-2-phenyl-1H-imidazole-4-carbonitrile
Example 394: 5-Chloro-3-[5-(3-chloro-phenyl)-2-phenyl-4-(2H-tetrazol-5-yl)-imidazol-1-yl]-2-methoxy-pyridine
Example 395: 1-(6-Carboxymethyl-3-chloro-2-fluoro-phenyl)-5-(3-chloro-4-fluoro-phenyl)-2-cyclohexyl-1H-imidazole-4-carboxylic acid ethyl ester
Example 396: 1-(6-tert-Butoxycarbonylmethyl-3-chloro-2-fluoro-phenyl)-5-(3-chloro-4-fluoro-phenyl)-2-cyclohexyl-1H-imidazole-4-carboxylic acid ethyl ester
Example 397: -(6-tert-Butoxycarbonylmethyl-3-chloro-2-fluoro-phenyl)-5-(3-chloro-4-fluoro-phenyl)-2-cyclohexyl-1H-imidazole-4-carboxylic acid
Example 398: 1-(6-Carboxymethyl-3-chloro-2-fluoro-phenyl)-5-(3-chloro-4-fluoro-phenyl)-2-cyclohexyl-1H-imidazole-4-carboxylic acid
Example 399: {2-[4-Carbamoyl-5-(3-chloro-4-fluoro-phenyl)-2-cyclohexyl-imidazol-1-yl]-4-chloro-3-fluoro-phenyl}-acetic acid tert-butyl ester
Example 400: {2-[4-Carbamoyl-5-(3-chloro-4-fluoro-phenyl)-2-cyclohexyl-imidazol-1-yl]-4-chloro-3-fluoro-phenyl}-acetic acid methyl ester
Example 401: {2-[4-Carbamoyl-5-(3-chloro-4-fluoro-phenyl)-2-cyclohexyl-imidazol-1-yl]-4-chloro-3-fluoro-phenyl}-acetic acid
Example 402: N′-{5-[5-(3-Chloro-4-fluoro-phenyl)-1-(3-chloro-2-fluoro-phenyl)-2-cyclohexyl-1H-imidazol-4-yl]-[1,3,4]oxadiazol-2-yl}-N,N-dimethyl-ethane-1,2-diamine
Example 403: {5-[5-(3-Chloro-4-fluoro-phenyl)-1-(3-chloro-2-fluoro-phenyl)-2-cyclohexyl-1H-imidazol-4-yl]-[1,3,4]oxadiazol-2-yl}-(3-methoxy-propyl)-amine
Example 404: Benzyl-{5-[5-(3-chloro-4-fluoro-phenyl)-1-(3-chloro-2-fluoro-phenyl)-2-cyclohexyl-1H-imidazol-4-yl]-[1,3,4]oxadiazol-2-yl}-amine
Example 405: {4-Chloro-2-[5-(3-chloro-4-fluoro-phenyl)-2-cyclohexyl-4-hydrazinocarbonyl-imidazol-1-yl]-3-fluoro-phenyl}-acetic acid tert-butyl ester
Example 406: 1-(5-Chloro-2-methyl-phenyl)-5-(2-chloro-pyridin-4-yl)-2-(3,4-dimethyl-phenyl)-1H-imidazole-4-carboxylic acid ethyl ester
Example 407: 1-(5-Chloro-2-methyl-phenyl)-5-(2-chloro-pyridin-4-yl)-2-(3,4-dimethyl-phenyl)-1H-imidazole-4-carboxylic acid
Example 408: 1-(5-Chloro-2-methyl-phenyl)-2-(3-chloro-phenyl)-5-phenyl-1H-pyrrole-3-carboxylic acid ethyl ester
Example 409: 1-(5-Chloro-2-methyl-phenyl)-2-(3-chloro-phenyl)-5-phenyl-1H-pyrrole-3-carboxylic acid
Example 410: 4-(3-Chloro-2-fluoro-phenyl)-3-(3-chloro-phenyl)-5-phenyl-1H-pyrrole-2-carboxylic acid methyl ester
Example 411: 1-(3-Chloro-2-fluoro-phenyl)-2-(3-chloro-phenyl)-5-(3-nitro-phenyl)-1H-pyrrole-3-carboxylic acid ethyl ester
Example 412: 5-(3-Amino-phenyl)-1-(3-chloro-2-fluoro-phenyl)-2-(3-chloro-phenyl)-1H-pyrrole-3-carboxylic acid ethyl ester
Example 413: 5-(3-Amino-phenyl)-1-(3-chloro-2-fluoro-phenyl)-2-(3-chloro-phenyl)-1H-pyrrole-3-carboxylic acid
Example 414: 1-(3-Chloro-2-fluoro-phenyl)-2-(3-chloro-phenyl)-5-(3-dimethylamino-phenyl)-1H-pyrrole-3-carboxylic acid ethyl ester
Example 415: 1-(3-Chloro-2-fluoro-phenyl)-2-(3-chloro-phenyl)-5-(3-dimethylamino-phenyl)-1H-pyrrole-3-carboxylic acid
Example 416: 1-[5-Chloro-2-(2-hydroxy-ethyl)-phenyl]-2-(3-chloro-phenyl)-5-phenyl-1H-pyrrole-3-carboxylic acid
Example 417: 2-(3-Chloro-4-fluoro-phenyl)-1-[5-chloro-2-(2-hydroxy-ethyl)-phenyl]-5-phenyl-1H-pyrrole-3-carboxylic acid ethyl ester
Example 418: 2-(3-Chloro-4-fluoro-phenyl)-1-[5-chloro-2-(2-hydroxy-ethyl)-phenyl]-5-phenyl-1H-pyrrole-3-carboxylic acid
Example 419: 1-(2-Carboxymethyl-5-chloro-phenyl)-2-(3-chloro-4-fluoro-phenyl)-5-phenyl-1H-pyrrole-3-carboxylic acid ethyl ester
Example 420: 1-(5-Chloro-2-methylcarbamoylmethyl-phenyl)-2-(3-chloro-phenyl)-5-phenyl-1H-pyrrole-3-carboxylic acid ethyl ester
Example 421: 1-(5-Chloro-2-methylcarbamoylmethyl-phenyl)-2-(3-chloro-phenyl)-5-phenyl-1H-pyrrole-3-carboxylic acid
Example 422: 1-(2-Carbamoylmethyl-5-chloro-phenyl)-2-(3-chloro-phenyl)-5-phenyl-1H-pyrrole-3-carboxylic acid ethyl ester
Example 423: 1-(2-Carbamoylmethyl-5-chloro-phenyl)-2-(3-chloro-phenyl)-5-phenyl-1H-pyrrole-3-carboxylic acid
Example 424: 1-(2-Carbamoylmethyl-5-chloro-phenyl)-2-(3-chloro-4-fluoro-phenyl)-5-phenyl-1H-pyrrole-3-carboxylic acid
Example 425: 2-(3-Chloro-4-fluoro-phenyl)-1-[5-chloro-2-(2-hydroxy-ethyl)-phenyl]-5-phenyl-1H-pyrrole-3-carboxylic acid amide
Example 426: 4-(5-Chloro-2-methoxy-phenyl)-5-(3-chloro-phenyl)-1-phenyl-1H-pyrazole-3-carboxylic acid lithium salt
Example 427: 4-(3-Chloro-2-fluoro-phenyl)-3-(3-chloro-phenyl)-5-phenyl-1H-pyrrole-2-carboxylic acid
Example 428: 5-(3-Chloro-phenyl)-4-(3,4-dichloro-phenyl)-1-phenyl-1H-pyrazole-3-carboxylic acid
Example 429: 4-(3-Chloro-4-fluoro-phenyl)-5-(3-chloro-phenyl)-1-phenyl-1H-pyrazole-3-carboxylic acid
Example 430: 4,5-Bis-(3-chloro-phenyl)-1-cyclohexyl-1H-pyrazole-3-carboxylic acid
Example 431: 4-(5-Chloro-2-methoxy-phenyl)-5-(3-chloro-phenyl)-1-m-tolyl-1H-pyrazole-3-carboxylic acid lithium salt
Example 432: 4,5-Bis-(3-chloro-phenyl)-1-m-tolyl-1H-pyrazole-3-carboxylic acid
Example 433: 5-(3-Chloro-phenyl)-4-(3,4-dichloro-phenyl)-1-m-tolyl-1H-pyrazole-3-carboxylic acid
Example 434: 4-(3-Chloro-4-fluoro-phenyl)-5-(3-chloro-phenyl)-1-m-tolyl-1H-pyrazole-3-carboxylic acid
Example 435: 1-[5-(3-Chloro-4-fluoro-phenyl)-4-(3-chloro-2-fluoro-phenyl)-3-m-tolyl-pyrazol-1-yl]-2-hydroxy-ethanone
Example 436: 2-[5-(3-Chloro-4-fluoro-phenyl)-1-(3-chloro-2-fluoro-phenyl)-2-phenyl-1H-imidazol-4-yl]-4-methyl-oxazole
Example 437: 5-(3-Chloro-4-fluoro-phenyl)-1-(3-chloro-2-fluoro-phenyl)-2-phenyl-1H-imidazole-4-carboxylic acid (2-oxo-propyl)-amide
Example 438: 1-(5-Chloro-2-methyl-phenyl)-5-(3-chloro-phenyl)-2-phenyl-1H-imidazol-4-ylamine
Example 439: [1-(5-Chloro-2-methyl-phenyl)-5-(3-chloro-phenyl)-2-phenyl-1H-imidazol-4-yl]-carbamic acid tert-butyl ester
Example 440: N-[1-(5-Chloro-2-methyl-phenyl)-5-(3-chloro-phenyl)-2-phenyl-1H-imidazol-4-yl]-acetamide
Example 441: [5-(3-Chloro-2-fluoro-phenyl)-1-(3-chloro-phenyl)-2-phenyl-1H-imidazol-4-yl]-methanol
Example 442: [5-(3-Chloro-4-fluoro-phenyl)-1-(3-chloro-2-fluoro-phenyl)-2-phenyl-1H-imidazol-4-yl]-methanol
Example 443: [5-(3-Chloro-4-fluoro-phenyl)-1-(3-chloro-2-fluoro-phenyl)-2-phenyl-1H-imidazol-4-yl]-acetonitrile
Example 444: 5-[5-(3-Chloro-4-fluoro-phenyl)-1-(3-chloro-2-fluoro-phenyl)-2-phenyl-1H-imidazol-4-ylmethyl]-2H-tetrazole
Example 445: [1-(3-Chloro-2-fluoro-phenyl)-5-(5-chloro-2-methoxy-phenyl)-2-phenyl-1H-imidazol-4-yl]-methanol
Example 446: [1-(3-Chloro-2-fluoro-phenyl)-5-(5-chloro-2-methoxy-phenyl)-2-phenyl-1H-imidazol-4-yl]-acetonitrile
Example 447: [1-(3-Chloro-2-fluoro-phenyl)-5-(5-chloro-2-methoxy-phenyl)-2-phenyl-1H-imidazol-4-yl]-acetic acid
Example 448: 1-[3-(3-Chloro-4-fluoro-phenyl)-4-(3-chloro-2-fluoro-phenyl)-5-m-tolyl-pyrazol-1-yl]-2-hydroxy-ethanone
Example 449: 1-(5-Chloro-2-oxo-1,2,-dihydro-pyridin-3-yl)-5-(3-chloro-phenyl)-2-phenyl-2-phenyl-1-H-imidazole carboxylic acid
Example 450: 5-Chloro-3-[5-(3-chloro-phenyl)-2-phenyl-4-(1H-tetrazol-5-yl)-imidazol-1-yl]-1H-pyridin-2-one
Example 451: 1-(3-Chloro-2-fluoro-phenyl)-5-(3-chloro-phenyl)-2-phenyl-1-H-imidazole-4-sulfonic acid amide
Example 452: 1-(3-Chloro-2-fluoro-phenyl)-5-(3-chloro-phenyl)-2-phenyl-1-H-imidazole-4-sulfonic acid (2-methoxy-ethyl)-amide
Example 453: 1-(3-Chloro-2-fluoro-phenyl)-5-(3-chloro-phenyl)-2-phenyl-1H-imidazole-4-sulfonic acid methylamide
Example 454: 1-(3-Chloro-2-fluoro-phenyl)-5-(3-chloro-4-fluoro-phenyl)-2-phenyl-4-(1H-pyrrol-2-yl)-1-H-imidazole
Example 455: 1-(2-tert-Butoxycarbonylmethyl-5-chloro-phenyl)-5-(3-chloro-4-fluoro-phenyl)-2-cyclohexyl-1H-imidazole-4-carboxylic acid
Example 456: {2-[4-Carbamoyl-5-(3-chloro-4-fluoro-phenyl)-2-cyclohexyl-imidazol-1-yl]-4-chloro-phenyl}-acetic acid tert-butyl ester
Example 457: {2-[4-Carbamoyl-5-(3-chloro-4-fluoro-phenyl)-2-cyclohexyl-imidazol-1-yl]-4-chloro-phenyl}-acetic acid
Example 458: 1-(2-Carbamoylmethyl-5-chloro-phenyl)-5-(3-chloro-4-fluoro-phenyl)-2-cyclohexyl-1H-imidazole-4-carboxylic acid amide
Example 459: {4-Chloro-2-[5-(3-chloro-4-fluoro-phenyl)-4-cyano-2-cyclohexyl-imidazol-1-yl]-phenyl}-acetic acid tert-butyl ester
Example 460: {4-Chloro-2-[5-(3-chloro-4-fluoro-phenyl)-2-cyclohexyl-4-(2H-tetrazol-5-yl)-imidazol-1-yl]-phenyl}-acetic acid tert-butyl ester
Example 461: {4-chloro-2-[5-(3-chloro-4-fluoro-phenyl)-2-cyclohexyl-4-(2H-tetrazol-5-yl)-imidazol-1-yl]-phenyl}-acetic acid
Example 462: 2-{4-Chloro-2-[5-(3-chloro-4-fluoro-phenyl)-2-cyclohexyl-4-(2H-tetrazol-5-yl)-imidazol-1-yl]-phenyl}-N-(4-methoxy-benzyl)-acetamide
Example 463: 5-(3-Chloro-4-fluoro-phenyl)-1-(4-chloro-pyridin-2-yl)-2-m-tolyl-1H-imidazole-4-carboxylic acid ethyl ester
Example 464: 4-[5-(3-Chloro-4-fluoro-phenyl) 1-(3-chloro-2-fluoro-phenyl)-2-phenyl-1H-imidazol-4-yl]-isoxazole
Example 465: 5-(3-Chloro-4-fluoro-phenyl)-1-(4-chloro-pyridin-2-yl)-2-m-tolyl-1H-imidazole-4-carboxylic acid
Example 466: 3-[5-(3-Chloro-4-fluoro-phenyl)-1-(3-chloro-2-fluoro-phenyl)-2-phenyl-1H-imidazol-4-yl]-5-methyl-[1,2,4]oxadiazole
Example 467: {2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-acetic acid tert-butyl ester
Example 468: {2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-acetic acid
Example 469: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-methyl-acetamide
Example 470: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-tert-butyl-acetamide
Example 471: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-ethyl-N-phenethyl-acetamide
Example 472: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(2,2-dimethoxy-ethyl)-acetamide
Example 473: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(4-fluoro-phenyl)-acetamide
Example 474: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-isobutyl-acetamide
Example 475: {4-Chloro-2-[5-(3-chloro-4-fluoro-phenyl)-2-cyclohexyl-4-(5-methyl-[1,3,4]oxadiazol-2-yl)-imidazol-1-yl]-phenyl}-acetic acid
Example 476: 2-{4-Chloro-2-[5-(3-chloro-4-fluoro-phenyl)-2-cyclohexyl-4-(5-methyl-[1,3,4]oxadiazol-2-yl)-imidazol-1-yl]-phenyl}-1-piperidin-1-yl-ethanone
Example 477: 2-{4-Chloro-2-[5-(3-chloro-4-fluoro-phenyl)-2-cyclohexyl-4-(5-methyl-[1,3,4]oxadiazol-2-yl)-imidazol-1-yl]-phenyl}-N-methyl-N-pyridin-3-ylmethyl-acetamide
Example 478: {4-Chloro-2-[5-(3-chloro-4-fluoro-phenyl)-2-cyclohexyl]-4-hydrazinocarbonyl-imidazol-1-yl]-phenyl}-acetic acid tert-butyl ester
Example 479: {2-[4-(5-Amino-[1,3,4]oxadiazol-2-yl)-5-(3-chloro-2-fluoro-phenyl)-2-(cyclohexyl)-imidazol-1-yl]-4-chloro-phenyl}-acetic acid tert butylester
Example 480: {2-[4-(5-Amino-[1,3,4]oxadiazol-2-yl)-5-(3-chloro-2-fluoro-phenyl)-2-(cyclohexyl)-imidazol-1-yl]-4-chloro-phenyl}acetic acid
Example 481: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-methyl-N-pyridin-3-ylmethyl-acetamide
Example 482: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-methyl-acetamide
Example 483: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-tert-butyl-acetamide
Example 484: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-1-morpholin-4-yl-ethanone
Example 485: 5-(3-Chloro-4-fluoro-phenyl)-1-(3-chloro-2-fluoro-phenyl)-2-cyclohexyl-1H-imidazole-4-carboxylic acid hydrazide
Example 486: 5-(3-Chloro-4-fluoro-phenyl)-1-(3-chloro-2-fluoro-phenyl)-2-phenyl-1H-imidazole-4-carboxylic acid amide
Example 487: 5-(3-Chloro-4-fluoro-phenyl)-1-(3-chloro-2-fluoro-phenyl)-2-phenyl-1H-imidazole-4-carbonitrile
Example 488: 5-[5-(3-Chloro-4-fluoro-phenyl)-1-(3-chloro-2-fluoro-phenyl)-2-phenyl-1H-imidazol-4-yl]-2H-tetrazole
Example 489: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-(2-dimethylamino-ethyl)-acetamide
Example 490: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-(3-methoxy-propyl)-acetamide
Example 491: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-pyridin-4-ylmethyl-acetamide
Example 492: 2-(2-[4-(5-Amino-[1,3,4]oxadiazol-2-yl)-5-(3-chloro-4-fluoro-phenyl)-2-cyclohexyl-imidazol-1-yl]-4-chloro-phenyl}-N-(4,4-dimethyl-pentyl)-acetamide
Example 493: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-cyclohexylmethyl-acetamide
Example 494: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-(3-hydroxy-2,2-dimethyl-propyl)-acetamide
Example 495: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-1-[4-(4-methyl-piperazin-1-yl)-piperidin-1-yl]-ethanone
Example 496: 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-(2-phenylamino-ethyl)-acetamide
Example 497: 2-[5-(3-Chloro-4-fluoro-phenyl)-1-(3-chloro-2-fluoro-phenyl)-2-cyclohexyl-1H-imidazol-4-yl]-5-methyl-[1,3,4]oxadiazole
Example 498: 5-[5-(3-Chloro-4-fluoro-phenyl)-1-(3-chloro-2-fluoro-phenyl)-2-cyclohexyl-1H-imidazol-4-yl]-3H-[1,3,4]oxadiazol-2-one
Example 499: 5-[5-(3-Chloro-4-fluoro-phenyl)-1-(3-chloro-2-fluoro-phenyl)-2-cyclohexyl-1H-imidazol-4-yl]-[1,3,4]oxadiazol-2-ylamine
Example 500: N-{3-[5-(3-Chloro-4-fluoro-phenyl)-1-(3-chloro-2-fluoro-phenyl)-5-methyl-[1,2,4]oxadiazol-3-yl)-1H-imidazol-2-yl]-phenyl}-acetamide
Example 501: 1-(6-Carboxymethyl-3-chloro-2-fluoro-phenyl)-5-(3-chloro-4-fluoro-phenyl)-2-cyclohexyl-1H-imidazole-4-carboxylic acid benzyl ester
Example 502: 1-(6-tert-Butoxycarbonylmethyl-3-chloro-2-fluoro-phenyl)-5-(3-chloro-4-fluoro-phenyl)-2-cyclohexyl-1H-imidazole-4-carboxylic acid benzyl ester
Example 503: 1-(6-Carbamoylmethyl-3-chloro-2-fluoro-phenyl)-5-(3-chloro-4-fluoro-phenyl)-2-cyclohexyl-1H-imidazole-4-carboxylic acid amide
Example 504: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(3-hydroxy-2,2-dimethyl-propyl)-acetamide
Example 505: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(2-hydroxy-1,1-dimethyl-ethyl)-acetamide
Example 506: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-phenyl-acetamide
Example 507: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-cyclohexyl-acetamide
Example 508: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-cyclopentyl-acetamide
Example 509: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(tetrahydro-pyran-4-yl)-acetamide
Example 510: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl)-N-(3-fluoro-phenyl)-acetamide
Example 511: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(1-hydroxymethyl-cyclopropyl)-acetamide
Example 512: [2-(2-{(2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-acetylamino)-ethyl]-trimethyl-ammonium trifluoracetate
Example 513: [2-(2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-acetylamino)-2-methyl-propyl]-carbamic acid tert-butyl ester
Example 514: N-(2-Amino-1,1-dimethyl-ethyl)-2-{2-[4-(5-amino-[1,3,4]oxadiazol-2-yl)-5-(3-chloro-4-fluoro-phenyl)-2-cyclohexyl-imidazol-1-yl]-4-chloro-3-fluoro-phenyl}-acetamide
Example 515: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-1-(3,3-dimethyl-azetidin-1-yl)-ethanone
Example 516: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-(1,1,2-trimethyl-propyl)-acetamide
Example 517: 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-1-(3-methyl-azetidin-1-yl)-ethanone
Example 518: 1-(6-Carboxymethyl-3-chloro-2-fluoro-phenyl)-5-(3-chloro-4-fluoro-phenyl)-2-cycloheptyl-1H-imidazole-4-carboxylic acid benzyl ester
Example 519: 1-(6-tert-Butoxycarbonylmethyl-3-chloro-2-fluoro-phenyl)-5-(3-chloro-4-fluoro-phenyl)-2-cycloheptyl-1H-imidazole-4-carboxylic acid benzyl ester
Example 520: 1-(6-tert-Butoxycarbonylmethyl-3-chloro-2-fluoro-phenyl)-5-(3-chloro-4-fluoro-phenyl)-2-cycloheptyl-1H-imidazole-4-carboxylic acid
Example 521: {2-[4-(5-Amino-[1,3,4]oxadiazol-2-yl)-5-(3-chloro-4-fluoro-phenyl)-2-cycloheptyl-imidazol-1-yl]-4-chloro-3-fluoro-phenyl}-acetic acid tert-butyl ester
Example 522: {2-[4-(5-Amino-[1,3,4]oxadiazol-2-yl)-5-(3-chloro-4-fluoro-phenyl)-2-cycloheptyl-imidazol-1-yl]-4-chloro-3-fluoro-phenyl}-acetic acid
Example 523: 2-{2-[4-(5-Amino-[1,3,4]oxadiazol-2-yl)-5-(3-chloro-4-fluoro-phenyl)-2-cycloheptyl-imidazol-1-yl]-4-chloro-3-fluoro-phenyl}-N-tert-butyl-acetamide
Example 524: 2-{2-[4-(5-Amino-[1,3,4]oxadiazol-2-yl)-5-(3-chloro-4-fluoro-phenyl)-2-cycloheptyl-imidazol-1-yl]-4-chloro-3-fluoro-phenyl}-N-(1,1-dimethyl-propyl)-acetamide
Example 525: 2-{2-[4-(5-Amino-[1,3,4]oxadiazol-2-yl)-5-(3-chloro-4-fluoro-phenyl)-2-cycloheptyl-imidazol-1-yl]-4-chloro-3-fluoro-phenyl}-1-azetidin-1-yl-ethanone

As already indicated above, p53 refers to the human protein itself as described by Matlashewski et al. in EMBO J. 3, 3257-62 (1984) or related family members (e.g. p73 as described in Kaghad et al. in Cell 90, 809-19 (1997) and p63 as described in Yang et al in Mol Cell 2, 305-16 (1998)) (named also p53 wild type herein) or to any variant thereof (e.g. a splice variant, mutant, fragment or isoform due to deletion, insertion and/or exchange of one or more, e.g. one to 200, of the amino acids) that is still capable to retain preferably at least 1%, more preferably at least 5%, yet more preferably at least 10%, 20%, 30%, 40%, 50% or more than 50% of the p53 activity in growth suppression, e.g. in the growth suppression assay described in Pietenpol et al., Proc. Nat. Acad. Sci. USA 91, 1998-2002 (1994) and, if compared with the corresponding sequence of p53 wild type, shows at least 20%, more preferably at least 25% identity with the full sequence, e.g. at least 90% identity with a partial sequence thereof. Where not mentioned otherwise, p53 generally relates to TP53, p53, TP73, p73, TP63, TP73L, p63, or variants thereof, respectively, as just defined.

As already indicated above, MDM2 (especially when mentioned as MDM2 or variants thereof) generally refers to all genes and/or proteins encoded thereof with the names MDM2, Mdm2, HDM2, Hdm2, or a variant thereof. MDM4 (especially when mentioned as MDM4 or variants thereof) refers to all genes and/or proteins encoded thereof with the names MDM4, Mdm4, HDM4, Hdm4, MDMX, MdmX, HDMX, HdmX, or a variant thereof.

MDM2 specifically relates to MDM2 as described in EMBO J. 10, 1565-9, Fakharzadeh et al., 1991, a variant thereof refers to a variant thereof which still binds to p53 in the assay system described below (e.g. a splice variant, isoform, fragment, mutant or oncogene due to deletion, insertion and/or exchange of one or more, e.g. one to 430, of the amino acids), corresponding to the full length proteins as originally described, preferably at least with 0.5%, more preferably at least with 5%, 10%, 20%, 30%, 40% or especially 50% or more of the affinity of MDM2 to p53, and have at least 20%, more preferably at least 25%, sequence identity to MDM2 or to HDM2 as originally described or as mentioned below specifically. Where not mentioned otherwise, MDM2 generally relates to MDM2, Mdm2, HDM2 or Hdm2, or variants thereof, respectively, as just defined.

MDM4 specifically relates to MDM4 as described in Genomics 43, 34-42, Shvarts et al., 1997, a variant thereof refers to a variant thereof which still binds to p53 in the assay system described below (e.g. a splice variant, isoform, fragment, mutant or oncogene due to deletion, insertion and/or exchange of one or more, e.g. one to 430, of the amino acids), corresponding to the full length proteins as originally described, preferably at least with 0.5%, more preferably at least with 5%, 10%, 20%, 30%, 40% or especially 50% or more of the affinity of MDM4 to p53, and have at least 20%, more preferably at least 25%, sequence identity to MDM4, to MDMX, to HDM4 or to HDM2 as originally described or as mentioned below specifically. Where not mentioned otherwise, MDM4 generally relates to MDM4, Mdm4, HDM4, Hdm4, MDMX, MdmX, HDMX or HdmX, or variants thereof, respectively, as just defined.

The percentage of sequence identity, often also termed homology, between a protein and a variant thereof is preferably determined by a computer program commonly employed for this purpose, such as the Gap program (Wisconsin Sequence Analysis Package, Version 8 for Unix, Genetics Computer Group, University Reseach Park, Madison Wis., USA, which uses the algorithm of Smith and Waterman (Adv. Appl. Math. 2: 482-489 (1981), especially using an affine gap search with a gap open penalty of 12 and a gap extension penalty of 1.

“Variants thereof” where mentioned means one or more variant(s).

A proto-oncogene is a normal gene that can become an oncogene, either after mutation or increased expression. Proto-oncogenes code for proteins that help to regulate cell growth and differentiation. Proto-oncogenes are often involved in signal transduction and execution of mitogenic signals, usually through their protein products. Upon activation, a proto-oncogene (or its product) becomes a tumor inducing agent, an oncogene.

Compounds of the formula (I) may have different isomeric forms. As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns 1 to 12 of the periodic table. In certain embodiments, the salts are derived from lithium, sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

For isolation or purification purposes it is also possible to use pharmaceutically unacceptable salts, for example picrates or perchlorates. For therapeutic use, only pharmaceutically acceptable salts or free compounds are employed.

In view of the close relationship between the novel compounds of the formula (I) in free form and those in the form of their salts, including those salts that can be used as intermediates, for example in the purification or identification of the novel compounds, any reference to the compounds or a compound of the formula (I) hereinbefore and hereinafter is to be understood as referring to the compound in free form and/or also to one or more salts thereof, as appropriate and expedient, as well as to one or more solvates, e.g. hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F³¹P, ³²P, ³⁵S, ³⁸Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of the formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of the formula (I) that contain groups capable of acting as donors and/or acceptors for hydrogen bonds may be capable of forming co-crystals with suitable co-crystal formers. These co-crystals may be prepared from compounds of the formula (I) by known co-crystal forming procedures. Such procedures include grinding, heating, co-subliming, co-melting, or contacting in solution compounds of the formula (I) with the co-crystal former under crystallization conditions and isolating co-crystals thereby formed. Suitable co-crystal formers include those described in WO 2004/078163. Hence the invention further provides co-crystals comprising a compound of the formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by the dysregulation of the p53/MDM2 ratio, or (ii) associated with the dysregulation of the p53/MDM2 ratio, or (iii) characterized by the dysregulation of the MDM2/p53 ratio; or (2) reducing or inhibiting the activity of the p53/MDM2 interaction. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the p53/MDM2 interaction.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O, O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Compounds of the present invention are either obtained in the free form, as a salt thereof, or as prodrug derivatives thereof.

When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the omega-(amino, mono- or di-lower alkylamino, carboxy, lower alkoxycarbonyl)-lower alkyl esters, the alpha-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

Quite unexpectedly, it has now been found that the compounds of the formula (I) have advantageous pharmacological properties and disturb the binding interaction (also referred to herein as p53/MDM2 and p53/MDM4 interaction or as p53/MDM2 interaction solely) between p53 on the one side and MDM2 and/or MDM4 or (especially oncogenic) variants thereof which still are capable of binding to p53, on the other side.

In another embodiment of the invention there is provided a pharmaceutical composition comprising a compound of formula (I) as described herein, or a tautomer, and/or a N-oxide, and/or a pharmaceutically acceptable salt thereof, and at least one pharmaceutically acceptable carrier material.

In another embodiment of the invention there is provided a method of modulating the activity of MDM2 and/or MDM4, or variants thereof, wherein the method comprises administering to the subject a therapeutically effective amount of a compound of formula (I) as defined herein, or a tautomer, and/or a N-oxide, and/or a pharmaceutically acceptable salt thereof.

In another embodiment, the invention provides a method of treating a disorder or a disease in a subject mediated by the activity of MDM2 and/or MDM4, or variants thereof comprising administering to the subject a therapeutically effective amount of a compound of formula (I) as defined herein, or a tautomer, and/or a N-oxide, and/or a pharmaceutically acceptable salt thereof. In particular, the disorder or a disease is selected from a proliferative disease.

The efficacy of the compounds of the formula (I) and salts thereof as modulators affecting the interaction between can be demonstrated as shown in WO 98/01467 (which especially regarding the assays is included herein by reference) or preferably follows:

### Fluorescence Polarisation Assay

The inhibition of p53-Hdm2 interaction is measured by fluorescence polarization. Fluorescence polarization measures the rotational movement of molecules in a homogeneous suspension. For this assay, Hdm2 protein (amino acids 2-188) is combined with a Cy5-labelled p53-derived peptide optimised for Hdm2 binding (J. Med. Chem. 2000, 43, 3205-3208). Upon excitation of the Cy5 fluorescent ligand with linearly polarized light, the peptide rotates faster and emits light which is perpendicularly polarized. If the peptide is bound by Hdm2, rotation will slow down and the perpendicular component will decrease. Disruption of the formation of the peptide-Hdm2 complex due to an inhibitor molecule binding to the p53 binding site of Hdm2 results in faster rotation of the peptide. The ratiometric polarization assay readout is calculated from the parallel and perpendicular components of the fluorescence light with respect to the polarization of the excitation light.

The test is performed by combining 7 μl compounds diluted in dimethyl sulfoxide (DMSO) (10% final concentration) with 31.5 μl Hdm2(2-188) (final concentration 3 nM) in reaction buffer (PBS, 0.1% CHAPS, 1 mM DTT (dithiothreitol)). The solution is allowed to pre-incubate for 5 minutes at room temperature, followed by addition of 31.5 μl peptide in reaction buffer (final concentration 1 nM), and a further 5 minutes of incubation. A final volume of 20 μl (in triplicate) is distributed into small volume black 384-well plates (Greiner Bio-One GmbH, Frickenhausen, Germany). For measurement of samples, an Analyst AD multimode microplate reader (Molecular Devices Corporation, Sunnyvale, Calif., USA) with the following settings is used: Dichroic mirror 650 nm, Excitation 630 nm, Emission 695 nm. Raw values are expressed as percent of DMSO control, where background (reaction buffer with peptide but no Hdm2) is subtracted first from raw values. IC50 values are calculated by curve fitting using XLfit. If not specified, reagents were purchased from Sigma Chemical Co.

Compounds described in the present invention display inhibition of p53-Hdm2 interaction at IC50s from around 0.0003 to 60 μM, preferably ranging from 0.0003 to 25 μM, more preferably from 0.0003 to 10 μM.

### Time Resolved Fluorescence Energy Transfer (TR-FRET) Assay

The inhibition of p53-Hdm2 and p53-Hdm4 interactions is measured by time resolved fluorescence energy transfer (TR-FRET). Fluorescence energy transfer (or Foerster resonance energy transfer) describes an energy transfer between donor and acceptor fluorescent molecules. For this assay, MDM2 protein (amino acids 2-188) and MDM4 protein (amino acids 2-185), tagged with a C-terminal Biotin moiety, are used in combination with a Europium labeled streptavidin (Perkin Elmer, Inc., Waltham, Mass., USA) serving as the donor fluorophore. The p53 derived, Cy5 labeled peptide Cy5-TFSDLWKLL (p53 aa18-26) is the energy acceptor. Upon excitation of the donor molecule at 340 nm, binding interaction between MDM2 or MDM4 and the p53 peptide induces energy transfer and enhanced response at the acceptor emission wavelength at 665 nm. Disruption of the formation of the p53-MDM2 or p53-MDM4 complex due to an inhibitor molecule binding to the p53 binding site of MDM2 or MDM4 results in increased donor emission at 615 nm. The ratiometric FRET assay readout is calculated from the raw data of the two distinct fluorescence signals measured in time resolved mode (countrate 665 nm/countrate 615 nm×1000).

The test is performed in white 1536w microtiterplates (Greiner Bio-One GmbH, Frickenhausen, Germany) in a total volume of 3.1 μl by combining 100 nl of compounds diluted in 90% DMSO/10% H₂O (3.2% final DMSO concentration) with 2 μl Europium labeled streptavidin (final concentration 2.5 nM) in reaction buffer (PBS, 125 mM NaCl, 0.001% Novexin (consists of carbohydrate polymers (Novexin polymers), designed to increase the solubility and stability of proteins; Novexin Ltd., Cambridgeshire, United Kingdom), Gelatin 0.01%, 0.2% Pluronic (block copolymer from ethylenoxide and propyleneoxide, BASF, Ludwigshafen, Germany), 1 mM DTT), followed by the addition of 0.5 μl MDM2-Bio or MDM4-Bio diluted in assay buffer (final concentration 10 nM). Allow the solution to pre-incubate for 15 minutes at room temperature, followed by addition of 0.5 μl Cy5-p53 peptide in assay buffer (final concentration 20 nM). Incubate at room temperature for 10 minutes prior to reading the plate. For measurement of samples, an Analyst GT multimode microplate reader (Molecular Devices) with the following settings is used: Dichroic mirror 380 nm, Excitation 330 nm, Emission Donor 615 nm and Emission Acceptor 665 nm. IC50 values are calculated by curve fitting using XLfit. If not specified, reagents are purchased from Sigma Chemical Co, St. Louis, Mo., USA.

The present invention also relates to novel aspects of the above described assays.

Compounds described in the present invention preferably display inhibition of p53-Hdm2 interaction at IC50s of 0.005 to 100 μM, e.g. from 10 nM to 50 μM, preferably <10 μM, more preferably <1 μM.

Compounds described in the present invention preferably display inhibition of p53-Hdm4 interaction at IC50s of 0.005 to 100 μM.

Inhibitions of p53-Hdm2 and p53-Hdm4 by representative compounds in the present invention are displayed in Table 2 hereinbelow.

Having regard to their inhibitory effect on p53/MDM2 and/or p53/MDM4 interaction, compounds of the formula (I) in free or pharmaceutically acceptable salt form, are useful in the treatment of conditions which are mediated by the activity (including normal activity or especially overactivity) of MDM2 and/or MDM4, or variants thereof, respectively, as described, such as proliferative and/or inflammatory conditions, e.g. by activation of the P53/MDM2 interaction, and/or that are responsive (meaning especially in a therapeutically beneficial way) to inhibition of the p53/MDM2 interaction, most especially a disease or disorder as mentioned hereinbelow.

Preferred is a compound of the formula (I) for use or the use thereof in the treatment of a disease or disorder that responds to treatment with a compound of the formula (I), especially selected from disease that is based on dysregulation of cell cycle or especially apoptosis: e.g. diseases involving the immune system, e.g. autoimmune diseases or immune diseases resulting due to transplantation (such as rheumatoid arthritis, graft-versus-host disease, systemic lupus erythematosus, Sjögren's syndrome, multiple sclerosis, Hashimoto's thyreoiditis, polymyositis), chronic inflammatory conditions, such as asthma, osteoarthritis, atherosclerosis, Morbus Crohn or inflammatory or allergic con-ditions of the skin, for example psoriasis, contact dermatitis, atopic dermatitis, alopecia greata, erythema multiforma, dermatitis herpetiformis, scleroderma, vitiligo, hypersensitivity angiitis, urticaria, bullous pemphigoid, pemphigus, epidermolysis bullosa acquisita, or other inflammatory or allergic conditions of the skin, hyperproliferative disorders, (e.g. Li-Fraumeni syndrome, cancer or tumor diseases, such as benign or malignant tumors, a sarcoma, such as liposarcoma, rhabdomyosarcoma or bone cancer, e.g. osteosarcomas, a carcinoma, such as of the brain, kidney, liver, adrenal gland, bladder, breast, gastric, ovary, colon, rectum, prostate, pancreas, lung, vagina or thyroid, a glioblastoma, a multiple myeloma, a gastrointestinal cancer, especially colon carcinoma or colorectal adenoma, a tumor of the head and neck, a melanoma, a prostate hyperplasia, a neoplasia, a neoplasia of epithelial character, a leukemia or a lymphoma, such as of B- or T-cell origin, and metastases in other organs), viral infections (e.g. herpes, papilloma, HIV, Kaposi's, viral hepatitis) or other diseases, for example those in which the p53/MDM2 and/or p53/MDM4 interaction is dysregulated and/or that are responsive to inhibition of the p53/MDM2 interaction and/or p53/MDM4 interaction.

The invention especially relates to the use of a compound of the formula (I) (or a pharmaceutical formulation comprising a compound of the formula (I)) in the treatment of one or more of the diseases mentioned above and below where the disease(s) respond or responds (in a beneficial way, e.g. by partial or complete removal of one or more of its symptoms up to complete cure or remission) to an inhibition of the p53/MDM2 interaction, especially where the involved MDM2 or MDM4 and/or variant shows (e.g. in the context of other regulatory mechanisms, due to overexpression, to mutation or the like) inadequately high or more higher than normal activity.

The invention can also relate to the use of a compound of the formula (I) to induce cell cycle deceleration or preferably arrest and/or apoptosis in cells containing p53 or variants thereof that are still functional, for sensitizing cells to one or more additional pharmaceutically active agents, such as inducers of apoptosis and/or of cell cycle deceleration or arrest, and to chemoprotection of normal cells through the induction of cell cycle deceleration or arrest prior to treatment with one or more other chemotherapeutic agents, to the use in rendering normal cells resistant to chemotherapeutic agents and/or treatments, and/or the use in protecting cells from toxic side effects of chemotherapeutic agents or treatments, such as side effects resulting in mucositis, stomatitis, xerostomia, gastrointestinal disorders and/or alopecia.

All these aspects are preferred embodiments of the present invention.

There are also experiments that can demonstrate the antitumor activity of compounds of the formula (I) in vivo.

For example, female Harlan (Indianapolis, Ind., USA) athymic nu/nu mice with s.c. transplanted human osteosarcoma SJSA-1 tumors can be used to determine the anti-tumor activity of p53/MDM2 interaction inhibitors. On day 0, with the animals under peroral Forene® (1-chloro-2,2,2-trifluoroethyldifluormethylether, Abbot, Wiesbaden, Germany) narcosis, 3×10⁶ cells are injected under the skin on the animals' left flank.

When tumors reach a volume of 100 mm³, the mice are divided at random into groups of 6-8 animals and treatment commences. The treatment is carried out for a 2-3 weeks period with peroral, intravenous or intra-peritoneal administration twice daily (or less frequently) of a compound of the formula (I) in a suitable vehicle at defined doses. The tumors are measured twice a week with a slide gauge and the volume of the tumors is calculated.

As an alternative to cell line SJSA-1, other cell lines may also be used in the same manner, for example,
the HCT116 colon carcinoma cell line (ATCC No. CCL-247);
the LNCaP clone FGC prostate carcinoma cell line (ATCC No. CRL-1740);
the RKO colon carcinoma cell line (ATCC No. CRL-2577);
the HT1080 fibrosarcoma cell line (ATCC No. CCL-121);
the A375 malignant melanoma cell line (ATCC No. CRL-1619),
the NCI-H460 large cell lung carcinoma cell line (ATCC No. HTB-177);
the JEG-3 choriocarcinoma (ATCC No. HTB-36)
the ZR-75-1 breast ductal carcinoma (ATCC No. CRL-1500)

A compound of the formula (I) may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibittors; mTOR inhibitors, such as RAD001; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies, such as HCD122; heparanase inhibitors; inhibitors of Ras oncogenic isoforms; telomerase inhibitors; proteasome inhibitors; compounds used in the treatment of hematologic malignancies, such as fludarabine; compounds which target, decrease or inhibit the activity of Flt-3, such as PKC412; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics and AUY922; temozolomide (TEMODAL™); kinesin spindle protein inhibitors, such as SB715992 or SB743921 from GlaxoSmithKline, or pentamidine/chlorpromazine from CombinatoRx; PI3K inhibitors, such as BEZ235; RAF inhibitors, such as RAF265; MEK inhibitors such as ARRY 142886 from Array PioPharma, AZD6244 from AstraZeneca, PD181461 from Pfizer, leucovorin, EDG binders, antileukemia compounds, ribonucleotide reductase inhibittors, S-adenosylmethionine decarboxylase inhibitors, regulators of apoptosis, antiproliferative antibodies or other chemotherapeutic compounds. Further, alternatively or in addition they may be used in combination with other tumor treatment approaches, including surgery, ionizing radiation, photodynamic therapy, implants, e.g. with corticosteroids, hormones, or they may be used as radiosensitizers. Also, in anti-inflammatory and/or antiproliferative treatment, combination with anti-inflammatory drugs is included. Combination is also possible with antihistamine drug substances, bronchodilatatory drugs, NSAID or antagonists of chemokine receptors.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX™), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is mar-keted, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL™. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, protein tyrosine kinase and/or serine and/or threonine kinase inhibitors or lipid kinase inhibitors, e.g.,

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, i.e C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
h) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
i) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; BEZ235 (a PI3K inhibitor) or AT7519 (CDK inhibitor);
j) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-[[(2,5-dihydroxyphenyl)methyl]amino]-benzoic acid adamantyl ester; NSC 680410, adaphostin);
k) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
l) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF;
m) compounds targeting, decreasing or inhibiting the activity of PI3K, such as BEZ235 or BKM120;
n) compounds targeting, decreasing or inhibiting the activity of the cyclin dependent kinase family, such as PD 0332991.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, or CDC25, e.g. okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX™), rofecoxib (VIOXX™), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune™), everolimus (Certican™ or Afinitor™), CCI-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetrazolyle derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, TKI258, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG), a geldanamycin derivative; other geldanamycin related compounds; radicicol and HDAC inhibitors. An example HSP90 inhibitor is AUY922.

The term “regulators of apoptosis” as used herein includes, but is not limited to, compounds targeting, decreasing or inhibiting the activity of Bcl2 family members (such as ABT-263) and IAP family members (such as AEG40826); or inducing apoptosis by known or unknown mechanism(s) of action (e.g. TRAIL antibody, DR5 antibody).

The term “antiproliferative antibodies” as used herein includes, but is not limited to, trastuzumab (Herceptin™), Trastuzumab-DM1, erbitux, bevacizumab (Avastin™), rituximab (Rituxan™), PRO64553 (anti-CD40), 2C4 Antibody and HCD122 antibody (anti-CD40). By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the formula (I) can be used in combination with standard leukemia therapies, especially in combination with therapies used for the treatment of AML. In particular, compounds of the formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate.

Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A, LDH589 disclosed in WO 02/22577 and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt.

Somatostatin receptor antagonists as used herein refer to compounds which target, treat or inhibit the somatostatin receptor such as octreotide, and SOM230 (pasireotide).

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term “EDG binders” as used herein refers a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as e.g. VISUDYNE™ and porfimer sodium.

Angiostatic steroids as used herein refers to compounds which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as e.g. fluocinolone, dexamethasone.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code nos., generic or trade names may be taken from the actual edition of the standard compendium “The Merck Index” or from databases, e.g. Patents International (e.g. IMS World Publications).

None of the quotations of references made within the present disclosure is to be understood as an admission that the references cited are prior art that would negatively affect the patentability of the present invention.

### Pharmaceutical Formulations, Uses and Methods

The above-mentioned compounds, which can be used in combination with a compound of the formula (I), can be prepared and administered as described in the art, such as in the documents cited above.

The invention also provides a pharmaceutical preparation, comprising a compound of the formula (I) as defined herein, and/or an N-oxide or a tautomer thereof, and/or a pharmaceutically acceptable salt of such a compound, or a hydrate or solvate thereof (all referred to often as “a compound of the formula (I)” merely herein), and at least one pharmaceutically acceptable carrier.

A compound of the formula (I) can be administered alone or in combination with one or more other therapeutic compounds, possible combination therapy taking the form of fixed combinations or the administration of a compound of the invention and one or more other therapeutic (including prophylactic) compounds being staggered or given independently of one another, or the combined administration of fixed combinations and one or more other therapeutic compounds. A compound of the formula (I) can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

The dosage of the active ingredient depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

The dose of a compound of the formula (I) or a pharmaceutically acceptable salt thereof to be administered to warm-blooded animals, for example humans of approximately 70 kg body weight, is preferably from approximately 3 mg to approximately 15 g, more preferably from approximately 10 mg to approximately 3 g, yet more preferably from approximately 50 mg to 1.5 g per person per day, undivided in 1 dose or divided preferably into 2 to 4, e.g. 2 or 3, single doses which may, for example, be of the same size. Usually, children receive half of the adult dose.

The compounds of the formula (I) may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The invention relates also to pharmaceutical compositions comprising an effective amount, especially an amount effective in the treatment of one of the above-mentioned disorders, of a compound of the formula (I) and/or an N-oxide or a tautomer thereof, and/or a pharmaceutically acceptable salt thereof, together with one or more pharmaceutically acceptable carriers that are suitable for topical, enteral, for example oral or rectal, or parenteral administration and that may be inorganic or organic, solid or liquid. There can be used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting compounds and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectionning, dissolving or lyophilising processes, and comprise approximately from 1% to 99%, especially from approximately 1% to approximately 20%, active ingredient(s).

Additionally, the present invention provides a compound of the formula (I), and/or an N-oxide or a tautomer thereof, and/or a pharmaceutically acceptable salt thereof, for use in a method for the treatment of the human or animal body, especially for the treatment of a disease mentioned herein, most especially in a patient requiring such treatment.

The present invention also relates to the use of a compound of the formula (I) and/or an N-oxide or a tautomer thereof, and/or a pharmaceutically acceptable salt of such a compound, for the preparation of a medicament for the treatment especially of a proliferative disease, especially cancer.

Furthermore, the invention relates to a method for the treatment of a proliferative disease which responds to an inhibition of the p53/MDM2 interaction, which comprises administering a compound of the formula (I), and/or an N-oxide or a tautomer thereof, and/or a pharmaceutically acceptable salt thereof, wherein the radicals and symbols have the meanings as defined above, to a warm-blooded animal requiring such treatment, especially in a quantity effective against said disease and/or capable of inhibiting the p53/MDM2 interaction in said warm-blooded animal.

Furthermore, the invention relates to a pharmaceutical composition for treatment of solid or liquid tumors in warm-blooded animals, including humans, comprising an antiproliferativly effective dose of a compound of the formula (I) as described above or a pharmaceutically acceptable salt of such a compound together with a pharmaceutical carrier.

### Synthesis of Compounds of the Formula (I)

Typically, the compounds of the formula (I) can be prepared according to the Schemes provided infra.

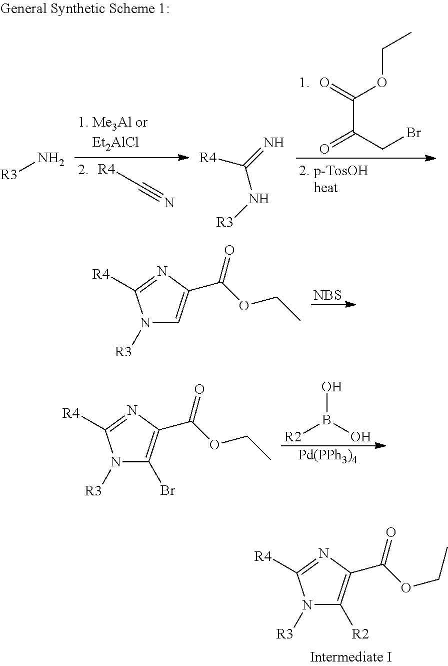

Intermediates I were mainly prepared according to published literature procedures WO2005/009974, WO2005/086836, WO2005/087229, J. Med. Chem. 2005, 48, 2638-2645.

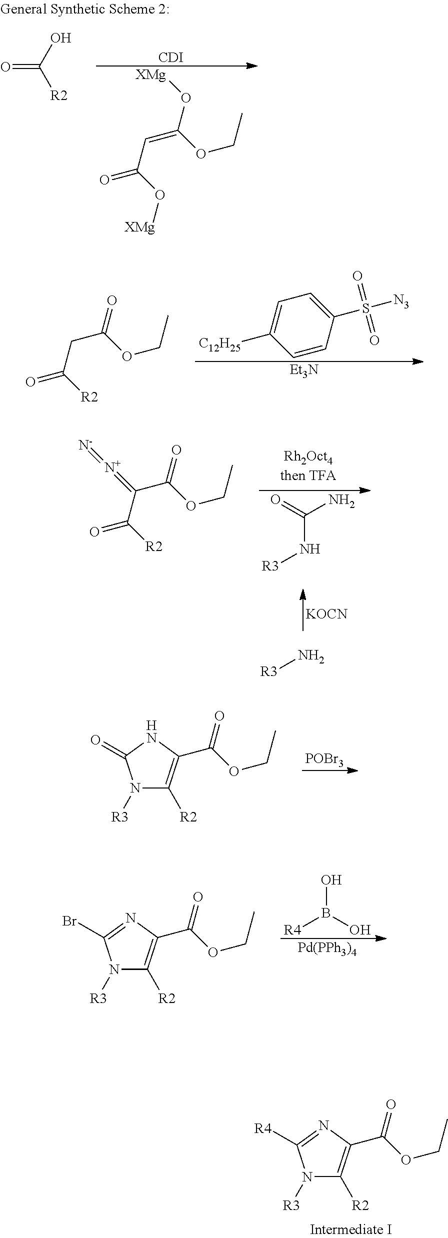

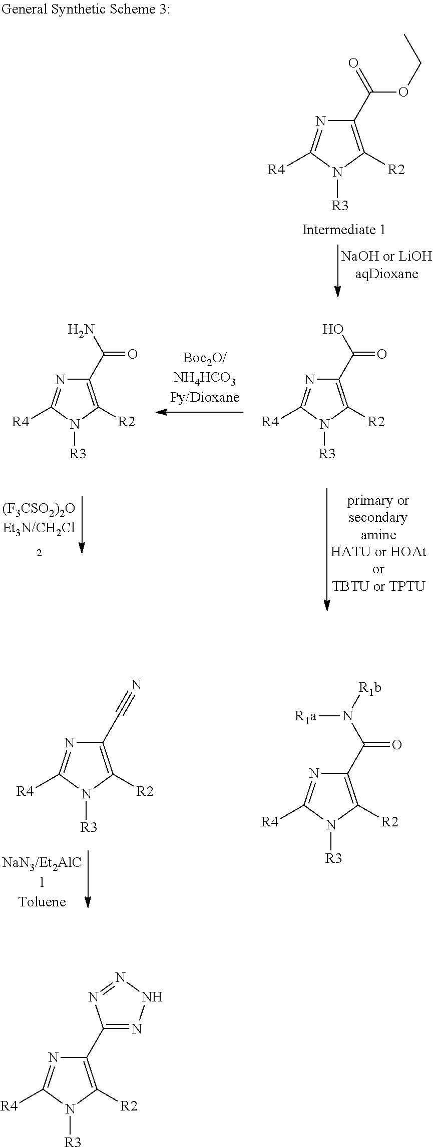

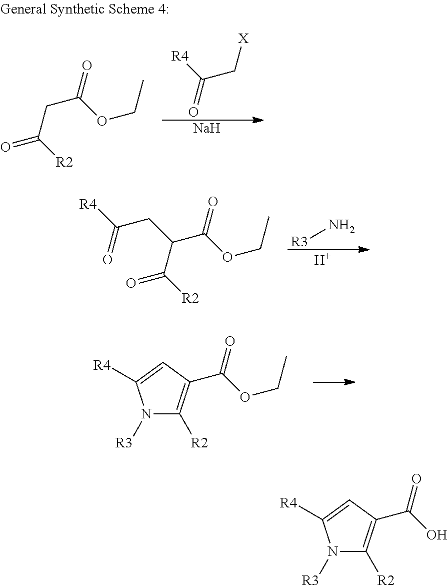

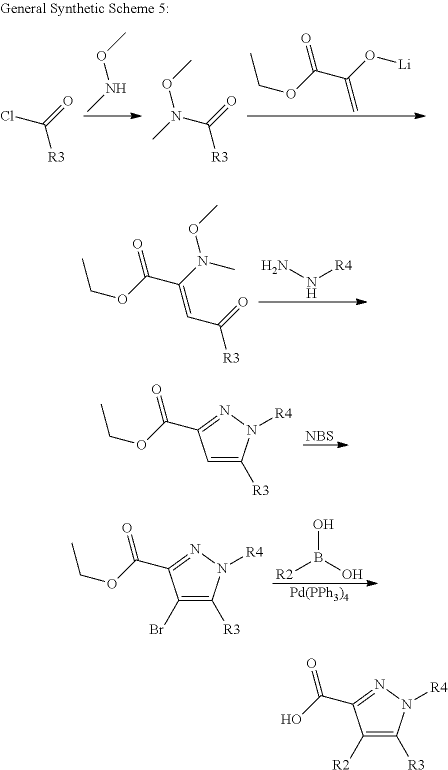

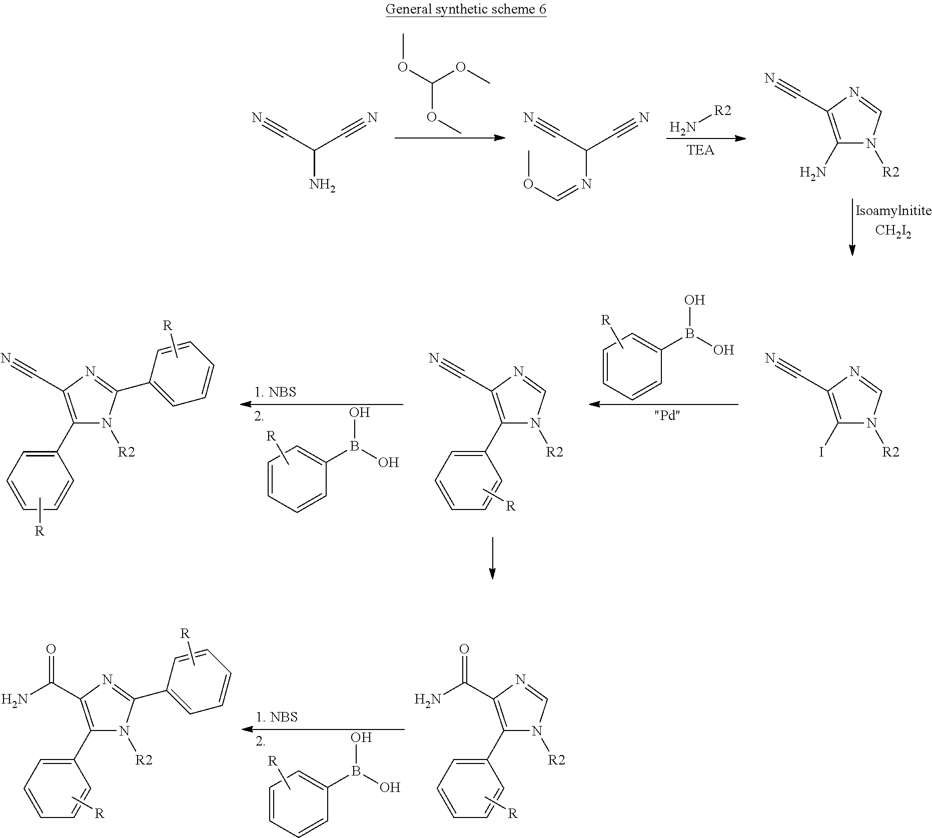

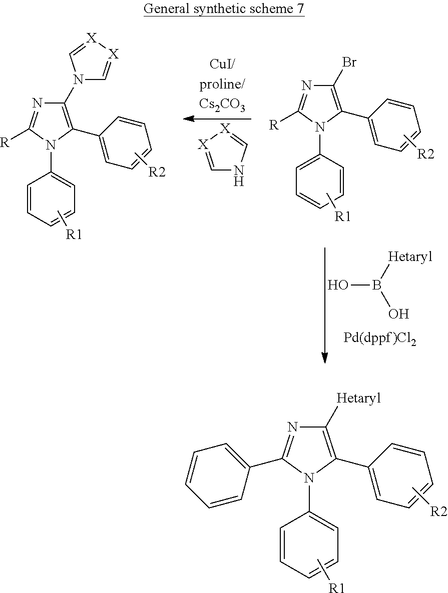

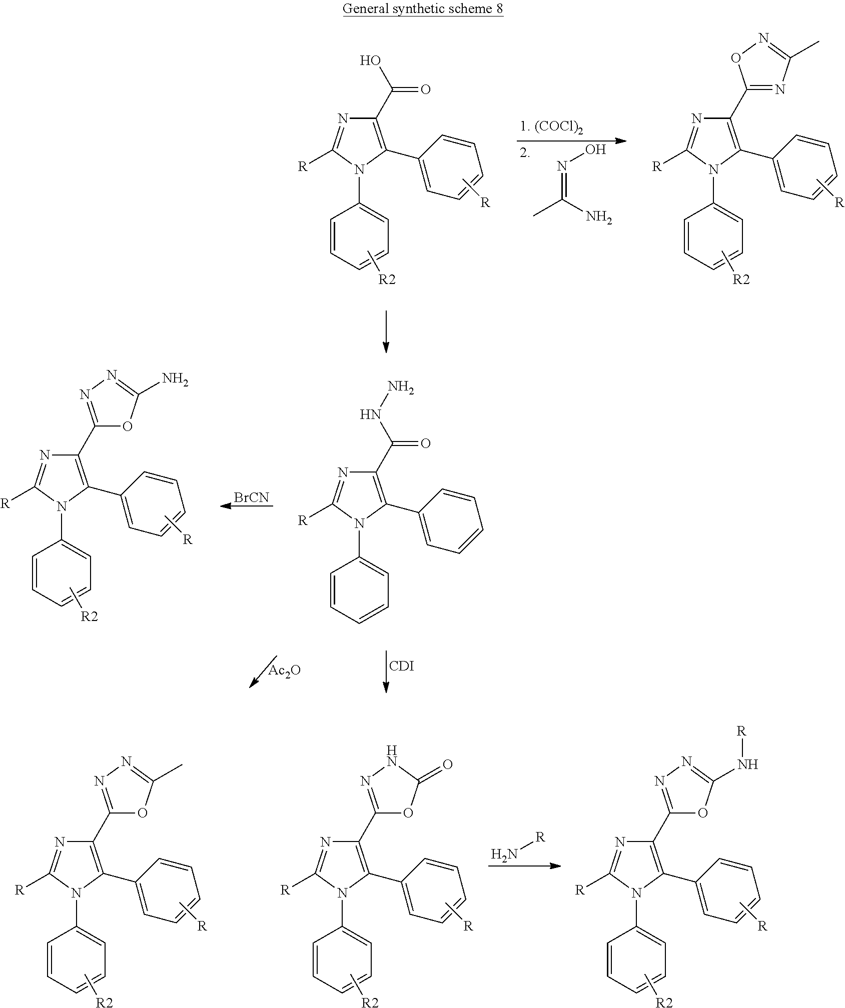

Additional processes to make compounds of the invention are provided below:

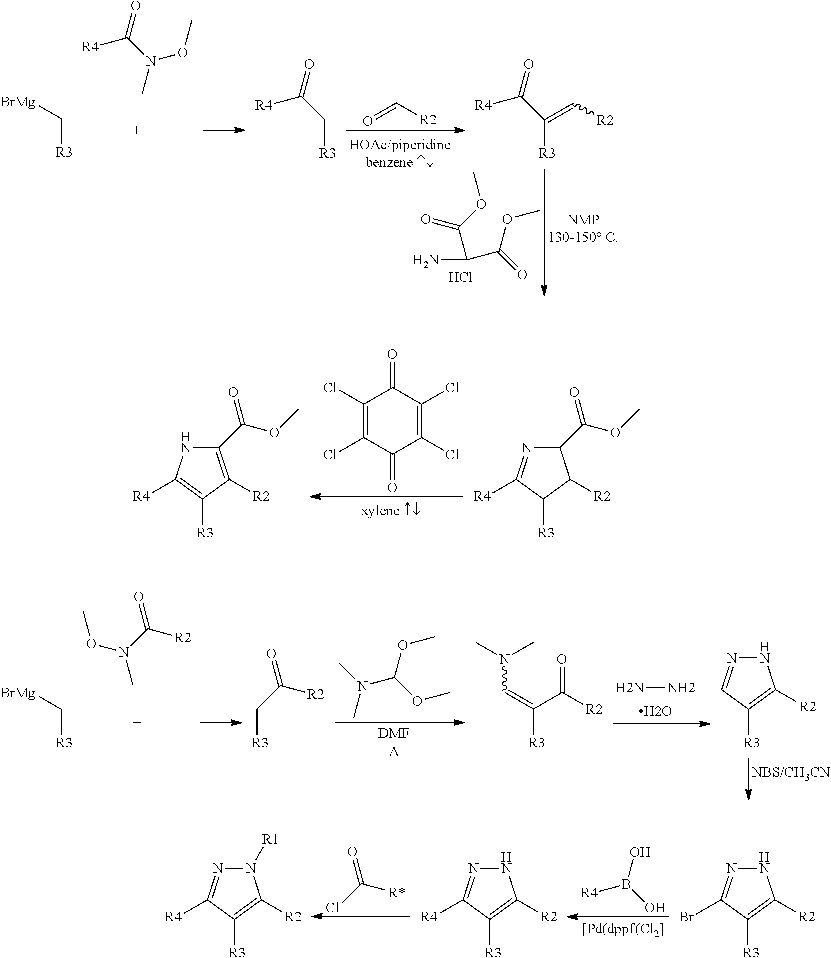

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known to those skilled in the art.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described herein above.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

### EXAMPLES

### Abbreviations

Ac acetyl
AcOH acetic acid
Anal. elemental analysis (for indicated atoms, difference between calculated and measured values ≦0.4%)
aq. aqueous
Boc tert-butoxycarbonyl
Brine saturated (at rt) sodium chloride solution
^{t}Bu tert-butyl
Celite trademark of Celite Corp. (World Minerals Inc.), Santa Barbara, Calif., USA, for filtering aid based on kieselguhr
CH₃CN acetonitrile
conc. concentrated
DCE dichloroethane
DCM dichloromethane
DMAP 4-dimethylaminopyridine
DMF N,N-dimethylformamide
DMSO dimethylsulfoxide
DPPA diphenylphosphorylazide
ES-MS electrospray mass spectrometry
Et ethyl
Et₂AlCl diethyl aluminium chloride
Et₃N triethylamine
Et₂O diethyl ether
EtOAc ethyl acetate
EtOH ethanol
h hour(s)
HATU O-(7-azabenzotriazol-1-yl)-N,N,N′,N′-tetramethyluronium-hexafluorophosphat
HBr hydrogen bromide
HCl hydrogen chloride
HOAt 1-hydroxy-7-azabenzotriazole
HPLC high-pressure liquid chromatography
HyFlo diatomaceous earth based filtering aid, trademark of Johns Manville Corp., Denver, Colo., USA
HV High vacuum
iPr isopropyl
KHMDS potassium hexamethyldisilazide
KO^{t}Bu potassium-tert-butoxylate
KOH potassium hydroxide
LAH lithium aluminium hydride
Me methyl
MeOH methanol
MgSO₄ magnesium sulfate
min minute(s)
ml milliliter(s)
mmol millimol(s)
mp melting point
MPLC medium pressure liquid chromatography
MS Mass Spectrometry
NaOH sodium hydroxide
NaOMe sodium methoxylate
NaOEt sodium ethoxylate
Na₂SO₄ sodium sulfate
NBS N-bromosuccinimide
NCS N-chlorosuccinimide
NIS N-iodosuccinimide
NMM 4-methylmorpholine
NMR nuclear magnetic resonance
NMP 1-Methyl-pyrrolidin-2-one
Ph phenyl
PG protecting group
POCl₃ phosphorus (III)oxychloride
p-TosOH p-toluensulfonic acid
Pd(PPh₃)₄ Palladium (0) tetrakis triphenylphospine complex
rt (or RT) room temperature
TBAF tetrabutylammonium fluoride
TBAHS tetrabutylammonium hydrosulfate
TBTU O-(benzotriazol-1-yl)-N,N,N′,N′-tetramethylammonium tetrafluoroborate
^{t}BuOH tert.butanol
TEA triethyl amine
TFA trifluoroacetic acid
TFAA trifluoroacetic anhydride
THF tetrahydrofurane (dest. from Na/benzophenone)
TLC thin layer chromatography
TMS trimethylsilyl
TPTU O-[2-oxo-1(2H)-pyridyl]-N,N,N′,N′-tetramethyluronium-tetrafluoroborate [125700-71-2]
t_{Ret} retention time (_{A}t_{Ret}: retension time condition A)
UV ultraviolet

Where no specific source is indicated, starting materials are obtainable from customary suppliers, such as Sigma-Aldrich, St. Louis, USA, from Fluka, Switzerland, Buchs, from Merck, Darmstadt, FRG, or from providers indicated specifically.

### Synthesis

Flash chromatography is performed by using silica gel (Merck; 40-63 μm). For thin layer chromatography, pre-coated silica gel (Merck 60 F254; Merck KgaA, Darmstadt, Germany)) plates are used; the R_{f} values which indicate the ratio of the distance moved by each substance to the distance moved by the eluent front. ¹NMR measurements are performed on a Varian Gemini 400 spectrometer using tetramethylsilane as internal standard. Chemical shifts (δ) are expressed in ppm downfield from tetramethylsilane. Electrospray mass spectra are obtained with a Fisons Instruments VG Platform II.

Commercially available solvents and chemicals are used for syntheses. Unless otherwise indicated, reactions are carried out at rt under an inert atmosphere of N₂.

### HPLC Condition A:

### Column: Speed ROD RP18e, 50×4.6 mm.

Flow rate: 1.3 ml/min
Mobile phase: A) TFA/water (0.1/100, v/v), B) TFA/acetonitrile (0.1/100, v/v)
Gradient: linear gradient from 0% B to 100% B in 6 min then 2 min 100% B

### Detection: UV at 215 nm

### HPLC Condition B:

### System: HPLC; Acquity, Waters

### Column: BEH C18 1.7 μM.

Flow rate: 1.0 ml/min
Mobile phase: A) TFA/water (0.1/100, v/v), B) TFA/acetonitrile (0.1/100, v/v)
Gradient: linear gradient from 2% B to 100% B in 1.6 min then 0.4 min 100% B

### Detection: UV at 215 nm

### HPLC Condition C:

### System: Waters Alliance

### Column: Sunfire™ C18, 4.6×20 mm, 3.5 μM.

Flow rate: 3.0 ml/min.
Mobile phase: A) TFA/water (0.1/100, v/v), B) Acetonitrile.
Gradient: linear gradient 5-100% B and A in 4 min+0.5 min B.

### Detection: UV at 215 nm to 400.0 nm.

### MPLC (Medium Pressure Liquid Chromatography):

Combi Flash system: System: Combi Flash Companion from Isco, Inc.; columns: RediSep® flash column, Teledyne Isco, filled with 4 g, 12 g, 40 g or 120 g of SiO₂; application to column: either mixture is dissolved as a concentrated solution in eluent, or a solution of the mixture is concentrated together with SiO₂ in vacuo and applied as powder
Reversed phase chromatography: Gilson system GX-281: reversed phase Nucleosil C18; mobile phase: A) TFA/water (0.1/100, v/v), B) acetonitrile; linear gradient from 5% B to 100% B; mixture applied as solution in NMP; basic products are obtained as TFA-salts by concentration and/or lyophilisation, or as free base after neutralization with NaHCO₃, partial concentration and filtration or extraction with EtOAc or CH₂Cl₂.
   prep-HPLC: Waters HPLC prep-system, UV detector Waters 2487 Dual A Absorbance Detector or MS detector Waters micromassZQ, reversed phase column SunFire™ Prep, C18 OBD, 100×30 mm, 5 mm, or 100×19 mm, 5 μm, gradient elution (CH₃CN/water with 0.1% TFA), generally product obtained as a TFA salt after lyophilization.

### Example 1

### 1-(5-Chloro-2-methyl-phenyl)-5-(3-chloro-phenyl)-2-(3,4-dimethyl-phenyl)-1H-imidazole-4-carboxylic acid ethylamide

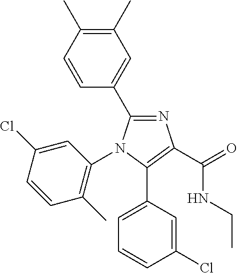

A solution of Example 5 (0.3 mmol), N-methyl-morpholin (171 μl, 1.55 mmol), ethylamine (2 M in THF; 384 μl, 0.768 mmol) and TPTU (200.8 mg, 0.676 mmol) in DMF (4 ml) is stirred for 16 h at rt. The mixture is diluted with EtOAc and water, the aq. layer is separated off and extracted twice with EtOAc. The organic phases are washed twice with H₂O and brine, dried (Na₂SO₄) and concentrated. Reversed phase chromatography gives the title compound. mp: 141-143° C.; MS: [M+1]⁺=478/480; HPLC: _{B}t_{Ret}=1.41 min; ¹H NMR (CD₃OD) δ 8.19 (m, HN, partially exchanged by D), 7.44 (s, 1H), 7.3 (m, 4H), 7.23 (t, 1H), 7.16 (m, 2H), 7.04 (s, 2H), 3.37 (m, 2H), 2.23 (s, H₃C), 2.19 (s, H₃C), 1.78 (s, H₃C), 1.22 (t, H₃C).

### Example 2

### 5-[1-(5-Chloro-2-methyl-phenyl)-5-(3-chloro-phenyl)-2-(3,4-dimethyl-phenyl)-1H-imidazol-4-yl]-tetrazole

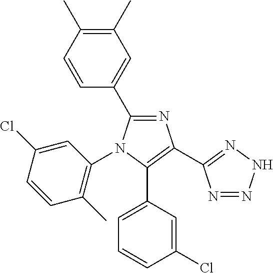

A suspension of NaN₃ (371.9 mg, 5.72 mmol) in toluene (325 μl) is cooled in an ice bath. Then Et₂AlCl (1.8 M in toluene; 3.18 ml, 5.72 mmol) is added and the mixture is stirred for 3 h at rt. The mixture is cooled to 0° C. again and Example 3 (190 mg, 0.44 mmol) and toluene (2 ml) are added in 2 portions. The mixture is allowed to slowly warm up to rt. After 3 h, the suspension is poured into water (100 ml) and EtOAc (100 ml). After stirring for 10 min, the precipitate is filtered off and washed with EtOAc. The aq. layer is separated off from the filtrate and extracted twice with EtOAc. The organic phases are washed with H₂O and brine, dried (Na₂SO₄) and concentrated. Reversed phase chromatography gives the title compound. ES-MS: [M+1]⁺=475/477; HPLC: _{B}t_{Ret}=1.35 min; ¹H NMR (DMSO-d₆) δ 7.79 (s, 1H), 7.38 (m, 2H), 7.30 (s, 1H), 7.22 (m, 2H), 7.18 and 7.15 (2m, 2H), 7.01 (d, 1H), 6.92 (d, 1H), 2.17 (s, H₃C), 2.14 (s, H₃C), 1.71 (s, H₃C).

### Example 3

### 1-(5-chloro-2-methyl-phenyl)-5-(3-chloro-phenyl)-2-(3,4-dimethyl-phenyl)-1H-imidazole-4-nitrile

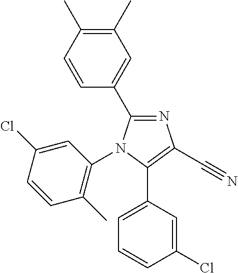

To an ice cooled solution of Example 4 (426 mg, 0.95 mmol) in CH₂Cl₂ (9.5 ml) and Et₃N (397 μl, 2.85 mmol), (F₃CSO₂)₂O (259 μl, 1.57 mmol) is added. The red solution is stirred for 5 min at 0° C. and 10 min at rt and then poured into a mixture of sat. NaHCO₃ (50 ml) solution and water (100 ml). This mixture is extracted with 3 portions of EtOAc. The organic phases are washed with H₂O and brine, dried (Na₂SO₄) and partially concentrated. The crystallized title compound is filtered off and washed with EtOAc. mp: 218° C.; ES-MS: [M+1]⁺=432/434; IR: 2232 cm⁻¹; ¹H NMR (DMSO-d₆) δ 7.89 (s, 1H), 7.49 and 7.45 (2m, 3H), 7.43 (s, 1H), 7.29 (m, 2H), 7.25 (s, 1H), 7.07 (d, 1H), 6.91 (d, 1H), 2.18 (s, H₃C), 2.14 (s, H₃C), 1.71 (s, H₃C).

### Example 4

### 1-(5-Chloro-2-methyl-phenyl)-5-(3-chloro-phenyl)-2-(3,4-dimethyl-phenyl)-1H-imidazole-4-carboxylic acid amide

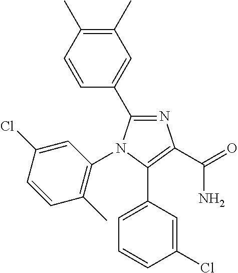

To Example 5 (580 mg, 1.28 mmol) in dioxane (8 ml), di-tert-butyl-dicarbonate (559 mg, 2.56 mmol) and pyridine (103 μl, 1.28 mmol) are added. After stirring for 15 min at rt, H₄NHCO₃ (202 mg, 2.56 mmol) is added and the mixture is stirred for 16 h at 40° C. Another 280 mg di-tert.-butyl-dicarbonate and 101 mg H₄NHCO₃ are added and stirring is continued for additional 4 h at 40° C. The mixture is diluted with EtOAc and water, the aq. layer is separated off and extracted twice with EtOAc. The organic phases are washed with H₂O and brine, dried (Na₂SO₄) and concentrated. Crystallization from EtOAc/hexane gives the title compound. ES-MS: [M+1]⁺=450/452; HPLC: _{B}t_{Ret}=1.31 min; ¹H NMR (CD₃OD) δ 7.44 (s, 1H), 7.3 (m, 4H), 7.22 (t, 1H), 7.16 (m, 2H), 7.04 (s, 2H), 2.23 (s, H₃C), 2.19 (s, H₃C), 1.78 (s, H₃C).

### Example 5

### 1-(5-Chloro-2-methyl-phenyl)-5-(3-chloro-phenyl)-2-(3,4-dimethyl-phenyl)-1H-imidazole-4-carboxylic acid

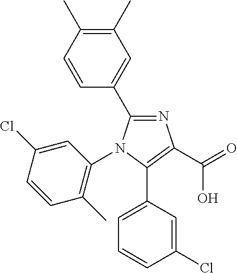

Example 6 (794 mg, 1.66 mmol) is dissolved in dioxane (12 ml). After adding a solution of LIOH H₂O (83.5 mg, 1.99 mmol) in H₂O (3 ml), it is stirred for ½ h at it and 4 h at 60° C. and finally concentrated in vacuo. Reversed phase chromatography, partial concentration of the product containing fractions and collection of the resulting precipitate gives the title compound. ES-MS: [M−1]=449/451; HPLC: _{B}t_{Ret}=1.22 min. ¹H NMR (DMSO d₆) δ 12.3 (sb, 1H), 7.81 (s, 1H), 7.41 (s, 1H), 7.36 (d, 1H), 7.33 (m, 1H), 7.28 (t, 1H), 7.27 (m, 1H), 7.23 (m, 1H), 7.21 (d, 1H), 7.03 (d, 1H), 6.91 (d, 1H), 2.17 (s, H₃C), 2.14 (s, H₃C), 1.69 (s, H₃C).

### Example 6

### 1-(5-Chloro-2-methyl-phenyl)-5-(3-chloro-phenyl)-2-(3,4-dimethyl-phenyl)-1H-imidazole-4-carboxylic acid ethyl ester

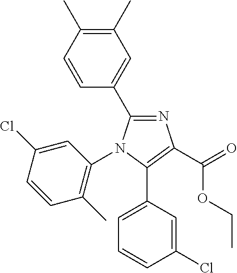

A solution of Intermediate 6.1 (1.57 g, 3.5 mmol) in dioxane (35 ml) and H₂O (17.5 ml) is degassed by repeated evacuation and flushing with N₂. Then K₃PO₄ (4.24 g, 20 mmol), 3-chloro-phenyl boronic acid (1.56 g, 10 mmol) and Pd(PPh₃)₄ (578 mg, 0.5 mmol) are added. Stirring for 6 h at 85° C. produces a red-brown solution. After cooling the mixture to ambient temperature it is diluted with EtOAc and water, the aq. layer is separated off and extracted twice with EtOAc. The organic phases are washed with H₂O and brine, dried (Na₂SO₄) and concentrated. Combi Flash chromatography (DCM/hexane 1:1→DCM→DCM/Et₂O 4:1) and crystallization from hexane gives the title compound. ES-MS: [M+1]⁺=479/481; HPLC: _{B}t_{Ret}=1.39 min. ¹H NMR (DMSO-d₆) δ 7.82 (s, 1H), 7.44 (s, 1H), 7.37 (m, 2H), 7.30 (t, 1H), 7.26 (s, 1H), 7.22 (d, 1H), 7.21 (d, 1H), 7.04 (d, 1H), 6.91 (d, 1H), 4.12 (m, 2H), 2.17 (s, H₃C), 2.14 (s, H₃C), 1.70 (s, H₃C), 1.08 (t, H₃C).

### Intermediate 6.1

### 5-Bromo-1-(5-chloro-2-methyl-phenyl)-2-(3,4-dimethyl-phenyl)-1H-imidazole-4-carboxylic acid ethyl ester

Intermediate 6.2 (4.8 g, 13 mmol) is dissolved in CH₃CN (30 ml) at rt and NBS (2.3 g, 13 mmol) is added. The reaction mixture is stirred at ambient temperature for 16 h and the volume is reduced to approx. 5 ml under reduced pressure. EtOAc is added and the organic layer is washed successively with NaHCO₃, H₂O and brine, dried over Na₂SO₄ and concentrated. The crude product is treated with diethyl ether. Insolubles are removed by filtration and the mother liquor is concentrated to give the title compound as a yellow foam. ES-MS: M+=448.9; HPLC: _{A}t_{Ret}=5.23 min. ¹H NMR (CD₃OD) 7.53-7.51 (m, 2H), 7.39 (d, 1H), 7.22 (s, 1H), 7.02-6.99 (m, 2H), 4.40 (q, 2H), 2.28 (s, 3H), 2.19 (s, 3H), 1.83 (s, 3H), 1.40 (t, 3H).

### Intermediate 6.2

### 1-(5-Chloro-2-methyl-phenyl)-2-(3,4-dimethyl-phenyl)-1H-imidazole-4-carboxylic acid ethyl ester

Intermediate 6.3 (5.0 g, 13.0 mmol) is dissolved in toluene (80 ml) and p-TosOH (600 mg, 3.6 mmol) is added at rt. The reaction mixture is then heated to 60° C. and stirred for 12 h. It is allowed to cool to ambient temperature again, diluted with EtOAc and the organic layer is subsequently washed with H₂O, aq NaHCO₃ and brine, dried and concentrated to give the crude product as a yellow foam. ES-MS: M+=371.2; HPLC: _{A}t_{Ret}=4.71 min. ¹H NMR (CD₃OD) 7.99 (s, 1H), 7.42-7.39 (m, 2H), 7.34 (d, 1H), 7.21 (s, 1H), 7.01-6.98 (m, 2H), 4.38 (q, 2H), 2.24 (s, 3H), 2.17 (s, 3H), 1.83 (s, 3H), 1.39 (t, 3H).

### Intermediate 6.3

### 1-(5-Chloro-2-methyl-phenyl)-2-(3,4-dimethyl-phenyl)-4-hydroxy-4,5-dihydro-1H-imidazole-4-carboxylic acid ethyl ester

Intermediate 6.4 (3.5 g, 13 mmol) is dissolved in THF/H₂O (1:1; 40 ml) at rt. Ethyl bromo pyvurate (85% purity grade, 2.2 ml, 15.6 mmol) and NaHCO₃ (4.5 g, 54 mmol) are added successively and stirring is continued at rt temperature for 12 h. The reaction mixture is concentrated under reduced pressure and taken up in EtOAc. The organic layer is washed with H₂O and brine, dried over MgSO₄ and concentrated to give the title compound as a white foam. ES-MS: M+=389.1; HPLC: _{A}t_{Ret}=3.65 min. ¹H NMR (CD₃OD) 7.24 (s, 1H), 7.21-7.17 (m, 2H), 7.13-7.00 (m, 3H), 4.31 (q, 2H), 2.24 (s, 6H), 2.20-2.18 (m, 2H), 2.18 (s, 3H), 1.58 (t, 3H).

### Intermediate 6.4

### N-(5-Chloro-2-methyl-phenyl)-3,4-dimethyl-benzamidine

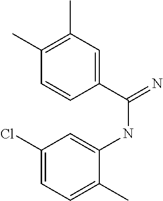

2-Methyl-5-chloroaniline (5.0 g, 35 mmol) is dissolved in toluene (100 ml) and cooled to 0° C. To this solution trimethyl aluminium (2M solution in toluene; 17.5 ml, 35 mmol) is added dropwise. After completion of the addition the reaction mixture is stirred at ambient temperature for 1 h. After 2.5 h 3,4-dimethyl benzonitrile (5.5 g, 42 mmol) is added and stirring is continued for 20 h at 80° C. The reaction is then allowed to cool to ambient temperature. Celite is added and the reaction is carefully quenched by dropwise addition of DCM/MeOH (2:1). The resulting precipitate is filtered off and washed repeatedly with DCM/MeOH (2:1). The collected filtrates are concentrated. The resulting crude product is titurated with hexanes/EtOAc and washed with cold hexanes to give the title compound as a white solid. ES-MS: M+=275.1; HPLC: _{A}t_{Ret}=2.93 min. ¹H NMR (CD₃OD) 7.61 (s, 1H), 7.59 (d, 1H), 7.21 (d, 1H), 7.19 (d, 1H), 6.99 (d, 1H), 6.84 (s, 1H), 2.36 (s, 3H), 2.34 (s, 3H), 2.14 (s, 3H).

### Example 7

### 5-(3-Chloro-4-fluoro-phenyl)-1-(3-chloro-2-fluoro-phenyl)-2-thiophen-3-yl-1H-imidazole-4-carboxylic acid ethyl ester

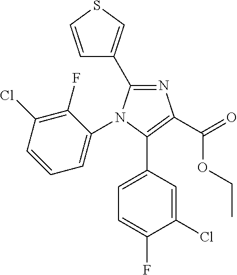

A suspension of Intermediate 7.1 (476 mg, 1.00 mmol) in dioxane (10 ml) and H₂O (5 ml) is degassed by repeated evacuation and flushing with N₂. Then K₃PO₄ (1.21 g, 5.7 mmol), thiophen-3-boronic acid (365 mg, 2.85 mmol) and Pd(PPh₃)₄ (164 mg, 0.142 mmol) are added. Stirring for 7 h at 85° C. produces a red solution. After cooling the mixture to rt it is diluted with EtOAc and water, the aq. layer is separated off and extracted twice with EtOAc. The organic phases are washed with H₂O and brine, dried (Na₂SO₄) and concentrated. Combi Flash chromatography (hexane/EtOAc 9:1→1:2) and crystallization from hexane gives the title compound. mp: 207-209° C.; ES-MS: [M+1]⁺=4791481; HPLC: _{B}t_{Ret}=1.34 min; ¹H NMR (DMSO d₆) 7.75 (t, 1H), 7.68 (t, 1H), 7.61 (d, 1H), 7.58 (m, 1H), 7.36 (m, 2H), 7.26 (m, 2H), 7.12 (d, 1H), 4.13 (m, 2H), 1.09 (t, 3H).

### Intermediate 7.1

### 2-Bromo-5-(3-chloro-4-fluoro-phenyl)-1-(3-chloro-2-fluoro-phenyl)-1H-imidazole-4-carboxylic acid ethyl ester

To a solution of Intermediate 7.2 (1098 mg, 2.66 mmol) in toluene (50 ml), POBr₃ (1.525 g, 5.32 mmol) is added. This solution is stirred for 22 h at 110° C. Then a second portion of POBr₃ (1.525 g, 5.32 mmol) is added and stirring is continued for 22 h at 110° C. The reaction mixture is poured into water (100 ml) and sat. NaHCO₃ (150 ml) and extracted with 3 portions of EtOAc. The organic phases are washed with H₂O and brine, dried (Na₂SO₄) and concentrated. Combi Flash chromatography (hexane/EtOAc 19:1→1:2) and crystallization from EtOAc gives the title compound. ES-MS: [M+1]⁺=475/477/479; HPLC: _{B}t_{Ret}=1.32 min; ¹H NMR (DMSO-d₆) δ 7.78 (m, 1H), 7.67 (m, 1H), 7.63 (m, 1H), 7.38 (m, 2H), 7.25 (m, 1H), 4.11 (q, 2H), 1.08 (t, H₃C).

### Intermediate 7.2

### 5-(3-Chloro-4-fluoro-phenyl)-1-(3-chloro-2-fluoro-phenyl)-2-oxo-2,3-dihydro-1H-imidazole-4-carboxylic acid ethyl ester

Intermediate 7.3 (913 mg, 4.84 mmol) is added to a solution of Intermediate 7.4 (1.191 g, 4.4 mmol) in 1,2-dichloro-ethane (22 ml) and toluene (22 ml). The suspension is degassed by repeated evacuation and flushing with N₂. Then Rh₂Oct₄ ([Cas: 73482-96-9]; 68.5 mg, 0.088 mmol), is added and the mixture is heated up to 80° C. N₂ is split off and a green solution is formed. After 1 h, the educts are consumed and an intermediate is formed (MS: [M+1]⁺=431/433). The solution is cooled in an ice bath, then TFA (5.28 ml) is added to eliminate H₂O from that intermediate to form the desired imidazole. After stirring for ½ h in the ice bath, 17 h at rt and finally 3 h at 50° C., the elimination is complete. The solution is diluted with EtOAc and water/sat. Na₂CO₃ 1:1 (200 ml), the aq. layer is separated off and extracted twice with EtOAc. The organic phases are washed with H₂O and brine, dried (Na₂SO₄) and concentrated. Combi Flash chromatography (hexane/EtOAc 4:1→3:7) and crystallization from EtOAc/hexane gives the title compound. ES-MS: [M+1]⁺=413/415; HPLC: _{B}t_{Ret}=1.18 min; ¹H NMR (DMSO-d₆) δ 11.43 (sb, HN), 7.65 (m, 1H), 7.62 (t, 1H), 7.39 (t, 1H), 7.34 (t, 1H), 7.25 (t, 1H), 7.20 (m, 1H), 4.08 (q, 2H), 1.06 (t, H₃C).

### Intermediate 7.3

### (3-Chloro-2-fluoro-phenyl)-urea

3-Chloro-2-fluoro-aniline (7.28 g, 50 mmol) is mixed with water (72 ml). Then a solution of KOCN (4.14 g, 51 mmol) in H₂O/AcOH 9:1 (144 ml) is added. During stirring at rt, a precipitation is formed. After 16 h, 4.14 g KOCN and 7 ml AcOH are added. The suspension is stirred for another 4 h, then filtered and the crude product washed with water. Column chromatography (SiO₂; CH₂Cl₂/Et₂O/acetone 66:33:1) and crystallization from acetone/toluene gives the title compound. ES-MS: [M−1]=187/189; HPLC: _{B}t_{Ret}=0.77 min. Alternatively the product can be recrystallized from NMP (120° C.→rt); ¹H NMR (DMSO-d₆) δ 8.49 (sb, HN), 8.08 (m, 1H), 7.08 (m, 1H), 7.06 (m, 1H), 6.25 (sb, H₂N).

### Intermediate 7.4

### 3-(3-Chloro-4-fluoro-phenyl)-2-diazo-3-oxo-propionic acid ethyl ester

To a solution of Intermediate 7.5 (1.29 g, 5.27 mmol) in toluene (20 ml), Et₃N (0.88 ml, 6.32 mmol) and 4-dodecyl-benzenesulfonyl azide ([Cas: 79791-38-1]; 2.22 g, 6.32 mmol) are added. The clear solution is stirred over night at rt and then diluted with EtOAc and water/sat. NaHCO₃ 1:1. The aq. layer is separated off and extracted twice with EtOAc. The organic phases are washed with H₂O and brine, dried (Na₂SO₄) and concentrated. Combi Flash chromatography (hexane/EtOAc (99:1→9:1) gives the title compound as an oil that solidifies slowly in the fridge. ES-MS: [M+1]⁺=271/273; TLC(hexane/EtOAc 9:1): R_{f}=0.26; IR: [cm⁻¹] 2154 s, 2130 m, 1314 s.

### Alternative Method:

To an ice-cooled solution of Intermediate 7.5 (18.8 g, 77 mmol) in CH₃CN (730 ml), p-acetamido-benzenesulfonyl azide (18.46 g, 77 mmol) and Et₃N (32.1 ml, 231 mmol) are added. The suspension is stirred for 3 h at rt, then diluted with Et₂O/hexane 1:1 (280 ml) and finally filtered. The filtrate is concentrated in vacuo and the residue purified by column chromatography (SiO₂; hexane/EtOAc 199:1→9:1); ¹H NMR (DMSO-d₈) δ 7.85 (m, 1H), 7.63 (m, 1H), 7.51 (d t, 1H), 4.16 (m, 2H), 1.17 (dt, H₃C).

### Intermediate 7.5

### 3-(3-Chloro-4-fluoro-phenyl)-3-oxo-propionic acid ethyl ester

Carbonyl-di-imidazole (62.1 g, 368 mmol) is added to an ice cooled solution of 3-chloro-4-fluoro-benzoic acid (59 g, 331 mmol) in THF (600 ml). Stirring for 23 h at rt gives a solution of activated ester. In a separate vessel, propanedioic acid 1-ethyl ester potassium salt (54 g, 317 mmol) is suspended in THF (600 ml) and cooled in an ice bath. A THF solution of ⁱPrMgCl (2 M; 159 ml, 318 mmol) is added during 15 min. The mixture is stirred for 20 min at 0° C., 90 min at rt and finally 45 min at 50° C. and then cooled again in the ice bath. Now the solution of the activated ester is added dropwise at 0-2° C., forming a suspension which is stirred afterwards for 16 h at rt and 0.5 h at 50° C. After cooling the beige suspension in an ice-bath, 1 N HCl (600 ml) is added (pH: 6-7). The resulting red solution is stirred for 0.5 h and finally extracted with 2 portions of EtOAc (4 l). The organic layers are washed twice with H₂O (3 l) and brine (1 l), dried (Na₂SO₄) and concentrated. Column chromatography (SO₂; EtOAc/hexane 1:6) and stirring in heptane (0.1 l) gives the title compound. mp: 39-40° C.; ES-MS: [M+1]⁺=245/247; TLC(hexanelEtOAc 9:1): R_{f}=0.26; ¹H NMR (DMSO-d₆) δ 8.16 (m, 1H), 7.97 (m, 1H), 7.59 (t, 1H), 4.22 (s, 2H), 4.10 (q, 2H), 1.16 (t, H₃C).

### Example 8

### 1-(3-Chloro-2-fluoro-phenyl)-2-(3-chloro-phenyl)-5-phenyl-1H-pyrrole-3-carboxylic acid

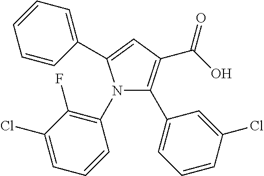

Example 9 (120 mg, 0.2 mmol) is dissolved in dioxane (2 ml) and H₂O (1 ml). LiOH*H₂O (32 mg, 0.8 mmol) is added and the mixture is stirred at 60° C. for 24 h. It is allowed to cool to rt, diluted with EtOAc and washed with sat. aq. NH₄Cl solution dried over Na₂SO₄ and concentrated and dried under high vacuum to give the title compound as a white solid. ES-MS: M+=427.8; ¹HNMR (MeOH d₄) 7.39 (dd, 1H), 7.26-7.12 (m, 10H), 7.04 (dd, 1H), 6.88 (s, 1H).

### Example 9

### 1-(3-Chloro-2-fluoro-phenyl)-2-(3-chloro-phenyl)-5-phenyl-1H-pyrrole-3-carboxylic acid ethyl ester

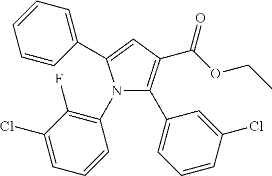

Intermediate 9.1 (180 mg, 0.55 mmol) is dissolved in EtOH (1 ml) and toluene (1 ml). p-TosOH (13 mg, 0.05 mmol) and 3-chloro-2-fluoroaniline (300 mg, 2.2 mmol) are added and the reaction mixture is heated with stirring to reflux for 24 h. It is allowed to cool to rt, diluted with EtOAc and the organic layer is washed with aq. NaHCO₃ solution and brine, dried over Na₂SO₄ and concentrated. The remaining crude product is purified by flash chromatography (SiO₂, hexane/EtOAc, gradient 0-10% EtOAc) to give the title compound as a white solid. ES-MS: M+=455.8. ¹HNMR (CDCl₃) 7.28-7.21 (m 7H), 7.16-7.12 (m, 3H), 6.94-6.90 (m, 3H), 4.17 (q, 2H), 1.18 (t, 3H).

### Intermediate 9A

### 2-(3-Chloro-benzoyl)-4-oxo-4-phenyl-butyric acid ethyl ester

3-(3-Chloro-phenyl)-3-oxo-propionic acid ethyl ester (500 mg, 2.2 mmol) is dissolved in THF (20 ml). The solution is cooled to 0° C. and NaH (60% oil dispersion, 105 mg, 2.2 mmol) is added. The reaction mixture is allowed to warm to it and stirred for 1 h. A solution of 2-bromo-1-phenyl-ethanone (440 mg, 2.2 mmol) in THF (10 ml) is added at it and stirring continued for 1 h, while a yellow precipitate forms. The reaction is quenched by addition of aq. NH₄Cl (1N) and EtOAc. The organic layer is separated, dried over Na₂SO₄, concentrated and dried under high vacuum to give the title compound as a yellow solid. ES-MS: M+=345.0; ¹HNMR (CDCl₃) δ 8.07 (s, 1H), 8.03-8.00 (m, 3H), 7.59 (d, 2H), 7.47-7.44 (m, 3H), 5.04 (dd, 1H), 4.17 (q, 2H), 3.87 (dd, 1H), 3.73 (dd, 1H), 1.18 (t, 3H).

### Example 10

### 4,5-Bis-(3-chloro-phenyl)-1-phenyl-1H-pyrazole-3-carboxylic acid

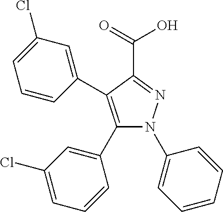

In a sealed reaction flask, a mixture of Intermediate 10.1 (40 mg, 0.099 mmol, 1.0 equiv.), 3-chlorophenylboronic acid (20 mg, 0.128 mmol, 1.3 equiv.) and PdCl₂(PPh₃)₂ (3.5 mg, 0.005 mmol, 0.05 equiv.) in Na₂CO₃ 2M in water (0.39 ml) and DME (1 ml) was heated at 150° C. for 17 min under microwave irradiation (following these reaction conditions, hydrolyzes of the carboxylic ester occurred quantitatively). The reaction mixture was diluted into DCM and washed with water. The aqueous phase was further extracted with DCM and the combined organic fractions were evaporated to dryness. The resulting residue was purified by reversed phase prep-HPLC (Waters system, gradient elution, water with 0.1% TFA/MeCN) to yield the title compound as a colorless solid. ES-MS: [M+H]⁺=409.0; ¹H NMR (400 MHz, CD₃OD) δ 7.01 (m, 1H), 7.10 (m, 1H), 7.15 (m, 1H), 7.21-7.45 (m, 10H).

### Intermediate 10.1

### 4-Bromo-5-(3-chloro-phenyl)-1-phenyl-1H-pyrazole-3-carboxylic acid ethyl ester

To a solution of Intermediate 10.2 (430 mg, 1.3 mmol, 1.0 equiv.) in DMF (4 ml) was added NBS (284 mg, 1.5 mmol, 1.15 equiv.) at RT. The reaction mixture was heated at 50° C. for 3 h, then cooled to RT, diluted into Et₂O (100 ml) and washed successively with Na₂CO₃ 2M in water (40 ml), water (2×40 ml) and brine (40 ml). The organic layer was dried over Na₂SO₄, filtered and concentrated to dryness to yield the crude title compound (544 mg, 1.3 mmol, quant.) as a brownish oil, which was used in the next step without further purification. LCMS: _{C}t_{Ret}=2.89 min; MS: m/z 407.0 [M+H]⁺; ¹H NMR (400 MHz, CDCl₃) δ 1.47 (t, J=7.1, 3H), 4.51 (q, J=7.1, 2H), 7.11-7.15 (m, 1H), 7.26-7.41 (m, 8H).

### Intermediate 10.2

### 5-(3-Chloro-phenyl)-1-phenyl-1H-pyrazole-3-carboxylic acid ethyl ester

In a sealed reaction flask, a stirred solution of Intermediate 10.3 (485 mg, 1.6 mmol, 1.0 equiv.) and phenylhydrazine (0.2 ml, 2.0 mmol, 1.2 equiv.) in DCE (15 ml) was heated at 100° C. for 6 h. The reaction mixture was cooled to RT and concentrated under vacuum.

The resulting residue was purified by Combi-Flash Companion™ (Isco Inc.) column chromatography (SiO₂; gradient elution, heptane I AcOEt 95:5→4:1) to yield the title compound as an orange oil. TLC: R_{F}=0.29 (heptane/AcOEt 4:1); LCMS: _{C}t_{Ret}=2.73 min; MS: m/z 327.1 [M+H]⁺; ¹H NMR (400 MHz, CDCl₃) δ 1.45 (t, J=7.1, 3H), 4.48 (q, J=7.1, 2H), 7.04-7.07 (m, 1H), 7.08 (s, 1H), 7.22-7.41 (m, 8H).

### Intermediate 10.3

### 4-(3-Chloro-phenyl)-2-(methoxy-methyl-amino)-4-oxo-but-2-enoic acid ethyl ester

To a solution of ethyl propiolate (1.0 ml, 10.0 mmol, 2.0 equiv.) in anhydrous THF (10 ml) was added LiHMDS (1M in THF, 10.0 ml, 10.0 mmol, 2.0 equiv.) dropwise at −78° C. (dry ice I acetone bath). The reaction mixture was stirred at −78° C. for 30 min before a solution of Intermediate 10.4 (1.0 g, 5.0 mmol, 1.0 equiv.) in anhydrous THF (5 ml) was added slowly. The resulting mixture was stirred at −78° C. for 10 min then allowed to warm to −40° C. during 1 h and stirred at that temperature for additional 30 min. The reaction mixture was quenched by the addition of HCl 2M in water (5 ml), then diluted into Et₂O (200 ml) and washed with water (2×80 ml). The manic layer was dried over Na₂SO₄, filtered and evaporated. The resulting residue was purified by Combi-Flash Companion™ (Isco Inc.) column chromatography (SiO₂; gradient elution, heptane I AcOEt 95:5→6:4) to yield the title compound as an orange oil. TLC: R_{F}=0.26 (heptane I AcOEt 7:3); LCMS: _{C}t_{Ret}=2.23 min; MS: m/z 298.0 [M+H]⁺; ¹H NMR (400 MHz, CDCl₃) δ 1.41 (t, J=7.1, 3H), 3.19 (s, 3H), 3.76 (s, 3H), 4.47 (q, J=7.1, 2H), 5.99 (s, 1H), 7.38 (m, 1H), 7.48 (m, 1H), 7.79 (m, 1H), 7.89 (m, 1H).

### Intermediate 10.4

### 3-Chloro-N-methoxy-N-methyl-benzamide

To a mixture of O,N-dimethyl-hydroxylamine hydrochloride (602 mg, 6.1 mmol, 1.1 equiv.) and Et₃N (1.9 ml, 13.8 mmol, 2.5 equiv.) in DCM (15 ml) was added 3-chlorobenzoyl chloride (0.73 ml, 5.5 mmol, 1.0 equiv.) dropwise at 0° C. (ice bath). The resulting yellow mixture was stirred at 0° C. for 10 min then allowed to warm to RT and stirred for additional 30 min. The reaction mixture was diluted into AcOEt (60 ml) and washed successively with HCl 1M in water (2×30 ml), Na₂CO₃ 2M in water (2×30 ml) and brine (30 ml). The organic layer was dried over Na₂SO₄, filtered and evaporated to dryness to yield the crude title compound as a yellow oil, which was used in the next step without further purification. LCMS: _{C}t_{Ret}=1.58 min; m/z 200.1 [M+H]⁺; ¹H NMR (400 MHz, CDCl₃) δ 3.38 (s, 3H), 3.57 (s, 3H), 7.36 (m, 1H), 7.45 (m, 1H), 7.59 (m, 1H), 7.69 (m, 1H).

TABLE 1
of further examples: Most of examples and intermediates were synthesized by
analogy methods like Example 1 to Example 10. Additional information (synthetic
methods for examples and intermediates) can be found following the table.

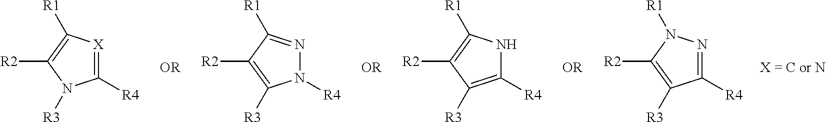

| | | | | | | Analysis |
|---|---|---|---|---|---|---|
| | | | | | | MS/ |
| Ex- | | | | | | HPLC/ |
| am- | Core | | | | | TLC/ |
| ple | template | R1 | R2 (A) | R3 (B) | R4 | NMR/IR |
| 11 | 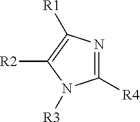 | 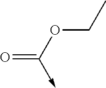 | 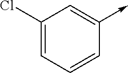 | 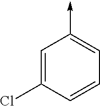 | 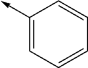 | M+ = 439.0 _{A}t_{Ret} = 2.33 min |
| 12 | 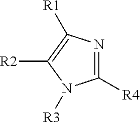 | 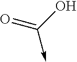 | 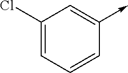 | 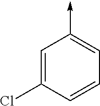 | 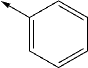 | M+ = 411.4 _{A}t_{Ret} = 4.67 min |
| 13 | 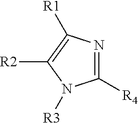 | 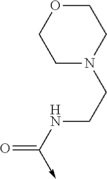 | 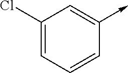 | 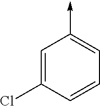 | 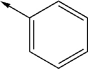 | M+ = 523.3 _{A}t_{Ret} = 4.12 min |
| 14 | 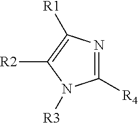 | 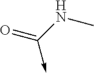 | 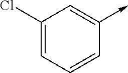 | 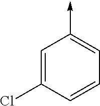 | 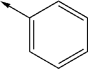 | M+ = 424.0 _{A}t_{Ret} = 5.17 min |
| 15 | 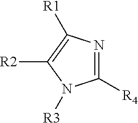 | 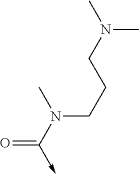 | 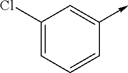 | 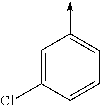 | 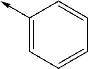 | M+ = 509.3 _{A}t_{Ret} = 4.33 min |
| 16 | 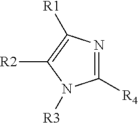 | 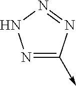 | 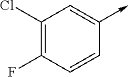 | 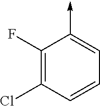 | 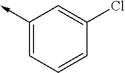 | M + H = 503/505 _{B}t_{Ret} = 1.38 min |
| 17 | 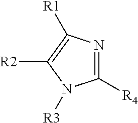 | 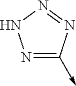 | 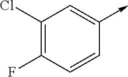 | 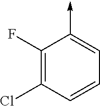 | 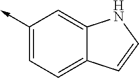 | M + H = 508/510 _{B}t_{Ret} = 1.27 min |
| 18 | 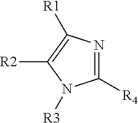 | 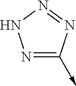 | 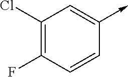 | 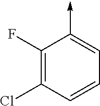 | 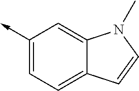 | M + H = 522/524 _{B}t_{Ret} = 1.34 min |
| 19 | 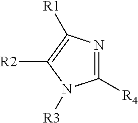 | 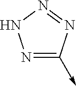 | 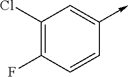 | 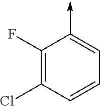 | 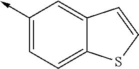 | M − H = 523/525 _{B}t_{Ret} = 1.37 min |
| 20 | 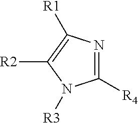 | 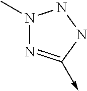 | 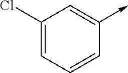 | 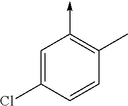 | 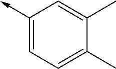 | TLC (DCM/ EtOAc = 19/1) Rf = 0.32 _{B}t_{Ret} = 1.34 min |
| 21 | 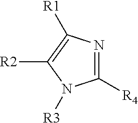 | 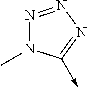 | 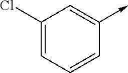 | 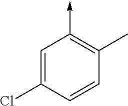 | 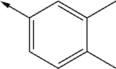 | TLC/ DCM/ EtOAc = 19/1) Rf = 0.52 _{B}t_{Ret} = 1.45 min |
| 22 | 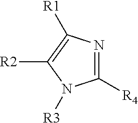 | 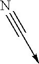 | 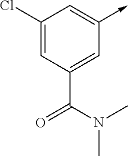 | 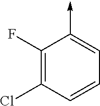 | 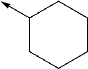 | M+ = 487.0 _{A}t_{Ret} = 5.42 min |
| 23 | 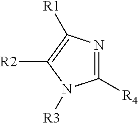 | 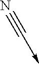 | 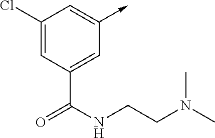 | 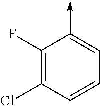 | 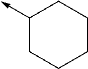 | M+ = 530.0 _{A}t_{Ret} = 4.47 min |
| 24 | 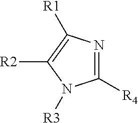 | 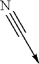 | 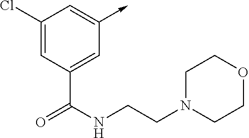 | 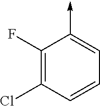 | 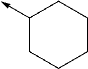 | M+ = 572.1 _{A}t_{Ret} = 4.53 min |
| 25 | 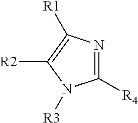 | 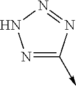 | 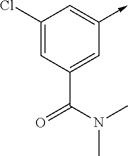 | 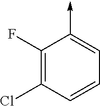 | 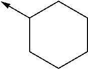 | M+ = 530.0 _{A}t_{Ret} = 4.91 min |
| 26 | 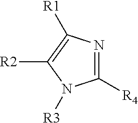 | 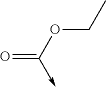 | 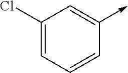 | 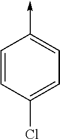 | 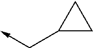 | M+ = 416.9 _{A}t_{Ret} = 4.92 min |
| 27 | 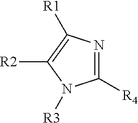 | 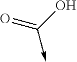 | 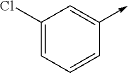 | 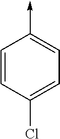 | 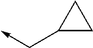 | M− = 385.1 _{A}t_{Ret} = 4.35 min |
| 28 | 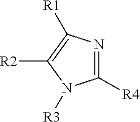 | 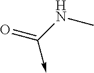 | 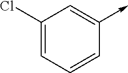 | 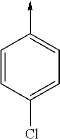 | 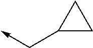 | M+ = 401.9 _{A}t_{Ret} = 4.65 min |
| 29 | 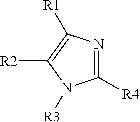 | 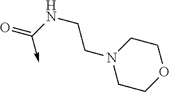 | 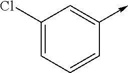 | 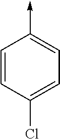 | 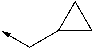 | M+ = 500.6 _{A}t_{Ret} = 4.39 min |
| 30 | 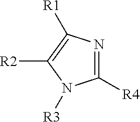 | 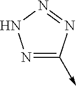 | 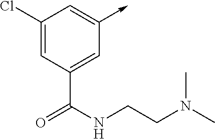 | 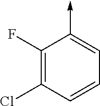 | 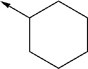 | M+ = 573.3 _{A}t_{Ret} = 4.18 min |
| 31 | 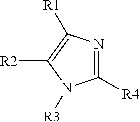 | 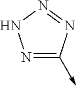 | 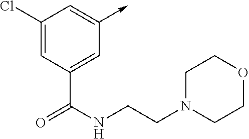 | 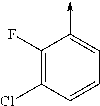 | 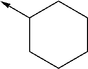 | M+ = 613.3 _{A}t_{Ret} = 4.23 min |
| 32 | 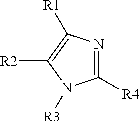 | 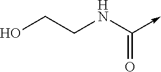 | 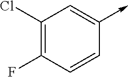 | 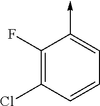 | 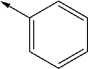 | M+ = 489.8 _{A}t_{Ret} = 4.88 min |
| 33 | 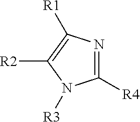 | 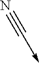 | 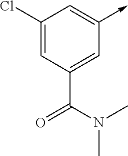 | 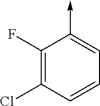 | 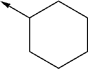 | M+ = 487.0 _{A}t_{Ret} = 5.42 min |
| 34 | 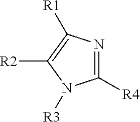 | 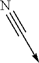 | 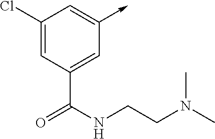 | 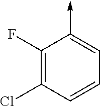 | 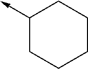 | M+ = 530.0 _{A}t_{Ret} = 4.47 min |
| 35 | 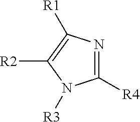 | 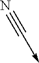 | 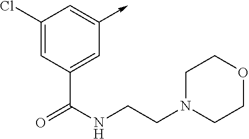 | 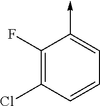 | 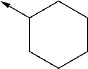 | M+ = 572.1 _{A}t_{Ret} = 4.53 min |
| 36 | 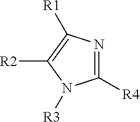 | 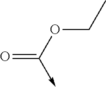 | 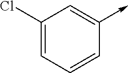 | 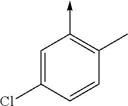 | 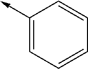 | M+ = 453.0 _{A}t_{Ret} = 5.61 min |
| 37 | 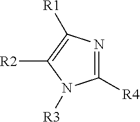 | 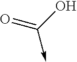 | 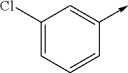 | 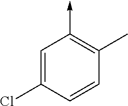 | 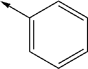 | M+ = 424.9 _{A}t_{Ret} = 4.82 min |
| 38 | 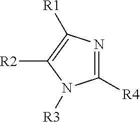 | 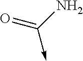 | 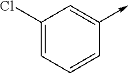 | 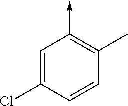 | 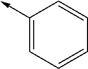 | M+ = 423.9 _{A}t_{Ret} = 5.13 min |
| 39 | 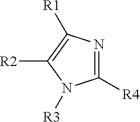 | 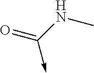 | 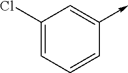 | 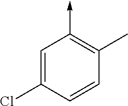 | 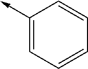 | M+ = 437.9 _{A}t_{Ret} = 5.33 min |
| 40 | 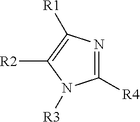 | 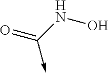 | 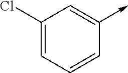 | 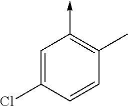 | 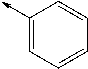 | M+ = 437.9 _{A}t_{Ret} = 6.65 min |
| 41 | 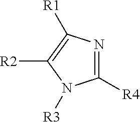 | 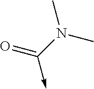 | 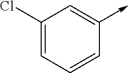 | 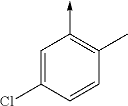 | 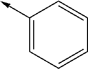 | M+ = 451.9 _{A}t_{Ret} = 5.09 min |
| 45 | 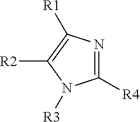 | 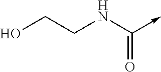 | 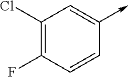 | 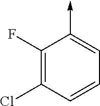 | 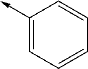 | M+ = 489.8 _{A}t_{Ret} = 4.88 min |
| 46 | 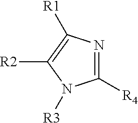 | 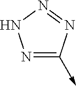 | 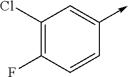 | 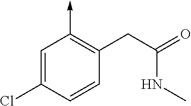 | 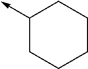 | M + H = 528/530 _{B}t_{Ret} = 1.18 min |
| 47 | 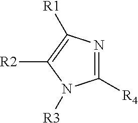 | 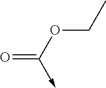 | 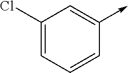 | 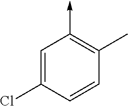 | 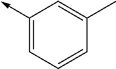 | M+ = 466.8 _{A}t_{Ret} = 5.70 min |
| 48 | 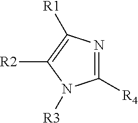 | 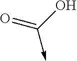 | 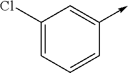 | 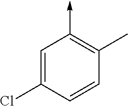 | 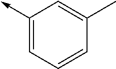 | M+ = 438.9 _{A}t_{Ret} = 5.01 min |
| 49 | 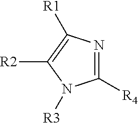 | 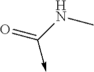 | 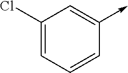 | 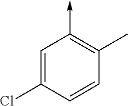 | 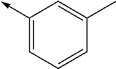 | M+ = 451.9 _{A}t_{Ret} = 5.59 min |
| 50 | 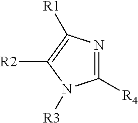 | 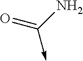 | 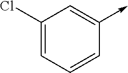 | 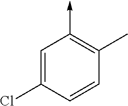 | 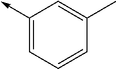 | M+ = 437.8 _{A}t_{Ret} = 5.36 min |
| 51 | 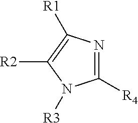 | 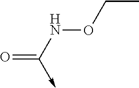 | 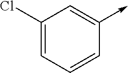 | 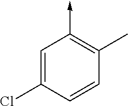 | 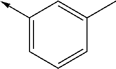 | M+ = 481.9 _{A}t_{Ret} = 5.63 min |
| 52 | 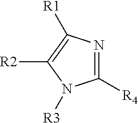 | 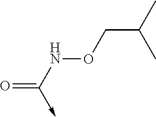 | 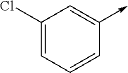 | 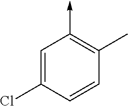 | 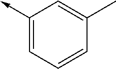 | M+ = 509.9 _{A}t_{Ret} = 6.12 min |
| 53 | 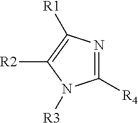 | 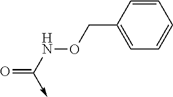 | 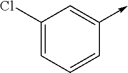 | 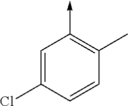 | 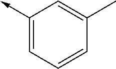 | M+ = 543.8 _{A}t_{Ret} = 6.00 min |
| 54 | 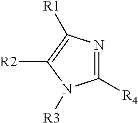 | 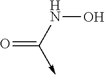 | 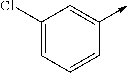 | 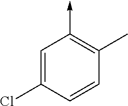 | 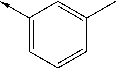 | M+ = 453.8 _{A}t_{Ret} = 5.09 min |
| 55 | 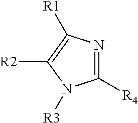 | 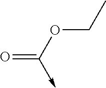 | 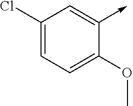 | 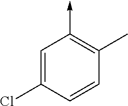 | 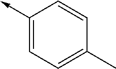 | M+ = 496.3 _{A}t_{Ret} = 5.61 min |
| 56 | 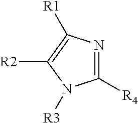 | 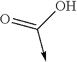 | 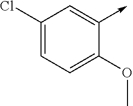 | 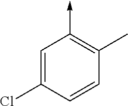 | 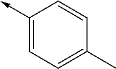 | M+ = 468.7 _{A}t_{Ret} = 4.95 min |
| 57 | 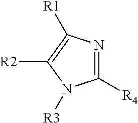 | 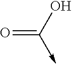 | 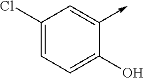 | 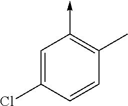 | 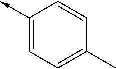 | M+ = 454.6 _{A}t_{Ret} = 4.57 min |
| 58 | 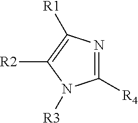 | 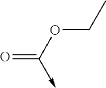 | 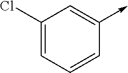 | 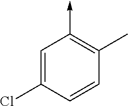 | 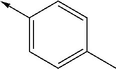 | M+ = 466.9 _{A}t_{Ret} = 5.67 min |
| 59 | 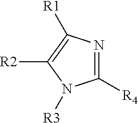 | 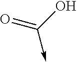 | 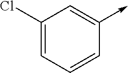 | 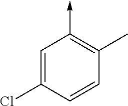 | 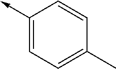 | M+ = 438.9 _{A}t_{Ret} = 4.96 min |
| 60 | 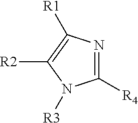 | 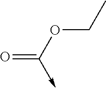 | 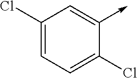 | 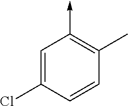 | 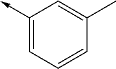 | M+ = 500.9 _{A}t_{Ret} = 6.06 min |
| 61 | 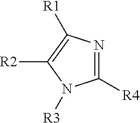 | 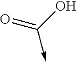 | 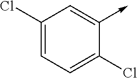 | 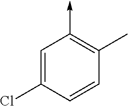 | 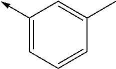 | M+ = 472.7 _{A}t_{Ret} = 5.31 min |
| 62 | 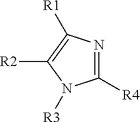 | 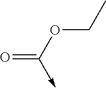 | 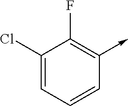 | 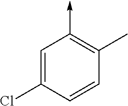 | 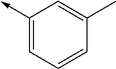 | M+ = 484.7 _{A}t_{Ret} = 5.87 min |
| 63 | 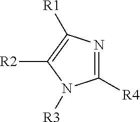 | 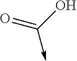 | 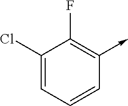 | 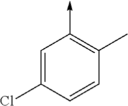 | 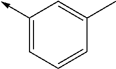 | M+ = 456.7 _{A}t_{Ret} = 5.11 min |
| 64 | 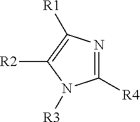 | 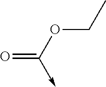 | 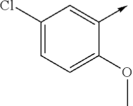 | 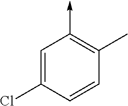 | 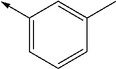 | M+ = 496.7 _{A}t_{Ret} = 5.63 min |
| 65 | 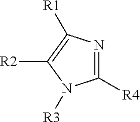 | 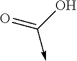 | 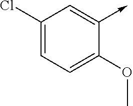 | 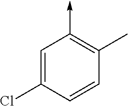 | 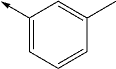 | M+ = 468.9 _{A}t_{Ret} = 4.90 min |
| 66 | 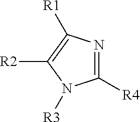 | 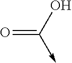 | 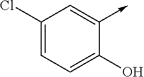 | 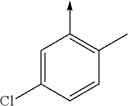 | 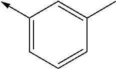 | M+ = 454.6 _{A}t_{Ret} = 4.59 min |
| 67 | 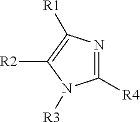 | 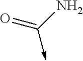 | 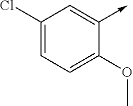 | 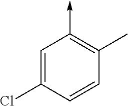 | 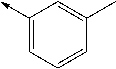 | M+ = 467.6 _{A}t_{Ret} = 5.20 min |
| 68 | 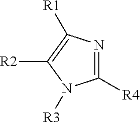 | 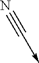 | 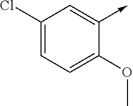 | 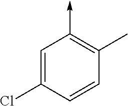 | 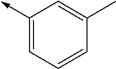 | M+ = 449.7 _{A}t_{Ret} = 5.94 min |
| 69 | 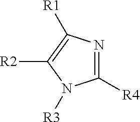 | 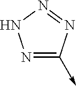 | 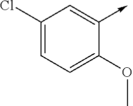 | 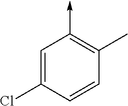 | 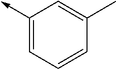 | M+ = 492.9 _{A}t_{Ret} = 5.45 min |
| 70 | 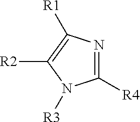 | 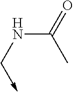 | 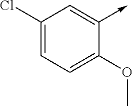 | 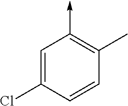 | 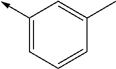 | M+ = 469.0 _{A}t_{Ret} = 4.57 min |
| 71 | 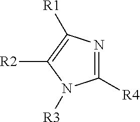 | 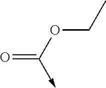 | 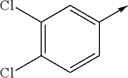 | 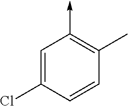 | 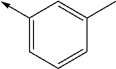 | M+ = 500.7 _{A}t_{Ret} = 6.05 min |
| 72 | 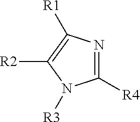 | 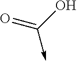 | 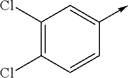 | 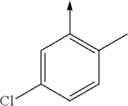 | 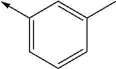 | M+ = 472.6 _{A}t_{Ret} = 5.31 min |
| 73 | 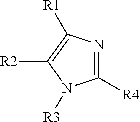 | 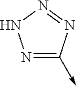 | 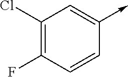 | 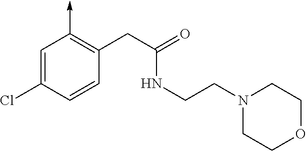 | 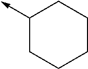 | M + H = 627/629 _{B}t_{Ret} = 1.04 min |
| 74 | 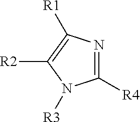 | 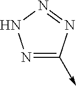 | 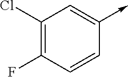 | 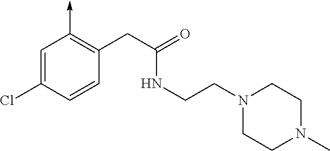 | 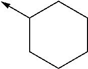 | M + H = 640/642 _{B}t_{Ret} = 0.98 min |
| 75 | 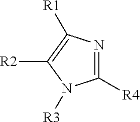 | 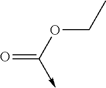 | 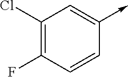 | 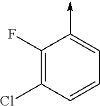 | 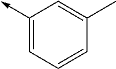 | M+ = 488.6 _{A}t_{Ret} = 5.82 min |
| 76 | 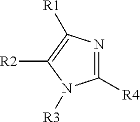 | 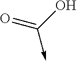 | 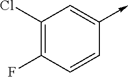 | 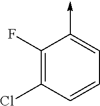 | 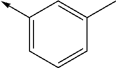 | M+ = 460.6 _{A}t_{Ret} = 5.10 min |
| 77 | 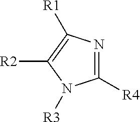 | 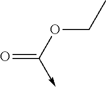 | 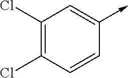 | 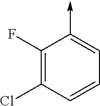 | 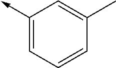 | M+ = 504.6 _{A}t_{Ret} = 6.13 min |
| 78 | 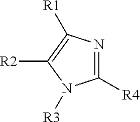 | 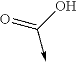 | 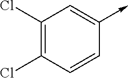 | 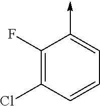 | 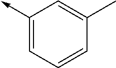 | M+ = 476.6 _{A}t_{Ret} = 5.24 min |
| 79 | 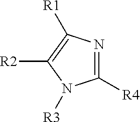 | 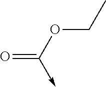 | 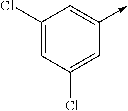 | 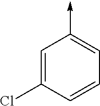 | 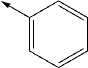 | M+ = 472.8 _{A}t_{Ret} = 5.84 min |
| 80 | 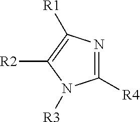 | 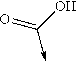 | 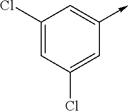 | 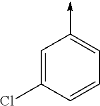 | 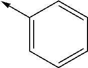 | M+ = 444.9 _{A}t_{Ret} = 5.06 min |
| 81 | 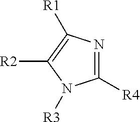 | 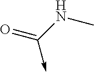 | 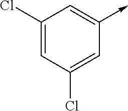 | 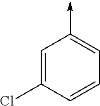 | 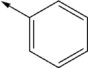 | M+ = 457.9 _{A}t_{Ret} = 5.57 min |
| 82 | 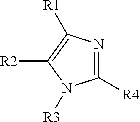 | 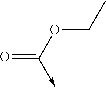 | 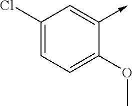 | 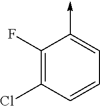 | 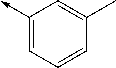 | M+ = 500.63 _{A}t_{Ret} = 5.59 min |
| 83 | 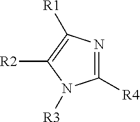 | 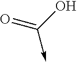 | 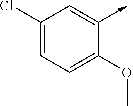 | 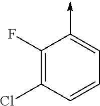 | 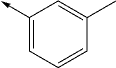 | M+ = 472.6 _{A}t_{Ret} = 4.94 min |
| 84 | 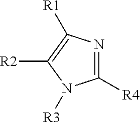 | 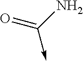 | 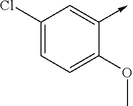 | 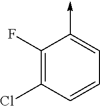 | 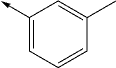 | M+ = 471.6 _{A}t_{Ret} = 5.13 min |
| 85 | 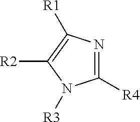 | 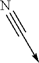 | 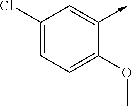 | 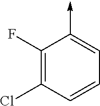 | 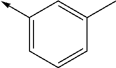 | M+ = 453.8 _{A}t_{Ret} = 5.80 min |
| 86 | 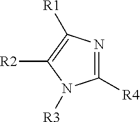 | 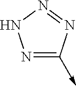 | 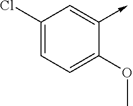 | 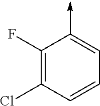 | 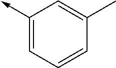 | M− = 494.8 _{A}t_{Ret} = 5.30 min |
| 87 | 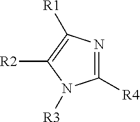 | 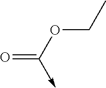 | 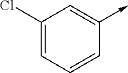 | 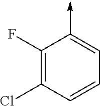 | 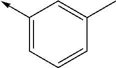 | M+ = 470.6 _{A}t_{Ret} = 5.75 min |
| 88 | 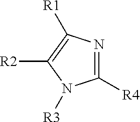 | 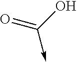 | 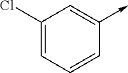 | 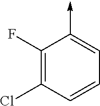 | 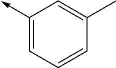 | M+ = 442.7 _{A}t_{Ret} = 5.00 min |
| 89 | 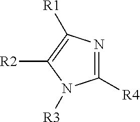 | 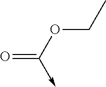 | 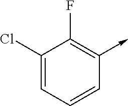 | 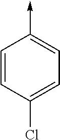 | 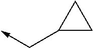 | M+ = 434.9 _{A}t_{Ret} = 4.83 min |
| 90 | 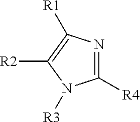 | 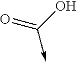 | 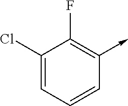 | 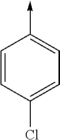 | 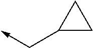 | M+ = 406.9 _{A}t_{Ret} = 4.10 min |
| 91 | 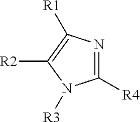 | 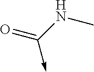 | 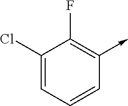 | 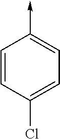 | 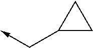 | M+ = 419.9 _{A}t_{Ret} = 4.61 min |
| 92 | 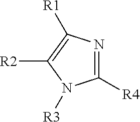 | 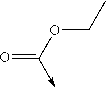 | 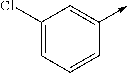 | 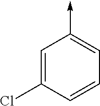 | 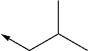 | M+ = 418.9 _{A}t_{Ret} = 4.75 min |
| 93 | 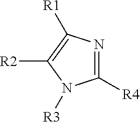 | 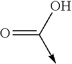 | 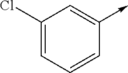 | 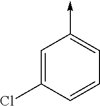 | 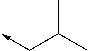 | M+ = 389.9 _{A}t_{Ret} = 4.01 min |
| 94 | 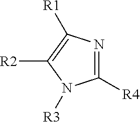 | 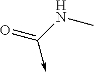 | 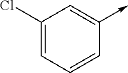 | 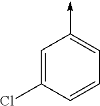 | 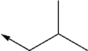 | M+ = 403.9 _{A}t_{Ret} = 4.51 min |
| 95 | 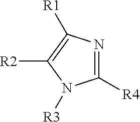 | 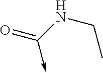 | 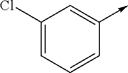 | 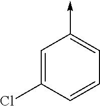 | 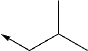 | M+ = 417.9 _{A}t_{Ret} = 4.66 min |
| 96 | 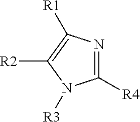 | 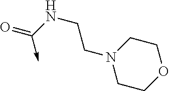 | 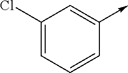 | 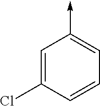 | 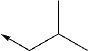 | M+ = 503.1 _{A}t_{Ret} = 4.17 min |
| 97 | 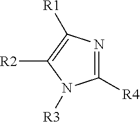 | 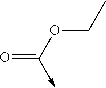 | 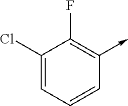 | 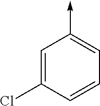 | 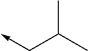 | M+ = 436.9 _{A}t_{Ret} = 4.93 min |
| 98 | 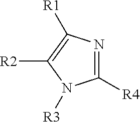 | 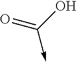 | 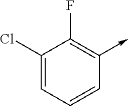 | 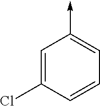 | 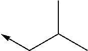 | M+ = 408.9 _{A}t_{Ret} = 4.11 min |
| 99 | 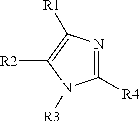 | 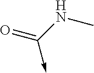 | 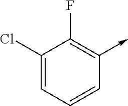 | 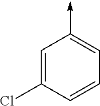 | 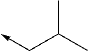 | M+ = 421.9 _{A}t_{Ret} = 4.79 min |
| 100 | 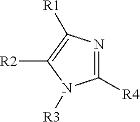 | 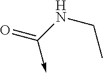 | 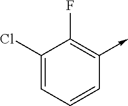 | 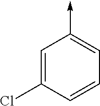 | 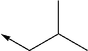 | M+ = 436.0 _{A}t_{Ret} = 5.31 min |
| 101 | 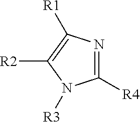 | 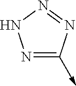 | 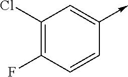 | 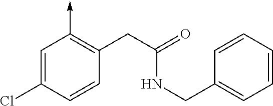 | 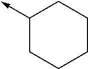 | M + H = 604/606 _{B}t_{Ret} = 1.34 min |
| 102 | 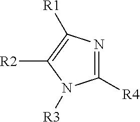 | 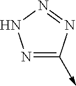 | 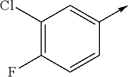 | 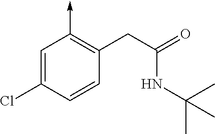 | 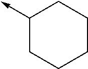 | M + H = 570/572 _{B}t_{Ret} = 1.37 min |
| 103 | 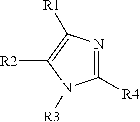 | 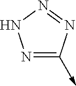 | 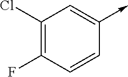 | 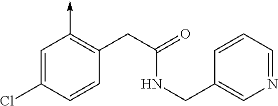 | 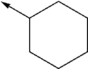 | M + H = 605/607 _{B}t_{Ret} = 1.05 min |
| 104 | 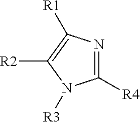 | 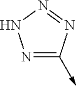 | 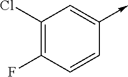 | 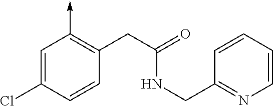 | 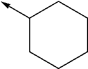 | M + H = 605/607 _{B}t_{Ret} = 1.06 min |
| 105 | 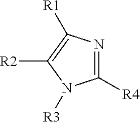 | 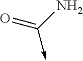 | 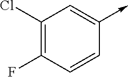 | 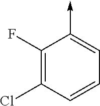 | 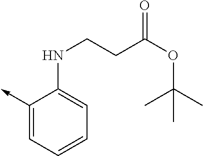 | M + H = 587/589 _{B}t_{Ret} = 1.44 min |
| 106 | 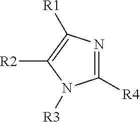 | 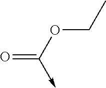 | 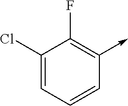 | 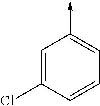 | 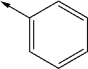 | M+ = 457.0 _{A}t_{Ret} = 5.55 min |
| 107 | 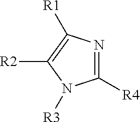 | 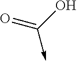 | 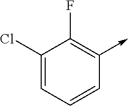 | 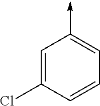 | 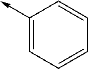 | M+ = 428.8 _{A}t_{Ret} = 4.01 min |
| 108 | 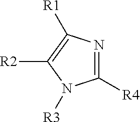 | 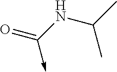 | 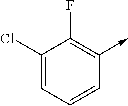 | 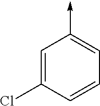 | 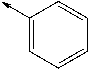 | M+ = 472.0 _{A}t_{Ret} = 5.65 min |
| 109 | 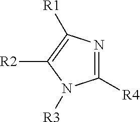 | 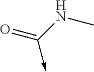 | 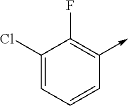 | 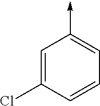 | 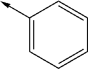 | M+ = 441.9 _{A}t_{Ret} = 5.30 min |
| 110 | 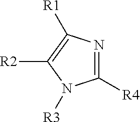 | 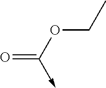 | 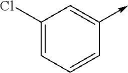 | 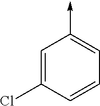 | 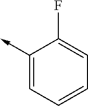 | M+ = 456.9 _{A}t_{Ret} = 5.51 min |
| 111 | 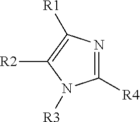 | 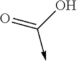 | 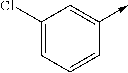 | 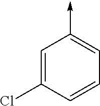 | 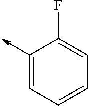 | M− = 425.1 _{A}t_{Ret} = 4.81 min |
| 112 | 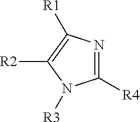 | 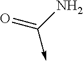 | 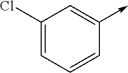 | 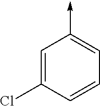 | 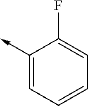 | M− = 427.9 _{A}t_{Ret} = 4.93 min |
| 113 | 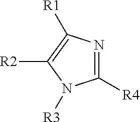 | 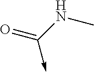 | 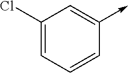 | 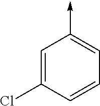 | 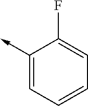 | M+ = 441.9 _{A}t_{Ret} = 5.17 min |
| 114 | 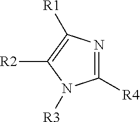 | 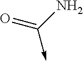 | 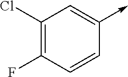 | 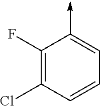 | 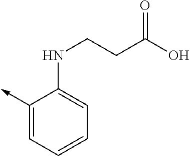 | M + H = 531/533 _{B}t_{Ret} = 1.20 min |
| 115 | 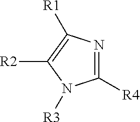 | 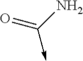 | 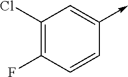 | 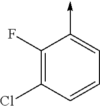 | 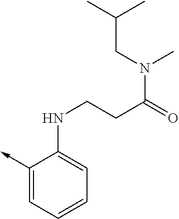 | M + H = 600/602 _{B}t_{Ret} = 1.34 min |
| 116 | 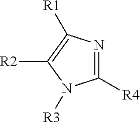 | 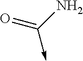 | 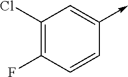 | 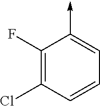 | 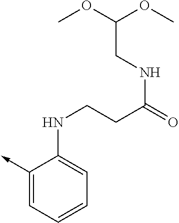 | M + H = 618/620 _{B}t_{Ret} = 1.19 min |
| 117 | 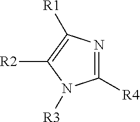 | 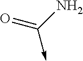 | 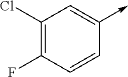 | 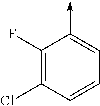 | 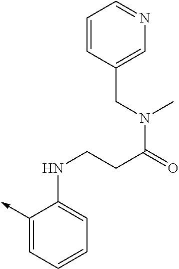 | M + H = 635/637 _{B}t_{Ret} = 1.06 min |
| 118 | 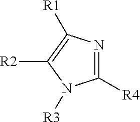 | 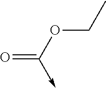 | 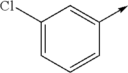 | 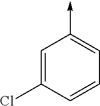 | 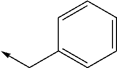 | M+ = 452.9 _{A}t_{Ret} = 5.35 min |
| 119 | 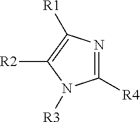 | 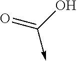 | 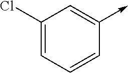 | 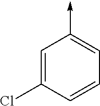 | 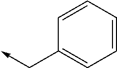 | M+ = 424.9 _{A}t_{Ret} = 4.61 min |
| 120 | 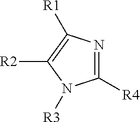 | 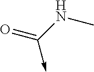 | 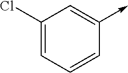 | 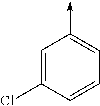 | 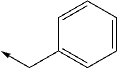 | M+ = 438.0 _{A}t_{Ret} = 5.15 min |
| 121 | 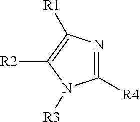 | 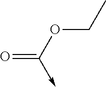 | 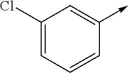 | 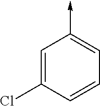 | 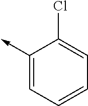 | M+ = 427.0 _{A}t_{Ret} = 5.61 min |
| 122 | 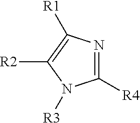 | 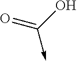 | 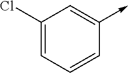 | 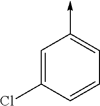 | 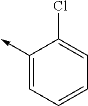 | M+ = 444.8 _{A}t_{Ret} = 4.99 min |
| 123 | 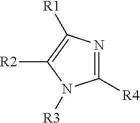 | 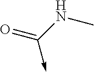 | 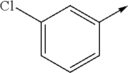 | 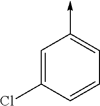 | 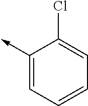 | M+ = 457.9 _{A}t_{Ret} = 5.36 min |
| 124 | 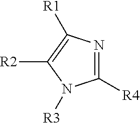 | 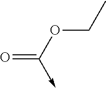 | 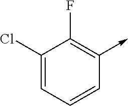 | 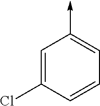 | 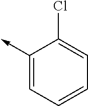 | M+ = 490.9 _{A}t_{Ret} = 5.64 min |
| 125 | 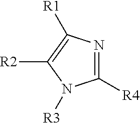 | 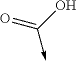 | 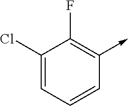 | 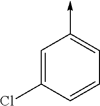 | 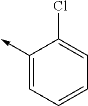 | M+ = 462.8 _{A}t_{Ret} = 4.97 min |
| 126 | 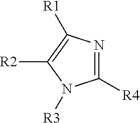 | 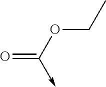 | 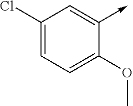 | 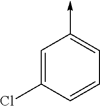 | 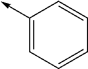 | M+ = 471.0 _{A}t_{Ret} = 5.35 min |
| 127 | 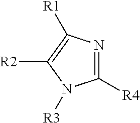 | 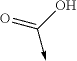 | 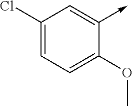 | 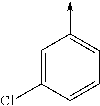 | 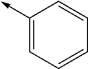 | M+ = 440.9 _{A}t_{Ret} = 4.64 min |
| 128 | 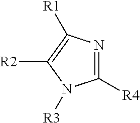 | 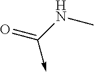 | 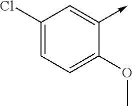 | 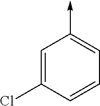 | 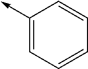 | M+ = 453.9 _{A}t_{Ret} = 5.06 min |
| 129 | 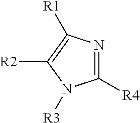 | 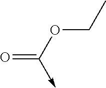 | 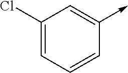 | 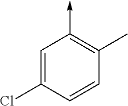 | 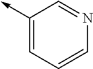 | M+ = 453.1 _{A}t_{Ret} = 4.63 min |
| 130 | 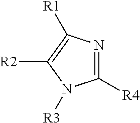 | 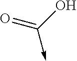 | 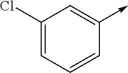 | 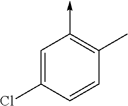 | 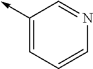 | M+ = 426.0 _{A}t_{Ret} = 4.05 min |
| 131 | 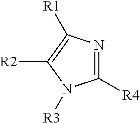 | 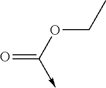 | 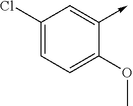 | 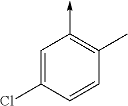 | 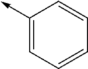 | M+ = 482.9 _{A}t_{Ret} = 5.50 min |
| 132 | 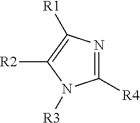 | 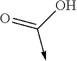 | 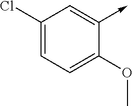 | 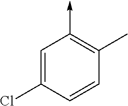 | 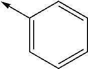 | M+ = 454.9 _{A}t_{Ret} = 4.83 min |
| 133 | 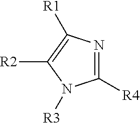 | 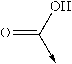 | 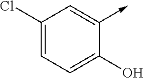 | 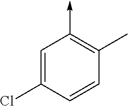 | 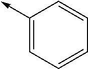 | M+ = 440.8 _{A}t_{Ret} = 4.55 min |
| 134 | 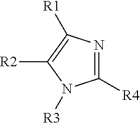 | 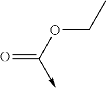 | 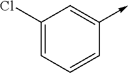 | 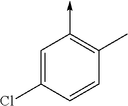 | 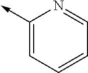 | M+ = 453.9 _{A}t_{Ret} = 5.49 min |
| 135 | 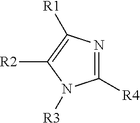 | 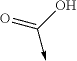 | 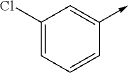 | 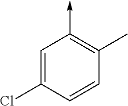 | 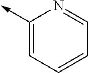 | M− = 423.9 _{A}t_{Ret} = 4.66 min |
| 136 | 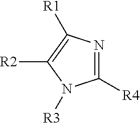 | 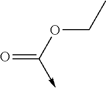 | 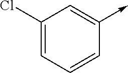 | 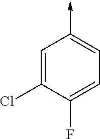 | 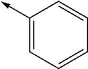 | M+ = 457.0 _{A}t_{Ret} = 5.49 min |
| 137 | 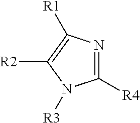 | 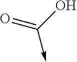 | 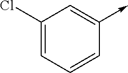 | 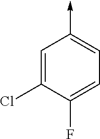 | 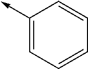 | M+ = 428.9 _{A}t_{Ret} = 4.81 min |
| 138 | 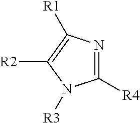 | 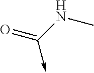 | 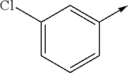 | 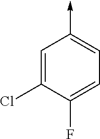 | 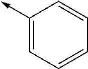 | M+ = 441.9 _{A}t_{Ret} = 5.28 min |
| 139 | 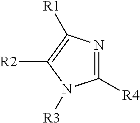 | 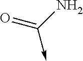 | 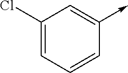 | 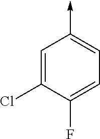 | 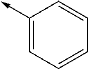 | M+ = 427.9 _{A}t_{Ret} = 5.05 min |
| 140 | 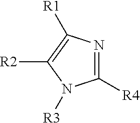 | 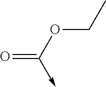 | 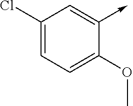 | 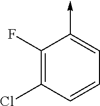 | 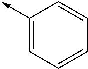 | M+ = 486.8 _{A}t_{Ret} = 5.45 min |
| 141 | 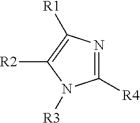 | 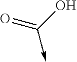 | 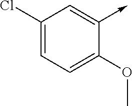 | 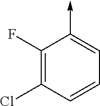 | 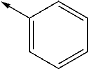 | M+ = 458.7 _{A}t_{Ret} = 4.75 min |
| 142 | 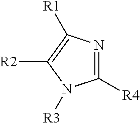 | 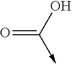 | 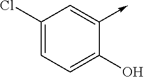 | 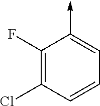 | 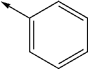 | M+ = 441.0 _{A}t_{Ret} = 4.48 min |
| 143 | 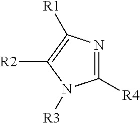 | 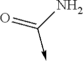 | 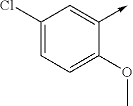 | 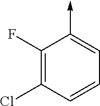 | 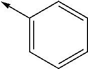 | M+ = 457.8 _{A}t_{Ret} = 4.98 min |
| 144 | 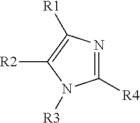 | 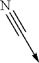 | 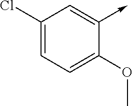 | 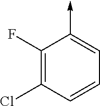 | 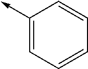 | M+ = 439.8 _{A}t_{Ret} = 5.62 min |
| 145 | 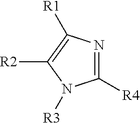 | 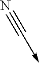 | 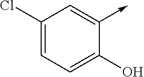 | 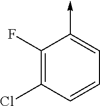 | 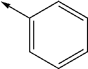 | M+ = 426.7 _{A}t_{Ret} = 5.37 min |
| 146 | 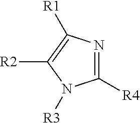 | 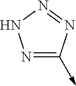 | 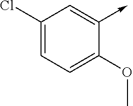 | 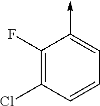 | 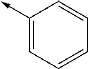 | M+ = 482.9 _{A}t_{Ret} = 5.14 min |
| 147 | 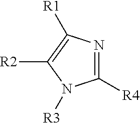 | 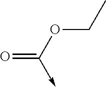 | 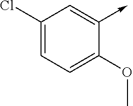 | 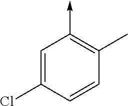 | 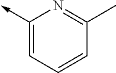 | M+ = 499.2 _{A}t_{Ret} = 5.33 min |
| 148 | 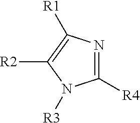 | 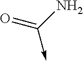 | 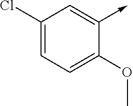 | 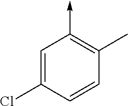 | 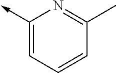 | M+ = 468.8 _{A}t_{Ret} = 4.73 min |
| 149 | 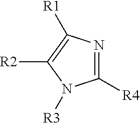 | 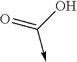 | 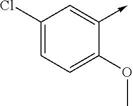 | 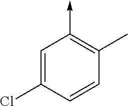 | 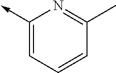 | M− = 466.1 _{A}t_{Ret} = 4.64 min |
| 150 | 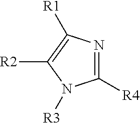 | 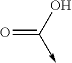 | 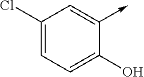 | 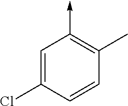 | 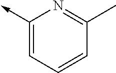 | M+ = 455.8 _{A}t_{Ret} = 4.27 min |
| 151 | 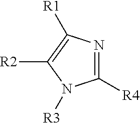 | 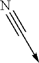 | 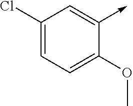 | 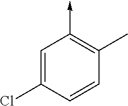 | 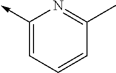 | M+ = 450.9 _{A}t_{Ret} = 5.81 min |
| 152 | 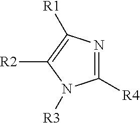 | 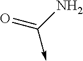 | 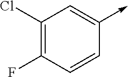 | 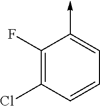 | 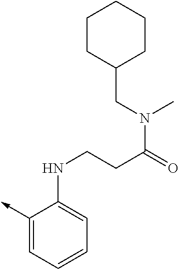 | M + H = 640/642 _{B}t_{Ret} = 1.45 min |
| 153 | 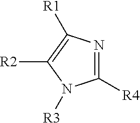 | 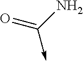 | 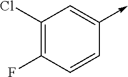 | 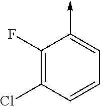 | 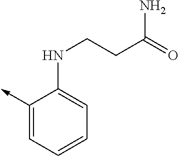 | M + H = 530/532 _{B}t_{Ret} = 1.14 min |
| 154 | 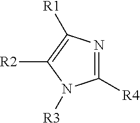 | 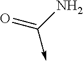 | 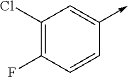 | 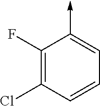 | 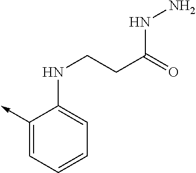 | M + H = 545/547 _{B}t_{Ret} = 1.05 min |
| 155 | 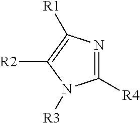 | 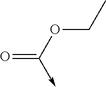 | 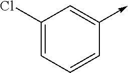 | 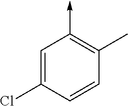 | 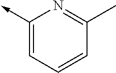 | M+ = 467.8 _{A}t_{Ret} = 5.44 min |
| 156 | 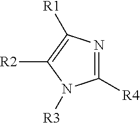 | 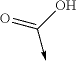 | 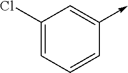 | 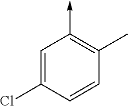 | 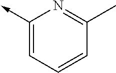 | M+ = 439.9 _{A}t_{Ret} = 4.70 min |
| 157 | 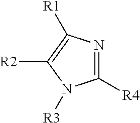 | 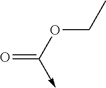 | 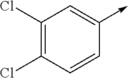 | 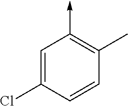 | 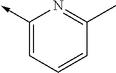 | M+ = 503.3 _{A}t_{Ret} = 5.94 min |
| 158 | 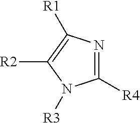 | 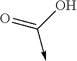 | 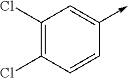 | 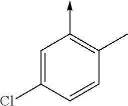 | 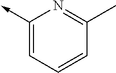 | M− = 473.9 _{A}t_{Ret} = 5.07 min |
| 159 | 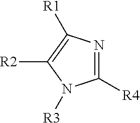 | 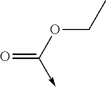 | 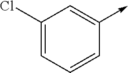 | 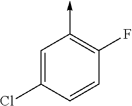 | 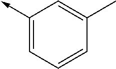 | M+ = 470.6 _{A}t_{Ret} = 5.71 min |
| 160 | 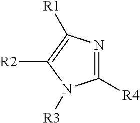 | 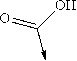 | 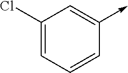 | 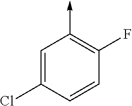 | 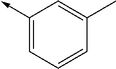 | M+ = 442.6 _{A}t_{Ret} = 4.90 min |
| 161 | 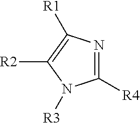 | 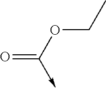 | 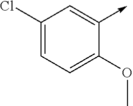 | 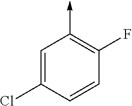 | 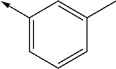 | M+ = 500.8 _{A}t_{Ret} = 5.61 min |
| 162 | 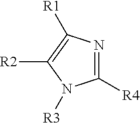 | 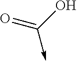 | 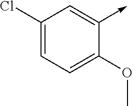 | 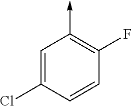 | 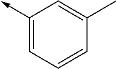 | M+ = 472.9 _{A}t_{Ret} = 4.95 min |
| 163 | 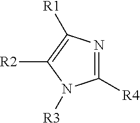 | 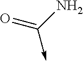 | 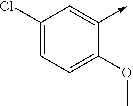 | 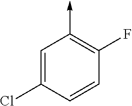 | 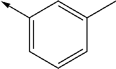 | M+ = 471.8 _{A}t_{Ret} = 5.11 min |
| 164 | 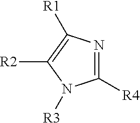 | 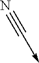 | 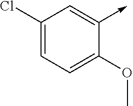 | 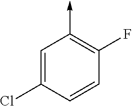 | 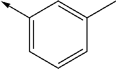 | M+ = 453.9 _{A}t_{Ret} = 5.78 min |
| 165 | 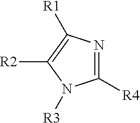 | 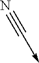 | 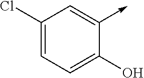 | 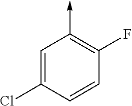 | 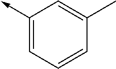 | M+ = 439.7 _{A}t_{Ret} = 5.63 min |
| 166 | 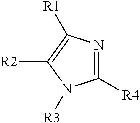 | 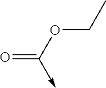 | 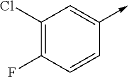 | 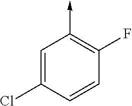 | 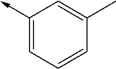 | M+ = 460.6 _{A}t_{Ret} = 5.78 min |
| 167 | 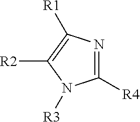 | 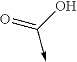 | 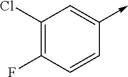 | 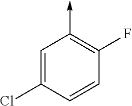 | 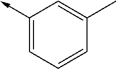 | M+ = 509.3 _{A}t_{Ret} = 5.01 min |
| 168 | 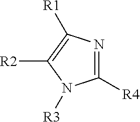 | 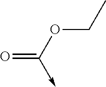 | 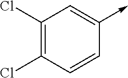 | 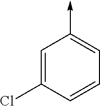 | 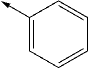 | M+ = 472.5 _{A}t_{Ret} = 5.71 min |
| 169 | 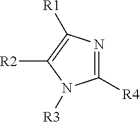 | 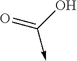 | 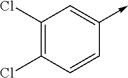 | 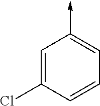 | 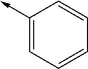 | M+ = 444.9 _{A}t_{Ret} = 5.05 min |
| 170 | 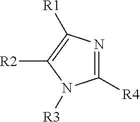 | 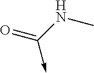 | 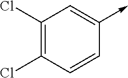 | 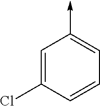 | 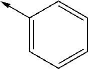 | M+ = 457.9 _{A}t_{Ret} = 5.52 min |
| 171 | 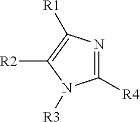 | 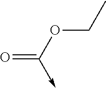 | 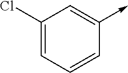 | 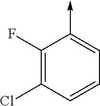 | 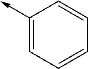 | M+ = 456.8 _{A}t_{Ret} = 5.61 min |
| 172 | 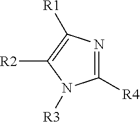 | 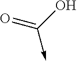 | 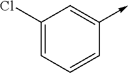 | 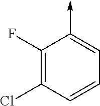 | 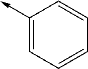 | M− = 425.19 _{A}t_{Ret} = 4.03 min |
| 173 | 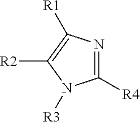 | 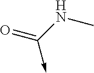 | 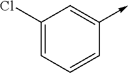 | 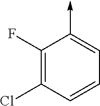 | 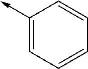 | M+ = 441.7 _{A}t_{Ret} = 5.20 min |
| 174 | 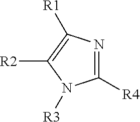 | 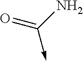 | 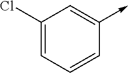 | 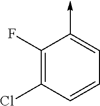 | 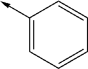 | M+ = 427.7 _{A}t_{Ret} = 5.03 min |
| 175 | 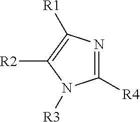 | 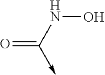 | 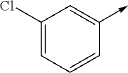 | 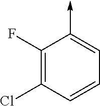 | 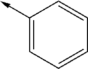 | M− = 441.0 _{A}t_{Ret} = 6.36 min |
| 176 | 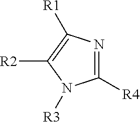 | 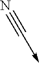 | 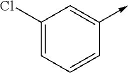 | 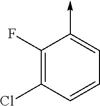 | 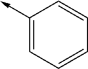 | M+ = 506.6 _{A}t_{Ret} = 6.64 min |
| 177 | 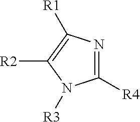 | 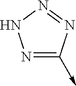 | 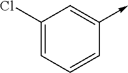 | 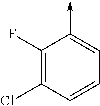 | 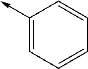 | M+ = 453.0 _{A}t_{Ret} = 5.02 min |
| 178 | 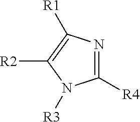 | 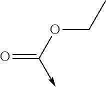 | 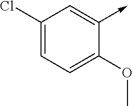 | 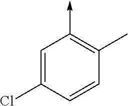 | 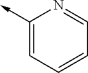 | M+ = 483.7 _{A}t_{Ret} = 5.33 min |
| 179 | 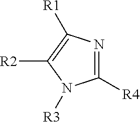 | 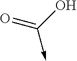 | 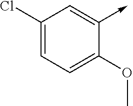 | 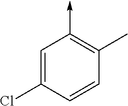 | 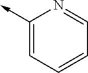 | M+ = 455.9 _{A}t_{Ret} = 4.61 min |
| 180 | 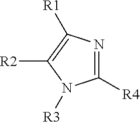 | 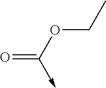 | 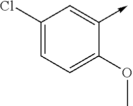 | 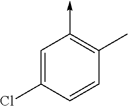 | 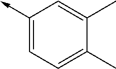 | M+ = 510.8 _{A}t_{Ret} = 5.64 min |
| 181 | 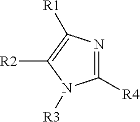 | 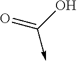 | 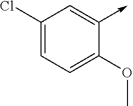 | 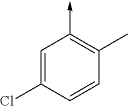 | 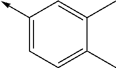 | M+ = 482.8 _{A}t_{Ret} = 5.20 min |
| 182 | 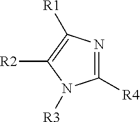 | 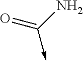 | 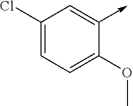 | 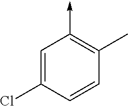 | 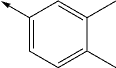 | M+ = 482.0 _{A}t_{Ret} = 5.38 min |
| 183 | 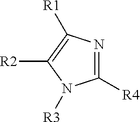 | 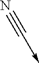 | 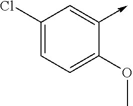 | 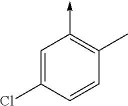 | 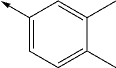 | M+ = 463.8 _{A}t_{Ret} = 6.01 min |
| 184 | 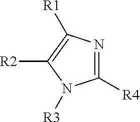 | 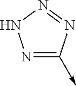 | 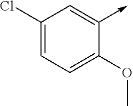 | 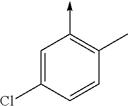 | 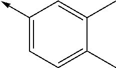 | M+ = 506.6 _{A}t_{Ret} = 5.50 min |
| 185 | 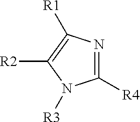 | 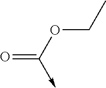 | 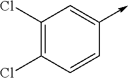 | 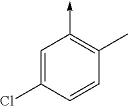 | 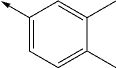 | M+ = 514.7 _{A}t_{Ret} = 5.49 min |
| 186 | 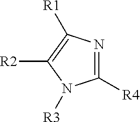 | 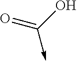 | 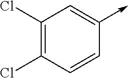 | 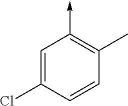 | 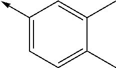 | M+ = 486.8 _{A}t_{Ret} = 5.38 min |
| 187 | 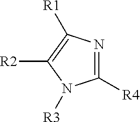 | 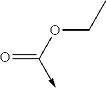 | 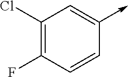 | 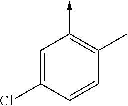 | 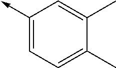 | M+ = 498.8 _{A}t_{Ret} = 5.95 min |
| 188 | 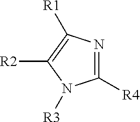 | 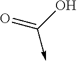 | 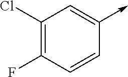 | 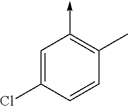 | 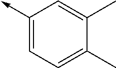 | M+ = 471.0 _{A}t_{Ret} = 5.21 min |
| 189 | 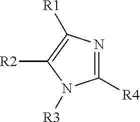 | 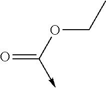 | 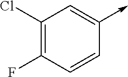 | 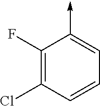 | 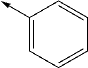 | M+ = 474.6 _{A}t_{Ret} = 5.63 min |
| 190 | 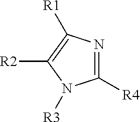 | 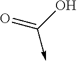 | 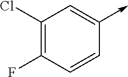 | 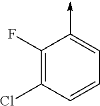 | 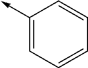 | M− = 444.7 _{A}t_{Ret} = 4.88 min |
| 191 | 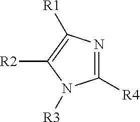 | 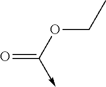 | 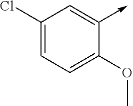 | 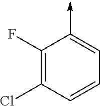 | 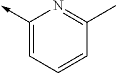 | M+ = 501.8 _{A}t_{Ret} = 5.57 min |
| 192 | 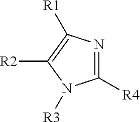 | 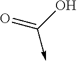 | 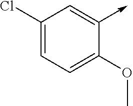 | 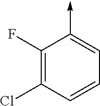 | 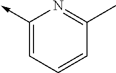 | M− = 471.8 _{A}t_{Ret} = 4.88 min |
| 193 | 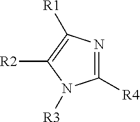 | 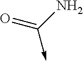 | 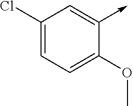 | 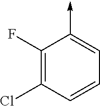 | 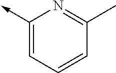 | M− = 470.9 _{A}t_{Ret} = 4.03 min |
| 194 | 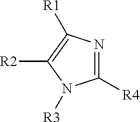 | 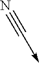 | 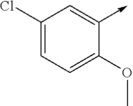 | 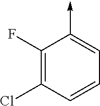 | 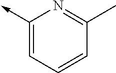 | M+ = 454.8 _{A}t_{Ret} = 5.63 min |
| 195 | 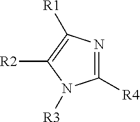 | 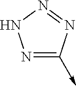 | 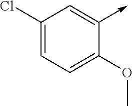 | 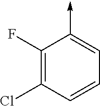 | 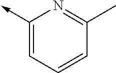 | M− = 493.9 _{A}t_{Ret} = 5.26 min |
| 196 | 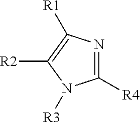 | 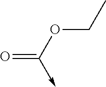 | 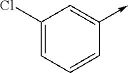 | 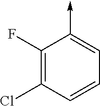 | 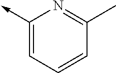 | M+ = 471.7 _{A}t_{Ret} = 5.63 min |
| 197 | 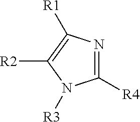 | 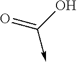 | 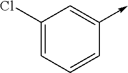 | 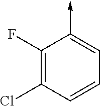 | 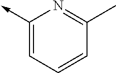 | M− = 440.0 _{A}t_{Ret} = 4.91 min |
| 198 | 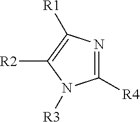 | 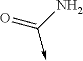 | 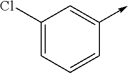 | 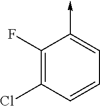 | 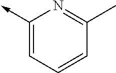 | M+ = 442.8 _{A}t_{Ret} = 5.01 min |
| 199 | 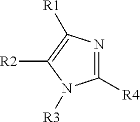 | 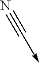 | 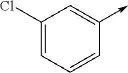 | 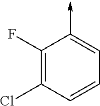 | 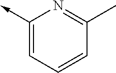 | M+ = 424.9 _{A}t_{Ret} = 5.86 min |
| 200 | 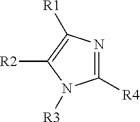 | 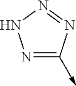 | 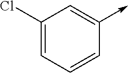 | 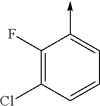 | 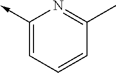 | M− = 465.8 _{A}t_{Ret} = 5.25 min |
| 201 | 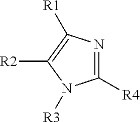 | 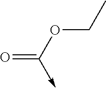 | 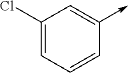 | 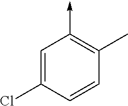 | 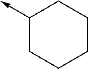 | M + H = 457/459 _{B}t_{Ret} = 1.32 min |
| 202 | 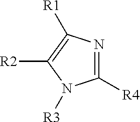 | 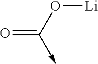 | 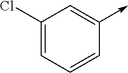 | 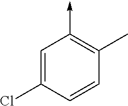 | 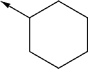 | M + H = 429/431 _{B}t_{Ret} = 1.15 min |
| 203 | 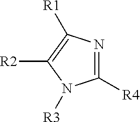 | 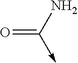 | 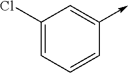 | 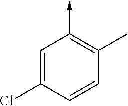 | 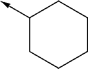 | M + H = 428/430 _{B}t_{Ret} = 1.25 min |
| 204 | 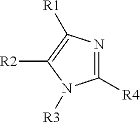 | 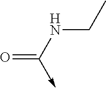 | 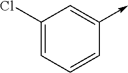 | 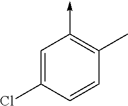 | 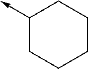 | M + H = 456/458 _{B}t_{Ret} = 1.34 min |
| 205 | 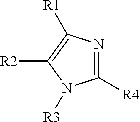 | 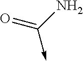 | 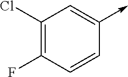 | 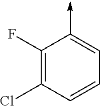 | 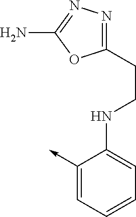 | M + H = 570/572 _{B}t_{Ret} = 1.11 min |
| 206 | 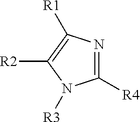 | 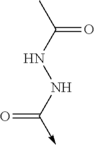 | 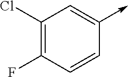 | 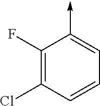 | 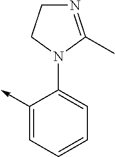 | M + H = 583/585 _{B}t_{Ret} = 0.99 min |
| 207 | 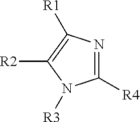 | 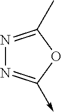 | 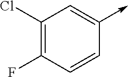 | 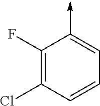 | 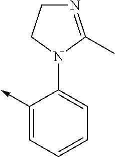 | M + H = 565/567 _{B}t_{Ret} = 1.06 min |
| 208 | 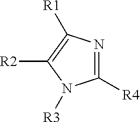 | 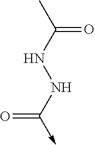 | 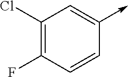 | 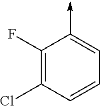 | 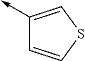 | M + H = 507/509 _{B}t_{Ret} = 1.17 min |
| 209 | 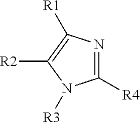 | 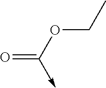 | 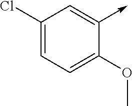 | 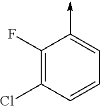 | 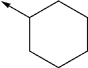 | M + H = 491/493 _{B}t_{Ret} = 1.31 min |
| 210 | 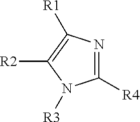 | 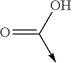 | 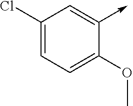 | 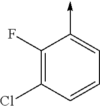 | 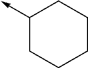 | M + H = 463/465 _{B}t_{Ret} = 1.14 min |
| 211 | 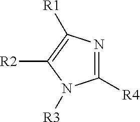 | 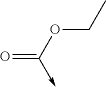 | 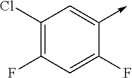 | 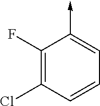 | 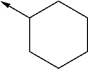 | M + H = 497/499 _{B}t_{Ret} = 1.41 min |
| 212 | 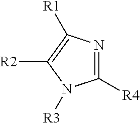 | 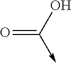 | 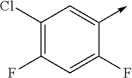 | 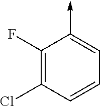 | 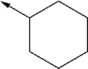 | M + H = 469/471 _{B}t_{Ret} = 1.23 min |
| 213 | 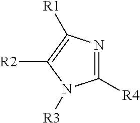 | 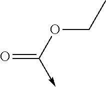 | 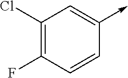 | 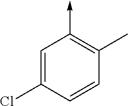 | 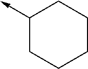 | M + H = 475/477 _{B}t_{Ret} = 1.34 min |
| 214 | 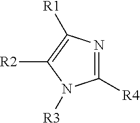 | 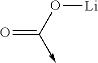 | 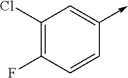 | 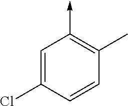 | 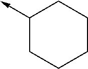 | M + H = 447/449 _{B}t_{Ret} = 1.18 min |
| 215 | 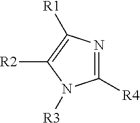 | 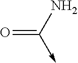 | 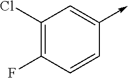 | 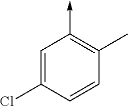 | 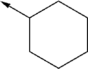 | M + H = 446/448 _{B}t_{Ret} = 1.28 min |
| 216 | 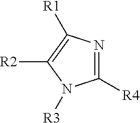 | 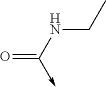 | 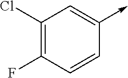 | 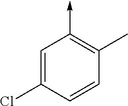 | 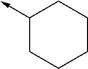 | M + H = 474/476 _{B}t_{Ret} = 1.38 min |
| 217 | 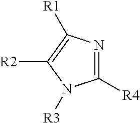 | 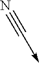 | 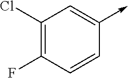 | 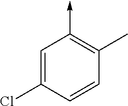 | 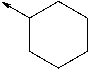 | M + H = 428/430 _{B}t_{Ret} = 1.49 min |
| 218 | 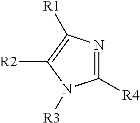 | 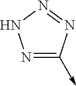 | 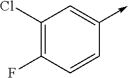 | 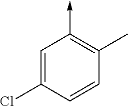 | 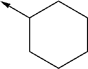 | M + H = 471/73 _{B}t_{Ret} = 1.34 min |
| 219 | 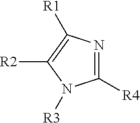 | 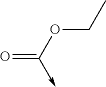 | 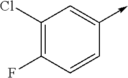 | 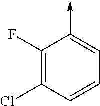 | 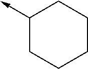 | M + H = 479/481 _{B}t_{Ret} = 1.38 min |
| 220 | 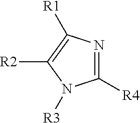 | 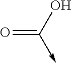 | 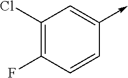 | 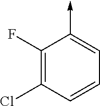 | 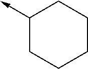 | M + H = 451/453 _{B}t_{Ret} = 1.19 min |
| 221 | 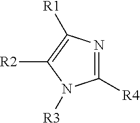 | 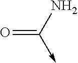 | 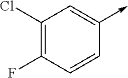 | 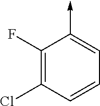 | 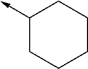 | M + H = 450/452 _{B}t_{Ret} = 1.29 min |
| 222 | 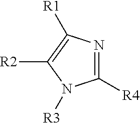 | 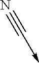 | 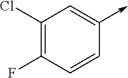 | 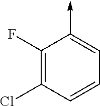 | 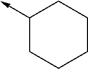 | M + H = 432/434 _{B}t_{Ret} = 1.47 min |
| 223 | 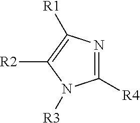 | 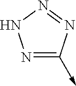 | 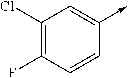 | 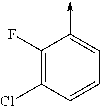 | 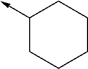 | M + H = 475/477 _{B}t_{Ret} = 1.33 min |
| 224 | 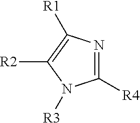 | 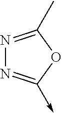 | 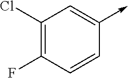 | 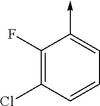 | 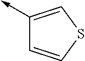 | M + H = 489/591 _{B}t_{Ret} = 1.28 min |
| 225 | 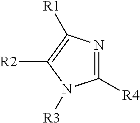 | 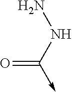 | 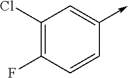 | 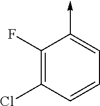 | 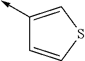 | M + H = 465/467 _{B}t_{Ret} = 1.14 min |
| 226 | 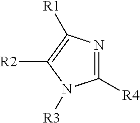 | 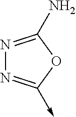 | 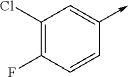 | 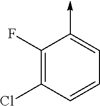 | 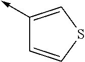 | M + H = 490/592 _{B}t_{Ret} = 1.15 min |
| 227 | 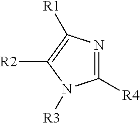 | 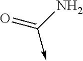 | 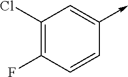 | 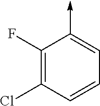 | 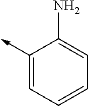 | M + H = 459/461 _{B}t_{Ret} = 1.16 min |
| 228 | 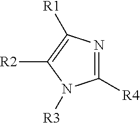 | 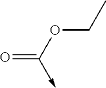 | 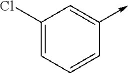 | 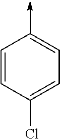 | 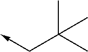 | M + H = 431/433 _{B}t_{Ret} = 1.27 min |
| 229 | 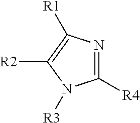 | 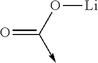 | 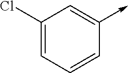 | 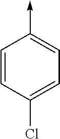 | 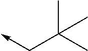 | M + H = 403/405 _{B}t_{Ret} = 1.13 min |
| 230 | 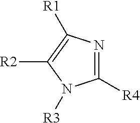 | 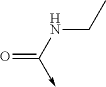 | 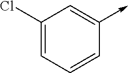 | 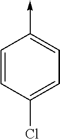 | 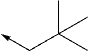 | M + H = 430/432 _{B}t_{Ret} = 1.30 min |
| 231 | 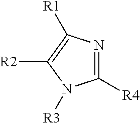 | 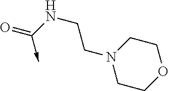 | 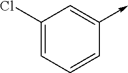 | 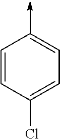 | 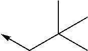 | M + H = 515/517 _{B}t_{Ret} = 1.17 min |
| 232 | 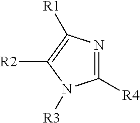 | 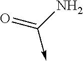 | 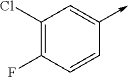 | 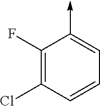 | 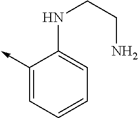 | M + H = 502/504 _{B}t_{Ret} = 1.03 min |
| 233 | 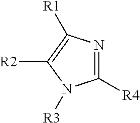 | 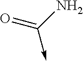 | 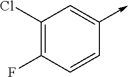 | 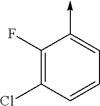 | 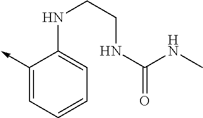 | M + H = 559/561 _{B}t_{Ret} = 1.12 min |
| 234 | 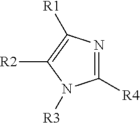 | 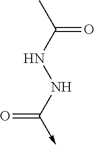 | 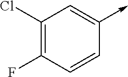 | 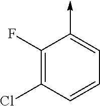 | 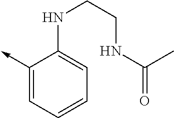 | M + H = 601/603 _{B}t_{Ret} = 1.11 min |
| 235 | 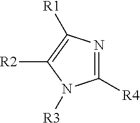 | 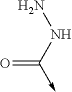 | 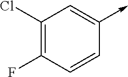 | 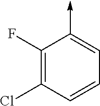 | 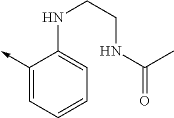 | M + H = 559/561 _{B}t_{Ret} = 1.06 min |
| 236 | 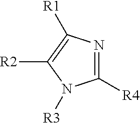 | 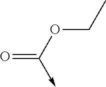 | 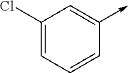 | 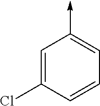 | 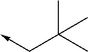 | M + H = 431/433 _{B}t_{Ret} = 1.25 min |
| 237 | 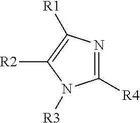 | 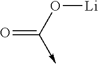 | 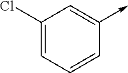 | 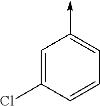 | 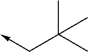 | M + H = 403/405 _{B}t_{Ret} = 1.09 min |
| 238 | 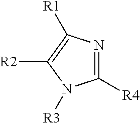 | 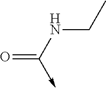 | 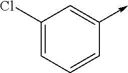 | 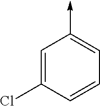 | 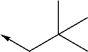 | M + H = 430/432 _{B}t_{Ret} = 1.28 min |
| 239 | 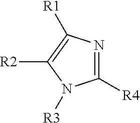 | 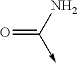 | 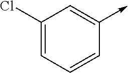 | 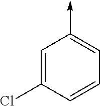 | 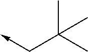 | M + H = 402/404 _{B}t_{Ret} = 1.18 min |
| 240 | 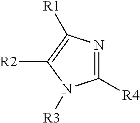 | 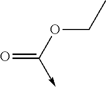 | 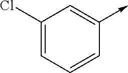 | 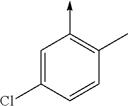 | 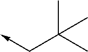 | M + H = 445/447 _{B}t_{Ret} = 1.32 min |
| 241 | 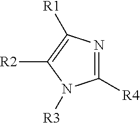 | 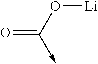 | 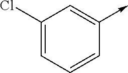 | 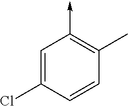 | 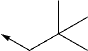 | M + H = 417/419 _{B}t_{Ret} = 1.14 min |
| 242 | 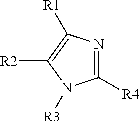 | 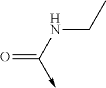 | 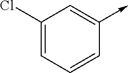 | 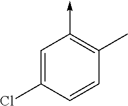 | 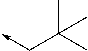 | M + H = 444/446 _{B}t_{Ret} = 1.36 min |
| 243 | 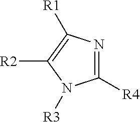 | 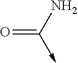 | 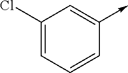 | 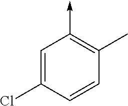 | 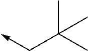 | M + H = 416/418 _{B}t_{Ret} = 1.26 min |
| 244 | 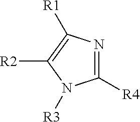 | 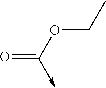 | 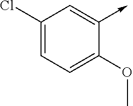 | 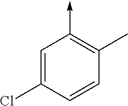 | 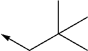 | M + H = 475/477 _{B}t_{Ret} = 1.28 min |
| 245 | 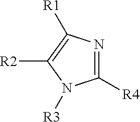 | 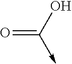 | 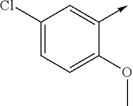 | 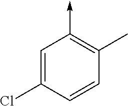 | 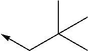 | M + H = 447/449 _{B}t_{Ret} = 1.13 min |
| 246 | 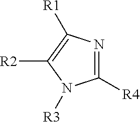 | 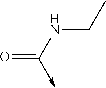 | 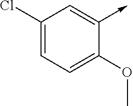 | 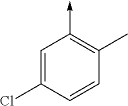 | 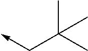 | M + H = 474/476 _{B}t_{Ret} = 1.30 min |
| 247 | 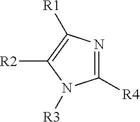 | 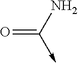 | 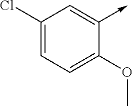 | 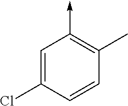 | 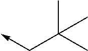 | M + H = 446/448 _{B}t_{Ret} = 1.22 min |
| 248 | 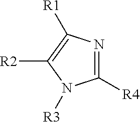 | 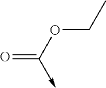 | 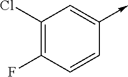 | 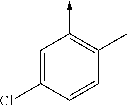 | 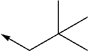 | M + H = 463/465 _{B}t_{Ret} = 1.34 min |
| 249 | 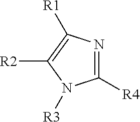 | 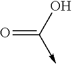 | 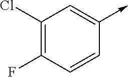 | 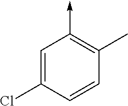 | 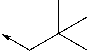 | M + H = 435/437 _{B}t_{Ret} = 1.16 min |
| 250 | 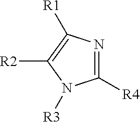 | 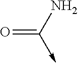 | 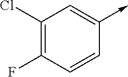 | 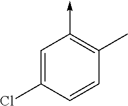 | 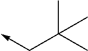 | M + H = 434/436 _{B}t_{Ret} = 1.29 min |
| 251 | 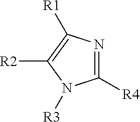 | 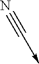 | 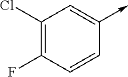 | 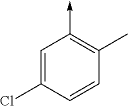 | 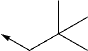 | M + H = 416/418 TLC (DCM/ EtOAc = 1/1) Rf = 0.61 |
| 252 | 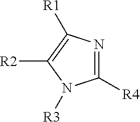 | 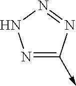 | 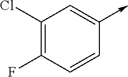 | 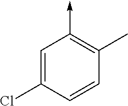 | 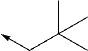 | M + H = 459/461 _{B}t_{Ret} = 1.34 min |
| 253 | 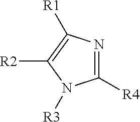 | 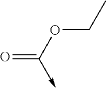 | 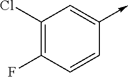 | 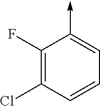 | 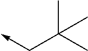 | M + H = 467/469 _{B}t_{Ret} = 1.36 min |
| 254 | 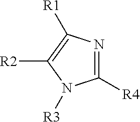 | 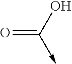 | 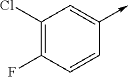 | 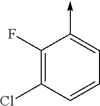 | 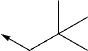 | M + H = 439/441 _{B}t_{Ret} = 1.17 min |
| 255 | 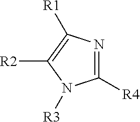 | 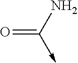 | 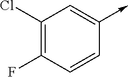 | 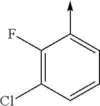 | 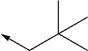 | M + H = 438/440 _{B}t_{Ret} = 1.27 min |
| 256 | 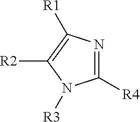 | 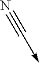 | 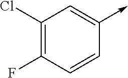 | 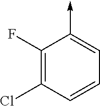 | 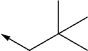 | M + H = 420/422 _{B}t_{Ret} = 1.45 min |
| 257 | 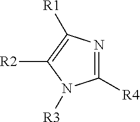 | 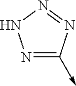 | 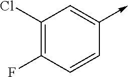 | 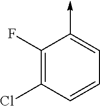 | 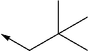 | M + H = 463/465 _{B}t_{Ret} = 1.31 min |
| 258 | 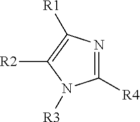 | 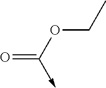 | 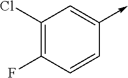 | 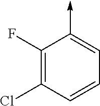 | 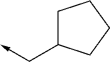 | M + H = 479/481 _{B}t_{Ret} = 1.36 min |
| 259 | 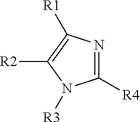 | 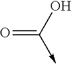 | 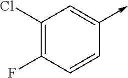 | 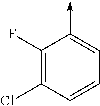 | 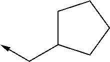 | M + H = 451/453 _{B}t_{Ret} = 1.18 min |
| 260 | 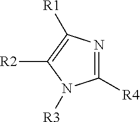 | 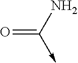 | 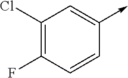 | 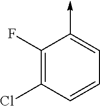 | 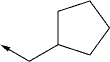 | M + H = 450/452 _{B}t_{Ret} = 1.28 min |
| 261 | 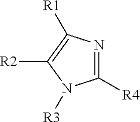 | 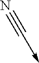 | 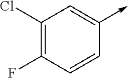 | 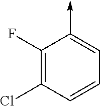 | 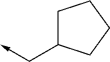 | M + H = 432/434 _{B}t_{Ret} = 1.47 min |
| 262 | 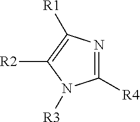 | 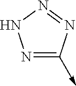 | 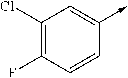 | 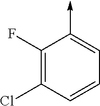 | 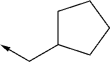 | M + H = 475/477 _{B}t_{Ret} = 1.33 min |
| 263 | 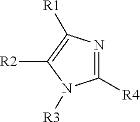 | 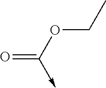 | 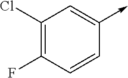 | 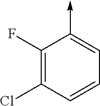 | 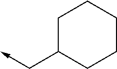 | M + H = 493/495 _{B}t_{Ret} = 1.39 min |
| 264 | 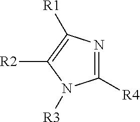 | 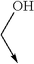 | 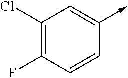 | 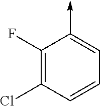 | 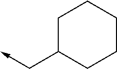 | M + H = 451/453 _{B}t_{Ret} = 1.17 min |
| 265 | 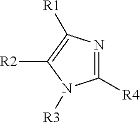 | 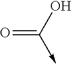 | 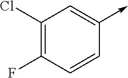 | 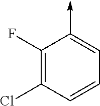 | 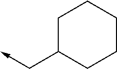 | M + H = 465/467 _{B}t_{Ret} = 1.21 min |
| 266 | 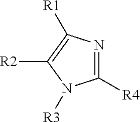 | 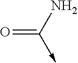 | 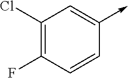 | 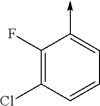 | 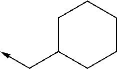 | M + H = 464/466 _{B}t_{Ret} = 1.33 min |
| 267 | 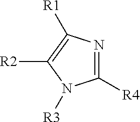 | 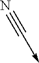 | 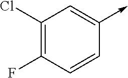 | 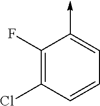 | 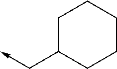 | M + H = 446/448 _{B}t_{Ret} = 1.51 min |
| 268 | 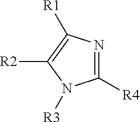 | 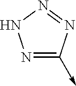 | 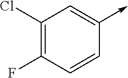 | 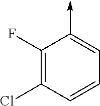 | 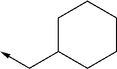 | M + H = 489/491 _{B}t_{Ret} = 1.37 min |
| 269 | 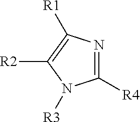 | 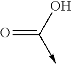 | 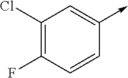 | 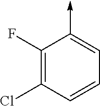 | 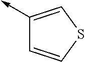 | M + H = 451/453 _{B}t_{Ret} = 1.16 min |
| 270 | 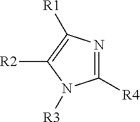 | 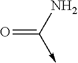 | 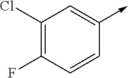 | 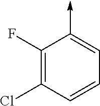 | 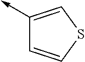 | M + H = 450/542 _{B}t_{Ret} = 1.20 min |
| 271 | 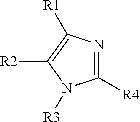 | 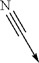 | 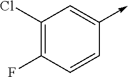 | 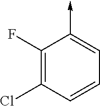 | 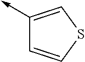 | M + H = 432/434 _{B}t_{Ret} = 1.36 min |
| 272 | 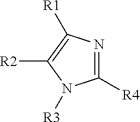 | 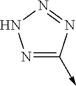 | 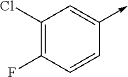 | 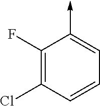 | 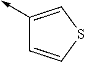 | M + H = 475/477 _{B}t_{Ret} = 1.24 min |
| 273 | 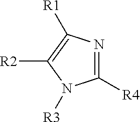 | 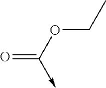 | 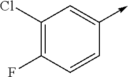 | 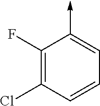 | 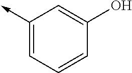 | M + H = 489/491 _{B}t_{Ret} = 1.25 min |
| 274 | 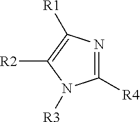 | 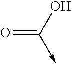 | 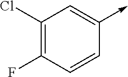 | 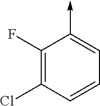 | 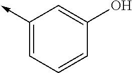 | M + H = 461/463 _{B}t_{Ret} = 1.09 min |
| 275 | 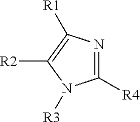 | 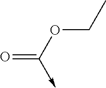 | 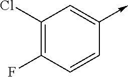 | 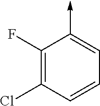 | 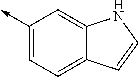 | M + H = 512/514 _{B}t_{Ret} = 1.29 min |
| 276 | 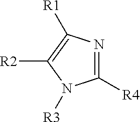 | 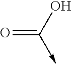 | 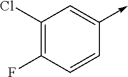 | 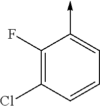 | 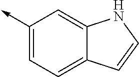 | M + H = 484/486 _{B}t_{Ret} = 1.13 min |
| 277 | 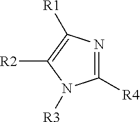 | 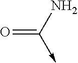 | 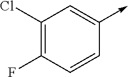 | 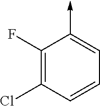 | 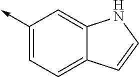 | M + H = 483/485 _{B}t_{Ret} = 1.23 min |
| 278 | 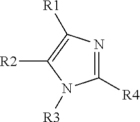 | 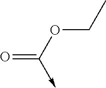 | 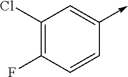 | 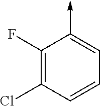 | 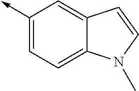 | M + H = 526/528 _{B}t_{Ret} = 1.31 min |
| 279 | 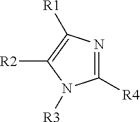 | 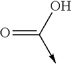 | 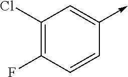 | 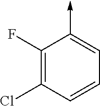 | 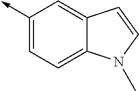 | M + H = 498/500 _{B}t_{Ret} = 1.14 min |
| 280 | 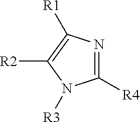 | 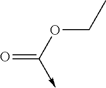 | 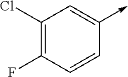 | 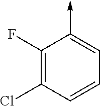 | 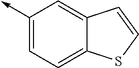 | M + H = 529/531 _{B}t_{Ret} = 1.40 min |
| 281 | 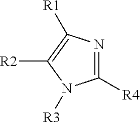 | 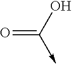 | 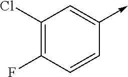 | 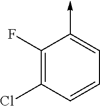 | 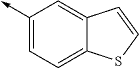 | M + H = 501/503 _{B}t_{Ret} = 1.24 min |
| 282 | 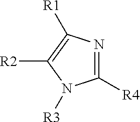 | 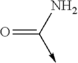 | 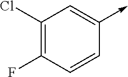 | 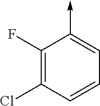 | 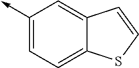 | M + H = 500/502 _{B}t_{Ret} = 1.36 min |
| 283 | 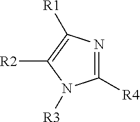 | 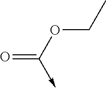 | 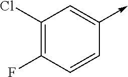 | 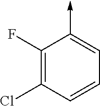 | 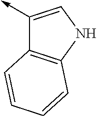 | M + H = 512/514 _{B}t_{Ret} = 1.26 min |
| 284 | 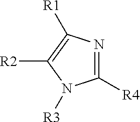 | 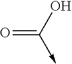 | 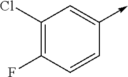 | 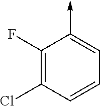 | 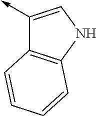 | M + H = 484/486 _{B}t_{Ret} = 1.09 min |
| 285 | 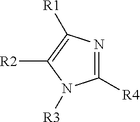 | 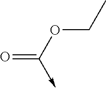 | 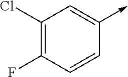 | 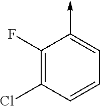 | 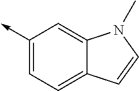 | M + H = 526/528 _{B}t_{Ret} = 1.34 min |
| 286 | 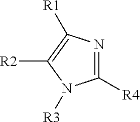 | 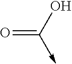 | 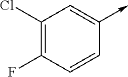 | 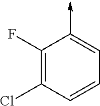 | 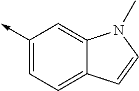 | M + H = 498/500 _{B}t_{Ret} = 1.19 min |
| 287 | 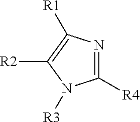 | 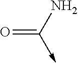 | 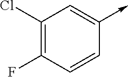 | 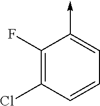 | 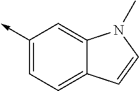 | M + H = 497/499 _{B}t_{Ret} = 1.29 min |
| 288 | 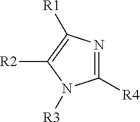 | 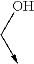 | 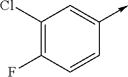 | 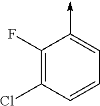 | 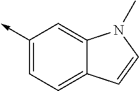 | M + H = 484/486 _{B}t_{Ret} = 1.13 min |
| 289 | 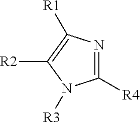 | 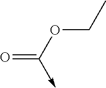 | 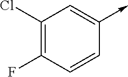 | 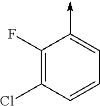 | 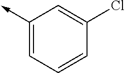 | M + H = 507/509 _{B}t_{Ret} = 1.42 min |
| 290 | 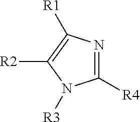 | 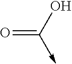 | 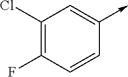 | 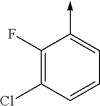 | 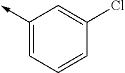 | M + H = 479/481 _{B}t_{Ret} = 1.24 min |
| 291 | 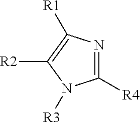 | 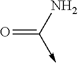 | 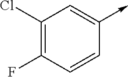 | 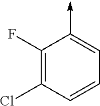 | 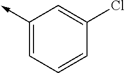 | M + H = 478/480 _{B}t_{Ret} = 1.36 min |
| 292 | 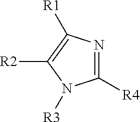 | 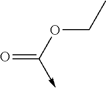 | 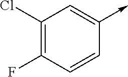 | 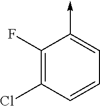 | 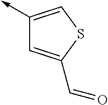 | M + H = 507/509 TLC (hexane/ EtOAc = 1:1) Rf = 0.48 |
| 293 | 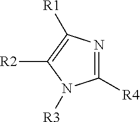 | 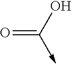 | 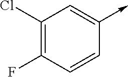 | 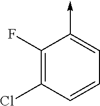 | 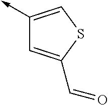 | M + H = 479/481 mp = 157- 160° C. |
| 294 | 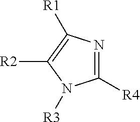 | 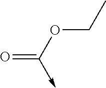 | 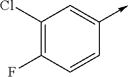 | 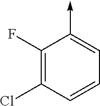 | 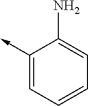 | M + H = 488/490 _{B}t_{Ret} = 1.33 min |
| 295 | 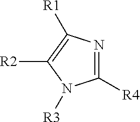 | 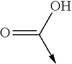 | 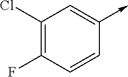 | 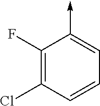 | 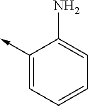 | M + H = 460/462 _{B}t_{Ret} = 1.14 min |
| 296 | 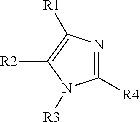 | 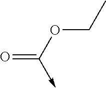 | 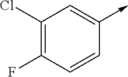 | 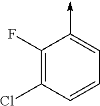 | 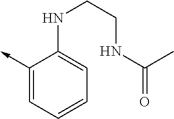 | M + H = 573/575 _{B}t_{Ret} = 1.31 min |
| 297 | 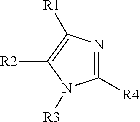 | 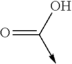 | 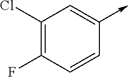 | 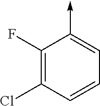 | 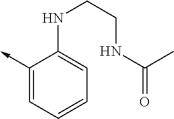 | M + H = 545/547 _{B}t_{Ret} = 1.16 min |
| 298 | 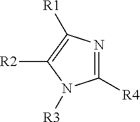 | 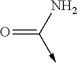 | 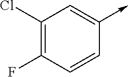 | 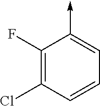 | 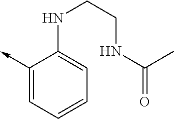 | M + H = 544/546 _{B}t_{Ret} = 1.16 min |
| 299 | 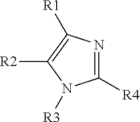 | 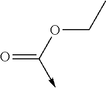 | 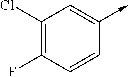 | 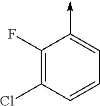 | 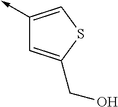 | M + H = 509/511 _{B}t_{Ret} = 1.29 min |
| 300 | 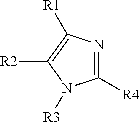 | 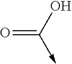 | 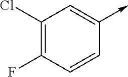 | 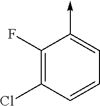 | 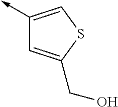 | M + H = 481/483 _{B}t_{Ret} = 1.09 min |
| 301 | 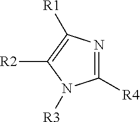 | 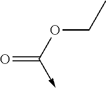 | 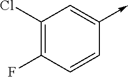 | 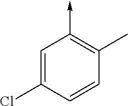 | 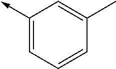 | M + H = 483/485 _{B}t_{Ret} = 1.47 min |
| 302 | 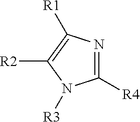 | 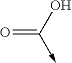 | 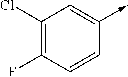 | 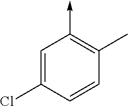 | 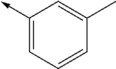 | M + H = 455/457 _{B}t_{Ret} = 1.27 min |
| 303 | 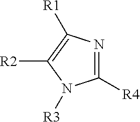 | 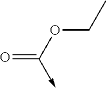 | 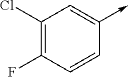 | 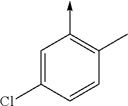 | 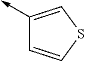 | M + H = 475/477 _{B}t_{Ret} = 1.42 min |
| 304 | 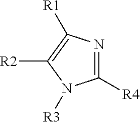 | 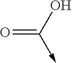 | 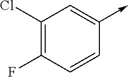 | 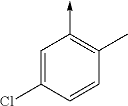 | 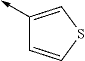 | M + H = 447/449 _{B}t_{Ret} = 1.22 min |
| 305 | 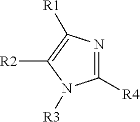 | 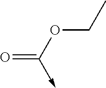 | 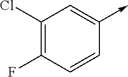 | 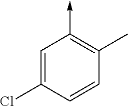 | 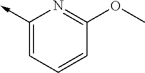 | M + H = 500/502 _{B}t_{Ret} = 1.51 min |
| 306 | 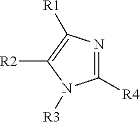 | 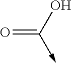 | 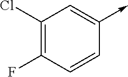 | 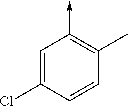 | 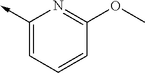 | M + H = 472/474 _{B}t_{Ret} = 1.32 min |
| 307 | 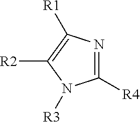 | 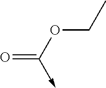 | 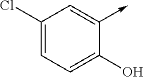 | 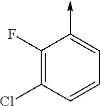 | 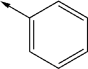 | M+ = 472.8 _{A}t_{Ret} = 5.07 min |
| 308 | 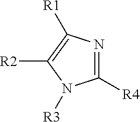 | 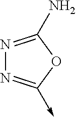 | 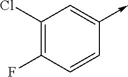 | 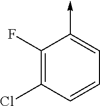 | 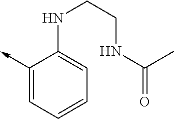 | M + H = 584/586 _{B}t_{Ret} = 1.11 min |
| 309 | 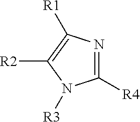 | 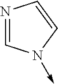 | 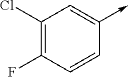 | 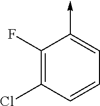 | 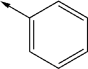 | M + 1 = 468.9 _{A}t_{Ret} = 4.42 min. |
| 310 | 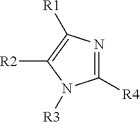 | 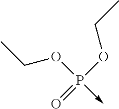 | 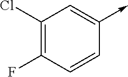 | 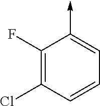 | 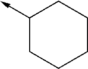 | M + 1 = 543/545 _{B}t_{Ret} = 1.41 min |
| 311 | 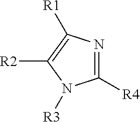 | 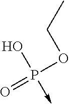 | 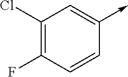 | 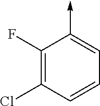 | 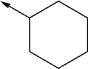 | M + 1 = 515/517 _{B}t_{Ret} = 1.08 min |
| 312 | 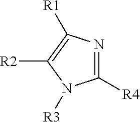 | 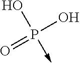 | 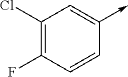 | 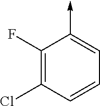 | 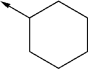 | M + 1 = 487/489 _{B}t_{Ret} = 1.00 min |
| 313 | 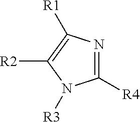 | 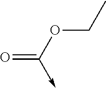 | 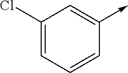 | 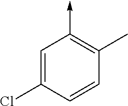 | 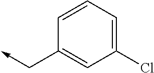 | M+ = 500.0 _{A}t_{Ret} = 5.79 min |
| 314 | 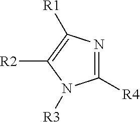 | 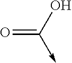 | 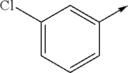 | 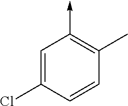 | 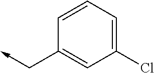 | M+ = 472.9 _{A}t_{Ret} = 5.09 min |
| 315 | 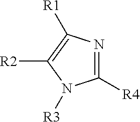 | 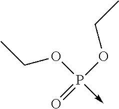 | 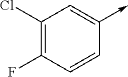 | 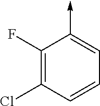 | 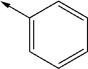 | M + 1 = 537/539 _{B}t_{Ret} = 1.34 min |
| 316 | 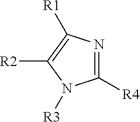 | 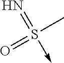 | 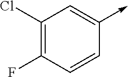 | 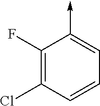 | 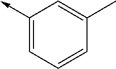 | [M + 1]+ = 492/494 _{B}t_{Ret} = 1.18 min |
| 317 | 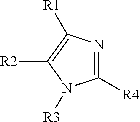 | 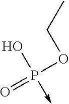 | 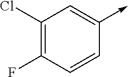 | 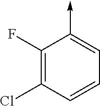 | 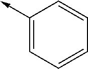 | M + 1 = 509/511 _{B}t_{Ret} = 1.03 min |
| 318 | 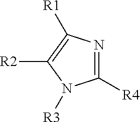 | 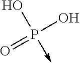 | 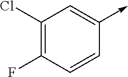 | 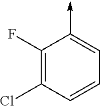 | 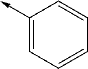 | M + 1 = 481/483 _{B}t_{Ret} = 0.94 min |
| 319 | 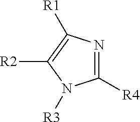 | 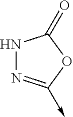 | 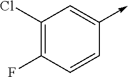 | 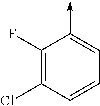 | 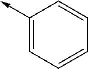 | M + 1 = 486.7 _{A}t_{Ret} = 5.08 min |
| 320 | 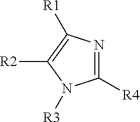 | 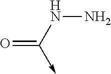 | 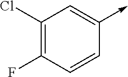 | 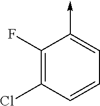 | 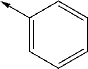 | M − 1 = 444.9 _{A}t_{Ret} = 4.33 min |
| 321 | 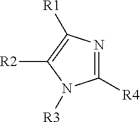 | 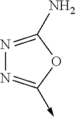 | 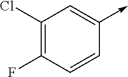 | 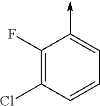 | 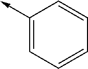 | M + 1 = 485.8 _{A}t_{Ret} = 4.73 min |
| 322 | 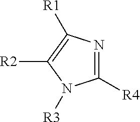 | 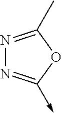 | 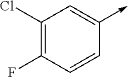 | 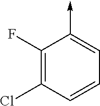 | 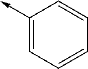 | M + 1 = 484.8 _{A}t_{Ret} = 5.27 min |
| 323 | 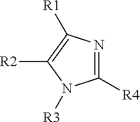 | 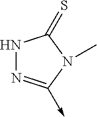 | 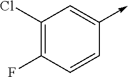 | 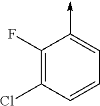 | 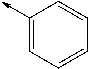 | M + 1 = 515.7 _{A}t_{Ret} = 5.25 min |
| 324 | 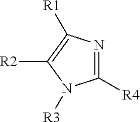 | 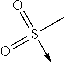 | 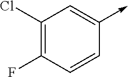 | 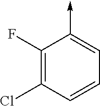 | 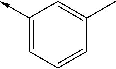 | [M + 1]+ = 493/495 _{B}t_{Ret} = 1.38 min |
| 325 | 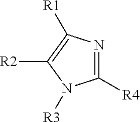 | 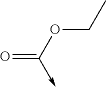 | 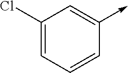 | 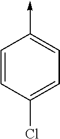 | 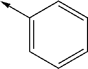 | M+ = 438.1 _{A}t_{Ret} = 5.44 min |
| 326 | 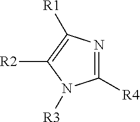 | 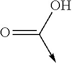 | 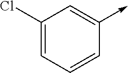 | 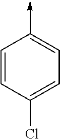 | 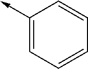 | M+ = 409.0 _{A}t_{Ret} = 4.73 min |
| 327 | 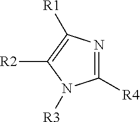 | 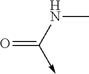 | 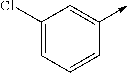 | 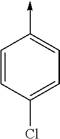 | 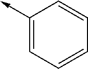 | M+ = 423.9 _{A}t_{Ret} = 5.26 min |
| 328 | 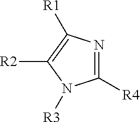 | 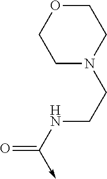 | 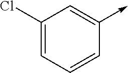 | 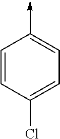 | 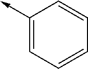 | M+ = 523.2 _{A}t_{Ret} = 4.57 min |
| 329 | 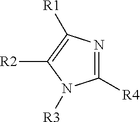 | 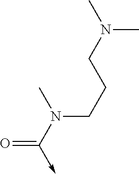 | 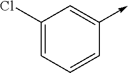 | 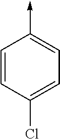 | 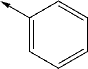 | M+ = 509.2 _{A}t_{Ret} = 4.45 min |
| 330 | 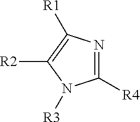 | 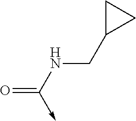 | 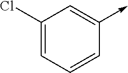 | 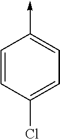 | 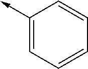 | M+ = 463.4 _{A}t_{Ret} = 5.69 min |
| 331 | 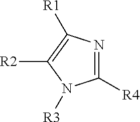 | 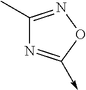 | 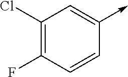 | 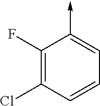 | 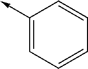 | M + 1 = 484.7 _{A}t_{Ret} = 5.59 min |
| 332 | 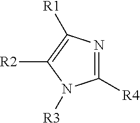 | 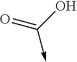 | 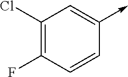 | 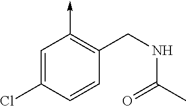 | 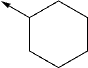 | M + 1 = 533.8 _{A}t_{Ret} = 4.75 min |
| 333 | 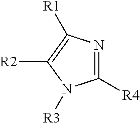 | 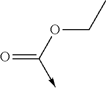 | 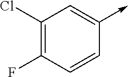 | 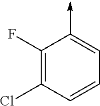 | 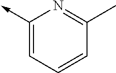 | M+ = 489.7 _{A}t_{Ret} = 5.66 min |
| 334 | 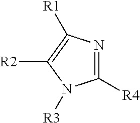 | 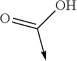 | 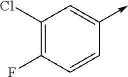 | 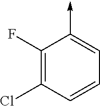 | 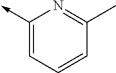 | M− = 458.0 _{A}t_{Ret} = 4.93 min |
| 335 | 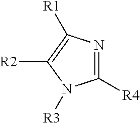 | 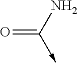 | 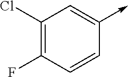 | 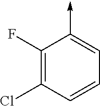 | 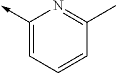 | M+ = 460.8 _{A}t_{Ret} = 5.00 min |
| 336 | 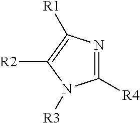 | 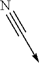 | 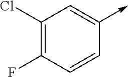 | 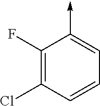 | 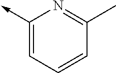 | M+ = 442.9 _{A}t_{Ret} = 5.84 min |
| 337 | 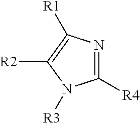 | 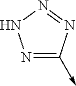 | 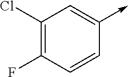 | 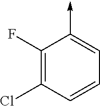 | 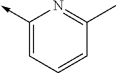 | M− = 483.9 _{A}t_{Ret} = 5.16 min |
| 338 | 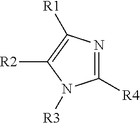 | 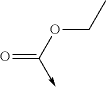 | 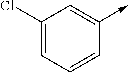 | 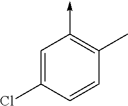 | 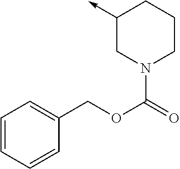 | M+ = 593.0 _{A}t_{Ret} = 5.73 min |
| 339 | 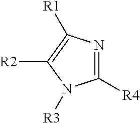 | 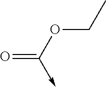 | 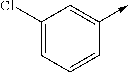 | 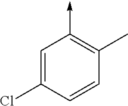 | 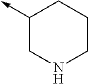 | M+ = 460.2 _{A}t_{Ret} = 4.35 min |
| 340 | 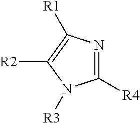 | 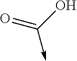 | 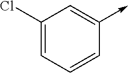 | 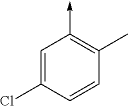 | 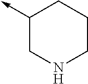 | M+ = 434.0 _{A}t_{Ret} = 5.19 min |
| 341 | 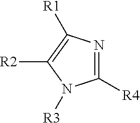 | 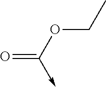 | 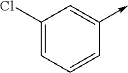 | 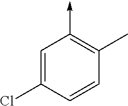 | 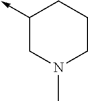 | M+ = 476.0 _{A}t_{Ret} = 4.41 min |
| 342 | 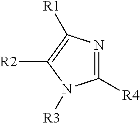 | 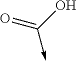 | 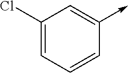 | 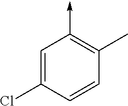 | 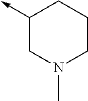 | M+ = 445.0 _{A}t_{Ret} = 3.96 min |
| 343 | 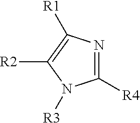 | 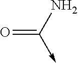 | 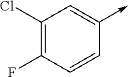 | 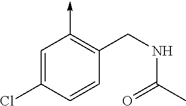 | 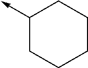 | M + 1 = 504.9: ¹H NMR: (DMSOd₆) 8.38 (bs, 1H), 7.80 (s, 1H), 7.58- 7.55 (m, 2H), 7.33- 7.21 (m, 3H), 3.42 (dd, 1H), 3.30 (dd, 1H), 2.22- 2.18 (m, 1H), 1.84 (s, 3H), 1.80-1.40 (m, 5H), 1.23- 0.99 (m, 5H) |
| 344 | 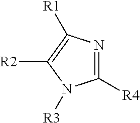 | 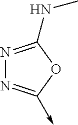 | 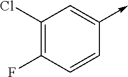 | 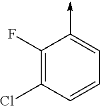 | 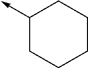 | M + 1 = 506.0; ¹H NMR: (CDCl₃) 7.52 (dd, 1H), 7.35 (dd, 1H), 7.20-7.14 (m, 2H), 7.10-7.01 (m, 2H), 3.03 (d, 3H), 2.38- 2.30 (m, 1H), 1.86- 1.74 (m, 6H), 1.68- 1.54 (m, 4H) |
| 345 | 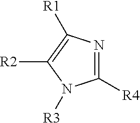 | 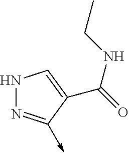 | 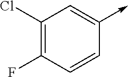 | 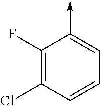 | 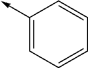 | M + 1 = 683.1 _{A}t_{Ret} = 4.06 min. |
| 346 | 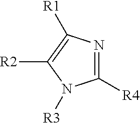 | 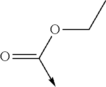 | 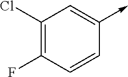 | 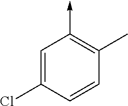 | 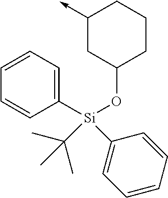 | M+ = 731.2 _{A}t_{Ret} = 7.41 min |
| 347 | 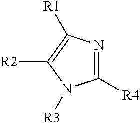 | 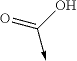 | 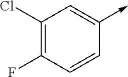 | 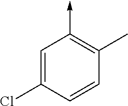 | 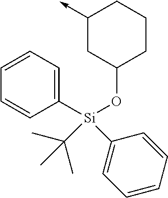 | M− = 699.0 _{A}t_{Ret} = 6.78 min |
| 348 | 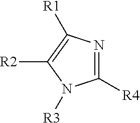 | 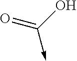 | 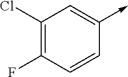 | 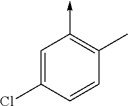 | 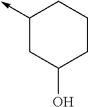 | M+ = 465.0 _{A}t_{Ret} = 4.26 min |
| 349 | 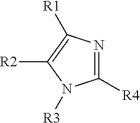 | 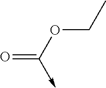 | 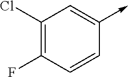 | 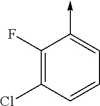 | 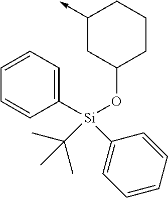 | M+ = 735.2 _{A}t_{Ret} = 7.64 min |
| 350 | 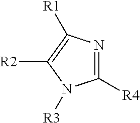 | 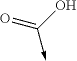 | 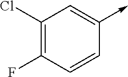 | 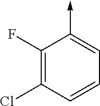 | 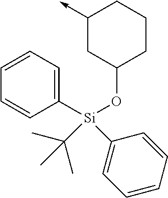 | M+ = 707.2 _{A}t_{Ret} = 6.94 min |
| 351 | 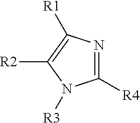 | 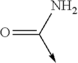 | 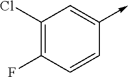 | 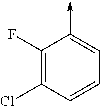 | 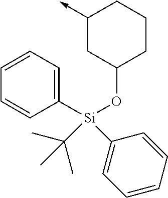 | M+ = 706.3 _{A}t_{Ret} = 7.34 min |
| 352 | 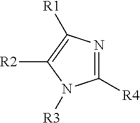 | 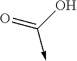 | 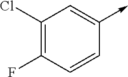 | 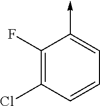 | 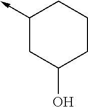 | M+ = 469.0 _{A}t_{Ret} = 4.29 min |
| 353 | 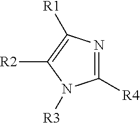 | 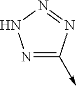 | 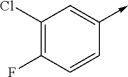 | 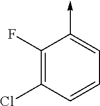 | 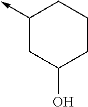 | M+ = 492.2 _{A}t_{Ret} = 4.70 min |
| 354 | 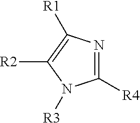 | 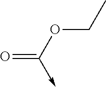 | 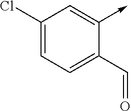 | 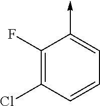 | 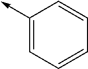 | M+ = 484.8 _{A}t_{Ret} = 5.57 min |
| 355 | 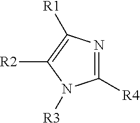 | 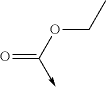 | 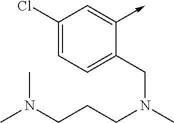 | 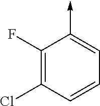 | 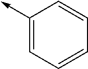 | M+ = 585.1 _{A}t_{Ret} = 4.12 min |
| 356 | 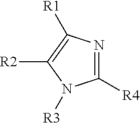 | 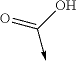 | 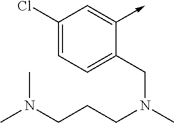 | 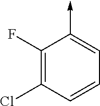 | 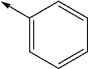 | M− = 555.1 _{A}t_{Ret} = 3.67 |
| 357 | 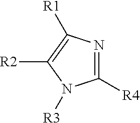 | 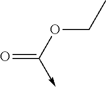 | 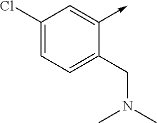 | 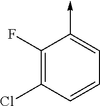 | 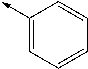 | M+ = 514.2 _{A}t_{Ret} = 4.51 min |
| 358 | 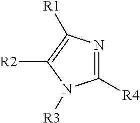 | 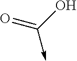 | 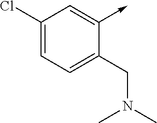 | 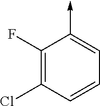 | 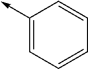 | M+ = 486.1 _{A}t_{Ret} = 4.10 min |
| 359 | 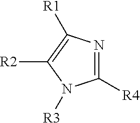 | 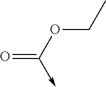 | 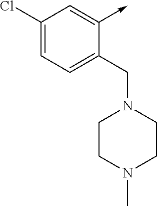 | 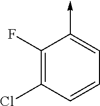 | 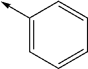 | M+ = 569.9 _{A}t_{Ret} = 4.60 min |
| 360 | 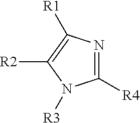 | 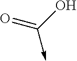 | 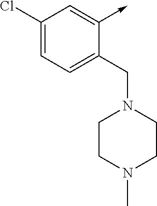 | 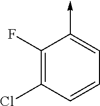 | 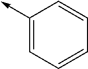 | M+ = 541.2 _{A}t_{Ret} = 4.08 min |
| 361 | 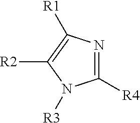 | 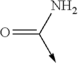 | 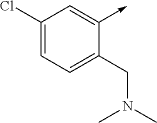 | 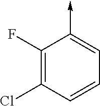 | 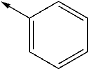 | M+ = 485.1 _{A}t_{Ret} = 4.32 min |
| 362 | 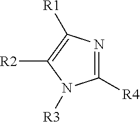 | 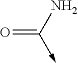 | 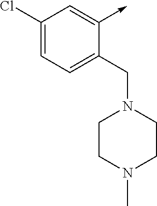 | 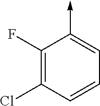 | 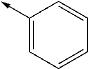 | M+ = 540.0 _{A}t_{Ret} = 4.10 min |
| 363 | 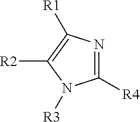 | 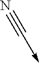 | 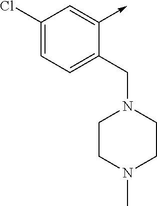 | 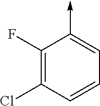 | 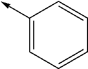 | M+ = 522.2 _{A}t_{Ret} = 4.58 min |
| 364 | 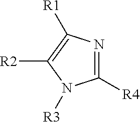 | 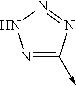 | 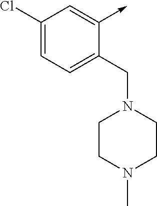 | 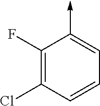 | 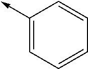 | M+ = 565.1 _{A}t_{Ret} = 4.31 min |
| 365 | 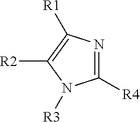 | 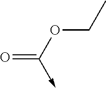 | 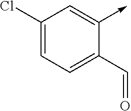 | 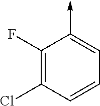 | 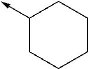 | M+ = 490.8 _{A}t_{Ret} = 5.88 min |
| 366 | 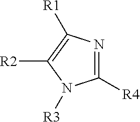 | 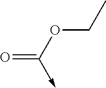 | 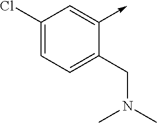 | 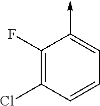 | 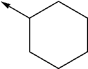 | M+ = 520.2 _{A}t_{Ret} = 4.73 min |
| 367 | 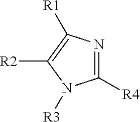 | 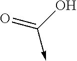 | 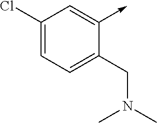 | 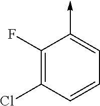 | 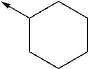 | M+ = 492.0 _{A}t_{Ret} = 4.01 min |
| 368 | 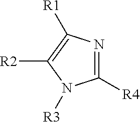 | 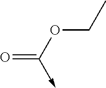 | 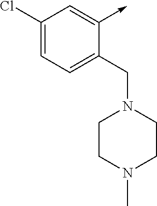 | 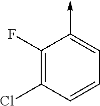 | 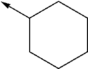 | M+ = 575.2 _{A}t_{Ret} = 4.77 min |
| 369 | 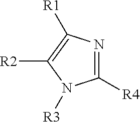 | 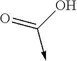 | 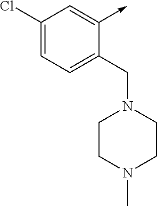 | 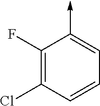 | 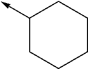 | M+ = 5.47.2 _{A}t_{Ret} = 3.88 min |
| 370 | 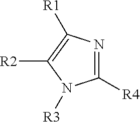 | 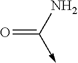 | 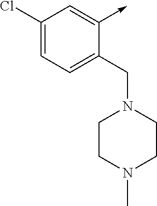 | 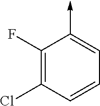 | 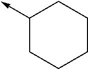 | M+ = 547.2 _{A}t_{Ret} = 3.89 min |
| 371 | 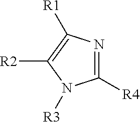 | 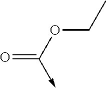 | 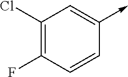 | 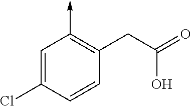 | 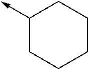 | M + H = 519/521 _{B}t_{Ret} = 1.23 min |
| 372 | 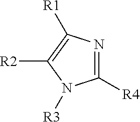 | 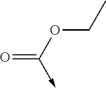 | 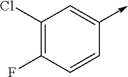 | 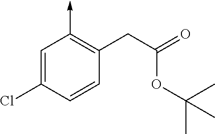 | 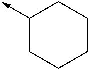 | M + H = 575/577 _{B}1_{Ret} = 1.51 min |
| 373 | 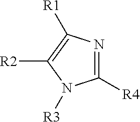 | 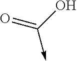 | 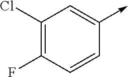 | 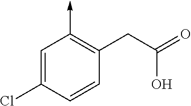 | 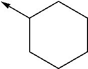 | M + H = 491/493 _{B}t_{Ret} = 1.07 min |
| 374 | 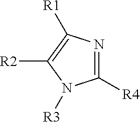 | 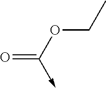 | 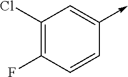 | 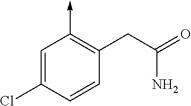 | 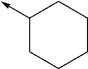 | M + H = 518/520 _{B}t_{Ret} = 1.17 min |
| 375 | 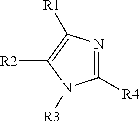 | 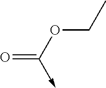 | 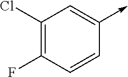 | 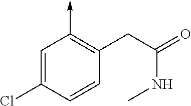 | 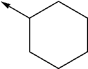 | M + H = 532/534 _{B}t_{Ret} = 1.21 min |
| 376 | 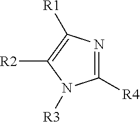 | 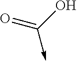 | 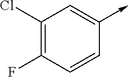 | 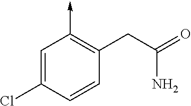 | 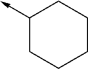 | M + H = 490/492 _{B}t_{Ret} = 1.01 min |
| 377 | 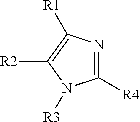 | 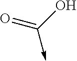 | 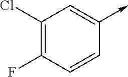 | 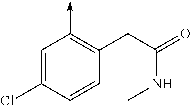 | 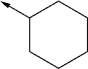 | M + H = 504/506 _{B}t_{Ret} = 1.05 min |
| 378 | 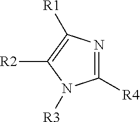 | 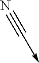 | 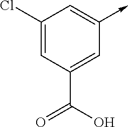 | 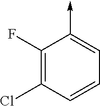 | 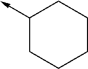 | M+ = 458.0 _{A}t_{Ret} = 5.46 min |
| 379 | 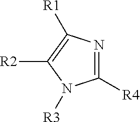 | 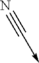 | 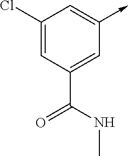 | 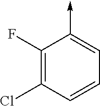 | 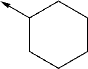 | M+ = 472.9 _{A}t_{Ret} = 5.30 min |
| 380 | 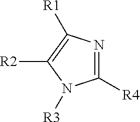 | 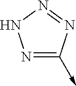 | 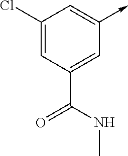 | 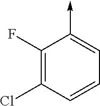 | 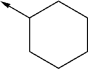 | M+ = 516.0 _{A}t_{Ret} = 4.85 min |
| 381 | 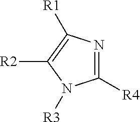 | 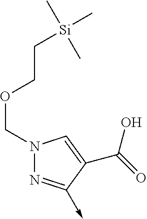 | 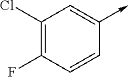 | 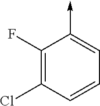 | 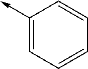 | M + 1 = 382.6 _{A}t_{Ret} = 5.26 min |
| 382 | 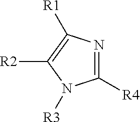 | 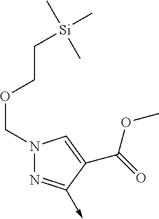 | 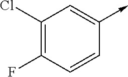 | 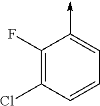 | 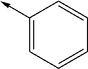 | _{A}t_{Ret} = 5.80 min. ¹H NMR CDCl₃) 8.10 (s, 1H), 7.44-7.40 (m, 4H), 7.31-7.23 (m, 4H), 7.20 (d, 1H), 7.09-7.06 (m, 1H), 6.94-6.89 (m, 1H), 5.47 (s, 2H), 4.09 (dd, 2H), 3.60 (dd, 2H), 1.69- 1.63 (m, 2H), 1.41- 1.30 (m, 4H), 1.19 (t, 3H), 0.98-0.84 (m, 7H), 0.01 (s, 9H). |
| 383 | 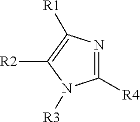 | 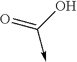 | 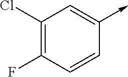 | 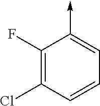 | 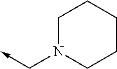 | M+ = 468.0 _{A}t_{Ret} = 4.06 min |
| 384 | 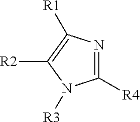 | 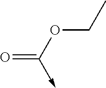 | 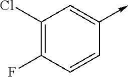 | 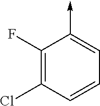 | 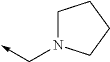 | M+ = 481.3 _{A}t_{Ret} = 530 min |
| 385 | 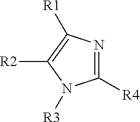 | 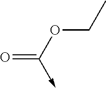 | 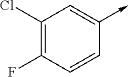 | 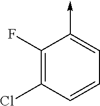 | 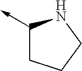 | M+ = 467.8 _{A}t_{Ret} = 4.44 min |
| 386 | 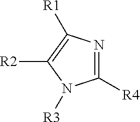 | 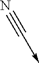 | 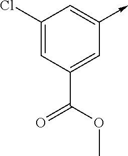 | 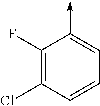 | 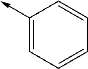 | M+ = 467.8 _{A}t_{Ret} = 5.90 min |
| 387 | 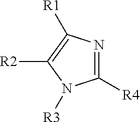 | 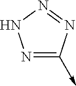 | 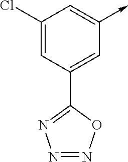 | 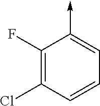 | 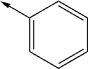 | M+ = 522.0 _{A}t_{Ret} = 5.50 min |
| 388 | 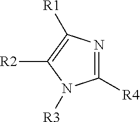 | 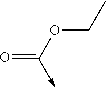 | 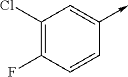 | 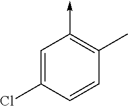 | 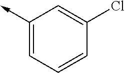 | M + H = 503/505 _{B}t_{Ret} = 1.52 min |
| 389 | 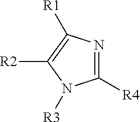 | 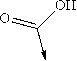 | 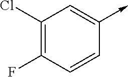 | 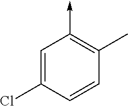 | 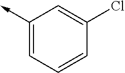 | M + H = 475/477 _{B}t_{Ret} = 1.34 min |
| 390 | 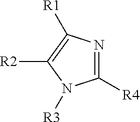 | 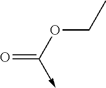 | 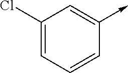 | 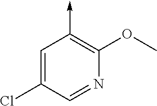 | 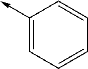 | M+ = 469.8 _{A}t_{Ret} = 5.38 min |
| 391 | 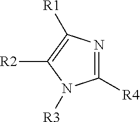 | 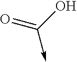 | 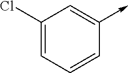 | 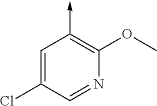 | 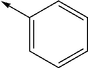 | M− = 441.9 _{A}t_{Ret} = 4.73 min |
| 392 | 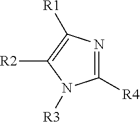 | 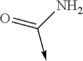 | 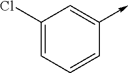 | 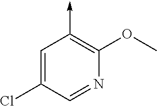 | 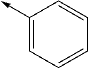 | M+ = 440.0 _{A}t_{Ret} = 4.85 min |
| 393 | 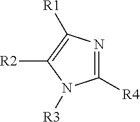 | 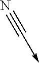 | 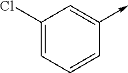 | 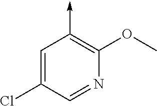 | 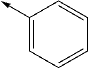 | M+ = 422.9 _{A}t_{Ret} = 5.81 min |
| 394 | 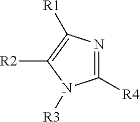 | 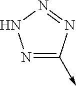 | 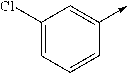 | 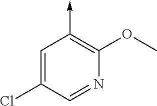 | 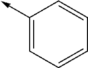 | M+ = 465.9 _{A}t_{Ret} = 5.28 min |
| 395 | 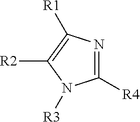 | 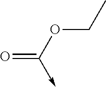 | 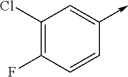 | 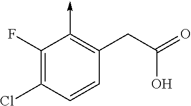 | 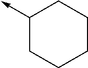 | M + 1 = 537/539 _{B}t_{Ret} = 1.33 min |
| 396 | 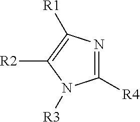 | 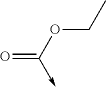 | 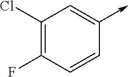 | 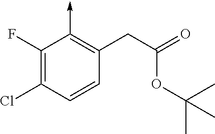 | 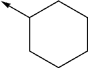 | M + 1 = 593/595 _{B}t_{Ret} = 1.60 min |
| 397 | 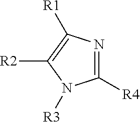 | 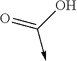 | 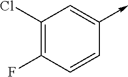 | 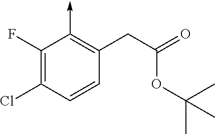 | 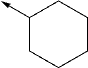 | M − 1 = 563/565 _{B}t_{Ret} = 1.41 min |
| 398 | 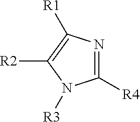 | 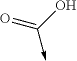 | 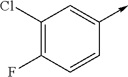 | 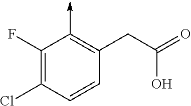 | 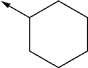 | [M − 1] − CO2 = 463/465 _{B}t_{Ret} = 1.13 min |
| 399 | 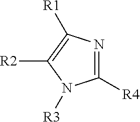 | 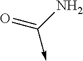 | 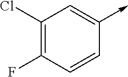 | 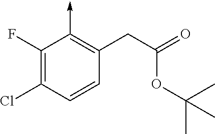 | 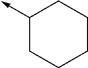 | M + 1 = 564/566 _{B}t_{Ret} = 1.47 min |
| 400 | 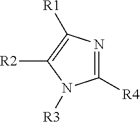 | 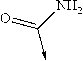 | 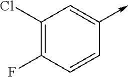 | 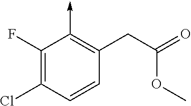 | 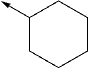 | M + 1 = 522/524 _{B}t_{Ret} = 1.33 min |
| 401 | 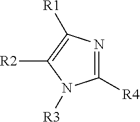 | 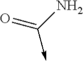 | 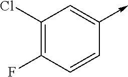 | 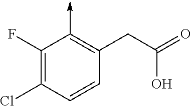 | 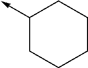 | M + 1 = 508/510 _{B}t_{Ret} = 1.18 min |
| 402 | 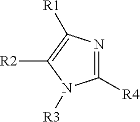 | 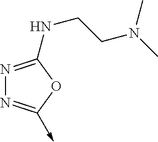 | 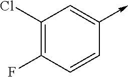 | 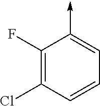 | 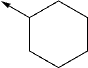 | M + H = 562.8 _{A}t_{Ret} = 4.70 min |
| 403 | 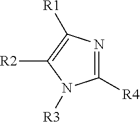 | 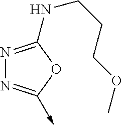 | 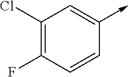 | 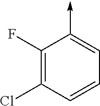 | 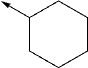 | M + H = 562.1 _{A}t_{Ret} = 5.38 min |
| 404 | 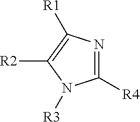 | 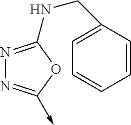 | 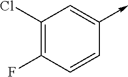 | 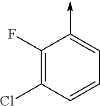 | 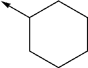 | M + H = 582.5 _{A}t_{Ret} = 5.81 min |
| 405 | 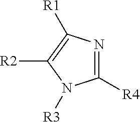 | 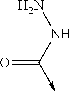 | 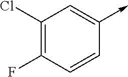 | 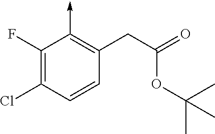 | 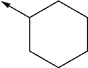 | M + 1 = 579/581 ¹H NMR (DMSO d₆) 9.05 (s, HN), 7.76 (t, 1H), 7.40 (d, 1H), 7.28 (m, 2H), 7.03 (m, 1H), 4.35 (s, H2N), 3.28 (d, 1H), 3.13 (d, 1H). 2.18 (m, 1H), 1.69 (m, 4H), 1.60 (m, 2H), 1.44 (m, 1H), 1.34 (s, 9H), 1.25-1.05 (m, 3H). |
| 406 | 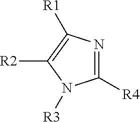 | 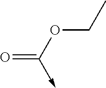 | 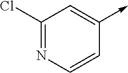 | 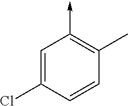 | 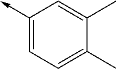 | M+ = 481.9 _{A}t_{Ret} = 5.59 min |
| 407 | 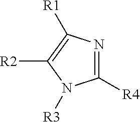 | 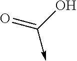 | 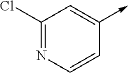 | 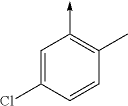 | 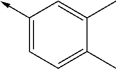 | M+ = 453.9 _{A}t_{Ret} = 4.91 min |
| 408 | 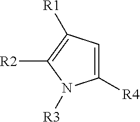 | 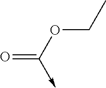 | 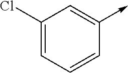 | 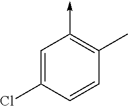 | 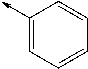 | M+ = 450.0 _{A}t_{Ret} = 6.97 min |
| 409 | 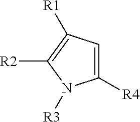 | 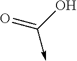 | 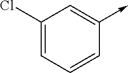 | 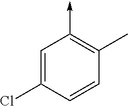 | 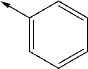 | M− = 421.9 _{A}t_{Ret} = 5.47 min |
| 410 | 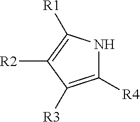 | 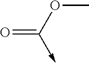 | 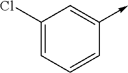 | 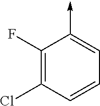 | 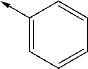 | [M + 1]+ = 440/442 _{B}t_{Ret} = 1.42 min |
| 411 | 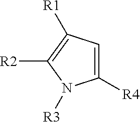 | 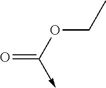 | 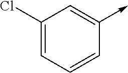 | 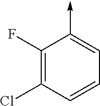 | 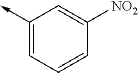 | M+ = 500.7 _{A}t_{Ret} = 6.38 min |
| 412 | 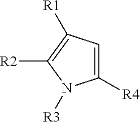 | 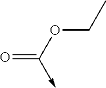 | 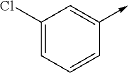 | 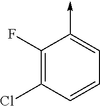 | 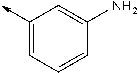 | M+ = 470.7 _{A}t_{Ret} = 4.88 min |
| 413 | 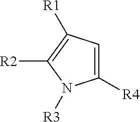 | 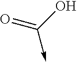 | 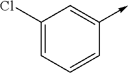 | 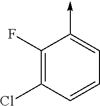 | 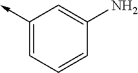 | M− = 440.8 _{A}t_{Ret} = 4.19 min |
| 414 | 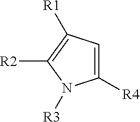 | 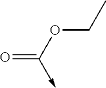 | 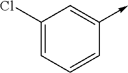 | 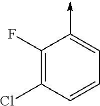 | 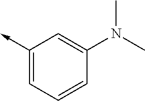 | M+ = 496.9 _{A}t_{Ret} = 5.36 min |
| 415 | 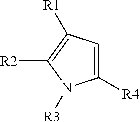 | 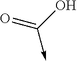 | 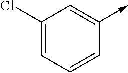 | 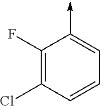 | 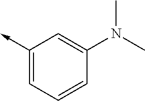 | M− = 440.8 _{A}t_{Ret} = 4.19 min |
| 416 | 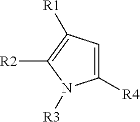 | 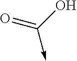 | 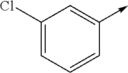 | 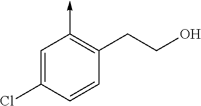 | 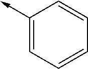 | M+ = 454.0 _{A}t_{Ret} = 5.06 min |
| 417 | 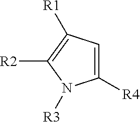 | 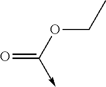 | 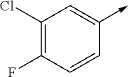 | 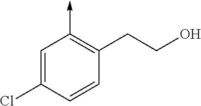 | 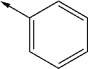 | M+ = 482.0 _{A}t_{Ret} = 5.94 min |
| 418 | 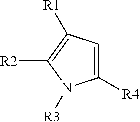 | 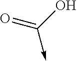 | 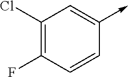 | 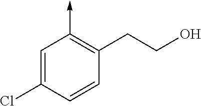 | 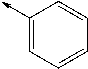 | M− = 468.1 _{A}t_{Ret} = 5.13 min |
| 419 | 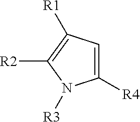 | 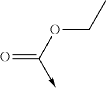 | 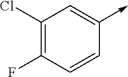 | 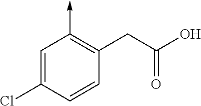 | 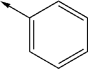 | M+ = 499.8 _{A}t_{Ret} = 5.97 min |
| 420 | 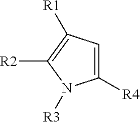 | 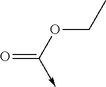 | 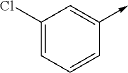 | 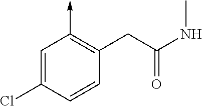 | 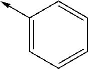 | M− = 507.0 _{A}t_{Ret} = 5.67 min |
| 421 | 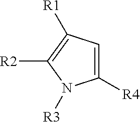 | 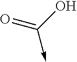 | 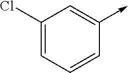 | 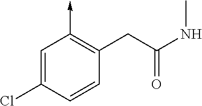 | 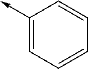 | M− = 477.0 _{A}t_{Ret} = 4.86 min |
| 422 | 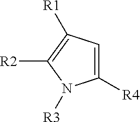 | 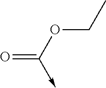 | 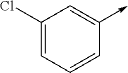 | 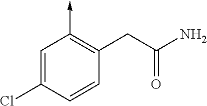 | 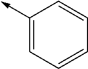 | M+ = 494.0 _{A}t_{Ret} = 6.97 min |
| 423 | 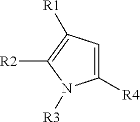 | 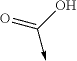 | 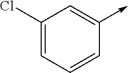 | 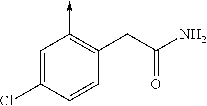 | 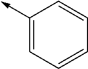 | M+ = 466.0 _{A}t_{Ret} = 4.95 min |
| 424 | 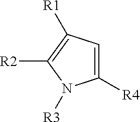 | 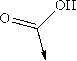 | 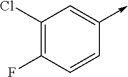 | 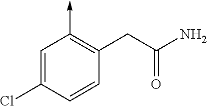 | 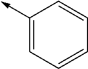 | M+ = 484.0 _{A}t_{Ret} = 4.72 min |
| 425 | 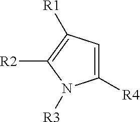 | 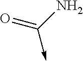 | 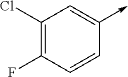 | 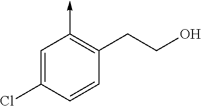 | 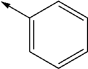 | M− = 467.1 _{A}t_{Ret} = 4.65 min |
| 426* | 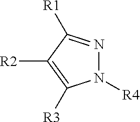 | 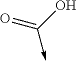 | 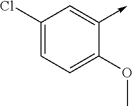 | 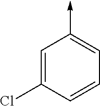 | 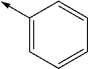 | [M + H]+ = 438.8 _{c}t_{Ret} = 1.30 min |
| 427 | 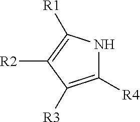 | 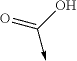 | 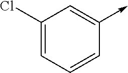 | 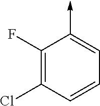 | 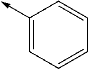 | [M + 1]+ = 426/428 _{B}t_{Ret} = 1.30 min |
| 428 | 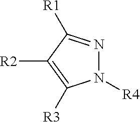 | 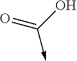 | 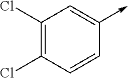 | 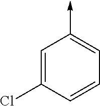 | 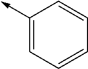 | [M + H]+ = 444.7 _{C}t_{Ret} = 2.80 min |
| 429 | 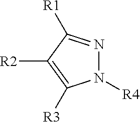 | 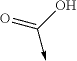 | 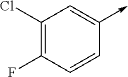 | 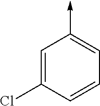 | 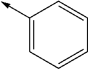 | [M + H]+ = 426.8 _{C}t_{Ret} = 2.67 min |
| 430 | 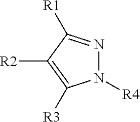 | 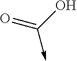 | 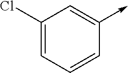 | 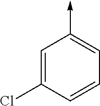 | 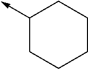 | [M + H]+ = 414.8 _{C}t_{Ret} = 2.87 min |
| 431* | 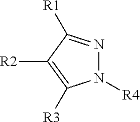 | 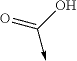 | 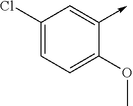 | 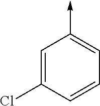 | 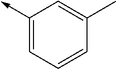 | [M + H]+ = 453.1 _{C}t_{Ret} = 2.72 min |
| 432 | 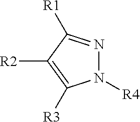 | 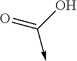 | 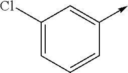 | 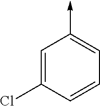 | 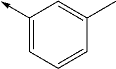 | [M + H]+ = 423.0 _{C}t_{Ret} = 2.76 min |
| 433 | 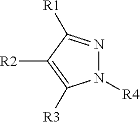 | 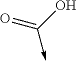 | 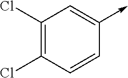 | 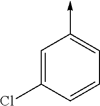 | 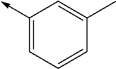 | [M + H]+ = 456.9 _{C}t_{Ret} = 2.93 min |
| 434 | 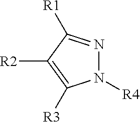 | 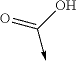 | 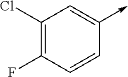 | 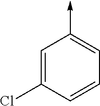 | 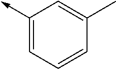 | [M + H]+ = 440.7 _{C}t_{Ret} = 2.83 min |
| 435 | 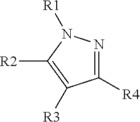 | 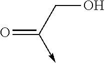 | 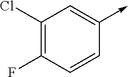 | 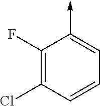 | 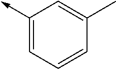 | IR: 1745 cm−1 (s) _{B}t_{Ret} = 1.49 min |
| 436 | 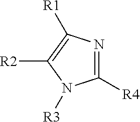 | 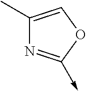 | 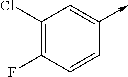 | 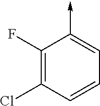 | 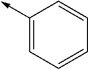 | M + 1 = 484.1 _{A}t_{Ret} = 5.56 min |
| 437 | 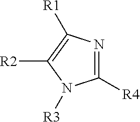 | 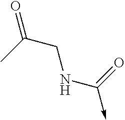 | 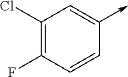 | 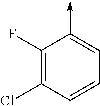 | 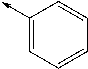 | M + 1 = 502.7 _{A}t_{Ret} = 5.20 min. |
| 438 | 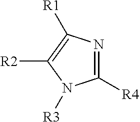 | 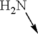 | 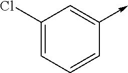 | 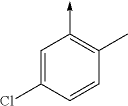 | 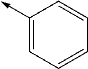 | M+ = 395.9 _{A}t_{Ret} = 4.56 min |
| 439 | 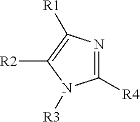 | 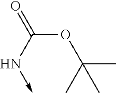 | 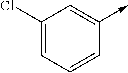 | 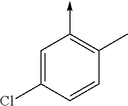 | 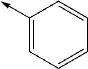 | M+ = 496.0 _{A}t_{Ret} = 5.34 min |
| 440 | 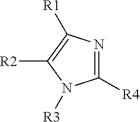 | 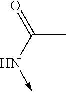 | 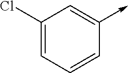 | 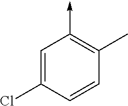 | 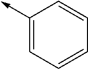 | M+ = 437.9 _{A}t_{Ret} = 4.60 min |
| 441 | 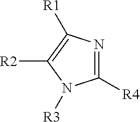 | 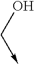 | 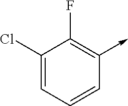 | 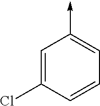 | 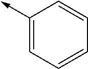 | M+ = 414.9 _{A}t_{Ret} = 4.20 min |
| 442 | 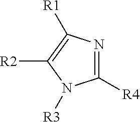 | 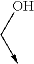 | 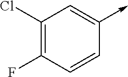 | 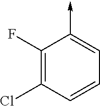 | 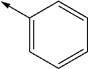 | M+ = 432.7 _{A}t_{Ret} = 4.38 min |
| 443 | 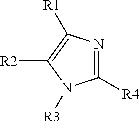 | 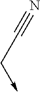 | 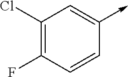 | 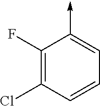 | 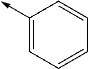 | M + Cl− = 405.8 _{A}t_{Ret} = 5.29 min |
| 444 | 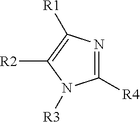 | 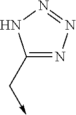 | 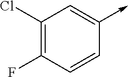 | 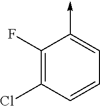 | 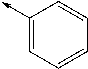 | M + Cl− = 451.0 _{A}t_{Ret} = 4.59 min |
| 445 | 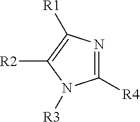 | 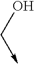 | 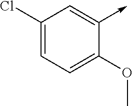 | 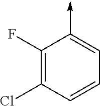 | 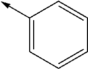 | M+ = 444.8 _{A}t_{Ret} = 4.25 min |
| 446 | 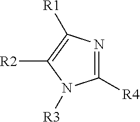 | 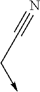 | 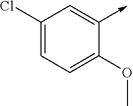 | 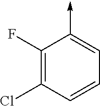 | 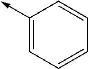 | M+ = 453.8 _{A}t_{Ret} = 5.11 min |
| 447 | 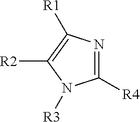 | 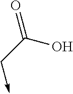 | 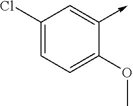 | 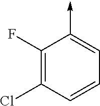 | 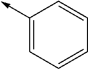 | M− = 469.9 _{A}t_{Ret} = 3.93 min |
| 448 | 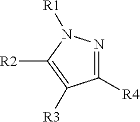 | 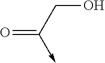 | 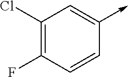 | 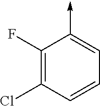 | 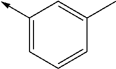 | _{B}t_{Ret} = 1.48 min TLC (hexane/ EtOAc 3:1): Rf = 0.34. |
| 449 | 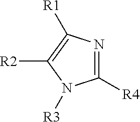 | 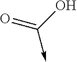 | 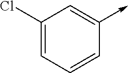 | 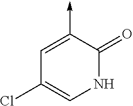 | 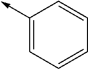 | M− = 423.9 _{A}t_{Ret} = 3.71 min |
| 450 | 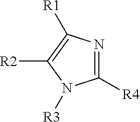 | 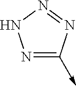 | 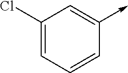 | 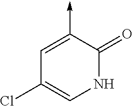 | 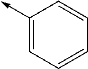 | M+ = 451.9 _{A}t_{Ret} = 2.03 min |
| 451 | 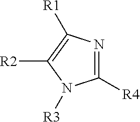 | 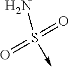 | 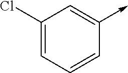 | 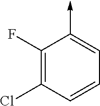 | 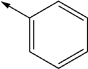 | M− = 461.9 _{A}t_{Ret} = 3.73 min |
| 452 | 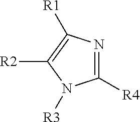 | 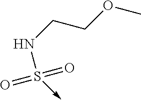 | 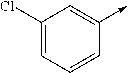 | 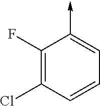 | 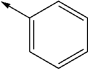 | M+ = 521.9 _{A}t_{Ret} = 4.16 min |
| 453 | 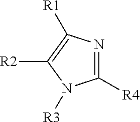 | 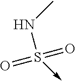 | 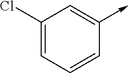 | 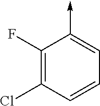 | 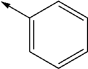 | M+ = 477.8 _{A}t_{Ret} = 4.31 min |
| 454 | 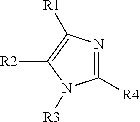 | 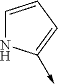 | 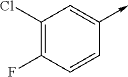 | 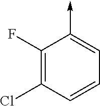 | 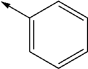 | M− = 465.9 _{A}t_{Ret} = 5.21 min |
| 455 | 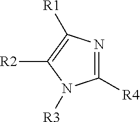 | 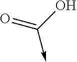 | 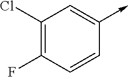 | 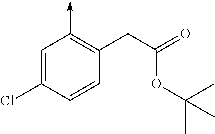 | 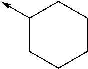 | M + H = 547/549 _{B}t_{Ret} = 1.35 min |
| 456 | 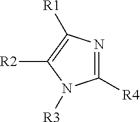 | 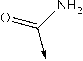 | 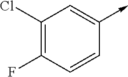 | 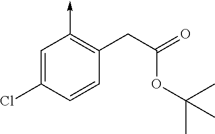 | 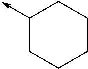 | M + H = 546/548 _{B}t_{Ret} = 1.47 min |
| 457 | 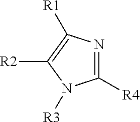 | 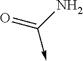 | 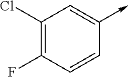 | 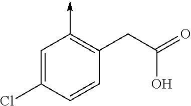 | 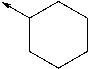 | M + H = 490/492 _{B}t_{Ret} = 1.14 min |
| 458 | 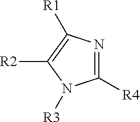 | 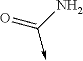 | 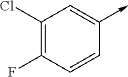 | 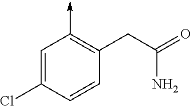 | 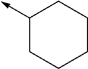 | M + H = 489/491 _{B}t_{Ret} = 1.07 min |
| 459 | 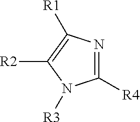 | 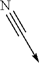 | 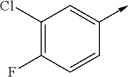 | 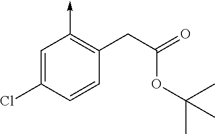 | 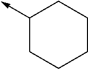 | M + H = 528/530 _{B}t_{Ret} = 1.65 min |
| 460 | 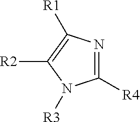 | 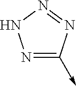 | 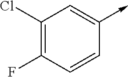 | 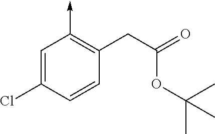 | 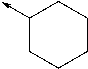 | M + H = 571/573 _{B}t_{Ret} = 1.50 min |
| 461 | 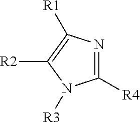 | 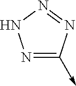 | 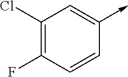 | 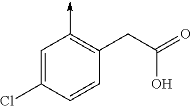 | 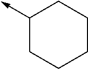 | M + H = 515/517 _{B}t_{Ret} = 1.20 min |
| 462 | 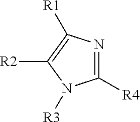 | 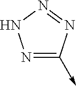 | 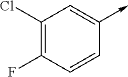 | 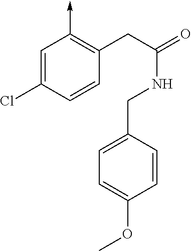 | 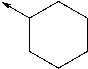 | M + H = 634/636 _{B}t_{Ret} = 1.33 min |
| 463 | 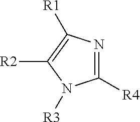 | 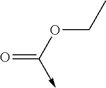 | 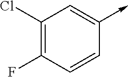 | 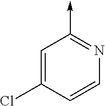 | 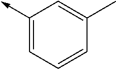 | [M + 1]+ = 470/472 _{B}t_{Ret} = 1.37 min |
| 464 | 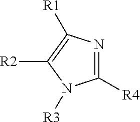 | 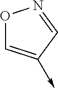 | 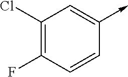 | 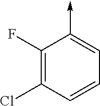 | 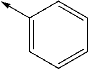 | M + 1 = 469.8. _{A}t_{Ret} = 5.43 min |
| 465 | 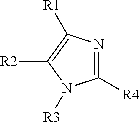 | 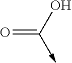 | 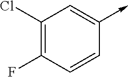 | 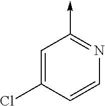 | 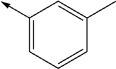 | [M + 1]+ = 442/444 _{B}t_{Ret} = 1.18 min |
| 466 | 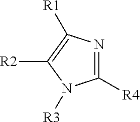 | 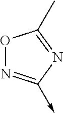 | 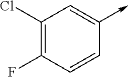 | 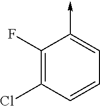 | 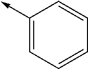 | M + 1 = 484.7 _{A}t_{Ret} = 5.44 min |
| 467 | 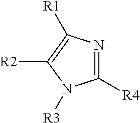 | 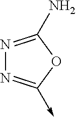 | 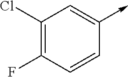 | 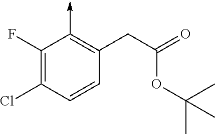 | 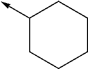 | M + 1 = 604/606 _{B}t_{Ret} = 1.36 min |
| 468 | 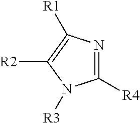 | 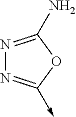 | 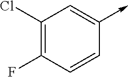 | 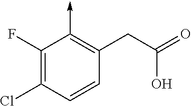 | 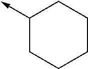 | M + 1 = 548/550 _{B}t_{Ret} = 1.11 min |
| 469 | 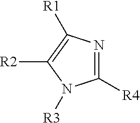 | 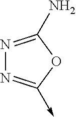 | 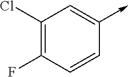 | 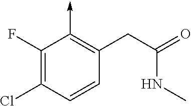 | 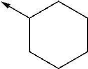 | M + 1 = 561/563 _{B}t_{Ret} = 1.11 min |
| 470 | 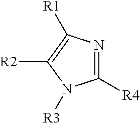 | 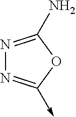 | 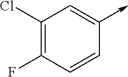 | 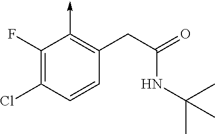 | 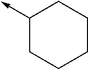 | M + 1 = 603/605 _{B}t_{Ret} = 1.30 min |
| 471 | 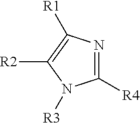 | 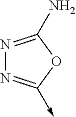 | 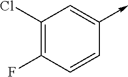 | 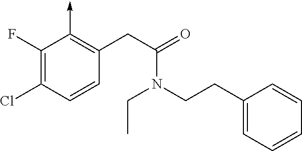 | 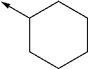 | M + 1 = 679/681 _{B}t_{Ret} = 1.36 min |
| 472 | 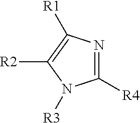 | 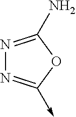 | 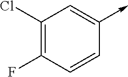 | 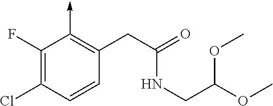 | 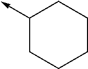 | M + 1 = 635/637 _{B}t_{Ret} = 1.17 min |
| 473 | 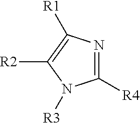 | 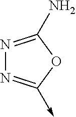 | 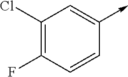 | 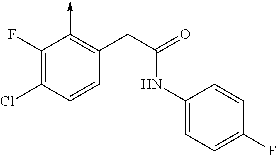 | 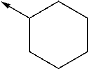 | M + 1 = 641/643 _{B}t_{Ret} = 1.34 min |
| 474 | 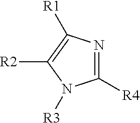 | 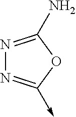 | 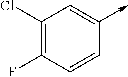 | 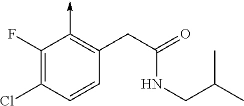 | 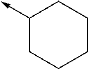 | M + 1 = 603/605 _{B}t_{Ret} = 1.26 min |
| 475 | 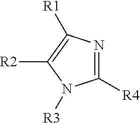 | 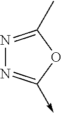 | 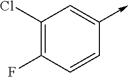 | 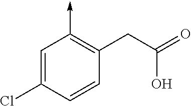 | 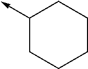 | M + 1 = 530.4 _{A}t_{Ret} = 4.98 min |
| 476 | 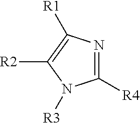 | 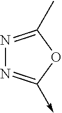 | 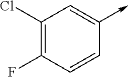 | 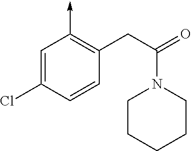 | 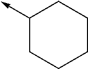 | M + 1 = 596/598 _{B}t_{Ret} = 1.35 min |
| 477 | 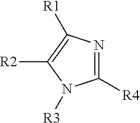 | 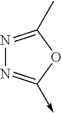 | 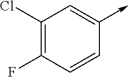 | 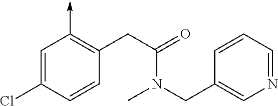 | 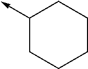 | M + 1 = 633/635 _{B}t_{Ret} = 1.07 min |
| 478 | 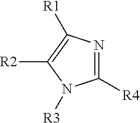 | 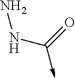 | 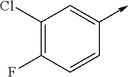 | 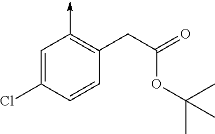 | 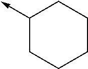 | M + 1 = 564.6 _{A}t_{Ret} = 5.23 min |
| 479 | 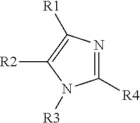 | 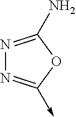 | 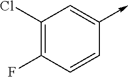 | 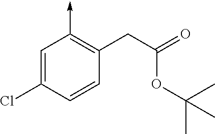 | 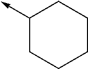 | M + 1 = 588.1 _{A}t_{Ret} = 5.55 min |
| 480 | 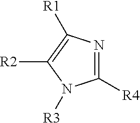 | 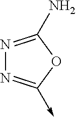 | 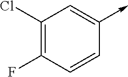 | 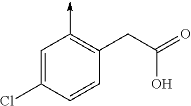 | 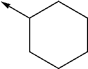 | M + 1 = 530/532 _{B}t_{Ret} = 1.08 min |
| 481 | 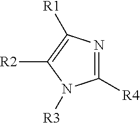 | 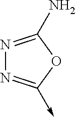 | 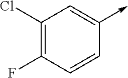 | 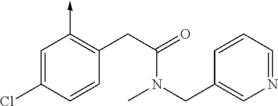 | 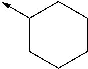 | M + 1 = 634/636 _{B}t_{Ret} = 0.98 min |
| 482 | 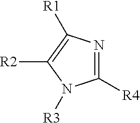 | 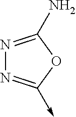 | 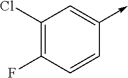 | 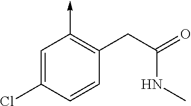 | 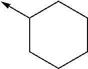 | M + 1 = 543/545 _{B}t_{Ret} = 1.07 min |
| 483 | 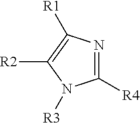 | 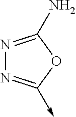 | 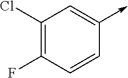 | 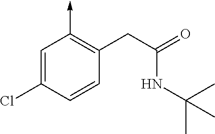 | 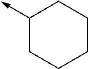 | M + 1 = 585/587 _{B}t_{Ret} = 1.24 min |
| 484 | 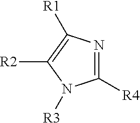 | 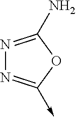 | 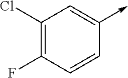 | 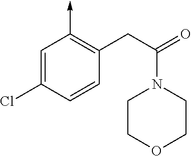 | 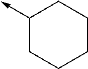 | M + 1 = 599/601 _{B}t_{Ret} = 1.09 min |
| 485 | 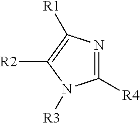 | 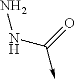 | 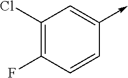 | 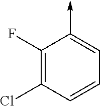 | 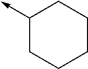 | ¹H NMR (CDCl₃) 8.40 (bs, 1H, NH), 7.52 (dd, 1H), 7.38 (d, 1H), 7.19- 7.09 (m, 2H), 7.07- 6.98 (m, 2H), 3.99 (bs, 2H, NH2), 2.37- 2.23 (m, 1H), 1.85- 1.62 (m, 6H), 1.38- 1.09 (m, 4H). _{A}t_{Ret} = 4.78 min |
| 486 | 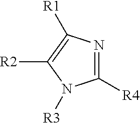 | 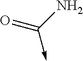 | 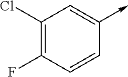 | 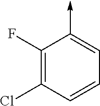 | 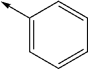 | M + 1 = 445.8 _{A}t_{Ret} = 5.16 min |
| 487 | 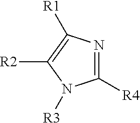 | 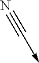 | 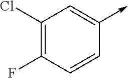 | 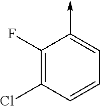 | 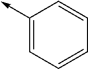 | M + 1 = 427.8 _{A}t_{Ret} = 5.84 min |
| 488 | 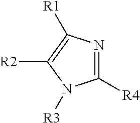 | 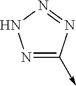 | 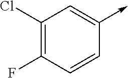 | 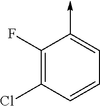 | 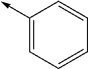 | M + 1 = 470.8 _{A}t_{Ret} = 5.33 min |
| 489 | 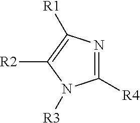 | 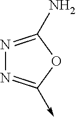 | 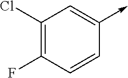 | 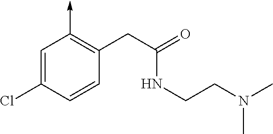 | 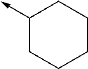 | M + 1 = 602.2 _{A}t_{Ret} = 4.09 min |
| 490 | 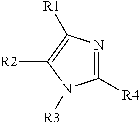 | 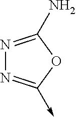 | 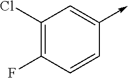 | 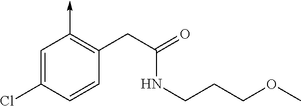 | 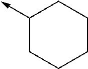 | M + 1 = 603.1 _{A}t_{Ret} = 4.65 min |
| 491 | 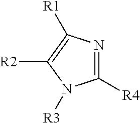 | 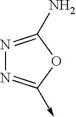 | 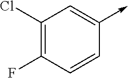 | 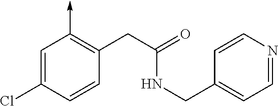 | 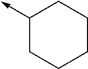 | M + 1 = 622.1 _{A}t_{Ret} = 4.16 min |
| 492 | 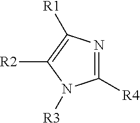 | 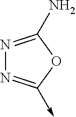 | 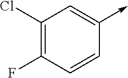 | 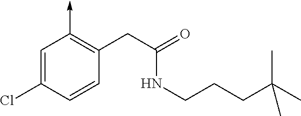 | 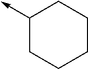 | M + 1 = 614.5 _{A}t_{Ret} = 5.28 min |
| 493 | 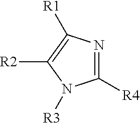 | 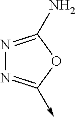 | 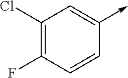 | 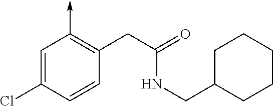 | 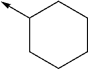 | M + 1 = 627.2 _{A}t_{Ret} = 5.35 min |
| 494 | 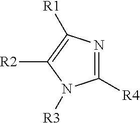 | 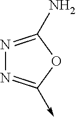 | 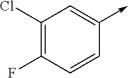 | 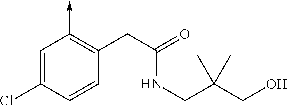 | 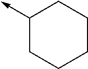 | M + 1 = 617.2 _{A}t_{Ret} = 4.66 min |
| 495 | 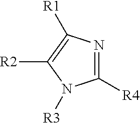 | 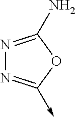 | 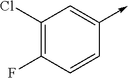 | 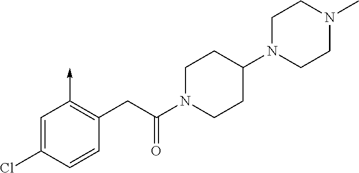 | 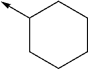 | M + 1 = 697.2 _{A}t_{Ret} = 3.99 min |
| 496 | 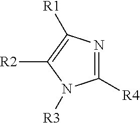 | 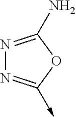 | 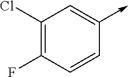 | 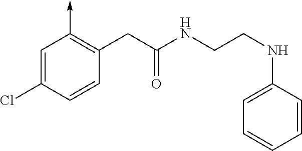 | 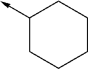 | M + 1 = 651.2 _{A}t_{Ret} = 4.15 min |
| 497 | 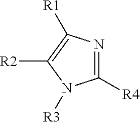 | 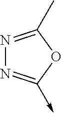 | 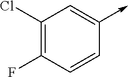 | 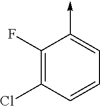 | 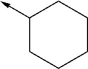 | M + 1 = 490.8 _{A}t_{Ret} = 5.74 min |
| 498 | 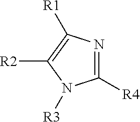 | 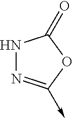 | 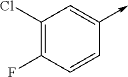 | 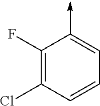 | 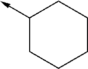 | M + 1 = 492.8 _{A}t_{Ret} = 5.51 min |
| 499 | 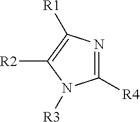 | 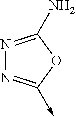 | 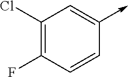 | 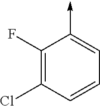 | 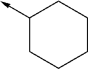 | M + 1 = 491.8 ¹H NMR (CDCl₃) 7.58 (dd, 1H), 7.34 (d, 1H), 7.21- 7.14 (m, 2H), 7.09- 7.01 (m, 2H), 5.10 (bs, 2H, NH2), 2.39- 2.26 (m, 1H), 2.84- 2.70 (m, 6H), 1.39- 1.03 (m, 4H). |
| 500 | 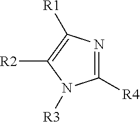 | 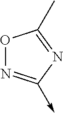 | 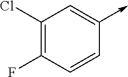 | 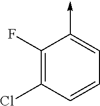 | 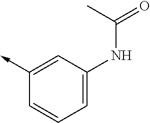 | M + 1 = 541.8 _{A}t_{Ret} = 4.59 min |
| 501 | 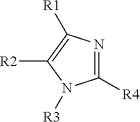 | 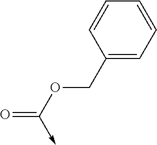 | 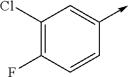 | 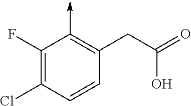 | 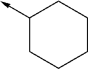 | M + 1 = 599/601 _{B}t_{Ret} = 1.42 min |
| 502 | 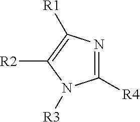 | 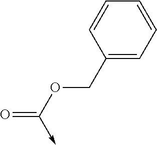 | 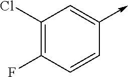 | 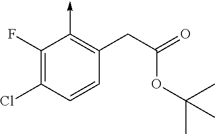 | 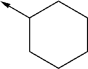 | M + 1 = 655/657 _{B}t_{Ret} = 1.65 min |
| 503 | 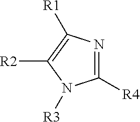 | 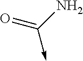 | 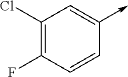 | 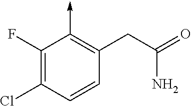 | 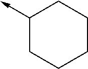 | M + 1 = 507/509 _{B}t_{Ret} = 1.14 min |
| 504 | 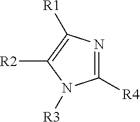 | 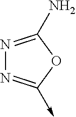 | 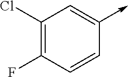 | 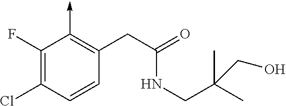 | 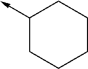 | M + 1 = 633/635 _{B}t_{Ret} = 1.16 min |
| 505 | 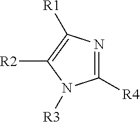 | 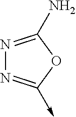 | 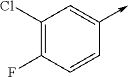 | 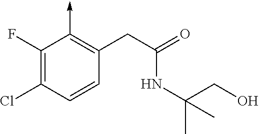 | 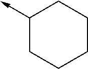 | M + 1 = 619.621 _{B}t_{Ret} = 1.14 min |
| 506 | 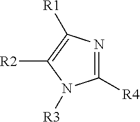 | 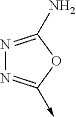 | 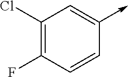 | 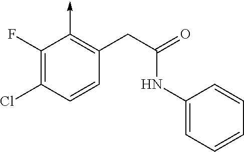 | 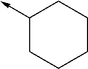 | M + 1 = 623/625 _{B}t_{Ret} = 1.29 min |
| 507 | 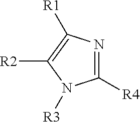 | 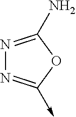 | 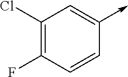 | 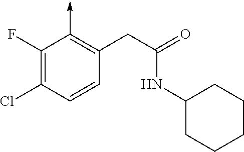 | 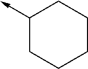 | M + 1 = 629/631 _{B}t_{Ret} = 1.30 min |
| 508 | 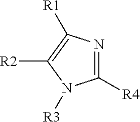 | 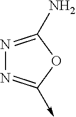 | 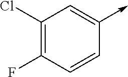 | 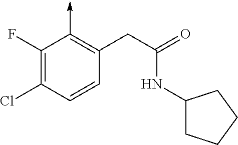 | 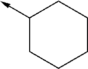 | M + 1 = 615/617 _{B}t_{Ret} = 1.26 min |
| 509 | 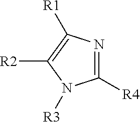 | 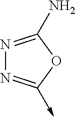 | 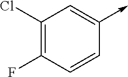 | 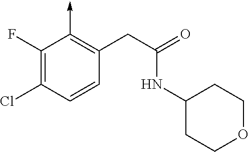 | 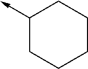 | M + 1 = 631/633 _{B}t_{Ret} = 1.15 min |
| 510 | 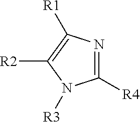 | 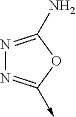 | 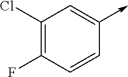 | 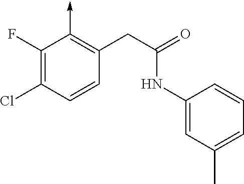 | 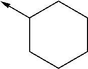 | M + 1 = 641/643 _{B}t_{Ret} = 1.32 min |
| 511 | 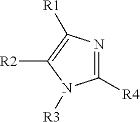 | 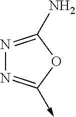 | 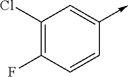 | 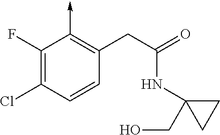 | 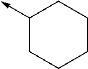 | M + 1 = 617/619 _{B}t_{Ret} = 1.10 min |
| 512 | 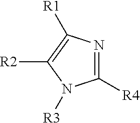 | 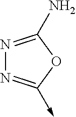 | 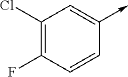 | 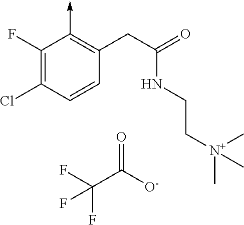 | 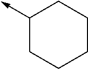 | M + 1 = 632/634 _{B}t_{Ret} = 1.00 min |
| 513 | 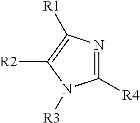 | 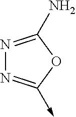 | 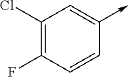 | 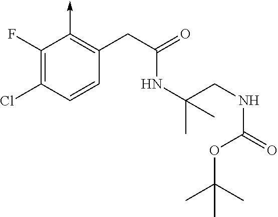 | 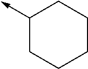 | M + 1 = 718/720 _{B}t_{Ret} = 1.37 min |
| 514 | 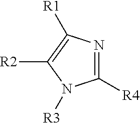 | 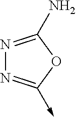 | 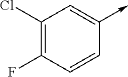 | 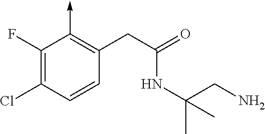 | 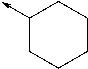 | M + 1 = 618/620 |
| 515 | 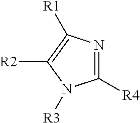 | 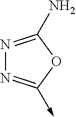 | 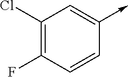 | 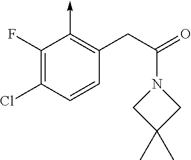 | 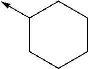 | M + 1 = 615/617 _{B}t_{Ret} = 1.25 min |
| 516 | 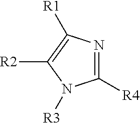 | 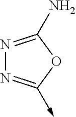 | 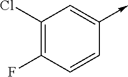 | 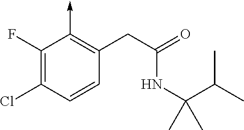 | 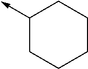 | M + 1 = 631/633 _{B}t_{Ret} = 1.39 min |
| 517 | 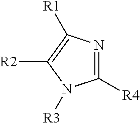 | 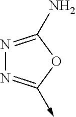 | 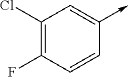 | 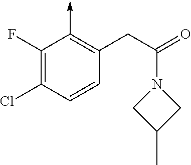 | 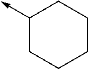 | M + 1 = 601/603 _{B}t_{Ret} = 1.19 min |
| 518 | 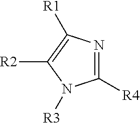 | 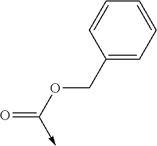 | 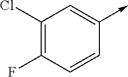 | 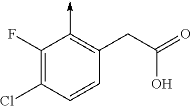 | 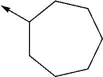 | M + 1 = 613/615 _{B}t_{Ret} = 1.45 min |
| 519 | 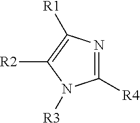 | 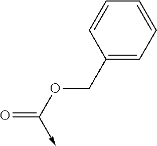 | 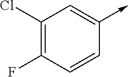 | 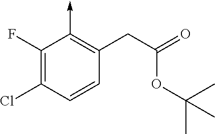 | 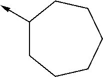 | M + 1 = 669/671 _{B}t_{Ret} = 1.68 min |
| 520 | 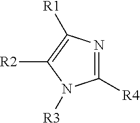 | 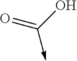 | 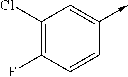 | 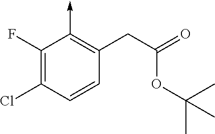 | 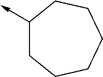 | M + 1 = 579/581 _{B}t_{Ret} = 1.46 min |
| 521 | 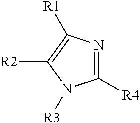 | 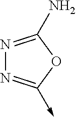 | 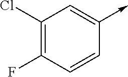 | 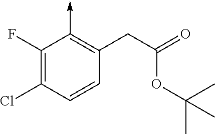 | 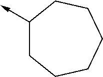 | M + 1 = 618/620 TLC (hex/ EtOAc = 1/9) Rf = 0.29 |
| 522 | 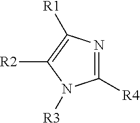 | 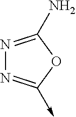 | 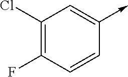 | 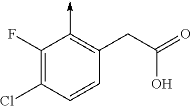 | 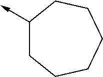 | M + 1 = 562/564 _{B}t_{Ret} = 1.19 min |
| 523 | 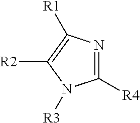 | 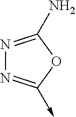 | 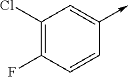 | 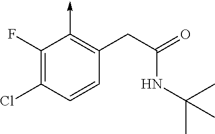 | 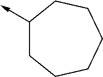 | M + 1 = 617/619 TLC (DCM/ MeOH = 9/1) Rf = 0.29 |
| 524 | 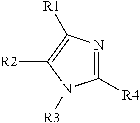 | 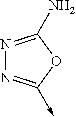 | 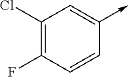 | 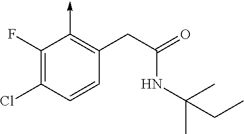 | 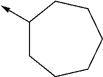 | M + 1 = 631/633 _{B}t_{Ret} = 1.40 min |
| 525 | 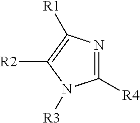 | 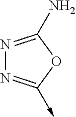 | 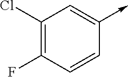 | 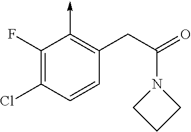 | 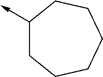 | M + 1 = 601/603 _{B}t_{Ret} = 1.19 min |

*Partial hydrolysis of the carboxylic ester was observed for these Examples. Therefore, a solution of the crude material in EtOH was heated at 80° C. for 1 h in presence of LiOH monohydrate (5 equiv.), then cooled to RT and neutralized by the addition of TFA. The resulting mixture was purified by reversed phase prep-HPLC (Waters system, gradient elution, water with 0.1% TFA/MeCN) to yield the title compound as a colorless solid.

### Intermediate 11.1

### 5-Bromo-1-(3-chloro-phenyl)-2-phenyl-1H-imidazole-4-carboxylic acid ethyl ester

The title compound is synthesized by bromination of Intermediate 11.2 analogously to the preparation of intermediate 6.1; ES-MS: M+=406.9; HPLC: _{A}t_{Ret}=5.23 min.

### Intermediate 11.2

### (3-Chloro-phenyl)-2-phenyl-1H-imidazole-4-carboxylic acid ethyl ester

The title compound is synthesized by dehydration of Intermediate 11.3 analogously to the preparation of Intermediate 6.2; ES-MS: M+=327.1; HPLC: _{A}t_{Ret}=4.71 min.

### Intermediate 11.3

### 1-(3-Chloro-phenyl)-4-hydroxy-2-phenyl-4,5-dihydro-1H-imidazole-4-carboxylic acid ethyl ester

The title compound is synthesized by cycloaddition of ethyl bromopyruvate and Intermediate 11.4 analogously to the preparation of Intermediate 6.3; ES-MS: M+=345.2; HPLC: _{A}t_{Ret}=3.65 min.

### Intermediate 11.4

### N-(3-Chloro-phenyl)-benzamidine

The title compound is synthesized by addition of 3-chloroaniline and benzonitrile analogously to the preparation of Intermediate 6.4; ES-MS: M+=231.1; HPLC: _{A}t_{Ref}=2.93 min.

### Example 16

### 5-[5-(3-Chloro-4-fluoro-phenyl)-1-(3-chloro-2-fluoro-phenyl)-2-(3-chloro-phenyl)-1H-imidazol-4-yl]-2H-tetrazole

A mixture of intermediate 16.1 (50 mg, 0.106 mmol), dioxane/water 2:1 (2.4 ml; degassed by repeated evacuation and flushing with N₂), K₃PO₄ (128 mg, 0.604 mmol), 3-chloro-phenyl boronic acid (47.2 mg, 0.302 mmol) and Pd(PPh₃)₄ (17.1 mg, 0.015 mmol) is stirred for 4 days at 85° C. After cooling the mixture to ambient temperature it is diluted with EtOAc and water, the aq. layer is separated off and extracted twice with EtOAc. The organic phases are washed with H₂O and brine, dried (Na₂SO₄) and concentrated. Reversed phase chromatography gives the title compound. ES-MS: [M+1]⁺=503/505; HPLC: _{B}t_{Ret}=1.38 min; ¹H NMR (DMSO d₆) δ 7.76 (t, 1H), 7.71 (t, 1H), 7.66 (dd, 1H), 7.57 (s, 1H), 7.52 (d, 1H), 7.43 (m, 2H), 7.35 (t, 1H), 7.33 (m, 1H), 7.26 (d, 1H).

### Intermediate 16.1

### 5-[2-Bromo-5-(3-chloro-4-fluoro-phenyl)-1-(3-chloro-2-fluoro-phenyl)-1H-imidazol-4-yl]-2H-tetrazole

A suspension of NaN₃ (1000 mg, 15.3 mmol) in toluene (1.3 ml) is cooled in an ice bath. Then Et₂AlCl (1.8 M in toluene; 8.5 ml, 15.3 mmol) is added and the mixture is stirred for 2½ h at rt and then cooled to 0° C. again. A suspension of Intermediate 16.2 (505 mg, 1.177 mmol) and toluene (7 ml) is added. The mixture is allowed to slowly warm up to rt. After 2 h, the reaction mixture is poured into water (500 ml), citric acid (8 g) and EtOAc. The aq. layer is separated off and extracted twice with EtOAc. The organic phases are washed with H₂O and brine, dried (Na₂SO₄) and concentrated, yielding the title compound which is used as such for the next step. ES-MS: [M+1]⁺=331/333; HPLC: _{B}t_{Ret}=1.25 min.

### Intermediate 16.2

### 2-Bromo-5-(3-chloro-4-fluoro-phenyl)-1-(3-chloro-2-fluoro-phenyl)-1H-imidazole-4-carbonitrile

A mixture of Intermediate 16.3 (510 mg, 1.392 mmol), toluene (26 ml) and POBr₃ (797 mg, 2.78 mmol) is stirred in a sealed tube for 7 days at 110° C., when another portion of POBr₃ (200 mg) is added. After totally 9 days at 110° C., the reaction mixture is poured into a 1:1 mixture of water and sat. NaHCO₃ (100 ml) and EtOAc. The aq. layer is separated off and extracted twice with EtOAc. The organic phases are washed with H₂O and brine and dried (Na₂SO₄). After addition of SiO₂ (2 g), the mixture is concentrated in vacuo and the resulting powder applied to a combi flash column (hexane/EtOAc 99:1→1:1), yielding the title compound. HPLC: _{B}t_{Ret}=1.41 min; ¹H NMR (DMSO d₆) δ 7.86 (t, 1H), 7.74 (t, 1H), 7.65 (dd, 1H), 7.52 (t, 1H), 7.46 (t, 1H), 7.34 (m, 1H).

### Intermediate 16.3

### 5-(3-Chloro-4-fluoro-phenyl)-1-(3-chloro-2-fluoro-phenyl)-2-oxo-2,3-dihydro-1H-imidazole-4-carbonitrile

A mixture of Intermediate 16.4 (777 mg, 2.023 mmol), toluene (39 ml) and POBr₃ (1160 mg, 4.05 mmol) is stirred for 3½ h at 110° C. Then the resulting solution is poured into a 1:1 mixture of water and sat. NaHCO₃ (100 ml) and EtOAc. The aq. layer is separated off and extracted twice with EtOAc. The organic phases are washed with H₂O and brine and dried (Na₂SO₄). After addition of SiO₂ (4 g), the mixture is concentrated in vacuo and the resulting powder applied to a combi flash column (hexane/EtOAc 99:1→1:1), yielding the title compound. ES-MS: [M−1]=364/366; HPLC: _{B}t_{Ret}=1.20 min.

### Intermediate 16.4

### 5-(3-Chloro-4-fluoro-phenyl)-1-(3-chloro-2-fluoro-phenyl)-2-oxo-2,3-dihydro-1H-imidazole-4-carboxylic acid amide

A mixture of Intermediate 16.5 (3.57 g, 9.27 mmol), dioxane (63 ml), di-tert.-butyl-dicarbonate (2.57 g, 11.77 mmol) and pyridine (749 μl, 9.27 mmol) is stirred for 15 min at rt. Then H₄NHCO₃ (930 mg, 11.77 mmol) is added and the mixture is stirred for 3½ h at 40° C. The mixture is diluted with EtOAc and water, the aq. layer is separated off and extracted twice with EtOAc. The organic phases are washed with H₂O and brine, dried (Na₂SO₄) and concentrated. To the resulting residue, dioxane (40 ml) and HCl (4 N in dioxane; 40 ml) are added. The brownish solution is stirred for 2½ h at rt and then diluted with water and EtOAc. The aq. layer is separated off and extracted twice with EtOAc. The organic phases are washed with H₂O and brine and dried (Na₂SO₄). After addition of SiO₂ (15 g), the mixture is concentrated in vacuo and the resulting powder applied to a combi flash column (DCM/EtOAc 99:1→1:4→EtOAc), yielding the title compound. ES-MS: [M+1]⁺=384/386; HPLC: _{B}t_{Ret}=1.00 min.

### Intermediate 16.5

### 5-(3-Chloro-4-fluoro-phenyl)-1-(3-chloro-2-fluoro-phenyl)-2-oxo-2,3-dihydro-1H-imidazole-4-carboxylic acid

A mixture of Intermediate 7.2 (4.3 g, 10.41 mmol), dioxane (84 ml), water (42 ml) and LiOH H₂O (0.48 g, 11.4 mmol) is stirred at 60° C. for 3% days. The reaction mixture is concentrated in vacuo and the resulting residue diluted with water (0.34 l). CH₃CN is then added to form a clear solution. This solution is acidified with HOAc and then partially concentrated in vacuo, leading to the crystallization of the title compound, which is filtered off and washed with water. ES-MS: [M+1]⁺=385/387; HPLC: _{B}t_{Ret}=1.04 min.

### Example 20 and 21

### 5-[1-(5-Chloro-2-methyl-phenyl)-5-(3-chloro-phenyl)-2-(3,4-dimethyl-phenyl)-1H-imidazol-4-yl]-2-methyl-2H-tetrazole (B) and 5-[1-(5-chloro-2-methyl-phenyl)-5-(3-chloro-phenyl)-2-(3,4-dimethyl-phenyl)-1H-imidazol-4-yl]-1-methyl-1H-tetrazole (A)

Example 2 (74.6 mg, 0.157 mmol) is dissolved in dioxane (0.6 ml). Then Cs₂CO₃ (153 mg, 0.471 mmol) is added, followed by a solution of methyl-iodide (2.36 ml; 0.1 M in dioxane) and the reaction vessel is sealed. After 3 d at rt, another 1 ml of the 0.1 M methyl-iodide solution is added and stirring continued for additional 24 h. The reaction mixture is diluted with water and EtOAc, the aq. layer separated off and extracted twice with EtOAc. The organic phases are washed with H₂O and brine, dried (Na₂SO₄) and concentrated. Combi Flash chromatography (DCM→DCM/EtOAc 9:1) gives title compound A. mp: 192-193° C.; ¹H-NMR (CD₃OD): δ ppm 4.38 [s, H₃C—N(1); NOE to H—C(2) of the 3-chloro-phenyl residue]; TLC(DCM/EtOAc 19:1): R_{f}=0.52; HPLC: _{B}t_{Ret}=1.45 min. Reversed phase chromatography of fractions containing a mixture of regio isomers gives title compound B. ¹H-NMR (CD₃OD): δ ppm 4.35 [s, H₃C—N(2)]; TLC(DCM/EtOAc 19:1): R_{f}=0.32; HPLC: _{B}t_{Ret}=1.34 min.

### Intermediate 26.1

### 5-Bromo-1-(4-chloro-phenyl)-2-cyclopropylmethyl-1H-imidazole-4-carboxylic acid ethyl ester

The title compound is synthesized by bromination of Intermediate 26.2 analogously to the preparation of Intermediate 6.1; ES-MS: M+=383.6; HPLC: _{A}t_{Ret}=4.98 min.

### Intermediate 26.2

### 1-(4-chloro-phenyl)-2-cyclopropylmethyl-1H-imidazole-4-carboxylic acid ethyl ester

The title compound is synthesized by dehydration of Intermediate 26.3 analogously to the preparation of Intermediate 6.2; ES-MS: M+=307.0; HPLC: _{A}t_{Ret}=3.96 min.

### Intermediate 26.3

### 1-(4-Chloro-phenyl)-2-cyclopropylmethyl-4-hydroxy-4,5-dihydro-1H-imidazole-4-carboxylic acid ethyl ester

The title compound is synthesized by cycloaddition of ethyl bromopyruvate and Intermediate 26.4 analogously to the preparation of Intermediate 6.3 as white solid; ES-MS: M+=325.0; HPLC: _{A}t_{Ret}=3.63 min.

### Intermediate 26.4

### N-(4-Chloro-phenyl)-2-cyclopropyl-acetamidine

The title compound is synthesized by addition of 4-chloroaniline and cyclopropylmethyl-carbonitrile analogously to the preparation of Intermediate 6.4; ES-MS: M+=209.2; HPLC: _{A}t_{Ret}=2.83 min.

### Intermediate 36.1

### 5-Bromo-1-(5-chloro-2-methyl-phenyl)-2-phenyl-1H-imidazole-4-carboxylic acid ethyl ester

The title compound is synthesized by bromination of Intermediate 36.2 analogously to the preparation of Intermediate 6.1; ES-MS: M+=420.9; HPLC: _{A}t_{Ret}=5.39 min.

### Intermediate 36.2

### 1-(5-chloro-2-methyl-phenyl)-2-phenyl-1H-imidazole-4-carboxylic acid ethyl ester

The title compound is synthesized by dehydration of Intermediate 36.3 analogously to the preparation of Intermediate 6.2; ES-MS: M+=343.0; HPLC: _{A}t_{Ret}=4.04 min.

### Intermediate 36.3

### 1-(5-Chloro-2-methyl-phenyl)-4-hydroxy-2-phenyl-4,5-dihydro-1H-imidazole-4-carboxylic acid ethyl ester

The title compound is synthesized by cycloaddition of ethyl bromopyruvate and Intermediate 36.4 analogously to the preparation of Intermediate 6.3; ES-MS: M+=361.1; HPLC: _{A}t_{Ret}=3.83 min.

### Intermediate 36.4

### N-(5-Chloro-2-methyl-phenyl)-benzamidine

The title compound is synthesized by addition of 5-chloro-2-methylaniline and benzonitrile analogously to the preparation of Intermediate 6.4; ES-MS: M+=245.2; HPLC: _{A}t_{Ret}=3.03 min.

### Example 46

### 2-{4-Chloro-2-[5-(3-chloro-4-fluoro-phenyl)-2-cyclohexyl-4-(2H-tetrazol-5-yl)-imidazol-1-yl]-phenyl}-N-methyl-acetamide

Example 461 (91 mg, 0.177 mmol) dissolved in DMF (2 ml), methylamine hydrochloride (14.3 mg, 0.212 mmol), Et₃N (0.418 ml, 3.0 mmol), DMAP (9.3 mg, 76 μmol) and 2,4,6-tripropyl-1,3,5,2,4,6-trioxatriphosphorinane-2,4,6-trioxide ([50% in DMF; 206 μl, 0.353 mmol) are converted to the title compound as described in Example 375. ES-MS: [M+1]⁺=528/530; HPLC: _{B}t_{Ret}=1.18 min; ¹H NMR (DMSO d₆) δ 7.86 (s, 1H), 7.80 (m, HN), 7.63 (d, 1H), 7.53 (d, 1H), 7.36 (d, 1H), 7.33 (d, 1H), 7.30 (m, 1H), 3.02 and 2.95 (2d, 2HCH), 2.47 (s, H₃C), 2.21 (m, 1H), 1.86 (d, 1H), 1.74 (m, 4H), 1.60 (d, 1H), 1.47 (q, 1H), 1.23-1.02 (m, 3H).

### Intermediate 47.1

### 5-Bromo-1-(5-chloro-2-methyl-phenyl)-2-m-tolyl-1H-imidazole-4-carboxylic acid ethyl ester

The title compound is synthesized by bromination of Intermediate 47.2 analogously to the preparation of Intermediate 6.1; ES-MS: M+=434.9; HPLC: _{A}t_{Ret}=5.59 min.

### Intermediate 47.2

### 1-(5-chloro-2-methyl-phenyl)-2-m-tolyl-1H-imidazole-4-carboxylic acid ethyl ester

The title compound is synthesized by dehydration of Intermediate 47.3 analogously to the preparation of Intermediate 6.2; ES-MS: M+=356.8; HPLC: _{A}t_{Ret}=5.05 min.

### Intermediate 47.3

### 1-(5-Chloro-2-methyl-phenyl)-4-hydroxy-2-m-tolyl-4,5-dihydro-1H-imidazole-4-carboxylic acid ethyl ester

The title compound is synthesized by cycloaddition of ethyl bromopyruvate and Intermediate 47.4 analogously to the preparation of Intermediate 6.3; ES-MS: M+=374.8; HPLC: _{A}t_{Ret}=4.07 min.

### Intermediate 47.4

### N-(5-Chloro-2-methyl-phenyl)-3-methyl-benzamidine

The title compound is synthesized by addition of 5-chloro-2-methylaniline and 3-methylphenylnitrile analogously to the preparation of Intermediate 6.4; ES-MS: M+=259.9; HPLC: _{A}t_{Ret}=3.34 min.

### Intermediate 55.1

### 5-Bromo-1-(5-chloro-2-methyl-phenyl)-2-p-tolyl-1H-imidazole-4-carboxylic acid ethyl ester

The title compound is synthesized by bromination of Intermediate 55.2 analogously to the preparation of Intermediate 6.1; ES-MS: M+=434.9; HPLC: _{A}t_{Ret}=5.59 min.

### Intermediate 55.2

### 1-(5-chloro-2-methyl-phenyl)-2-p-tolyl-1H-imidazole-4-carboxylic acid ethyl ester

The title compound is synthesized by dehydration of Intermediate 55.3 analogously to the preparation of Intermediate 6.2; ES-MS: M+=355.8.

### Intermediate 55.3

### 1-(5-Chloro-2-methyl-phenyl)-4-hydroxy-2-p-tolyl-4,5-dihydro-1H-imidazole-4-carboxylic acid ethyl ester

The title compound is synthesized by cycloaddition of ethyl bromopyruvate and Intermediate 55.4 analogously to the preparation of Intermediate 6.3; ES-MS: M+=375.0; NMR (MeOH d₄) δ 7.39 (d, 2H), 7.24-7.19 (m, 2H), 7.18 (d, 2H), 7.01 (s, 1H), 4.27 (q, 2H), 2.26 (s, 3H), 2.24 (s, 3H), 1.43 (t, 3H).

### Intermediate 55.4

### N-(5-Chloro-2-methyl-phenyl)-4-methyl-benzamidine

The title compound is synthesized by addition of 5-chloro-2-methylaniline and 4-methylphenylnitrile analogously to the preparation of Intermediate 6.4; ES-MS: M+=259.7; ¹H NMR (MeOH d₄) δ 7.79 (d, 2H), 7.38 (d, 2H), 7.25 (d, 1H), 7.05 (d, 1H), 6.89 (s, 1H), 2.42 (s, 3H), 2.19 (s, 3H).

### Example 57

### 5-(5-Chloro-2-hydroxy-phenyl)-1-(5-chloro-2-methyl-phenyl)-2-p-tolyl-1-H-imidazole-4-carboxylic acid

Example 56 (120 mg, 0.26 mmol) is dissolved in DCM (2 ml) and BBr₃ (1 M sol in DCM; 1.3 ml, 1.3 mmol) is added at rt. The reaction mixture is then stirred in a sealed tube at 45° C. for 4 h. It is allowed to cool to rt and diluted with DCM. The organic layer is washed with H₂O, dried over Na₂SO₄ and concentrated. The remaining crude product is purified by flash chromatography (SiO₂, DCM/MeOH, gradient 0-10% MeOH) to give the title compound as a beige solid. ES-MS: M+=454.6; HPLC: _{A}t_{Ret}=4.57 min.

### Example 70

### 5-[5-(5-Chloro-2-methoxy-phenyl)-1-(5-chloro-2-methyl-phenyl)-2-m-tolyl-1-H-imidazol-4-ylmethyl]-acetamide

Example 68 (90 mg, 0.20 mmol) is dissolved in THF (3 ml) and cooled to 0° C. LAH (50 mg, 1.20 mmol) is added and the reaction mixture is subsequently warmed to 40° C. and stirred at this temperature for 12 h. It is allowed to cool to rt and diluted with EtOAc. The organic layer is washed with H₂O and brine, dried over Na₂SO₄ and concentrated. The remaining yellow solid (79 mg, 0.17 mmol) is dissolved in DCM (3 ml). At rt TEA (56 μl, 0.34 mmol) and acetyl chloride (57 μl, 0.68 mmol) are added. The reaction mixture is stirred for 30 min at rt and then all volatiles are removed under reduced pressure. The remaining crude product is purified by flash chromatography (SiO₂, DCM/MeOH, gradient 0-5% MeOH) to give the title compound as a white solid. ES-MS: M+=496.0; HPLC: _{A}t_{Ret}=4.57 min.

### Intermediate 92.1

### 5-Bromo-1-(3-chloro-phenyl)-2-isobutyl-1H-imidazole-4-carboxylic acid ethyl ester

The title compound is synthesized by bromination of Intermediate 92.2 analogously to the preparation of Intermediate 6.1 ES-MS: M+=388.9; HPLC: _{A}t_{Ret}=5.15 min.

### Intermediate 92.2

### 1-(3-chloro-phenyl)-2-isobutyl-1H-imidazole-4-carboxylic acid ethyl ester

The title compound is synthesized by dehydration of Intermediate 92.3 analogously to the preparation of Intermediate 6.2; ES-MS: M+=307.2; HPLC: _{A}t_{Ret}=4.05 min.

### Intermediate 92.3

### 1-(3-Chloro-phenyl)-2-isobutyl-4-hydroxy-4,5-dihydro-1H-imidazole-4-carboxylic acid ethyl ester

The title compound is synthesized by cycloaddition of ethyl bromopyruvate and Intermediate 92.4 analogously to the preparation of Intermediate 6.3; ES-MS: M+=325.2; HPLC: _{A}t_{Ret}=3.70 min.

### Intermediate 92.4

### N-(3-Chloro-phenyl)-2-isobutyl-acetamidine

The title compound is synthesized by addition of 3-chloroaniline and isobutylnitrile analogously to the preparation of Intermediate 6.4; ES-MS: M+=211.1; HPLC: _{A}t_{Ret}=3.02 min.

### Intermediate 105.1

### 3-[2-(4,4,5,5-Tetramethyl-[1,3,2]dioxaborolan-2-yl)-phenylamino]-propionic acid tert-butyl ester

To a solution of 2-(2-aminophenyl)-4,4,5,5-tetramethyl-[1,3,2]dioxaborolane (2.19 g, 10 mmol) in THF (10 ml) and pyridine (966 μl, 12 mmol), NaI (30 mg, 0.2 mmol) and 3-bromo-propionic acid tert-butyl ester (1.67 ml, 10 mmol) are added. The mixture is stirred at 65° C. for 18 h, cooled down to rt and diluted with EtOAc and water. The aq. layer is separated off and extracted twice with EtOAc. The organic phases are washed with H₂O and brine, dried (Na₂SO₄) and concentrated. The crude product is re-dissolved in DCM (≈3 ml) and directly applied to a Combi Flash column (hexane/EtOAc 19:1→4:1) giving the title compound in a moderate yield. ¹H NMR (DMSO d₆) δ 7.40 (d, 1H), 7.23 (t, 1H), 6.51 (m, 2H), 5.89 (t, HN), 3.31 (m, 2H), 2.46 (t, 2H), 1.38 (s, 9H), 1.25 (s, 12H); TLC(hexane/EtOAc 4:1): R_{f}=0.63.

### Intermediate 110.1

### 5-Bromo-1-(3-chloro-phenyl)-2-(2-fluoro-phenyl)-1H-imidazole-4-carboxylic acid ethyl ester

The title compound is synthesized by bromination of Intermediate 110.2 analogously to the preparation of Intermediate 6.1; ES-MS: M+=424.9; HPLC: _{A}t_{Ref}=5.21 min.

### Intermediate 110.2

### 1-(3-chloro-phenyl)-2-(2-fluoro-phenyl)-1H-imidazole-4-carboxylic acid ethyl ester

The title compound is synthesized by dehydration of Intermediate 110.3 analogously to the preparation of Intermediate 6.2; ES-MS: M+=345.1; HPLC: _{A}t_{Ref}=4.82 min.

### Intermediate 110.3

### 1-(3-Chloro-phenyl)-2-(2-fluoro-phenyl)-4-hydroxy-4,5-dihydro-1H-imidazole-4-carboxylic acid ethyl ester

The title compound is synthesized by cycloaddition of ethyl bromopyruvate and Intermediate 110.4 analogously to the preparation of Intermediate 6.3; ES-MS: M+=365.0; HPLC: _{A}t_{Ref}=3.72 min.

### Intermediate 110.4

### N-(3-Chloro-phenyl)-2-fluoro-benzamidine

The title compound is synthesized by addition of 3-chloroaniline and 2-fluorobenzonitrile analogously to the preparation of Intermediate 6.4; ES-MS: M+=251.0; HPLC: _{A}t_{Ret}=2.98 min.

### Example 114

### 3-{2-[4-Carbamoyl-5-(3-chloro-4-fluoro-phenyl)-1-(3-chloro-2-fluoro-phenyl)-1H-imidazol-2-yl]-phenylamino}-propionic acid

HCl in dioxane (14 ml; 4 N) is added to a solution of Example 105 (0.45 g, 0.766 mmol) in dioxane (14 ml). During stirring for 18 h at rt, a suspension is formed, which is diluted with Et₂O and filtered. Washing of the solid with Et₂O gives the title compound as the hydrochloride salt; ES-MS: [M+1]⁺=531/533; HPLC: _{B}t_{Ret}=1.20 min.

### Example 115

### 5-(3-Chloro-4-fluoro-phenyl)-1-(3-chloro-2-fluoro-phenyl)-2-{2-[2-(isobutyl-methyl-carbamoyl)-ethylamino]-phenyl}-1H-imidazole-4-carboxylic acid amide

To Example 114 (82 mg, 0.154 mmol) dissolved in DMF (1.5 ml), N-methyl-isobutylamine (13.5 mg, 0.154 mmol), Et₃N (215 μl, 1.54 mmol), DMAP (8.1 mg, 0.066 mmol) and 2,4,6-tripropyl-1,3,5,2,4,6-trioxatriphosphorinane-2,4,6-trioxide ([68957-94-8] 50% in DMF; 0.18 ml, 0.31 mmol) are added. The solution is stirred for ¾ h at rt and then poured into EtOAc and water. The aq. layer is separated off and extracted twice with EtOAc. The organic phases are washed with water and brine, dried (Na₂SO₄) and concentrated. Reversed phase chromatography gives the title compound. ES-MS: [M+1]⁺=600/602; HPLC: _{B}t_{Ret}=1.34 min; ¹H NMR (DMSO d₆) δ 7.96 (s, HNH), 7.63 (t, 1H), 7.53 (m, 2H), 7.41 (s, HNH), 7.37 (t, 1H), 7.32 (t, 1H), 7.24 (t, 1H), 7.19 (m, 1H), 7.12 (t, 1H), 6.76 (m, 1H), 6.63 (t, 1H), 6.31 (t, 1H), 3.37 (m, 2H), 3.12 (dd, 2H), 2.94 and 2.83 (2s, H₃C—N), 2.64 (m, 2H), 1.86 (m, 1H), 0.80 (d, 2H₃C).

### Intermediate 118.1

### 2-Benzyl-5-bromo-1-(3-chloro-phenyl)-1H-imidazole-4-carboxylic acid ethyl ester

The title compound is synthesized by bromination of Intermediate 118.2 analogously to the preparation of Intermediate 6.1; ES-MS: M+=421.0; HPLC: _{A}t_{Ret}=5.28 min.

### Intermediate 118.2

### 2-Benzyl-1-(3-chloro-phenyl)-1H-imidazole-4-carboxylic acid ethyl ester

The title compound is synthesized by dehydration of Intermediate 118.3 analogously to the preparation of Intermediate 6.2: ES-MS: M+=341.0; HPLC: _{A}t_{Ret}=4.62 min.

### Intermediate 118.3

### 2-Benzyl-1-(3-chloro-phenyl)-4-hydroxy-4,5-dihydro-1H-imidazole-4-carboxylic acid ethyl ester

The title compound is synthesized by cycloaddition of ethyl bromopyruvate and Intermediate 118.4 analogously to the preparation of Intermediate 6.3; ES-MS: M+=359.2; HPLC: _{A}t_{Ret}=3.90 min.

### Intermediate 118.4

### N-(3-Chloro-phenyl)-2-phenyl-acetamidine

The title compound is synthesized by addition of 3-chloroaniline and phenylacetonitrile analogously to the preparation of Intermediate 6.4; ES-MS: M+=245.2; HPLC: _{A}t_{Ret}=3.25 min.

### Intermediate 121.1

### 5-Bromo-1-(3-chloro-phenyl)-2-(2-chloro-phenyl)-1H-imidazole-4-carboxylic acid ethyl ester

The title compound is synthesized by bromination of Intermediate 121.2 analogously to the preparation of Intermediate 6.1; ES-MS: M+=440.0; ¹H NMR (CDCl₃) δ 7.42-7.38 (m, 3H), 7.36-7.24 (m, 4H), 7.06 (d, 1H), 4.43 (q, 2H), 1.42 (1, 3H).

### Intermediate 121.2

### 1-(3-chloro-phenyl)-2-(2-chloro-phenyl)-1H-imidazole-4-carboxylic acid ethyl ester

The title compound is synthesized by dehydration of Intermediate 121.3 analogously to the preparation of Intermediate 6.2; ES-MS: M+=362.9; ¹H NMR (CDCl₃) δ 7.99 (s, 1H), 7.58 (d, 1H), 7.39-7.19 (m, 6H), 7.00 (d, 1H), 4.20 (q, 2H), 1.39 (t, 3H).

### Intermediate 121.3

### 1-(3-Chloro-phenyl)-2-(2-chloro-phenyl)-4-hydroxy-4,5-dihydro-1H-imidazole-4-carboxylic acid ethyl ester

The title compound is synthesized by cycloaddition of ethyl bromopyruvate and Intermediate 121.4 analogously to the preparation of Intermediate 6.3; ES-MS: M+=381.0; ¹H NMR (CDCl₃) δ 7.59 (d, 1H), 7.39-7.34 (m, 2H), 7.14-6.98 (m, 2H), 6.81 (s, 1H), 6.82 (d, 1H), 4.36 (q 2H), 1.37 (t, 3H).

### Intermediate 121.4

### N-(3-Chloro-phenyl)-2-chloro-benzamidine

The title compound is synthesized by addition of 3-chloroaniline and 2-chlorobenzonitrile analogously to the preparation of Intermediate 6.4; ES-MS: M+=267.0.

### Intermediate 129.1

### 5-Bromo-1-(5-chloro-2-methyl-phenyl)-2-pyridin-3-yl-1H-imidazole-4-carboxylic acid ethyl ester

The title compound is synthesized by bromination of Intermediate 129.2 analogously to the preparation of Intermediate 6.1; ES-MS: M+=421.3; HPLC: _{A}t_{Ret}=4.16 min.

### Intermediate 129.2

### 1-(5-Chloro-2-methyl-phenyl)-2-pyridin-3-yl-1H-imidazole-4-carboxylic acid ethyl ester

The title compound is synthesized by dehydration of Intermediate 129.3 analogously to the preparation of Intermediate 6.2; ES-MS: M+=342.1 HPLC: _{A}t_{Ret}=5.03 min.

### Intermediate 129.3

### 1-(5-Chloro-2-methyl-phenyl)-4-hydroxy-2-pyridin-3-yl-4,5-dihydro-1H-imidazole-4-carboxylic acid ethyl ester

The title compound is synthesized by cycloaddition of ethyl bromopyruvate and Intermediate 129.4 analogously to the preparation of Intermediate 6.3 ES-MS: M+=

### Intermediate 129.4

### N-(5-Chloro-2-methyl-phenyl)-nicotinamidine

The title compound is synthesized by addition of 5-chloro-2-methylaniline and nicotinonitrile analogously to the preparation of Intermediate 6.4; ES-MS: M+=248.0; ¹H NMR (MeOH_{d4}) δ 9.00 (s, 1H), 8.64 (d, 1H), 8.28 (d, 1H), 7.59 (dd, 1H), 7.19 (d, 1H), 7.02 (d, 1H), 6.82 (s, 1H), 2.19 (s, 3H).

### Intermediate 134.1

### 5-Bromo-1-(5-chloro-2-methyl-phenyl)-2-pyridin-2-yl-1H-imidazole-4-carboxylic acid ethyl ester

The title compound is synthesized by bromination of Intermediate 134.2 analogously to the preparation of Intermediate 6.1; ES-MS: M+=421.7; HPLC: _{A}t_{Ret}=5.20 min.

### Intermediate 134.2

### 1-(5-Chloro-2-methyl-phenyl)-2-pyridin-2-yl-1H-imidazole-4-carboxylic acid ethyl ester

Is obtained directly from Intermediate 134.3 under identical reaction conditions as described for formation of Intermediate 6.3; ES-MS: M+=342.1; HPLC: _{A}t_{Ret}=4.69 min.

### Intermediate 134.3

### N-(5-Chloro-2-methyl-phenyl)-pyridine-2-carboxamidine

The title compound is synthesized by addition of 5-chloro-2-methylaniline and pyridine-2-carbonitrile analogously to the preparation of Intermediate 6.4; ES-MS: M+=246.1; HPLC: _{A}t_{Ret}=2.87 min.

### Intermediate 136.1

### 5-Bromo-1-(3-chloro-4-fluoro-phenyl)-2-phenyl-1H-imidazole-4-carboxylic acid ethyl ester

The title compound is synthesized by bromination of Intermediate 136.2 analogously to the preparation of Intermediate 6.1; ES-MS: M+=424.9; HPLC: _{A}t_{Ret}=5.28 min.

### Intermediate 136.2

### 1-(3-Chloro-4-fluoro-phenyl)-2-phenyl-1H-imidazole-4-carboxylic acid ethyl ester

The title compound is synthesized by dehydration of Intermediate 136.3 analogously to the preparation of Intermediate 6.2; ES-MS: M+=345.1; HPLC: _{A}t_{Ret}=4.79 min.

### Intermediate 136.3

### 1-(3-Chloro-4-fluoro-phenyl)-4-hydroxy-2-phenyl-4,5-dihydro-1H-imidazole-4-carboxylic acid ethyl ester

The title compound is synthesized by cycloaddition of ethyl bromopyruvate and Intermediate 136.4 analogously to the preparation of Intermediate 6.3; ES-MS: M+=365.0; HPLC: _{A}t_{Ret}=3.80 min.

### Intermediate 136.4

### N-(3-Chloro-4-fluoro-phenyl)-benzamidine

The title compound is synthesized by addition of 3-chloro-4-fluoroaniline and benzonitrile analogously to the preparation of Intermediate 6.4; ES-MS: M+=356.8; HPLC: _{A}t_{Ret}=5.05 min.

### Intermediate 140.1

### 5-Bromo-1-(3-chloro-2-fluoro-phenyl)-2-phenyl-1H-imidazole-4-carboxylic acid ethyl ester

The title compound is synthesized by bromination of Intermediate 140.2 analogously to the preparation of Intermediate 6.1; ES-MS: M+=424.9; HPLC: _{A}t_{Ret}=5.19 min.

### Intermediate 140.2

### 1-(3-chloro-2-fluoro-phenyl)-2-phenyl-1H-imidazole-4-carboxylic acid ethyl ester

The title compound is synthesized by dehydration of Intermediate 140.3 analogously to the preparation of Intermediate 6.2; ES-MS: M+=346.9; HPLC: _{A}t_{Ret}=4.78 min.

### Intermediate 140.3

### 1-(3-Chloro-2-fluoro-phenyl)-4-hydroxy-2-phenyl-4,5-dihydro-1H-imidazole-4-carboxylic acid ethyl ester

The title compound is synthesized by cycloaddition of ethyl bromopyruvate and Intermediate 140.4 analogously to the preparation of Intermediate 6.3; ES-MS: M+=365.0; HPLC: _{A}t_{Ret}=3.65 min.

### Intermediate 140.4

### N-(3-Chloro-2-fluoro-phenyl)-benzamidine

The title compound is synthesized by addition of 3-chloro-2-fluoroaniline and benzonitrile analogously to the preparation of Intermediate 6.4; ES-MS: M+=249.1; HPLC: _{A}t_{Ret}=3.08 min.

### Intermediate 147.1

### 5-Bromo-1-(5-chloro-2-methyl-phenyl)-2-(6-methyl-pyridin-2-yl)-1H-imidazole-4-carboxylic acid ethyl ester

The title compound is synthesized by bromination of Intermediate 147.2 analogously to the preparation of Intermediate 6.1; ES-MS: M+=435.9; ¹H NMR (MeOH_{d4}) δ 7.91 (d, 1H), 7.72 (dd, 1H), 7.43 (d, 1H), 7.39 (d, 1H), 7.22 (s, 1H), 7.17 (d, 1H), 4.21 (q, 2H), 2.17 (s, 3H), 1.97 (s, 3H), 1.42 (t, 3H).

### Intermediate 147.2

### 1-(5-Chloro-2-methyl-phenyl)-2-(6-methyl-pyridin-2-yl)-1H-imidazole-4-carboxylic acid ethyl ester

The title compound is synthesized by dehydration of Intermediate 147.3 analogously to the preparation of Intermediate 6.2; ES-MS: M+=358.3; ¹H NMR (MeOH d4) δ 7.98 (s, 1H), 7.84 (d, 1H), 7.73 (dd, 1H), 7.41 (d, 1H), 7.31 (d, 1H), 7.17 (d, 1H), 4.40 (q 2H), 2.17 (s, 3H), 1.95 (s, 3H), 1.41 (t, 3H).

### Intermediate 147.3

### 1-(5-Chloro-2-methyl-phenyl)-4-hydroxy-2-(6-methyl-pyridin-2-yl)-4,5-dihydro-1H-imidazole-4-carboxylic acid ethyl ester

The title compound is synthesized by cycloaddition of ethyl bromopyruvate and Intermediate 147.4 analogously to the preparation of Intermediate 6.3; ES-MS: M+=374.1.

### Intermediate 147.4

### N-(5-Chloro-2-methyl-phenyl)-6-methyl-pyridine-2-carboxamidine

The title compound is synthesized by addition of 5-chloro-2-methylaniline and 6-Methyl-pyridine-2-carbonitrile analogously to the preparation of Intermediate 6.4; ES-MS: M+=262.0; ¹H NMR (MeOH d4) δ 8.13 (d, 1H), 7.81 (dd, 1H), 7.59 (d, 1H), 7.21 (d, 1H), 7.01 (d, 1H), 6.93 (s, 1H), 2.60 (s, 3H), 2.12 (s, 3H).

### Example 153

### 242-(2-Carbamoyl-ethylamino)-phenyl]-5-(3-chloro-4-fluoro-phenyl)-1-(3-chloro-2-fluoro-phenyl)-1H-imidazole-4-carboxylic acid amide

Example 114 is converted to the title compound analogously as described in Example 4; ES-MS: [M+1]⁺=530/532; HPLC: _{B}t_{Ret}=1.14 min

### Example 154

### 5-(3-Chloro-4-fluoro-phenyl)-1-(3-chloro-2-fluoro-phenyl)-2-[2-(2-hydrazinocarbonyl-ethylamino)-phenyl]-1H-imidazole-4-carboxylic acid amide

Example 114 is converted to the title compound analogously as described in Example 405; ES-MS: [M+1]⁺=545/547; HPLC: _{B}t_{Ret}=1.05 min

### Intermediate 159.1

### 5-Bromo-1-(5-chloro-2-fluoro-phenyl)-2-m-tolyl-1H-imidazole-4-carboxylic acid ethyl ester

The title compound is synthesized by bromination of Intermediate 159.2 analogously to the preparation of Intermediate 6.1; ES-MS: M+=438.9; ¹H NMR (MeOH d4) δ 7.71-7.68 (m, 1H), 7.38 (dd, 1H), 7.28 (s, 1H), 7.21-7.18 (m, 1H), 7.18 (d, 1H), 4.40 (q 2H), 2.23 (s, 3H), 1.40 (t, 3H).

### Intermediate 159.2

### 1-(5-Chloro-2-fluoro-phenyl)-2-m-tolyl-1H-imidazole-4-carboxylic acid ethyl ester

The title compound is synthesized by dehydration of Intermediate 159.3 analogously to the preparation of Intermediate 6.2; ES-MS: M+=361.1.

### Intermediate 159.3

### 1-(5-Chloro-2-fluoro-phenyl)-4-hydroxy-2-m-tolyl-4,5-dihydro-1H-imidazole-4-carboxylic acid ethyl ester

The title compound is synthesized by cycloaddition of ethyl bromopyruvate and Intermediate 159.4 analogously to the preparation of Intermediate 6.3; ES-MS: M+=361.1.

### Intermediate 159.4

### N-(5-Chloro-2-fluoro-phenyl)-3-methyl-benzamidine

The title compound is synthesized by addition of 5-chloro-2-fluoroaniline and 3-methylphenylnitrile analogously to the preparation of Intermediate 6.4; ES-MS: M+=265.0.

### Intermediate 191.1

### 5-Bromo-1-(3-chloro-2-fluoro-phenyl)-2-(6-methyl-pyridin-2-yl)-1H-imidazole-4-carboxylic acid ethyl ester

The title compound is synthesized by bromination of Intermediate 191.2 analogously to the preparation of Intermediate 6.1; ES-MS: M+=439.9; HPLC: _{A}t_{Ret}=5.06 min.

### Intermediate 191.2

### 1-(3-Chloro-2-fluoro-phenyl)-2-(6-methyl-pyridin-2-yl)-1H-imidazole-4-carboxylic acid ethyl ester

The title compound is obtained directly by reaction of Intermediate 191.3 with ethyl bromo pyruvate under conditions described for preparation of Intermediate 6.3. ES-MS: M+=361.9; HPLC: _{A}t_{Ret}=4.79 min.

### Intermediate 191.3

### N-(3-Chloro-2-fluoro-phenyl)-6-methyl-pyridine-2-carboxamidine

The title compound is synthesized by addition of 3-chloro-2-fluoroaniline and 6-Methyl-pyridine-2-carbonitrile analogously to the preparation of Intermediate 6.4; ES-MS: M+=264.1; HPLC: _{A}t_{Ret}=3.18 min.

### Intermediate 201.1

### 5-Bromo-1-(5-chloro-2-methyl-phenyl)-2-cyclohexyl-1H-imidazole-4-carboxylic acid ethyl ester

The title compound is synthesized by bromination of Intermediate 201.2 analogously to the preparation of Intermediate 6.1; ES-MS: M+H=425/427; HPLC: _{B}t_{Ret}=1.38 min.

### Intermediate 201.2

### 1-(5-Chloro-2-methyl-phenyl)-2-cyclohexyl-1H-imidazole-4-carboxylic acid ethyl ester

The title compound is synthesized by dehydration of Intermediate 201.3 analogously to the preparation of Intermediate 6.2; ES-MS: M+H=347/349; HPLC: _{B}t_{Ret}=1.13 min.

### Intermediate 201.3

### 1-(5-Chloro-2-methyl-phenyl)-2-cyclohexyl-4-hydroxy-4,5-dihydro-1H-imidazole-4-carboxylic acid ethyl ester

The title compound is synthesized by cycloaddition of Ethyl bromopyruvate and Intermediate 201.4 analogously to the preparation of Intermediate 6.3; ES-MS: M+H=365/367; HPLC: _{B}t_{Ret}=0.99 min.

### Intermediate 201.4

### N-(5-Chloro-2-methyl-phenyl)-cyclohexanecarboxamidine

The title compound is synthesized by addition of 5-chloro-2-methylaniline and cyclohexane-carbonitrile analogously to the preparation of Intermediate 6.4; ES-MS: M+H=251/253; TLC: (DCM/MeOH/NH₃^{aq}=9:1:0.1) Rf=0.27.

### Example 205

### 2-{2-[2-(5-Amino-[1,3,4]oxadiazol-2-yl)-ethylamino]-phenyl}-5-(3-chloro-4-fluoro-phenyl)-1-(3-chloro-2-fluoro-phenyl)-1H-imidazole-4-carboxylic acid amide

NaHCO₃ (9.6 mg, 114 μmol) dissolved in H₂O (0.65 ml) is added to a mixture of Example 154 (54 mg, 99 μmol) and dioxane (1.3 ml). After stirring for 10 min, cyanogen bromide (11.5 mg, 109 μmol) is added. Stirring at rt is continued for 2 h. Then the reaction mixture is diluted with EtOAc and water. The aq. layer is separated off and extracted twice with EtOAc. The organic phases are washed with water and brine, dried (Na₂SO₄) and partially concentrated. This lead to the crystallization of the title compound, which is filtered off, washed with hexane and dried; ES-MS: [M+1]⁺=570/572; HPLC: _{B}t_{Ret}=1.11 min; ¹H NMR (DMSO d₆) δ 7.80 (s, HNH), 7.61 (t, 1H), 7.53 (dd, 1H), 7.47 (t, 1H), 7.32 (t, 1H), 7.29 (s, HNH), 7.22 (t, 1H), 7.19 (m, 1H), 7.15 (t, 1H), 6.91 (s, H₂N), 6.73 (m, 3H), 6.38 (t, 1H), 3.47 (m, 2H), 2.95 (t, 2H).

### Example 206 and 207

### 5-(3-Chloro-4-fluoro-phenyl)-1-(3-chloro-2-fluoro-phenyl)-2-[2-(2-methyl-4,5-dihydro-imidazol-1-yl)-phenyl]-1H-imidazole-4-carboxylic acid N′-acetyl-hydrazide A and 2-{5-(3-Chloro-4-fluoro-phenyl)-1-(3-chloro-2-fluoro-phenyl)-2-[2-(2-methyl-4,5-dihydro-imidazol-1-yl)-phenyl]-1H-imidazol-4-yl}-5-methyl-[1,3,4]oxadiazole

POCl₃ (685 μl, 7.35 mmol) is added to a solution of Example 234 (137 mg, 228 μmol) in CH₃CN (13.7 ml). This mixture is stirred for 31/2 h at 46° C. and then poured into a mixture of ice (100 g), sat. NaHCO₃ solution (50 ml) and EtOAc (0.2 l). After stirring for 10 min, the aq. layer is separated off and extracted twice with EtOAc. The organic phases are washed with brine, dried (Na₂SO₄) and concentrated. Reversed phase chromatography separates A and B. A: ES-MS: [M+1]⁺=583/585; HPLC: _{B}t_{Ret}=0.99 min; ¹H NMR (DMSO d₆) δ 9.90 and 9.77 (2sb, 2HN), 7.63 (d, 1H), 7.57 (t, 1H), 7.55 (m, 1H), 7.45 (t, 1H), 7.35 (t, 1H), 7.33 (t, 1H), 7.28 (t, 1H), 7.17 (t, 1H), 7.14 (m, 1H), 7.09 (d, 1H), 3.52 (m, H₂C), 3.21 (m, HCH), 2.97 (m, HCH), 1.85 (s, H₃C), 1.21 (s, H₃C). B: ES-MS: [M+1]⁺=565/567; HPLC: _{B}t_{Ret}=1.06 min; ¹H NMR (DMSO d₆) 7.65 (m, 2H), 7.59 (t, 1H), 7.47 (t, 1H), 7.41 (t, 1H), 7.37 (t, 1H), 7.30 (m, 2H), 7.18 (t, 1H), 7.11 (d, 1H), 3.51 (m, H₂C), 3.21 (m, HCH), 2.96 (m, HCH), 2.51 (s, H₃C), 1.22 (s, H₃C).

### Intermediate 209.1

### 5-Bromo-1-(3-chloro-2-fluoro-phenyl)-2-cyclohexyl-1H-imidazole-4-carboxylic acid ethyl ester

The title compound is synthesized by bromination of Intermediate 209.2 analogously to the preparation of Intermediate 6.1; mp: 150-152° C.; ES-MS: M+H=429/431; HPLC: _{B}t_{Ret}=1.34.

### Intermediate 209.2

### 1-(3-Chloro-2-fluoro-phenyl)-2-cyclohexyl-1H-imidazole-4-carboxylic acid ethyl ester

The title compound is synthesized by dehydration of Intermediate 209.3 analogously to the preparation of Intermediate 6.2; ES-MS: M+H=351/353; HPLC: _{B}t_{Ret}=1.15 min.

### Intermediate 209.3

### 1-(3-Chloro-2-fluoro-phenyl)-2-cyclohexyl-4-hydroxy-4,5-dihydro-1H-imidazole-4-carboxylic acid ethyl ester

The title compound is synthesized by cycloaddition of Ethyl bromopyruvate and Intermediate 209.4 analogously to the preparation of Intermediate 6.3; ES-MS: M+H=369/371; HPLC: _{B}t_{Ret}=0.95 min.

### Intermediate 209.4

### N-(3-Chloro-2-fluoro-phenyl)-cyclohexanecarboxamidine

The title compound is synthesized by addition of 3-chloro-2-fluoroaniline and cyclohexane-carbonitrile analogously to the preparation of Intermediate 6.4; ES-MS: M+H=255/257; HPLC: _{B}t_{Ret}=0.80 min.

### Example 227

### 2-(2-Amino-phenyl)-5-(3-chloro-4-fluoro-phenyl)-1-(3-chloro-2-fluoro-phenyl)-1H-imidazole-4-carboxylic acid amide

The title compound is synthesized by Suzuki Coupling of Intermediate 227.1 with 2-(2-aminophenyl)-4,4,5,5-tetramethyl-[1,3,2]dioxaborolane as described in Example 7; ES-MS: [M+1]⁺=459/461; HPLC: _{B}t_{Ret}=1.16 min; ¹H NMR (DMSO d₆) δ 7.83 (s, HNH), 7.60 (t, 1H), 7.52 (m, 2H), 7.32 (t, 1H), 7.24 (m, HNH), 7.22 (t, 1H), 7.18 (m, 1H), 6.99 (t, 1H), 6.74 (d, 1H), 6.63 (d, 1H), 6.28 (t, 1H), 5.89 (s, H₂N).

### Intermediate 227.1

### 2-Bromo-5-(3-chloro-4-fluoro-phenyl)-1-(3-chloro-2-fluoro-phenyl)-1H-imidazole-4-carboxylic acid amide

To a solution of Intermediate 227.2 (1.16 g, 3.15 mmol) in CH₃CN (75 ml), NBS (2.8 g, 15.7 mmol) is added and the suspension is stirred for 5 d at rt. The reaction mixture is diluted with EtOAc and water, the aq. layer separated off and extracted twice with EtOAc. The organic phases are washed with H₂O and brine, dried (Na₂SO₄) and concentrated. Column chromatography (SiO₂; hexane/EtOAc 98:2→1:1) gives the title compound; ES-MS: [M+1]⁺=446/448/450; HPLC: _{B}t_{Ret}=1.21 min.

### Intermediate 227.2

### 5-(3-Chloro-4-fluoro-phenyl)-1-(3-chloro-2-fluoro-phenyl)-1H-imidazole-4-carboxylic acid amide

To a solution of Intermediate 227.3 (1.31 g, 3.74 mmol) in dioxane (50 ml), conc. ^{aq.}NH₃ (100 ml) and ^{aq.}H₂O₂ (30%, 10 ml) are added. After 7 h stirring at rt, the reaction mixture is diluted with EtOAc and water, the aq. layer separated off and extracted twice with EtOAc. The organic phases are washed with H₂O and brine, dried (Na₂SO₄) and concentrated. Column chromatography (SiO₂; hexane/EtOAc 3:7 EtOAc) gives the title compound; ES-MS: [M+1]⁺=368/370; HPLC: _{B}t_{Ret}=1.09 min.

### Intermediate 227.3

### 5-(3-Chloro-4-fluoro-phenyl)-1-(3-chloro-2-fluoro-phenyl)-1H-imidazole-4-carbonitrile

A solution of Intermediate 227.4 (0.50 g, 1.44 mmol) in dioxane (14 ml) and H₂O (7 ml) is degassed by repeated evacuation and flushing with N₂. Then K₃PO₄ (1.19 g, 5.61 mmol), 3-chloro-4-fluoro-phenyl boronic acid (0.502 g, 2.88 mmol) and Pd(PPh₃)₄ (166 mg, 0.144 mmol) are added. Stirring for 2½ h at 100° C. produces a yellow suspension. After cooling the mixture to ambient temperature it is diluted with EtOAc and water, the aq. layer is separated off and extracted twice with EtOAc. The organic phases are washed with H₂O and brine, dried (Na₂SO₄) and concentrated. Combi Flash chromatography (DCM→DCM/EtOAc 19:1) gives the title compound. ES-MS: r=350/352; HPLC: _{B}t_{Ret}=1.29 min; ¹H NMR (DMSO d₆) δ 8.38 (s, 1H), 7.79 (t, 1H), 7.65 (dd, 1H), 7.59 (t, 1H), 7.53 (t, 1H), 7.40 (t, 1H), 7.33 (m, 1H).

### Intermediate 227.4

### 1-(3-Chloro-2-fluoro-phenyl)-5-iodo-1H-imidazole-4-carbonitrile

Isopentyl nitrite (38.2 ml, 283 mmol) is added dropwise during 30 min to a suspension of Intermediate 227.5 (6.7 g, 28.3 mmol) in CH₂I₂ (34.3 ml, 425 mmol) at 10° C. Then the mixture is heated to 100° C. for ¾ h and cooled to rt again. The reaction mixture is diluted with EtOAc, sat. Na₂SO₃ solution and water, the aq. layer is separated off and extracted twice with EtOAc. The organic phases are washed with a mixture of H₂O and sat. Na₂SO₃, water and brine, dried (Na₂SO₄) and concentrated. The residue is re dissolved in DCM and after addition of SiO₂ (≈35 g) again concentrated. The resulting powder is applied to a column chromatography (SiO₂; hexane/EtOAc 3:1→2:1→3:2) yielding the title compound; mp: 182-183° C.; ES-MS: [M+1]⁺=348/350.

### Intermediate 227.5

### 6-Amino-1-(3-chloro-2-fluoro-phenyl)-1H-imidazole-4-carbonitrile

Intermediate 227.6 (8.9 g, 44.1 mmol) is added slowly to a mixture of amino-malononitrile p-toluenesulphonate (11.18 g, 44.1 mmol) and NaOAc (3.62 g, 44.1 ml) in HOAc (60 ml). The resulting suspension is stirred for 16 h at rt and then poured into water (0.6 l). Filtration and washing with a small portion of water, followed by hexane gives the title compound; ES-MS: [M+1]⁺=237/239; HPLC: _{B}t_{Ret}=0.83 min.

### Intermediate 227.6

### N-(3-Chloro-2-fluoro-phenyl)-formimidic acid ethyl ester

A solution of 3-chloro-2-fluoro-aniline (10 g, 69.3 mmol) in triethyl-orthoformate (35 ml) is stirred for 3.5 h in an oil bath of 150° C., while ≈14 ml of solvent are distilled off. The reaction mixture is then cooled to rt and filtered. The filtrate is concentrated (HV, 40° C.) and the residue used as such in the next step; ES-MS: [M+1]⁺=202/204; HPLC: _{B}t_{Ret}=0.92 min.

### Intermediate 228.1

### 5-Bromo-1-(4-chloro-phenyl)-2-(2,2-dimethyl-propyl)-1H-imidazole-4-carboxylic acid ethyl ester

The title compound is synthesized by bromination of Intermediate 228.2 analogously to the preparation of Intermediate 6.1; ES-MS: M+H=399/401; HPLC: _{B}t_{Ret}=1.34 min.

### Intermediate 228.2

### 1-(4-Chloro-phenyl)-2-(2,2-dimethyl-propyl)-1H-imidazole-4-carboxylic acid ethyl ester

The title compound is synthesized by dehydration of Intermediate 228.3 analogously to the preparation of Intermediate 6.2; ES-MS: M+H=321/323; HPLC: _{B}t_{Ret}=1.05 min.

### Intermediate 228.3

### 1-(4-Chloro-phenyl)-2-(2,2-dimethyl-propyl)-4-hydroxy-4,5-dihydro-1H-imidazole-4-carboxylic acid ethyl ester

The title compound is synthesized by cycloaddition of Ethyl bromopyruvate and Intermediate 228.4 analogously to the preparation of Intermediate 6.3; ES-MS: M+H=3391341; HPLC: _{B}t_{Ret}=0.94 min.

### Intermediate 228.4

### N-(4-Chloro-phenyl)-3,3-dimethyl-butyramidine

The title compound is synthesized by addition of 4-chloroaniline and Intermediate 228.5 analogously to the preparation of Intermediate 6.4; ES-MS: M+H=225/227; HPLC: _{B}t_{Ret}=0.77 min.

### Intermediate 228.5

### 3,3-Dimethyl-butyronitrile

1-Iodo-2,2-dimethyl-propane (12 ml, 90 mmol) is added to a suspension of tetraethylammonium-cyanide (28.3 g, 181 mmol) in dioxane (117 ml). This mixture is stirred under reflux conditions for 41/2 days. The cold suspension is filtered and the residue washed with dioxane, yielding a solution of the title compound in dioxane. ¹H-NMR (CDCl₃): δ ppm 2.21 (5, H₂C), 1.08 (s, 3H₃C).

### Example 232

### 2-[2-(2-Amino-ethylamino)-phenyl]-5-(3-chloro-4-fluoro-phenyl)-1-(3-chloro-2-fluoro-phenyl)-1H-imidazole-4-carboxylic acid amide

The title compound is synthesized by Suzuki Coupling of Intermediate 227.1 with Intermediate 296.2 as described in Example 7; ES-MS: [M+1]⁺=502/504; HPLC: _{B}t_{Ret}=1.03 min; ¹H NMR (DMSO d₆) δ 7.81 (s, HNH), 7.63 (t, 1H), 7.53 (m, 2H), 7.33 (t, 1H), 7.28 (s, HNH), 7.24 (t, 1H), 7.20 (m, 1H), 7.16 (m, HN), 7.12 (t, 1H), 6.69 (d, 1H), 6.67 (d, 1H), 6.33 (t, 1H), 3.13 (m, H₂C), 2.82 (m, H₂C).

### Example 233

### 5-(3-Chloro-4-fluoro-phenyl)-1-(3-chloro-2-fluoro-phenyl)-2-{2-[2-(3-methyl-ureido)-ethylamino]-phenyl}-1H-imidazole-4-carboxylic acid amide

Methyl-carbamic acid 2,5-dioxo-pyrrolidin-1-yl ester (56 mg, 0.325 mmol) is added to a solution of Example 232 (74 mg, 0.147 mmol) in THF (2 ml). After 20 h at rt, the reaction mixture is diluted with EtOAc and water, the aq. layer is separated off and extracted twice with EtOAc. The organic phases are washed with H₂O and brine, dried (Na₂SO₄) and concentrated. Combi Flash chromatography (DCM/MeOH/^{aq.}NH₃^{conc} 97:3:0.5→9:1:0.5) and precipitation from a solution in DCM with hexane gives the title compound. ES-MS: [M+1]⁺=559/561; HPLC: _{B}t_{Ret}=1.12 min.

### Example 234

### N-(2-{2-[4-(N′-Acetyl-hydrazinocarbonyl)-5-(3-chloro-4-fluoro-phenyl)-1-(3-chloro-2-fluoro-phenyl)-1H-imidazol-2-yl]-phenylamino}-ethyl)-acetamide

A mixture of Example 297 (49 mg, 90 μmol), NMM (250 μl, 0.22 mmol), DMAP (1 mg, 9 μmol) and HATU (44.4 mg, 117 μmol) in DMF (1 ml) is stirred for 5 min at rt. Then acetic acid hydrazide (8 mg, 108 μmol) is added. After 1 h at rt, the reaction mixture is diluted with EtOAc and water, the aq. layer is separated off and extracted twice with EtOAc. The organic phases are washed with H₂O and brine, dried (Na₂SO₄) and concentrated. Combi Flash chromatography (DCM→DCM/MeOH 9:1) gives the title compound. ES-MS: [M+1]⁺=601/603; HPLC: _{B}t_{Ret}=1.11 min.

### Intermediate 236.1

### 5-Bromo-1-(3-chloro-phenyl)-2-(2,2-dimethyl-propyl)-1H-imidazole-4-carboxylic acid ethyl ester

The title compound is synthesized by bromination of Intermediate 236.2 analogously to the preparation of Intermediate 6.1; ES-MS: M+H=399/401; HPLC: _{B}t_{Ret}=1.31 min.

### Intermediate 236.2

### 1-(3-Chloro-phenyl)-2-(2,2-dimethyl-propyl)-1H-imidazole-4-carboxylic acid ethyl ester

The title compound is synthesized by dehydration of Intermediate 236.3 analogously to the preparation of Intermediate 6.2; ES-MS: M+H=321/323; HPLC: _{B}t_{Ret}=1.06 min.

### Intermediate 236.3

### 1-(3-Chloro-phenyl)-2-(2,2-dimethyl-propyl)-4-hydroxy-4,5-dihydro-1H-imidazole-4-carboxylic acid ethyl ester

The title compound is synthesized by cycloaddition of ethyl bromopyruvate and Intermediate 236.4 analogously to the preparation of Intermediate 6.3; ES-MS: M+H=3391341; HPLC: _{B}t_{Ret}=0.94 min.

### Intermediate 236.4

### N-(3-Chloro-phenyl)-3,3-dimethyl-butyramidine

The title compound is synthesized by addition of 3-chloroaniline and Intermediate 228.5 analogously to the preparation of Intermediate 6.4; ES-MS: M+H=225/227; HPLC: _{B}t_{Ret}=0.78 min.

### Intermediate 240.1

### 5-Bromo-1-(5-chloro-2-methyl-phenyl)-2-(2,2-dimethyl-propyl)-1H-imidazole-4-carboxylic acid ethyl ester

The title compound is synthesized by bromination of Intermediate 240.2 analogously to the preparation of Intermediate 6.1; ES-MS: M+H=413/415; HPLC: _{B}t_{Ret}=1.38 min.

### Intermediate 240.2

### 1-(5-Chloro-2-methyl-phenyl)-2-(2,2-dimethyl-propyl)-1H-imidazole-4-carboxylic acid ethyl ester

The title compound is synthesized by dehydration of Intermediate 240.3 analogously to the preparation of Intermediate 6.2; ES-MS: M+H=335/337; HPLC: _{B}t_{Ret}=1.11 min.

### Intermediate 240.3

### 1-(5-Chloro-2-methyl-phenyl)-2-(2,2-dimethyl-propyl)-4-hydroxy-4,6-dihydro-1H-imidazole-4-carboxylic acid ethyl ester

The title compound is synthesized by cycloaddition of Ethyl bromopyruvate and Intermediate 240.4 analogously to the preparation of Intermediate 6.3; ES-MS: M+H=353/355; HPLC: _{B}t_{Ret}=0.96 min.

### Intermediate 240.4

### N-(5-Chloro-2-methyl-phenyl)-3,3-dimethyl-butyramidine

The title compound is synthesized by addition of 5-chloro-2-methyl-aniline and Intermediate 228.5 analogously to the preparation of Intermediate 6.4; ES-MS: M+H=321/323; HPLC: _{B}t_{Ret}=1.06 min.

### Intermediate 253.1

### 5-Bromo-1-(3-chloro-2-fluoro-phenyl)-2-(2,2-dimethyl-propyl)-1H-imidazole-4-carboxylic acid ethyl ester

The title compound is synthesized by bromination of Intermediate 253.2 analogously to the preparation of Intermediate 6.1; ES-MS: M+H=417/419; HPLC: _{B}t_{Ret}=1.32 min.

### Intermediate 253.2

### 1-(3-Chloro-2-fluoro-phenyl)-2-(2,2-dimethyl-propyl)-1H-imidazole-4-carboxylic acid ethyl ester

The title compound is synthesized by dehydration of Intermediate 253.3 analogously to the preparation of Intermediate 6.2; ES-MS: M+H=339/341; HPLC: _{B}t_{Ret}=1.12 min.

### Intermediate 253.3

### 1-(3-Chloro-2-fluoro-phenyl)-2-(2,2-dimethyl-propyl)-4-hydroxy-4,5-dihydro-1H-imidazole-4-carboxylic acid ethyl ester

The title compound is synthesized by cycloaddition of Ethyl bromopyruvate and Intermediate 253.4 analogously to the preparation of Intermediate 6.3; ES-MS: M+H=357/359; HPLC: _{B}t_{Ret}=0.93 min.

### Intermediate 253.4

### N-(3-Chloro-2-fluoro-phenyl)-3,3-dimethyl-butyramidine

The title compound is synthesized by addition of 3-chloro-2-fluoroaniline and Intermediate 228.5 analogously to the preparation of Intermediate 6.4; ES-MS: M+H 243/245; HPLC: _{B}t_{Ret}=0.80 min.

### Intermediate 258.1

### 5-Bromo-1-(3-chloro-2-fluoro-phenyl)-2-cyclopentylmethyl-1H-imidazole-4-carboxylic acid ethyl ester

The title compound is synthesized by bromination of Intermediate 258.2 analogously to the preparation of Intermediate 6.1; ES-MS: M+H=429/431; HPLC: _{B}t_{Ret}=1.33 min.

### Intermediate 258.2

### 1-(3-Chloro-2-fluoro-phenyl)-2-cyclopentylmethyl-1H-imidazole-4-carboxylic acid ethyl ester

The title compound is synthesized by dehydration of Intermediate 258.3 analogously to the preparation of Intermediate 6.2; ES-MS: M+H=351/353; HPLC: _{B}t_{Ret}=1.14 min.

### Intermediate 258.3

### 1-(3-Chloro-2-fluoro-phenyl)-2-cyclopentylmethyl-4-hydroxy-4,5-dihydro-1H-imidazole-4-carboxylic acid ethyl ester

The title compound is synthesized by cycloaddition of Ethyl bromopyruvate and Intermediate 258.4 analogously to the preparation of Intermediate 6.3; ES-MS: M+H=369/371; HPLC: _{B}t_{Ret}=0.96 min.

### Intermediate 258.4

### N-(3-Chloro-2-fluoro-phenyl)-2-cyclopentyl-acetamidine

The title compound is synthesized by addition of 3-chloro-2-fluoroaniline and cyclopentylmethyl-carbonitrile analogously to the preparation of Intermediate 6.4; TLC(DCM/MeOH/Et₃N=90:10:1) Rf=0.31; HPLC: _{B}t_{Ret}=0.84 min.

### Intermediate 263.1

### 5-Bromo-1-(3-chloro-2-fluoro-phenyl)-2-cyclohexylmethyl-1H-imidazole-4-carboxylic acid ethyl ester

The title compound is synthesized by bromination of Intermediate 263.2 analogously to the preparation of Intermediate 6.1; ES-MS: M+H=443/445; HPLC: _{B}t_{Ret}=1.39 min.

### Intermediate 263.2

### 1-(3-Chloro-2-fluoro-phenyl)-2-cyclohexylmethyl-1H-imidazole-4-carboxylic acid ethyl ester

The title compound is synthesized by dehydration of Intermediate 263.3 analogously to the preparation of Intermediate 6.2; ES-MS: M+H=365/367; HPLC: _{B}t_{Ret}=1.18 min.

### Intermediate 263.3

### 1-(3-Chloro-2-fluoro-phenyl)-2-cyclohexylmethyl-4-hydroxy-4,5-dihydro-1H-imidazole-4-carboxylic acid ethyl ester

The title compound is synthesized by cycloaddition of Ethyl bromopyruvate and Intermediate 263.4 analogously to the preparation of Intermediate 6.3; ES-MS: M+H=383/385; HPLC: _{B}t_{Ret}=1.01 min.

### Intermediate 263.4

### N-(3-Chloro-2-fluoro-phenyl)-2-cyclohexyl-acetamidine

The title compound is synthesized by addition of 3-chloro-2-fluoroaniline and cyclohexylmethyl-carbonitrile analogously to the preparation of Intermediate 6.4; TLC(DCM/MeOH/Et₃N=90:10:1) Rf=0.46; HPLC: _{B}t_{Ret}=0.89 min

### Example 264

### [5-(3-Chloro-4-fluoro-phenyl)-1-(3-chloro-2-fluoro-phenyl)-2-cyclohexylmethyl-1H-imidazol-4-yl]-methanol

5-(3-Chloro-4-fluoro-phenyl)-1-(3-chloro-2-fluoro-phenyl)-2-cyclohexylmethyl-1H-imidazole-4-carboxylic acid ethyl ester (Example 263; 60 mg, 0.122 mmol) is dissolved in ^{t}BuOH (3 ml). NaBH₄ (14 mg, 0.37 mmol) is added and the mixture is stirred for 4 h at 70° C. Another portion of 14 mg NaBH₄ is added and stirring at 70° C. continued for 16 h. The reaction mixture is diluted with water and EtOAc, the aq. layer separated off and extracted twice with EtOAc. The organic phases are washed with H₂O and brine, dried (Na₂SO₄) and concentrated. Reversed phase chromatography gives the title compound. ES-MS: [M+1]⁺=451/453; HPLC: _{B}t_{Ret}=1.17 min.

### Intermediate 296.1

### N-{2-[2-(4,4,5,5-Tetramethyl-(1,3,2]dioxaborolan-2-yl)-phenylamino}-ethyl)-acetamide

To an ice-cooled solution of Intermediate 296.2 (1.41 mmol) in DMF (3 ml) and pyridine (0.5 ml), acetic acid pentafluorophenyl ester (319 mg, 1.41 mmol) is added. This solution is stirred for 3 h at 0° C. and 16 h at rt. Then the reaction mixture is diluted with water and

EtOAc and the aq. phase extracted with 2 portions of EtOAc. The organic phases are washed with H₂O and brine, dried (Na₂SO₄) and concentrated. Combi Flash chromatography (product mixture applied as a solution in DCM to the conditioned column and eluated with DCM/EtOAc 85:15→3:7) gives the title compound. ES-MS: [M+1]⁺=305; HPLC: _{B}t_{Ret}=0.78 min.

### Intermediate 296.2

### N*1*-[2-(4,4,5,6-tetramethyl-[1,3,2]dioxaborolan-2-yl)-phenyl]-ethane-1,2-diamine

Hydrogenation of Intermediate 296.3 (0.46 g, 1.57 mmol) in MeOH (15 ml) in the presence of Raney-Nickel (≈0.5 g; B113W Degussa), filtration and concentration of the filtrate in vacuo gives the title compound. ES-MS: [M+1]⁺=263; HPLC: _{B}t_{Ret}=0.49 min.

### Intermediate 296.3

### (2-Nitro-ethyl)-[2-(4,4,5,5-tetramethyl-[1,3,2]dioxaborolan-2-yl)-phenyl]-amine

To an ice-cooled solution of 2-(4,4,5,5-tetramethyl-[1,3,2]dioxaborolan-2-yl)-phenylamine (438 mg, 2.0 mmol) and benzoic acid 2-nitro-ethylester (390 mg, 2.0 mmol) in toluene (10 ml), NMM (340 μl, 3.0 mmol) is added. This solution is stirred for 2 days at rt and then diluted with water and EtOAc. The aq. phase is separated off and extracted twice with EtOAc. The organic phases are washed with H₂O and brine, dried (Na₂SO₄) and concentrated. Combi Flash chromatography (product mixture applied as a solution in DCM to the conditioned column and eluated with DCM/hexane 1:9→DCM) gives the title compound. ES-MS: [M+1]⁺=293.

### Intermediate 299.1

### 5-Hydroxymethyl-thiophene-3-boronic acid

5-Formyl-thiophene-3-boronic acid (1.00 g, 6.28 mmol) is dissolved in THF (20 ml). NaBH₄ (743 mg, 18.8 mmol) is added and the mixture is stirred for 10 min at it. The reaction mixture is diluted with DCM and MeOH. After addition of SiO₂ (2 g), it is concentrated in vacuo. The resulting powder is submitted to a Combi Flash chromatography [EtOAc→EtOAc/(10% AcOH in EtOH) 1:1] yielding the title compound. MS: [M−1]=157.

### Intermediate 301.1

### 2-Bromo-5-(3-chloro-4-fluoro-phenyl)-1-(5-chloro-2-methyl-phenyl)-1H-imidazole-4-carboxylic acid ethyl ester

The title compound is synthesized by bromination of Intermediate 301.2 analogously to the preparation of Intermediate 7.1; ES-MS: [M+1]⁺=471/473/475; HPLC: _{B}t_{Ret}=1.41 min.

### Intermediate 301.2

### 5-(3-Chloro-4-fluoro-phenyl)-1-(5-chloro-2-methyl-phenyl)-2-oxo-2,3-dihydro-1H-imidazole-4-carboxylic acid ethyl ester

A suspension of Intermediate 301.3 (2.8 g, 15.0 mmol) in 1,2-dichloro-ethane (70 ml) and dioxane (70 ml) is degassed by repeated evacuation and flushing with N₂. The mixture is warmed up to 80° C., then Intermediate 7.4 (3.7 g, 13.7 mmol) is added, followed by Rh₂Oct₄ ([Cas: 73482-96-9]; 71 mg, 0.091 mmol). After 30 min and 1 h at 80° C., two additional portions of 71 mg Rh₂Oct₄ each are added. After totally 2 h, the suspension is cooled in an ice bath, then TFA (13.7 ml) is added and stirring is continued for 2 days at rt. The solution is diluted with EtOAc and water/sat. Na₂CO₃ 1:1, the aq. layer is separated off and extracted twice with EtOAc. The organic phases are washed with H₂O and brine, dried (Na₂SO₄) and concentrated. Combi Flash chromatography (DCM/EtOAc 99:1→4:1) gives the title compound. ES-MS: [M+1]⁺=409/411; HPLC: _{B}t_{Ret}=1.19 min.

### Intermediate 301.3

### (5-Chloro-2-methyl-phenyl)-urea

The title compound is synthesized by addition reaction of 5-chloro-2-methyl-aniline analogously to the preparation of Intermediate 7.3; ES-MS: [M+1]⁺=185/187; HPLC: _{B}t_{Ret}=0.78 min.

### Example 309

### 5′-(3-Chloro-4-fluoro-phenyl) 1-(3-chloro-2-fluoro-phenyl)-2′-phenyl-1H-[1,4]bisimidazolyl

Intermediate 454.1 (100 mg, 0.21 mmol) is dissolved in dioxane (2 mL) and imidazole (17 mg, 0.25 mmol), CuI (8 mg, 0.04 mmol), trans-1.2-cyclohexyldiamine (7.1 mg, 0.06 mmol) and K₃PO₄ (133 mg, 0.6 mmol) are added at rt. The reaction vessel is sealed and heated to 120° C. for 20 h. the reaction mixture is allowed to cool to rt and submitted t aqueous workup. The remaining crude material is purified by flash chromatography (SiO₂; hexanes/EtOAc, gradient: 0-40% EtOAc). ES-MS: [M+1]=468.9. HPLC: _{A}t_{Ret}=4.42 min.

### Example 310

### [5-(3-Chloro-4-fluoro-phenyl)-1-(3-chloro-2-fluoro-phenyl)-2-cyclohexyl-1H-imidazol-4-yl]-phosphonic acid diethyl ester

A mixture of Intermediate 310.1 (164 mg, 0.337 mmol), diethylphosphite (262 μl, 2.034 mmol), NEt₃ (141 μl, 1.012 mmol) and (Ph₃P)₄Pd (194 mg, 0.168 mmol) in toluene (2 ml; degassed by repeated evacuation and flushing with N₂) is stirred for 24 at 110° C. (toluene partly evaporated). The reaction mixture is diluted with EtOAc and water, the aq. layer separated off and extracted twice with EtOAc. The organic phases are washed with H₂O and brine, dried (Na₂SO₄) and concentrated. Reversed phase chromatography gives the title compound; ES-MS: [M+1]⁺=543/545; HPLC: _{B}t_{Ret}=1.41 min.

### Intermediate 310.1

### 4-Bromo-5-(3-chloro-4-fluoro-phenyl)-1-(3-chloro-2-fluoro-phenyl)-2-cyclohexyl-1H-imidazole

Intermediate 310.2 (246 mg, 0.605 mmol) is dissolved in CH₃CN (8 ml). Then NBS (151 mg, 0.847 mmol) is added and the mixture is stirred for 20 min at rt. The resulting solution is diluted with EtOAc and water, the aq. layer separated off and extracted twice with EtOAc. The organic phases are washed with H₂O and brine, dried (Na₂SO₄) and concentrated. Column chromatography (SiO₂; hexane/EtOAc 97:3→4:1) gives the title compound; ES-MS: [M+1]⁺=485/487/489; HPLC: _{B}t_{Ret}=1.58 min.

### Intermediate 310.2

### 5-(3-Chloro-4-fluoro-phenyl)-1-(3-chloro-2-fluoro-phenyl)-2-cyclohexyl-1H-imidazole

Example 220 (273 mg, 0.605 mmol) is heated up to 215° C. The resulting brown melting is kept at 215° C. for 1 h, cooled to RT and used as such in Step 310.1. ES-MS: [M+1]⁺=407/409; HPLC: _{B}t_{Ret}=1.13 min.

### Example 311 & 312

### [5-(3-Chloro-4-fluoro-phenyl)-1-(3-chloro-2-fluoro-phenyl)-2-cyclohexyl-1H-imidazol-4-yl]phosphonic acid monoethyl ester A and [5-(3-Chloro-4-fluoro-phenyl)-1-(3-chloro-2-fluoro-phenyl)-2-cyclohexyl-1H-imidazol-4-yl]-phosphonic acid B

Example 310 (28 mg, 0.052 mmol) is dissolved in DCM (10 ml) under N₂-atmosphere. Then Me₃SiBr (330 μl, 2.55 mmol) is added, the round bottom flask closed and the solution stirred for 9 h at rt. Then EtOH (5 ml) is added, the mixture stirred for 15 min and finally concentrated. Reversed phase chromatography gives B, followed by A as TFA salts. A: ES-MS: [M+1]⁺=515/517; HPLC: _{B}t_{Ret}=1.08 min; ¹H NMR (DMSO d₆) δ 7.75 (t, 1H), 7.72 (t, 1H), 7.47 (d, 1H), 7.38 (t, 1H), 7.34 (t, 1H), 7.15 (m, 1H), 3.87 (m, 2H), 2.35 (m, 1H), 1.83 (m, 1H), 1.70 (m, 2H), 1.59 (m, 4H), 1.1 (m, 3H), 1.07 (t, 3H). B: ES-MS: [M+1]⁺=487/489; HPLC: _{B}t_{Ret}=1.00 min; ¹H NMR (DMSO d₆) δ 7.76 (t, 1H), 7.70 (t, 1H), 7.47 (d, 1H), 7.36 (t, 1H), 7.33 (t, 1H), 7.16 (m, 1H), 2.38 (m, 1H), 1.84 (m, 1H), 1.71 (m, 2H), 1.62 (m, 4H), 1.3-1.0 (m, 3H).

### Intermediate 313.1

### 5-Bromo-2-(3-chloro-benzyl)-1-(5-chloro-2-methyl-phenyl)-1H-imidazole-4-carboxylic acid ethyl ester

The title compound is synthesized by bromination of Intermediate 313.2 analogously to the preparation of Intermediate 6.1; ES-MS: M+H=468.9; HPLC: _{A}t_{Ret}=5.69 min.

### Intermediate 313.2

### 2-(3-Chloro-benzyl)-1-(5-chloro-2-methyl-phenyl)-1H-imidazole-4-carboxylic acid ethyl ester

The title compound is synthesized by dehydration of Intermediate 313.3 analogously to the preparation of Intermediate 6.2; ES-MS: M+H=391.0; HPLC: _{A}t_{Ret}=5.12 min.

### Intermediate 313.3

### 2-(3-Chloro-benzyl)-1-(5-chloro-2-methyl-phenyl)-4-hydroxy-4,5-dihydro-1H-imidazole-4-carboxylic acid ethyl ester

The title compound is synthesized by cycloaddition of ethyl bromopyruvate and Intermediate 313.4 analogously to the preparation of Intermediate 6.3; ES-MS: M+H=408.9; HPLC: _{A}t_{Ret}=4.24 min.

### Intermediate 313.4

### N-(5-Chloro-2-methyl-phenyl)-2-(3-chloro-phenyl)-acetamidine

The title compound is synthesized by addition of 5-chloro-2-methylaniline and 3-chloro-phenylacetonitrile analogously to the preparation of Intermediate 6.4; ES-MS: M+H=295.0; HPLC: _{A}t_{Ref}=3.74 min.

### Example 316

### 5-(3-Chloro-4-fluoro-phenyl)-1-(3-chloro-2-fluoro-phenyl)-2-m-tolyl-1H-imidazole-4-NH-methyl-sulfoximine

[Synthesis analog to: O. G. Mancheño, O. Bistri and C. Bolm, Org. Lett. 9 (2007), 3809-3811]

To an ice-cooled solution of Intermediate 316.1 (28 mg, 54 μmol) in DCM (1 ml), TFAA (22.5 μl, 162 μmol) is added. The mixture is stirred for 3 h at it and then concentrated in vacuo [→5-(3-chloro-4-fluoro-phenyl)-1-(3-chloro-2-fluoro-phenyl)-2-m-tolyl-1H-imidazole-4-N-(trifluoracetyl)-methyl-sulfoximine: ES-MS: [M+1]⁺=588/590]. This residue is re-dissolved in MeOH (0.4 ml), cooled in an ice-bath and hydrolyzed with K₂CO₃ (37.3 mg, 0.27 mmol). After 2 h stirring at rt, the suspension is diluted with MeOH (0.6 ml), NMP (1 ml) and a few drops of AcOH and directly submitted to reversed phase chromatography, giving the title compound. ES-MS: [M+1]⁺=4921494; HPLC: _{B}t_{Ret}=1.18 min; ¹H NMR (DMSO d₆) 7.70, 7.66 and 7.57 (3m, 3H), 7.37 (t, 1H), 7.32 (m, 3H), 7.23 (m, 2H), 7.01 (m, 1H), 3.22 (s, H₃C—S), 2.26 (s, H₃C).

### Intermediate 316.1

### 5-(3-Chloro-4-fluoro-phenyl)-1-(3-chloro-2-fluoro-phenyl)-2-m-tolyl-1H-imidazole-4-N-(cyano)-methyl-sulfoximine

Intermediate 316.2 (507 mg, 1.01 mmol) is dissolved in EtOH (10 ml). Then K₂CO₃ (1.396 g, 10.1 mmol) and 3-chlorperbenzoic acid (622 mg, 5.05 mmol) are added. After stirring the mixture for 2 h at 50° C., another portion of 622 mg 3-chlorperbenzoic acid is added. Stirring is continued for 15 h at 50° C., then the suspension is dissolved in EtOAc and water. The aq. layer is separated off and extracted twice with EtOAc. The organic phases are washed with H₂O and brine, dried (Na₂SO₄) and concentrated. Combi Flash chromatography (DCM→DCM/acetone 17:3) gives the title compound as a cis/trans mixture. ES-MS: [M+1]⁺=517/519; HPLC: _{B}t_{Ret}=1.33/1.36 min. IR: 2197 cm⁻¹ (s). Additionally, the side product 5-(3-chloro-4-fluoro-phenyl)-1-(3-chloro-2-fluoro-phenyl)-4-methanesulfonyl-2-m-tolyl-1H-imidazole (see Example 324) can be isolated.

### Intermediate 316.2

### 5-(3-Chloro-4-fluoro-phenyl)-1-(3-chloro-2-fluoro-phenyl)-2-m-tolyl-1H-imidazole-4-N-(cyano)-methyl-sulfilimine

Intermediate 316.3 (808 mg, 1.75 mmol) and cyanamide (147 mg, 3.5 mmol) are dissolved in MeOH (10 ml) and cooled in an ice-bath. Then ^{t}BuOK (257 mg, 2.1 mmol) and NBS (685 mg, 3.85 mmol) are added. The reaction mixture is slowly warmed up to rt during 4 h and then poured into a mixture of 10% aq. Na₂S₂O₃ solution and EtOAc. The aq. layer is separated off and extracted twice with EtOAc. The organic phases are washed with H₂O and brine, dried (Na₂SO₄) and concentrated. Combi Flash chromatography (crude product applied as a solution in DCM to the conditioned column and eluated with DCM→DCM/acetone 17:3) gives the title compound as a cis/trans mixture. ES-MS: [M+1]⁺=501/503; TLC(DCM/acetone 19:1): R_{f}=0.22; HPLC: _{B}t_{Ret}=1.27/1.33 min. IR: 2145 cm⁻¹(s).

### Intermediate 316.3

### 5-(3-Chloro-4-fluoro-phenyl)-1-(3-chloro-2-fluoro-phenyl)-4-methylsulfanyl-2-m-tolyl-1H-imidazole

Crude Intermediate 316.4 (3.97 mmol) is dissolved in THF (25 ml). Then a solution of NaOH (238 mg, 5.96 mmol) in 3 ml water and methyl-iodide (297 μl, 4.76 mmol) are added. After 45 min at rt, the solution is diluted with EtOAc and water. The aq. layer is separated off and extracted twice with EtOAc. The organic phases are washed with H₂O and brine, dried (Na₂SO₄) and concentrated. Combi Flash chromatography [(hexane/DCM 1:1) (hexane/DCM 1:1)/EtOAc 9:1] gives the title compound. ES-MS: [M+1]⁺=461/463; HPLC: _{B}t_{Ret}=1.42 min.

### Intermediate 316.4

### 5-(3-Chloro-4-fluoro-phenyl)-1-(3-chloro-2-fluoro-phenyl)-2-m-tolyl-1H-imidazole-4-thiol

Intermediate 316.5 (950 mg, 3.97 mmol) is suspended in benzene (1.5 ml) under N₂-atmosphere. Then a solution of Intermediate 316.6 (1476 mg, 5.96 mmol) in benzene (1.5 ml) is added via syringe. The mixture is stirred for 60 h at 65° C., giving the title compound, which is used as such in the step described above. ES-MS: [M+1]⁺=447/449.

### Intermediate 316.5

### 2-(3-Chloro-4-fluoro-phenyl)-2-oxo-thioacetamide

To a solution of 3-chlor-4-fluorobenzoyl cyanide (2.4 g, 13.07 mmol) in dioxane (29 ml), NaHS (0.88 g, 15.7 mmol) and Et₂NH.HCl (1.72 g, 15.7 mmol) are added. This suspension is stirred for 5 h at rt, then filtered and washed with dioxane. The filtrate is concentrated in vacuo, the residue re-dissolved in EtOAc and water, the aq. layer separated off and extracted twice with EtOAc. The organic phases are washed with H₂O and brine, dried (Na₂SO₄) and concentrated. Column chromatography (SiO₂; DCM/EtOAc 99:1) gives the title compound. ES-MS: [M−1]=216/218; HPLC: _{B}t_{Ret}=1.00 min.

### Intermediate 316.6

### (3-Chloro-2-fluoro-phenyl)-(1-m-tolyl-methylidene]-amine

A mixture of m-tolyl-aldehyde (5 ml, 42.5 mmol) and 3-chloro-2-fluoraniline (4.67 ml, 42.5 mmol) in toluene (150 ml) is heated for 16 h under reflux conditions. This solution is concentrated in vacuo Kugelrohr distillation (200° C., 0.8 mbar) gives the title compound. ES-MS: [M+1]⁺=248/250.

### Example 319

### 5-[5-(3-Chloro-4-fluoro-phenyl)-1-(3-chloro-2-fluoro-phenyl)-2-phenyl-1H-imidazol-4-yl]-3H-[1,3,4]oxadiazol-2-one

CDI (28.2 mg, 0.174 mmol) is added to a solution of Example 320 (40 mg, 0.087 mmol) and TEA (0.027 ml, 0.192 mmol) in THF (1 ml) at 25° C. The reaction mixture is stirred for 12 h and then diluted with EtOAc and water. The organic layer is separated and dried and concentrated. The remaining crude product is purified by flash chromatography (SiO₂; DCM/MeOH; 0-5% MeOH) to provide the title compound as a white powder. [M+1]=486.7; HPLC: _{A}t_{Ret}=5.08 min. ¹HNMR (MeOH-d₄) 7.60-7.57 (m, 2H), 7.42-7.35 (m, 7H), 7.23-7.17 (m, 2H).

### Example 320

### 5-(3-chloro-4-fluoro-phenyl) 1-(5-chloro-2-fluoro-phenyl)-2-(phenyl)-1H-imidazole-4-carboxylic acid hydrazide

EDC (301 mg, 1.572 mmol) is added to a solution of Example 190 (350 mg, 0.786 mmol) in DMA (9 ml) at 25° C. followed by Et₃N (0.469 ml, 3.38 mmol). The reaction mixture is stirred for 1 hr. Then tert-butyl carbazate (520 mg, 3.8 mmol) is added and the reaction mixture is heated to 60° C. for 15 h. Hydrazine (1M THF) (15.8 ml, 15.8 mmol) is added and stirring continued for 20 hr at 60° C. The reaction mixture is diluted with EtOAc and sat. aqueous NaHCO₃. The organic layer is then washed with sat. aqueous NaCl solution, dried and concentrated. The crude product is purified by flash chromatography (SiO₂; DCM/MeOH; 0-5% MeOH). ES-MS: [M−1]=444.9; HPLC: _{A}t_{Ret}=4.33 min. ¹HNMR (DMSO-d₆) 7.61 (t, 1H), 7.59-7.55 (m, 2H), 7.39-7.30 (m, 6H), 7.24-7.18 (m, 2H).

### Example 321

### 5-[5-(3-Chloro-4-fluoro-phenyl)-1-(3-chloro-2-fluoro-phenyl)-2-phenyl-1H-imidazol-4-yl]-[1,3,4]oxadiazole-2-ylamine

BrCN (11.92 mg, 0.113 mmol) is added to a solution of Example 320 (47 mg, 0.102 mmol) and NaHCO₃ (10.32 mg, 0.123 mmol) in Dioxane (1 ml)/water (0.5 ml) at 25° C. The reaction mixture is stirred for 20 hr. It is then diluted with water. The precipitate (crude product) was collected by filtration and dried at high vacuum. The crude product is triturated with Et₂O to give the title compound as a white powder. [M+1]=485.8; HPLC: _{A}t_{Ret}=4.73 min. ¹HNMR (MeOH-d₄) 7.60-7.57 (m, 2H), 7.49-7.25 (m, 7H), 7.21-7.17 (m, 2H).

### Example 322

### 2-[5-(3-Chloro-4-fluoro-phenyl)-1-(3-chloro-2-fluoro-phenyl)-2-phenyl-1H-imidazol-4-yl]-5-methyl-[1,3,4]oxadiazole

Acetic anhydride (14.4 μl, 0.15 mmol) is added to a solution of Example 320 (35 mg, 0.08 mmol) in pyridine (616 μl, 7.6 mmol) and stirred for 1 hr at RT. Pyridine is evaporated and polyphosphoric acid (1 ml) is added to the reaction mixture and stirred for 2 hr at 120° C. It is submitted to aqueous workup and the crude material is purified by flash chromatography (SiO₂; DCM/MeOH; 0-5% MeOH). ES-MS: [M+1]=484.8; HPLC: _{A}t_{Ret}=5.27 min. ¹HNMR (MeOH-d₄) 7.62-7.58 (m, 2H), 7.49-7.25 (m, 7H), 7.21-7.19 (m, 2H).

### Example 323

### 5-[5-(3-Chloro-4-fluoro-phenyl)-1-(3-chloro-2-fluoro-phenyl)-2-phenyl-1H-imidazol-4-yl]-4-methyl-2,4,dihydro[1,2,4]triazole-thione

Methyl isothiocyanate (15.4 mg, 0.21 mmol) is added to a suspension of Example 320 (50 mg, 0.01 mmol) and K₂CO₃ (176 mg, 1.3 mmol) in H₂O (2 ml) and stirred for 15 hr at 115° C. The reaction mixture is diluted with EtOAc and water. The organic layer is washed with sat. aqueous NaCl and concentrated under reduced pressure to give the crude product, which is purified by flash chromatography (SiO₂; heptane/EtOAc; 0-40% EtOAc) to provide the title compound as a yellow powder. [M+1]=515.7; HPLC: _{A}t_{Ret}=5.25 min. ¹HNMR (MeOH-d₄) 7.61 (t, 1H) 7.58-7.30 (m, 7H), 7.22-7.07 (m, 3H), 3.81 (s, 3H).

### Example 324

### 5-(3-Chloro-4-fluoro-phenyl)-1-(3-chloro-2-fluoro-phenyl)-4-methanesulfonyl-2-m-tolyl-1H-imidazole

Isolated as a side product of Intermediate 316.1. ES-MS: [M+1]⁺=493/495; HPLC: _{B}t_{Ret}=1.38 min.

### Example 331

### 5-[5-(3-Chloro-4-fluoro-phenyl)-1-(3-chloro-2-fluoro-phenyl)-2-phenyl-1-imidazol-4-yl]-3-methyl-[1,2,4]oxadiazole

Oxalyl chloride (0.10 ml, 1.2 mmol) is added to a solution of Example 190 (53 mg, 0.12 mmol) in chloroform (5 ml) at 5° C. A drop of DMF is added and the reaction mixture is stirred for 2 hr at 25° C. The volatiles are evaporated. The residue is dissolved in toluene (5 ml, under argon). Acetamide oxime (17.6 mg, 0.24 mmol) and triethylamine (50 μL, 0.35 mmol) are added and stirring continued for 1.5 hr at rt. The reaction mixture is then heated to 130° C. for 20 hr. It is allowed to cool to rt, diluted with EtOAc and water. The organic layer is washed with sat NaCl, dried over Na₂SO₄ and concentrated to give the crude product which is purified by preparative TLC (CH₂Cl₂ 9:1 MeOH) to give the title compound as white powder. [M+1]=484.7; HPLC: _{A}t_{Ret}=5.59 min. ¹HNMR (CDCl₃) 7.53-7.43 (m, 2H) 7.42-7.22 (m, 7H), 7.19-6.99 (m, 2H), 2.41 (s, 3H).

### Example 332

### 1-(Acetylaminomethyl)-5-chlorophenyl]-5-(3-chloro-4-fluorophenyl)-cyclohexyl-1H-imidazole-4-carboxylic acid

LiOH*H₂O (12.6 mg, 0.3 mmol) is added to a solution of Intermediate 332.1 (80 mg, 0.15 mmol) in Dioxane (6 ml)/H₂O (1.5 ml) and heated for 1 hr at 60° C. LiOH*H₂O (12.6 mg, 0.3 mmol) was added and stirring continued for 1 h. The reaction mixture is diluted with EtOAc and citric acid. The organic layer is washed with sat NaCl. The aqueous layer is backextracted with EtOAc. The combined organic layers are dried over Na₂SO₄, and concentrated to give the title compound as a white powder. [M−1]=502.1; HPLC: _{A}t_{Ret}=4.14 min. ¹HNMR (MeOH-d₄) 7.61 (s, 1H), 7.59-7.57 (m, 1H), 7.48 (d, 1H), 7.29-7.20 (m, 1H), 7.15 (t, 1H), 3.99 (d, 1H), 3.76 (d, 1H), 3.61 (s, 3H), 2.39-2.23 (m, 1H), 1.98-1.65 (m, 5H), 1.39-1.15 (m, 4H).

### Intermediate 332.1

### 1-(Acetylaminomethyl)-5-chlorophenyl]-5-(3-chloro-4-fluorophenyl)-cyclohexyl-1H-imidazole-4-carboxylic acid ethylester

Acetyl chloride (26 μl, 0.36 mmol) is added to a solution of Intermediate 332.2 (90 mg, 0.18 mmol) and TEA (76 μL, 0.55 mmol) in CH₂Cl₂ (5 ml) at 25° C. The reaction is stirred for 3 h.

The reaction mixture is then quenched with water and diluted with EtOAc. The organic layer is washed with water and brine, dried and concentrated to give a white solid, which is titurated with MeOH/CH₂Cl₂ to give the title compound as a white powder. [M+1]=533.8; HPLC: _{A}t_{Ret}=4.75 min. ¹HNMR (CDCl₃) 7.41 (d, 1H), 7.38 (d, 1H), 7.21 (s, 1H), 7.03-6.99 (m, 2H). 5.51 (bs, 1H), 4.03 (dd, 1H), 3.63 (dd, 1H), 2.39-2.25 (m, 1), 1.99 (s, 3H), 1.95-1.54 (m, 5H), 1.23-1.01 (m, 4H).

### Intermediate 332.2

### 1-(2-Aminomethyl-5-chlorophenyl)-5-(3-chloro-4-fluorophenyl)-cyclohexyl-1H-imidazole-4-carboxylic acid ethylester

Intermediate 332.3 (100 mg, 0.21 mmol) is dissolved in a 4M solution of NH₃ in EtOH (6 mL). Raney-Nickel is added and the reaction mixture is placed under an atmosphere of H₂ under atmospheric pressure at rt. It is vigorously stirred for 18 h. After completion the catalyst is removed by filtration and washed with EtOH. Combined filtrate and washings are concentrated under reduced pressure and dried at high vacuum to give the title compound as yellow solid. [M+1]=491.9; HPLC: _{A}t_{Ret}=4.13 min.

### Intermediate 332.3

### 1-(5-Chloro-2-cyanophenyl)-5-(3-chloro-4-fluorophenyl)-2-cyclohexyl-1H-imidazole-4-carboxylic acid ethylester

Intermediate 332.4 (1.36 g, 3.1 mmol) is dissolved in Toluene (20 mL). Water (10 mL), 3-chloro-2-fluoro phenylboronic acid (0.81 g, 4.6 mmol), K₃PO₄ (2.64 g, 12.5 mmol) and Pd(PPh₃)₄ (0.36 g, 0.31 mmol) is added and the reaction mixture is heated to 100° C. for 1 h. After cooling to rt it is submitted to aqueous workup and the crude material purified by flash chromatography (SiO₂, heptan/EtOAc; gradient 5-20% EtOAc to give the title compound as a white powder. [M+1]=487.9; HPLC: _{A}t_{Ret}=5.43 min. ¹HNMR (CDCl₃) 7.65 (d, 1H), 7.60 (d, 1H), 7.41 (s, 1H), 7.39 (d, 1H), 7.17-7.07 (m, 2H).

### Intermediate 332.4

### 5-Bromo-1-(5-chloro-2-cyanophenyl)-2-cyclohexyl-1H-imidazole-4-carboxylic acid ethylester

Intermediate 332.5 (1.6 g, 4.5 mmol) is brominated in analogy to the procedure described for Intermediate 6.1. The crude product is purified by flash chromatography (SiO₂, heptan/EtOAc; gradient 1-20% EtOAc) to give the title compound as a yellow powder. [M+1]=437.9; HPLC: _{A}t_{Ret}=5.00 min. ¹HNMR (CDCl₃) 7.81 (d, 1H), 7.75 (d, 1H), 7.18 (s, 1H), 4.41 (q, 2H), 2.38-2.24 (m, 1H), 1.87-1.57 (m, 6H), 1.40 (t, 3H), 1.34-1.10 (m, 4H).

### Intermediate 332.5

### 1-(5-Chloro-2-cyanophenyl)-2-cyclohexyl-1H-imidazole-4-carboxylic acid ethylester

Intermediate 371.4 (7.3 g, 15.9 mmol) is dissolved in acetone (50 mL) and KCN (2.1 g, 31.8 mmol), Pd₂(dba)₃ (1.2 g, 1.3 mmol) and dppf (1.4 g, 2.6 mmol) is added. The reaction mixture is stirred in a sealed tube at 80° C. for 2 h. It is then allowed to cool to rt and submitted to aqueous workup. The crude material is purified by flash chromatography (SiO₂, heptan/EtOAc; gradient 1-20% EtOAc) to give the title compound as a white powder. [M+1]=359.7; HPLC: _{A}t_{Ret}=4.64 min.

### Intermediate 338.1

### 3-[5-Bromo-1-(5-chloro-2-methyl-phenyl)-4-ethoxycarbonyl-1H-imidazol-2-yl]-piperidine-1-carboxylic acid benzylester

The title compound is synthesized by bromination of Intermediate 338.2 analogously to the preparation of Intermediate 6.1; ES-MS: M+=561.8; HPLC: _{A}t_{Ret}=5.48 min.

### Intermediate 338.2

### 3-[1-(5-Chloro-2-methyl-phenyl)-4-ethoxycarbonyl-1H-imidazol-2-yl]-piperidine-1-carboxylic acid benzylester

The title compound is synthesized by dehydration of Intermediate 338.3 analogously to the preparation of Intermediate 6.2; ES-MS: M+=483.4; HPLC: _{A}t_{Ret}=5.23 min.

### Intermediate 338.3

### 3-[1-(5-Chloro-2-methyl-phenyl)-4-ethoxycarbonyl-4-hydroxy-4,5-dihydro-1H-imidazol-2-yl]-piperidine-1-carboxylic acid benzylester

The title compound is synthesized by cycloaddition of Ethyl bromopyruvate and Intermediate 338.4 analogously to the preparation of Intermediate 6.3; ES-MS: M+=502.2; HPLC: _{A}t_{Ret}=4.49 min.

### Intermediate 338.4

### 3-[5-chloro-2-methyl-phenyl)-carbamimidoyl]-piperidine-1-carboxylic acid benzylester

The title compound is synthesized by addition of 5-chloro-2-methyl-aniline and Intermediate 338.5 analogously to the preparation of Intermediate 6.4; ES-MS: M+=386.1; HPLC: _{A}t_{Ret}=4.01 min.

### Intermediate 338.5

### 3-Cyano-piperidine-1-carboxylic acid benzylester

The title compound is synthesized by dehydration of Intermediate 338.6 analogously to the preparation of Intermediate 346.5; ES-MS: M+=262.2 (M+H₂O); HPLC: _{A}t_{Ret}=4.18 min.

### Intermediate 338.6

### 3-Carbamoyl-piperidine-1-carboxylic acid benzylester

Piperidine carboxylic acid amide (2.0 g, 14.8 mmol) is dissolved in acetone (50 ml) at rt. H₂0 (50 ml), NaHCO₃ (2.5 g, 30.0 mmol) and N-(benzyloxycarbonyloxy) succinimide (4.6 g, 17.9 mmol) are added at rt and the reaction mixture is stirred for 24 h. Acetone is removed under reduced pressure, leading to precipitation of the title compound, which is isolated by filtration and dried at high vacuum. ES-MS: M+=263.2 (M+H2O); HPLC: _{A}t_{Ret}=3.49 min.

### Example 339

### 1-(5-Chloro-2-methyl-phenyl)-5-(3-chloro-phenyl)-2-piperidin-3-yl-1H-imidazole-4-carboxylic acid ethyl ester

Example 338 (145 mg, 0.24 mmol) is dissolved in MeOH (5 ml) and Pd—C (10% Fluka, 7.2 mg, 0.049 mmol) is added. The reaction mixture is then flushed with H₂ and stirred under H₂ atmosphere for 20 h at rt. It is filtered over a pad of celite and concentrated. The remaining crude product is purified by flash chromatography (SiO₂; DCM/MeOH; gradient 0-10% MeOH) to afford the title compound as yellow solid. ES-MS: M+=460.2; HPLC: _{A}t_{Ret}=4.35 min.

### Example 341

### 1-(5-Chloro-2-methyl-phenyl)-5-(3-chloro-phenyl)-2-(1-methyl-piperidin-3-yl)-1H-imidazole-4-carboxylic acid ethyl ester

Example 339 (96 mg, 0.21 mmol) is dissolved in DCE (5 ml). Formaldehyde (36% wt aq. solution, Fluka 41629; 17 μL, 0.21 mmol) and NaBH(OAc)₃ (67 mg, 0.32 mmol) are added and the reaction mixture is allowed to stir for 20 h at rt. It is diluted with EtOAc and the organic layer is washed with sat. aq. NaHCO₃ solution, dried over Na₂SO₄, concentrated and dried at high vacuum to give the title compound as a colorless solid. ES-MS: M+=476.0; HPLC: _{A}t_{Ret}=4.41 min.

### Example 344

### {5-[5-(3-Chloro-4-fluoro-phenyl) 1-(3-chloro-2-fluoro-phenyl)-2-(cyclohexyl)-1H-imidazole-4-yl]-[1,3,4]oxadiazol-2-yl}-methyl-amine

BOP (99 mg, 0.22 mmol) and DIPEA (107 μl, 0.61 mmol) are added to a solution of Example 498 (100 mg, 0.21 mmol) in DMF (5 ml) at 25° C. MeNH₂ (305 μl, 0.61 mmol, 2M solution in THF) is added and stirred for 1 h. The reaction mixture is diluted with EtOAc and water. The organic layer is washed with sat NaHCO₃ and sat aqueous NaCl. The aqueous layer was backextracted with EtOAc. The combined organic layers are dried over Na₂SO₄, filtered and concentrated to give the crude product which is purified flash chromatography (SiO₂; DCM/MeOH; 0-10% MeOH). ES-MS: [M+1]=506.0; ¹HNMR (CDCl₃) 7.52 (dd, 1H), 7.35 (dd, 1H), 7.20-7.14 (m, 2H), 7.10-7.01 (m, 2H), 3.03 (d, 3H), 2.38-2.30 (m, 1H), 1.86-1.74 (m, 6H), 1.68-1.54 (m, 4H).

### Example 345

### 3-[5-(3-Chloro-4-fluoro-phenyl) 1-(3-chloro-2-fluoro-phenyl)-2-(phenyl)-1H-imidazol-4-yl]-1-H-pyrazole-4-carboxylic acid (2-dimethylaminoethyl)-amide

Example 381 (96 mg, 1.5 mmol) is dissolved in chloroform and treated with oxalyl chloride (380 mg, 3.0 mmol) and 2 drops of dry DMF at rt. The reaction mixture is allowed to stir for 15 min. at it and then concentrated under reduced pressure. The remaining material is taken up in chloroform (5 mL) and treated with N,N-dimethyl ethyl amine (132 mg, 1.5 mmol) at rt. The reaction mixture is stirred for 12 h and concentrated. The remaining material is again dissolved in dioxane (5 ml) and treated with a solution of HCl in dioxane (4 M, 5 ml). The reaction mixture is then stirred for 1 h at 90° C. It is allowed to cool to rt and submitted to aqueous workup. The remaining crude material of the title compound is purified by preparative TLC (SiO2, DCM/MeOH; 9:1) to give the title compound as a white solid. ES-MS: [M+1]=683.1; HPLC: _{A}t_{Ret}=4.06 min.

### Intermediate 346.1

### 5-Bromo-2-[3-(tert-butyl-diphenyl-silanyloxy)-cyclohexyl]-1-(5

The title compound is synthesized by bromination of Intermediate 346.2 analogously to the preparation of Intermediate 6.1, except that the reaction is performed at 45° C.; ES-MS: M+=573.1 (M-C₂H₅); HPLC: _{A}t_{Ret}=6.01 min.

### Intermediate 346.2

### 2-[3-(tert-Butyl-diphenyl-silanyloxy)-cyclohexyl]-1-(5-chloro-2-methyl-phenyl)-1H-imidazole-4-carboxylic acid ethyl ester

The title compound is synthesized by dehydration of Intermediate 346.3 analogously to the preparation of Intermediate 6.2, except that the reaction is performed at 60° C.; ES-MS: M+=573.1 (M-C₂H₅); HPLC: _{A}t_{Ret}=6.01 min.

### Intermediate 346.3

### 2-[3-(tert-Butyl-diphenyl-silanyloxy)-cyclohexyl]-1-(5-chloro-2-methyl-phenyl)-4-hydroxy-4,5-dihydro-1H-imidazole-4-carboxylic acid ethyl ester

The title compound is synthesized by cycloaddition of Ethyl bromopyruvate and Intermediate 346.4 analogously to the preparation of Intermediate 6.3, except that the reaction is performed at 60° C.; ES-MS: M+=620.4; HPLC: _{A}t_{Ret}=5.63 min.

### Intermediate 346.4

### 3-(tert-Butyl-diphenyl-silanyloxy)-N-(5-chloro-2-methyl-phenyl)-cyclohexanecarboxamidine

5-Chlor-2-methylaniline (260 mg, 1.8 mmol) is dissolved in toluene (10 ml) and cooled to 0° C. At this temperature, Et₂AlCl (1.8M solution in toluene, 3.0 ml, 5.5 mmol) is added dropwise. The reaction mixture is allowed to warm to rt and stirred for 2 h. Then a solution of Intermediate 346.5 (734 mg, 2.0 mmol) in toluene (5 ml) is added and the reaction mixture is stirred at 60° C. for 12 h. It is cooled to ambient temperature, diluted with DCM/MeOH (8:2, 50 ml), filtered over a padofcelite and concentrated. The remaining crude product is purified by flash chromatography: (SiO₂, DCM/MeOH; gradient 0-5% MeOH) to give the title compound as a yellow oil. ES-MS: M+=507.1; HPLC: _{A}t_{Ret}=5.47 min.

### Intermediate 346.5

### 3-(tert-Butyl-diphenyl-silanyloxy)-cyclohexanecarbonitrile

Oxalylic chloride (4.4 ml, 52.4 mmol) is dissolved in DCM and cooled to −78° C. At this temperature a solution of Intermediate 346.6 (5.6 ml, 78.6 mmol) in DCM (10 ml) is added dropwise. The reaction mixture is allowed to stir 15 min. At −78° C. followed by dropwise addition of a solution of intermediate 42.8 (5.0 g, 13.1 mmol) in DCM (40 ml). Stirring is continued for 30 min. at −78° C. and then the reaction mixture is allowed to warm to rt and stirred for 12 h. It is diluted with EtOAc and submitted to aq. workup. The organic layer is separated, dried and concentrated. The residual crude product is purified by flash chromatography (SiO₂, DCM/MeOH; gradient 0-1% MeOH) to give the title compound as a yellow oil. ES-MS: M+=364.2; HPLC: _{A}t_{Ret}=6.57 min.

### Intermediate 346.6

### 3-(tert-Butyl-diphenyl-silanyloxy)-cyclohexanecarboyxic acid amide

Intermediate 346.7 (23.0 g, 56 mmol) is dissolved in toluene (60 ml) and treated with ammonium chloride (30 g, 560 mmol) and trimethyl aluminium (2M solution in toluene, 140 ml, 280 mmol) at rt. The reaction mixture is then allowed to stir at 90° C. for 1.5 h. It is allowed to cool to rt and diluted with DCM/MeOH (8:2), filtered over a pad of celite, concentrated and dried at high vacuum to give the title compound as white solid. ES-MS: M+=482.0; HPLC: _{A}t_{Ret}=5.59 min.

### Intermediate 346.7

### 3-(tert-Butyl-diphenyl-silanyloxy)-cyclohexanecarboyxlc acid ethyl ester

To a solution of Intermediate 346.8 (10.0 g, 58 mmol) in THF (50 ml), imidazole (4.3 g, 64 mmol) and tert-butyl-diphenylchlorsilane (16.3 ml, 64 mmol) are added at rt. The reaction mixture is then allowed to stir at 40° C. for 12 h. It is allowed to cool to rt and submitted to aq. work up and drying at high vacuum to give the title compound. ES-MS: M+=411.1; HPLC: _{A}t_{Ret}=7.00 min.

### Intermediate 346.8

### 3-Hydroxy-cyclohexanecarboyxic acid ethyl ester

3-Hydroxy benzoic acid ethylester (10.0 g, 60.2 mmol) is dissolved in EtOH (100 ml) and submitted to hydrogenation at atmospheric pressure in a Parr shaker in the presence of Nishimura's catalyst (Rh/PtO; Unicore; 2.0 g) at rt for 48 h. The reaction mixture is filtered over a pad of Celite, concentrated and dried at high vacuum to give the title compound as a white solid. ES-MS: M+=173.0.

### Example 348

### 5-(3-Chloro-4-fluoro-phenyl)-1-(5-chloro-2-methyl-phenyl)-2-(3-hydroxy-cyclohexyl)-1H-imidazole-4-carboxylic acid

Example 347 (103 mg, 0.15 mmol) is dissolved in THF and treated with TBAF (1 M solution in THF, 1.4 ml, 1.4 mmol) at rt. The reaction mixture is then allowed to stir at reflux for 12 h and cooled to rt again. It is diluted with EtOAc and the organic layer is washed with water and brine, dried and concentrated. The residual crude product is purified by reversed phase MPLC to give the title compound as yellow solid. ES-MS: M+=465.0; HPLC: _{A}t_{Ret}=4.26 min.

### Intermediate 349.1

### 5-Bromo-2-(3-(tert-butyl-diphenyl-silanyloxy)-cyclohexyl]-1,3-chloro-2-fluoro-phenyl)-1H-imidazole-4-carboxylic acid ethyl ester

The title compound is synthesized by bromination of Intermediate 349.2 analogously to the preparation of intermediate 6.1, except that the reaction is performed at 45° C.; ES-MS: M+=685.1; HPLC: _{A}t_{Ret}=7.41 min.

### Intermediate 349.2

### 2-[3-(tert-butyl-diphenyl-silanyloxy)-cyclohexyl]-1,3-chloro-2-fluoro-phenyl)-1H-imidazole-4-carboxylic acid ethyl ester

The title compound is synthesized by dehydration of Intermediate 349.3 analogously to the preparation of Intermediate 6.2, except that the reaction is performed at 60° C.; ES-MS: M+=607.2; HPLC: _{A}t_{Ret}=6.98 min.

### Intermediate 349.3

### 2-[3-(tert-Butyl-diphenyl-silanyloxy)-cyclohexyl]-1-(3-chloro-2-fluoro-phenyl)-4-hydroxy-4,5-dihydro-1H-imidazole-4-carboxylic acid ethyl ester

The title compound is synthesized by cycloaddition of Ethyl bromopyruvate and intermediate 349.4 analogously to the preparation of Intermediate 6.3, except that the reaction is performed at 60° C.; ES-MS: M+=625.2; HPLC: _{A}t_{Ret}=5.66 min.

### Intermediate 349.4

### 3-(tert-Butyl-diphenyl-silanyloxy)-N-(3-chloro-2-fluoro-phenyl)-cyclohexanecarboxamidine

The title compound is synthesized by addition of 3-chloro-2-fluoroaniline and Intermediate 346.5 analogously to the preparation of Intermediate 6.4; ES-MS: M−=507.2; HPLC: _{A}t_{Ret}=5.45 min.

### Intermediate 354.1

### 4-Chloro-2-(4,4,5,5-tetramethyl-[1,3,2]dioxaborolan-2-yl)-benzaldehyde

2-Bromo-4-chloro-benzaldeyde (250 mg, 0.96 mmol) is dissolved in DME (5 ml). Bis-pinacolato diboron (320 mg, 1.2 mmol), Pd (dppf)Cl₂*CH₂Cl₂ (78 mg, 0.09 mmol) and potassium acetate (280 mg, 2.8 mmol) are added at rt. The reaction mixture is flushed with argon and stirred at 80° C. for 20 h in a sealed tube. It is allowed to cool to rt again, diluted with EtOAc and the organic layer is washed with H₂O and brine, dried over Na₂SO₄ and concentrated. The remaining crude product is purified by flash chromatography (SiO₂; hexanes/EtOAc; gradient 0-60% EtOAc) to afford the title compound as a yellow solid. ¹H NMR (CDCl₃) δ 10.50 (s, 1H), 7.91 (d, 1H), 7.84 (s, 1H), 7.72 (d, 1H), 1.39 (s, 12H); HPLC: _{A}t_{Ret}=2.75 min.

### Example 371

### 1-(2-Carboxymethyl-5-chloro-phenyl)-5-(3-chloro-4-fluoro-phenyl)-2-cyclohexyl-1H-imidazole-4-carboxylic acid ethyl ester

The title compound is synthesized by Suzuki Coupling of Intermediate 371.1 with 2-(3-chloro-4-fluoro-phenyl)-4,4,5,5-tetramethyl-[1,3,2]dioxaborolane analogously to the preparation of Example 6; ES-MS: [M+1]⁺=5191521; HPLC: _{B}t_{Ret}=1.23 min; ¹H NMR (DMSO d₆) δ 12.57 (s, HO), 7.85 (s, 1H), 7.55 (d, 1H), 7.53 (d, 1H), 7.36 (d, 1H), 7.29 (t, 1H), 7.24 (m, 1H), 4.12 and 4.06 (2m, H₂C), 3.18 (d, 1H), 2.98 (d, 1H), 2.14 (m, 1H), 1.79 (m, 1H), 1.68 (m, 3H), 1.57 (m, 1H), 1.34 (m, 1H), 1.18 (m, 2H), 1.08 (t, 3H), 1.03 (m, 2H).

### Intermediate 371.1

### 5-Bromo-1-(2-carboxymethyl-5-chloro-phenyl)-2-cyclohexyl-1H-imidazole-4-carboxylic acid ethyl ester

The title compound is synthesized by bromination of Intermediate 371.2 analogously to the preparation of Intermediate 6.1; ES-MS: [M+1]⁺=469/471; HPLC: _{B}t_{Ret}=1.21 min.

### Intermediate 371.2

### 1-(2-Carboxymethyl-5-chloro-phenyl)-2-cyclohexyl-1H-imidazole-4-carboxylic acid ethyl ester

Borane-dimethylsulfide complex (2.66 ml, 90% pure, 25.2 mmol) is added dropwise to an ice-cooled solution of cyclohexene (5.76 ml, 56.8 mmol) in THF (50 ml). The resulting suspension is warmed up to rt, stirred for 3 h and then cooled to 0° C. A solution of Intermediate 371.3 (4.06 g, 9.46 mmol) in THF (70 ml) is added during 10 min. Stirring for 75 min at rt gives a solution. Then a mixture of sat. NaHCO₃ solution (67 ml) and

H₂O₂ (30%, 14.3 ml, 0.14 mol) is added during 20 min (cooling to keep the temperature below 50° C.). The mixture is stirred for 16 h at rt and then filtered. The filtrate is diluted with EtOAc and water, the aq. layer separated off and extracted twice with EtOAc. The organic phases are washed twice with H₂O/sat. NaHCO₃ solution 1:1 and discarded. The aq. layers are acidified with 2 N HCl and extracted with 3 portions of EtOAc. These EtOAc phases are washed with brine, dried (MgSO₄) and concentrated, giving the title compound. ES-MS: [M+1]⁺=391/393; HPLC: _{B}t_{Ret}=1.03 min.

### Intermediate 371.3

### 1-(5-Chloro-2-trimethylsilanylethynyl-phenyl)-2-cyclohexyl-1H-imidazole-4-carboxylic acid ethyl ester

A solution of Intermediate 371.4 (4.75 g, 10.35 mmol) in diethyl-amine (42 ml) is degassed by repeated evacuation and flushing with N₂. Then PdCl₂(PPh₃)₂ (145 mg, 0.207 mmol), CuI (39 mg, 0.207 mmol) and ethynyl-trimethyl-silane (1.58 ml, 11.39 mmol) are added. After 17 h stirring at rt, the suspension is diluted with EtOAc and water, the aq. layer separated off and extracted twice with EtOAc. The organic phases are washed with H₂O and brine, dried (Na₂SO₄) and concentrated. Column chromatography (SiO₂; applied as solution in DCM and eluated with DCM/EtOAc 95:1→925:75) gives the title compound. ES-MS: [M−1]=429/431; HPLC: _{B}t_{Ret}=1.44 min.

### Intermediate 371.4

### 1-(5-Chloro-2-iodo-phenyl)-2-cyclohexyl-1H-imidazole-4-carboxylic acid ethyl ester

The title compound is synthesized by dehydration of Intermediate 371.5 analogously to the preparation of Intermediate 6.2; ES-MS: [M+1]⁺=459/461; HPLC: _{B}t_{Ret}=1.27 min.

### Intermediate 371.5

### 1-(5-Chloro-2-iodo-phenyl)-2-cyclohexyl-4-hydroxy-4,5-dihydro-1H-imidazole-4-carboxylic acid ethyl ester

The title compound is synthesized by cycloaddition of ethyl bromopyruvate and Intermediate 371.6 analogously to the preparation of Intermediate 6.3; ES-MS: [M+1]⁺=477/479; HPLC: _{B}t_{Ret}=1.03 min.

### Intermediate 371.6

### N-(5-Chloro-2-iodo-phenyl)-2-cyclohexyl-acetamidine

The title compound is synthesized by addition of 5-chloro-2-iodo-aniline and cyclohexan-carbonitrile analogously to the preparation of Intermediate 395.6; ES-MS: [M+1]⁺=363/365; HPLC: _{B}t_{Ret}=0.86.

### Example 372

### 1-(2-tert-Butoxycarbonylmethyl-5-chloro-phenyl)-5-(3-chloro-4-fluoro-phenyl)-2-cyclohexyl-1H-imidazole-4-carboxylic acid ethyl ester

To Example 371 (95 mg, 0.183 mmol) in DCM (1 ml), a solution of 2,2,2-trichloro-acetimidic acid tert-butyl ester (66 μl, 0.37 mmol) in cyclohexane (0.73 ml) is added, followed by BF₃.Et₂O (3.7 μl, 29 μmol). After 90 min, another portion of 66 μl of 2,2,2-trichloro-acetimidic acid tert-butyl ester is added and the mixture is stirred for another 90 min at rt and then diluted with sat. NaHCO₃ solution and EtOAc. The separated aq. layer is extracted twice with EtOAc. The organic phases are washed with brine, dried (Na₂SO₄) and concentrated. Combi Flash chromatography (hexane/EtOAc 19:1→7:3) gives the title compound. ES-MS: [M+1]⁺=575/577; HPLC: _{B}t_{Ret}=1.51 min.

### Example 375

### 5-(3-Chloro-4-fluoro-phenyl)-1-(5-chloro-2-methylcarbamoylmethyl-phenyl)-2-cyclohexyl-1H-imidazole-4-carboxylic acid ethyl ester

To Example 371 (171 mg, 0.329 mmol) dissolved in DMF (4 ml), MeNH₂.HCl (67 mg, 1.0 mmol), Et₃N (1.95 ml, 14 mmol), DMAP (17.3 mg, 0.142 mmol) and 2,4,6-tripropyl-1,3,5,2,4,6-trioxatriphosphorinane-2,4,6-trioxide ([68957-94-8] 50% in DMF; 0.96 ml, 1.64 mmol) are added. The solution is stirred for 20 h at rt and then poured into EtOAc and water. The aq. layer is separated off and extracted twice with EtOAc. The organic phases are washed with water and brine, dried (Na₂SO₄) and concentrated. Reversed phase chromatography gives the title compound. ES-MS: [M+1]⁺=532/534; HPLC: _{B}t_{Ret}=1.21 min.

### Example 381

### 3-[5-(3-Chloro-4-fluoro-phenyl) 1-(3-chloro-2-fluoro-phenyl)-2-(phenyl)-1H-imidazol-4-yl]-1-(2-trimethylsilanyl-ethoxymethyl)-1-H-pyrazole-4-carboxylic acid

LiOH.H₂O (15.8 mg, 0.38 mmol) is added to a solution of Example 382 (63 mg, 0.09 mmol) in Dioxane (2 ml)/water (0.5 ml) at RT and stirred for 1 hr at 80° C. The reaction mixture is diluted with EtOAc and washed with citric acid (5% w/w, aqueous). The organic layer is dried over Na₂SO₄, filtered and concentrated. The remaining crude material is used without further purification in the next step. HPLC: _{A}t_{Ret}=6.51 min. MS: [M−1]=640.5.

### Example 382

### 3-[5-(3-Chloro-4-fluoro-phenyl) 1-(3-chloro-2-fluoro-phenyl)-2-phenyl-1H-imidazol-4-yl]-1-(2-trimethylsilanyl-ethoxymethyl)-1-H-pyrazole-4-carboxylic acid methyl ester

Intermediate 382.2 (222 mg, 0.32 mmol), Intermediate 382.1 (191 mg, 0.48 mmol), Pd(PPh₃)₂Cl₂ (45 mg, 0.06 mmol) and CuI (120 mg, 0.06 mmol) are suspended in acetonitrile (5 mL) and stirred at 100° C. for 12 h. The reaction mixture is allowed to cool to rt and submitted to aqueous work up. The remaining crude product is purified by flash chromatography (SiO₂; hexanes/EtOAc, gradient: 0-60% EtOAc) to give the title compound as a white powder. HPLC: _{A}t_{Ret}=5.80 min. ¹HNMR (CDCl₃) 8.10 (s, 1H), 7.44-7.40 (m, 4H), 7.31-7.23 (m, 4H), 7.20 (d, 1H), 7.09-7.06 (m, 1H), 6.94-6.89 (m, 1H), 5.47 (s, 2H), 4.09 (dd, 2H), 3.60 (dd, 2H), 1.69-1.63 (m, 2H), 1.41-1.30 (m, 4H), 1.19 (t, 3H), 0.98-0.84 (m, 7H), 0.01 (s, 9H).

### Intermediate 382.1

### 3-Iodo-1-(2-trimethylsilanyl-ethoxymethyl)-1-H-pyrazole-4-carboxylic acid methyl ester

3-Iodo-1H-pyrazole-4-carboxylic acid methyl ester (prepared according to J. Med. Chem. 2008, 51, 159) (1.5 g, 5.95 mmol) is dissolved in THF (15 mL) at rt. NaH (60% suspension in mineral oil; 0.36 g, 8.43 mmol) is added slowly at rt and stirring continued until no gas evolution was observed. SEMCI (1.3 g, 7.74 mmol) is added slowly and the reaction mixture allowed to stir for 12 h. It is then submitted to aquous workup and the crude product purified by flash chromatography (SiO₂; hexanes/EtOAc, gradient: 0-30%

### Intermediate 382.2

### 5-(3-Chloro-4-fluoro-phenyl) 1-(3-chloro-2-fluoro-phenyl)-2-phenyl-4-tributylstannyl-1H-imidazole

Intermediate 454.1 (455 mg, 0.95 mmol) is dissolved in Et₂O and cooled to −78° C. At this temperature TMEDA (286 μl, 1.9 mmol) and n-BuLi (1.6 M solution in hexanes, 770 μL, 1.2 mmol) are added and stirring continued for 1 h. Tributyl stannyl chloride (385 μL, 1.4 mmol) is added and the reaction mixture allowed to stir for another 4 h. It is diluted with EtOAc and quenched by addition of water. The organic layer is separated, dried and concentrated. The remaining crude product is purified by flash chromatography (SiO₂; hexanes/EtOAc 99:1). ES-MS: [M+1]=691.1; HPLC: _{A}t_{Ret}=6.36 min.

### Intermediate 383.1

### 1-(3-Chloro-2-fluoro-phenyl)-5-(3-chloro-4-fluoro-phenyl)-2-piperidin-1-ylmethyl-1H-imidazole-4-carboxylic acid ethyl ester

Intermediate 383.2 (200 mg, 0.40 mmol) is dissolved in THF (2 mL) and piperidine (174 mg, 2.04 mmol) is added at rt. The reaction is then stirred at 50° C. for 30 min. The reaction mixture is cooled, submitted to aqueous workup and concentrated. The remaining crude product is purified by flash chromatography (SiO₂, gradient DCM/MeOH 0-5% MeOH). ES-MS: M+=496.0; HPLC: _{A}t_{Ret}=4.51 min

### Intermediate 383.2

### 2-Bromomethyl-1-(3-chloro-2-fluoro-phenyl)-5-(3-chloro-4-fluoro-phenyl)-1H-imidazole-4-carboxylic acid ethyl ester

The compound from Example 381 (295 mg, 0.71 mmol) is dissolved in CCl₄ (8 mL) and treated with NBS (306 mg, 1.6 mmol) and AIBN (12 mg, 0.08 mmol) at rt. The reaction mixture is then stirred under reflux for 48 h. It is cooled to ambient temperature again and submitted to aqueous workup. After evaporation of all solvents and drying the crude product is obtained as a yellow oil containing a mixture of mono- and gem di-bromo intermediate which is directly submitted to the next step without further purification. ES-MS: M+=490.0; HPLC: _{A}t_{Ret}=5.26 min (mono bromo)

### Intermediate 385.1

### 2-((R)-1-Benzyloxycarbonyl-pyrrolidin-2-yl)-1-(3-chloro-2-fluoro-phenyl)-5-(3-chloro-4-fluoro-phenyl)-1H-imidazole-4-carboxylic acid ethyl ester

The product from Intermediate 385.2 (100 mg, 0.18 mmol) is dissolved in toluene (4 mL). 3-Chloro-4-fluoro benzene boronic acid (48 mg, 0.275 mmol), Pd (PPh₃)₄ (21 mg, 0.018 mmol), potassium phosphate (117 mg, 0.55 mmol) and water (2 mL) are added at rt and the reaction mixture is then stirred at 100° C. for 1 h in a sealed tube. It is allowed to cool to rt again, diluted with EtOAc and washed with water and brine. The organic layer is dried over Na₂SO₄ and concentrated. The residual crude product is purified by flash chromatography (SiO₂; 12 g; DCM/MeOH gradient 0-10% MeOH) to give the title compound as a white powder. ES-MS: M+=602.1; HPLC: _{A}t_{Ret}=5.91 min.

### Intermediate 385.2

### 2-((R)-1-Benzyloxycarbonyl-pyrrolidin-2-yl)-5-bromo-1-(3-chloro-2-fluoro-phenyl)-1H-imidazole-4-carboxylic acid ethyl ester

The product from Intermediate 385.3 (110 mg, 0.16 mmol) is dissolved in acetonitrile (5 mL) and NBS (43 mg, 0.24 mmol) is added at rt. The reaction mixture is allowed to stir for 20 h at rt and is then diluted with EtOAc and washed with water and brine. The organic layer is dried over Na₂SO₄ and concentrated. The remaining crude product is used for the next step without further purification. ES-MS: M+=552.0; HPLC: _{A}t_{Ret}=5.47 min.

### Intermediate 385.3

### 2-((R)-1-Benzyloxycarbonyl-pyrrolidin-2-yl)-1-(3-chloro-2-fluoro-phenyl)-1H-imidazole-4-carboxylic acid ethyl ester

The title compound is synthesized by dehydration of Intermediate 385.4 analogously to the preparation of Intermediate 6.2; ES-MS: [M+1]⁺=473.1; HPLC: _{A}t_{Ret}=5.10 min.

### Intermediate 385.4

### 2-((R)-1-Benzyloxycarbonyl-pyrrolidin-2-yl)-1-(3-chloro-2-fluoro-phenyl)-4-hydroxy-4,5-dihydro-1H-imidazole-4-carboxylic acid ethyl ester

The title compound is synthesized by cycloaddition of Ethyl bromo pyvurate and Intermediate 385.5 analogously to the preparation of Intermediate 6.3, by using triethylamine as base and THF as solvent at 60° C.; ES-MS: [M+1]⁺=490.1; HPLC: _{A}t_{Ret}=4.83 min.

### Intermediate 385.5

### (R)-2-[N-(3-Chloro-2-fluoro-phenyl)-carbamimidoyl]-pyrrolidine-1-carboxylic acid benzyl ester

The title compound is synthesized by addition of 2-fluoro-3-chloro-aniline and Intermediate 385.6 analogously to the preparation of Intermediate 6.4; ES-MS: [M+1]⁺=376.0; HPLC: _{A}t_{Ret}=3.88

### Intermediate 385.6

### (R)-2-Cyano-pyrrolidine-1-carboxylic acid benzyl ester

The title compound is synthesized by dehydration of Intermediate 385.7 analogously to the preparation of Intermediate 346.5; ES-MS: [M+1]⁺=231.1; HPLC: _{A}t_{Ret}=4.31 min.

### Intermediate 385.7

### (R)-2-Carbamoyl-pyrrolidine-1-carboxylic acid benzyl ester

The title compound is synthesized according to the procedure described for preparation of Example 4 from N-Carbobenzyloxy proline; ES-MS: [M+1]⁺=249.1; HPLC: _{A}t_{Ret}=3.80 min.

### Intermediate 386.1

### 3-Chloro-5-(4,4,5,5-tetramethyl-[1,3,2]dioxaborolan-2-yl)-benzoic acid methyl ester

Methyl 3-chloro-5-iodobenzoate (2.0 g, 6.75 mmol) is dissolved in dichloroethane (7 mL) and bis-pinacolato diboron (3.4 g, 13.49 mmol), Pd(dppf)Cl₂ (826 mg, 1.02 mmol) and potassium acetate (1.96 g, 20.24 mmol) are added and the reaction mixture is stirred at 90° C. for 20 h in a sealed tube. The reaction is allowed to cool to rt and diluted with DCM. The organic layer is washed with sat. aqueous NH₄Cl solution and brine, dried over Na₂SO₄ and concentrated. The crude product is purified by flash chromatography (SiO₂, hexanes/EtOAc, gradient 0-60% EtOAc) to give the title compound as a yellow solid. ES-MS: M+=396.5; HPLC: _{A}t_{Ret}=3.94 min.

### Intermediate 390.1

### 5-Bromo-1-(5-chloro-2-methoxy-pyridin-3-yl)-2-phenyl-1H-imidazole-4-carboxylic acid ethyl ester

The title compound is synthesized by bromination of Intermediate 390.2 analogously to the preparation of Intermediate 6.1; ES-MS: M+=437.9; HPLC: _{A}t_{Ret}=5.03 min.

### Intermediate 390.2

### 1-(5-Chloro-2-methoxy-pyridin-3-yl)-2-phenyl-1H-imidazole-4-carboxylic acid ethyl ester

The title compound is synthesized by dehydration of Intermediate 390.3 analogously to the preparation of Intermediate 6.2; ES-MS: M+=358.0; HPLC: _{A}t_{Ret}=4.65 min.

### Intermediate 390.3

### 1-(5-Chloro-2-methoxy-pyridin-3-yl)-4-hydroxy-2-phenyl-4,5-dihydro-1H-imidazole-4-carboxylic acid ethyl ester

The title compound is synthesized by cycloaddition of ethyl bromopyruvate and Intermediate 390.4 analogously to the preparation of Intermediate 6.3; ES-MS: M+=377.9; HPLC: _{A}t_{Ret}=3.67 min.

### Intermediate 390.4

### N-(5-Chloro-2-methoxy-pyridin-3-yl)-benzamidine

The title compound is synthesized by addition of Intermediate 390.5 and benzonitrile analogously to the preparation of Intermediate 6.4; ES-MS: M+=262.1; HPLC: _{A}t_{Ret} 1.88 min.

### Intermediate 390.5

### 5-Chloro-2-methoyx-pyridin-3-ylamine

Intermediate 390.6 (4.7 g, 24.9 mmol) is dissolved in EtOH (50 ml) transferred to a Parr shaker and submitted to hydrogenation under atmospheric pressure at rt in the presence of Raney-Nickel (0.7 g) as catalyst for 10 h. After completion the reaction mixture is filtered over a pad of Celite, concentrated and dried under high vacuum to give the title compound as a beige solid. ES-MS: M+=159.3; HPLC: _{A}t_{Ret}=2.09 min.

### Intermediate 390.6

### 5-Chloro-2-methoyx-3-nitro-pyridine

2,5-Dichloro-3-nitropyridine (5.0 g, 25.9 mmol) is dissolved in MeOH (50 ml) and treated with NaOMe (1.7 g, 31.1 mmol) at rt. The reaction mixture is then stirred at reflux for 14 h. It is cooled to it and diluted with EtOAc, washed with water and brine. The organic layer is dried over Na₂SO₄, concentrated and dried to give the title compound as a yellow solid. ES-MS: M+=189.9; HPLC: _{A}t_{Ret}=4.09 min.

### Example 395

### 1-(6-Carboxymethyl-3-chloro-2-fluoro-phenyl)-5-(3-chloro-4-fluoro-phenyl)-2-cyclohexyl-1H-imidazole-4-carboxylic acid ethyl ester

The title compound is synthesized by Suzuki Coupling of Intermediate 395.1 with 2-(3-chloro-4-fluoro-phenyl)-4,4,5,5-tetramethyl-[1,3,2]dioxaborolane at 100° C. during 18 h as described in Example. 6; ES-MS: [M+1]⁺=537/539; HPLC: _{B}t_{Ret}=1.33 min.

### Intermediate 395.1

### 5-Bromo-1-(6-carboxymethyl-3-chloro-2-fluoro-phenyl)-2-cyclohexyl-1H-imidazole-4-carboxylic acid ethyl ester

The title compound is synthesized by bromination of Intermediate 395.2 analogously to the preparation of Intermediate 6.1; ES-MS; [M+1]⁺=487/489; HPLC: _{B}t_{Ret}=1.24 min.

### Intermediate 395.2

### 1-(6-Carboxymethyl-3-chloro-2-fluoro-phenyl)-2-cyclohexyl-1H-imidazole-4-carboxylic acid ethyl ester

Hydroboration and oxidative work-up of Intermediate 395.3 analogously to the preparation of Intermediate 371.2 gives the title compound; ES-MS: [M+1]⁺=409/411; HPLC: _{B}t_{Ret}=1.10 min.

### Intermediate 395.3

### 1-(3-Chloro-2-fluoro-6-trimethylsilanylethynyl-phenyl)-2-cyclohexyl-1H-imidazole-4-carboxylic acid ethyl ester

A solution of Intermediate 395.4 (12.02 g, 25.2 mmol) in Et₃N (264 ml) is degassed by repeated evacuation and flushing with N₂. Then Pd(OAc)₂ (436 mg, 1.94 mmol), CuI (147 mg, 0.76 mmol), PPh₃ (1.00 g, 3.78 mmol) and ethynyl-trimethyl-silane (9.44 ml, 68.1 mmol) are added. After 20 h stirring at rt, the suspension is diluted with EtOAc and water, the aq. layer separated off and extracted twice with EtOAc. The organic phases are washed with H₂O and brine, dried (Na₂SO₄) and concentrated. Column chromatography (SiO₂; hexane/EtOAc 19:1→3:2) gives the title compound; ES-MS: [M−1]=447/449; HPLC: _{B}t_{Ret}=1.52 min; ¹H NMR (DMSO d₆) δ 7.97 (s, 1H), 7.82 (t, 1H), 7.54 (d, 1H), 4.22 (q, 2H), 2.26 (m, 1H), 1.8-1.4 (m, 7H), 1.24 (t, 3H), 1.2-1.0 (m, 3H), 0.01 (s, 9H).

### Intermediate 395.4

### 1-(3-Chloro-2-fluoro-6-iodo-phenyl)-2-cyclohexyl-1H-imidazole-4-carboxylic acid ethyl ester

The title compound is synthesized by dehydration of Intermediate 395.5 analogously to the preparation of Intermediate 6.2; ES-MS: [M+1]⁺=477/479; HPLC: _{B}t_{Ret}=1.34 min.

### Intermediate 395.5

### rac. 1-(3-Chloro-2-fluoro-6-iodo-phenyl)-2-cyclohexyl-4-hydroxy-4,5-dihydro-1H-imidazole-4-carboxylic acid ethyl ester

The title compound is synthesized by reaction of ethyl bromopyruvate and Intermediate 395.6 analogously to the preparation of Intermediate 6.3; ES-MS: [M+1]⁺=495/497.

### Intermediate 395.6

### N-(3-Chloro-2-fluoro-6-iodo-phenyl)-cyclohexanecarboxamidine

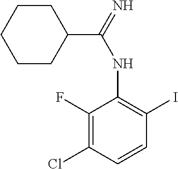

A mixture of Intermediate 395.7 (8.8 g, 32.4 mmol) and toluene (12 ml) is cooled to 0° C. Then trimethyl aluminium (2M solution in toluene; 29.2 ml, 58.4 mmol) is added dropwise. After completion of the addition the reaction mixture is stirred at RT for 2 h. Then cyclohexyl-carbonitrile (7.8 ml, 65 mmol) is added portionwise and stirring is continued for 24 h at 110° C. (additional portions of 3.5 ml each of cyclohexyl-carbonitrile are added after 16 h and 20 h). After cooling the reaction-mixture to RT, it is poured into 0.3 l of MeOH/DCM 1:2 and stirred for 1 h. DCM (0.2 l) and SiO₂ (50 g) are added and the mixture is concentrated in vacuo. The resulting powder is applied to a chromatography column (SiO₂). Eluation with hexane/EtOAc 9:1→3:2 gives the title compound; ES-MS: [M+1]⁺=381/383; HPLC: _{B}t_{Ret}=0.87; ¹H NMR (DMSO d₆) δ 7.52 (d, 1H), 6.84 (t, 1H), 3.28 (s, 2H), 2.13 (m, 1H), 1.84 (m, 2H), 1.73 (m, 2H), 1.55 (m, 3H), 1.2 (m, 3H).

### Intermediate 395.7

### 3-Chloro-2-fluoro-6-iodo-aniline NIS (47.2 g, 0.21 mol) is added to 3-chloro-2-fluoro-aniline (29.1 g, 0.20 mol) in DCM (300 ml). The suspension is stirred for 5 days at it and then diluted with EtOAc (200 ml).

After addition of hexane (2 l), the precipitate is filtered off and discarded. The filtrate is concentrated. Column chromatography (SiO₂; hexane/EtOAc 9:1→4:1) gives the title compound. ES-MS: [M−1]=270/272; TLC(hexane/EtOAc 4:1): R_{f}=0.61; ¹H NMR (DMSO d₆) δ 7.38 (d, 1H), 6.51 (t, 1H), 5.46 (s, H₂N).

### Example 396

### 1-(6-tert-Butoxycarbonylmethyl-3-chloro-2-fluoro-phenyl)-5-(3-chloro-4-fluoro-phenyl)-2-cyclohexyl-1H-imidazole-4-carboxylic acid ethyl ester

The title compound is synthesized from Example 395 as described in Example. 372; ES-MS: [M+1]⁺=593/595; HPLC: _{B}t_{Ret}=1.60 min; ¹H NMR (DMSO d₆) δ 7.75 (t, 1H), 7.48 (d, 1H), 7.31 (t, 1H), 7.29 (t, 1H), 7.11 (m, 1H), 4.09 (m, 2H), 3.34 (d, 1H), 3.16 (d, 1H), 2.18 (m, 1H), 1.7-1.0 (m, 10H), 1.35 (s, 9H), 1.08 (t, 3H).

### Example 397

### 1-(6-tert-Butoxycarbonylmethyl-3-chloro-2-fluoro-phenyl)-5-(3-chloro-4-fluoro-phenyl)-2-cyclohexyl-1H-imidazole-4-carboxylic acid

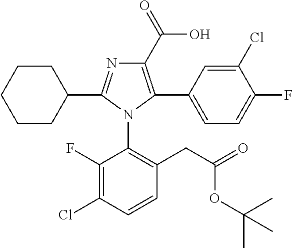

A mixture of 1-(6-tert-butoxycarbonylmethyl-3-chloro-2-fluoro-phenyl)-5-(3-chloro-4-fluoro-phenyl)-2-cyclohexyl-1H-imidazole-4-carboxylic acid benzyl ester (Ex. 502; 7.25 g, 11 mmol), MeOH (70 ml), HOAc (10 ml) and Pd/C (5% Engelhard 4522; 0.7 g) is hydrogenated under normal pressure at RT for 25 min. The catalyst is filtered off and the filtrate concentrated (RT; →HV). Reversed phase chromatography gives the title compound; ES-MS: [M+1]⁺=565/567; HPLC: _{B}t_{Ret}=1.41 min; ¹H NMR (DMSO d₆) δ 12.4 (s, HOOC), 7.75 (t, 1H), 7.42 (d, 1H), 7.29 (m, 2H), 7.11 (m, 1H), 3.3 (d, 1H), 3.15 (d, 1H), 2.17 (m, 1H), 1.7-1.0 (4m, 10H), 1.36 (s, Me₃C).

### Example 405

### {4-Chloro-2-[5-(3-chloro-4-fluoro-phenyl)-2-cyclohexyl-4-hydrazinocarbonyl-imidazol-1-yl]-3-fluoro-phenyl}-acetic acid tert-butyl ester

A mixture of Example 397 (1.1 g, 1.95 mmol), NMM (537 μl, 4.88 mmol) and HATU (961 mg, 2.53 mmol) in DMF (21 ml) is stirred for 5 min at rt. Then a 1 molar solution of hydrazine in THF (4.67 ml, 4.67 mmol) is added. After 2 h at rt, the solution is diluted with EtOAc and water, the aq. layer separated off and extracted twice with EtOAc. The organic phases are washed with H₂O and brine, dried (Na₂SO₄) and concentrated; ES-MS: [M+1]⁺=579/581; ¹H NMR (DMSO d₆) δ 9.05 (s, HN), 7.76 (t, 1H), 7.40 (d, 1H), 7.28 (m, 2H), 7.03 (m, 1H), 4.35 (s, H₂N), 3.28 (d, 1H), 3.13 (d, 1H), 2.18 (m, 1H), 1.69 (m, 4H), 1.60 (m, 2H), 1.44 (m, 1H), 1.34 (s, 9H), 1.25-1.05 (m, 3H).

### Example 410

### 4-(3-Chloro-2-fluoro-phenyl)-3-(3-chloro-phenyl)-5-phenyl-1H-pyrrole-2-carboxylic acid methyl ester

A solution of Intermediate 410.1 (0.58 g, 1.3 mmol) and chloranil (2,3,5,6-tetrachlor-1,4-benzochinone; 0.58 g) in xylene (20 ml) is stirred at 130° C. After 2.5 h another 0.29 g chloranil are added. After totally 6 h the mixture is cooled to rt, diluted with EtOAc and washed with water and brine. The aq. layers are re-extracted twice with EtOAc. The organic phases are dried (Na₂SO₄) and concentrated. Reversed phase chromatography gives the title compound. ES-MS: [M+1]⁺=440/442; TLC(hexane/EtOAc 4:1): R_{f}=0.26; HPLC: _{B}t_{Ret}=1.42 min; ¹H NMR (CDCl₃; rotamer signals) δ 9.34 (s, HN), 7.50 (m, 1H), 7.31, 7.26 and 7.17 (3m, 8H), 7.04 (d, 1H), 6.90 (m, 2H), 3.85 and 3.76 (2s, H₃C).

### Intermediate 410.1

### 4-(3-Chloro-2-fluoro-phenyl)-3-(3-chloro-phenyl)-5-phenyl-3,4-dihydro-2H-pyrrole-2-carboxylic acid methyl ester

A mixture of Intermediate 410.2 (0.53 g, 1.53 mmol) and 2-amino-malonic acid dimethyl ester hydrochloride (393 mg, 2.14 mmol) in NMP (2 ml) is stirred for 7 h at 130° C. Then another 393 mg 2-amino-malonic acid dimethyl ester hydrochloride are added. The mixture is stirred for 3 h at 150° C., when again 393 mg of 2-amino-malonic acid dimethyl ester hydrochloride are added. Stirring is continued at 150° C. for another hour. The mixture is cooled to rt and diluted with EtOAc (200 ml), water (100 ml) and sat. NaHCO₃ solution (50 ml). The aq. phase is separated off and extracted twice with EtOAc. The organic layers are washed with H₂O and brine, dried (Na₂SO₄) and concentrated. Water (5 ml) and sat. HCl^{aq.} (5 ml) are added to the residue and the mixture is stirred for 3 h at 100° C. After cooling to rt, it is diluted with water and then extracted with 3 portions of EtOAc. The organic layers are washed with H₂O and brine, dried (Na₂SO₄) and concentrated, giving 4-(3-chloro-2-fluoro-phenyl)-3-(3-chloro-phenyl)-5-phenyl-3,4-dihydro-2H-pyrrole-2-carboxylic acid (ES-MS: [M+1]⁺=428/430).

This crude acid is dissolved in MeOH (25 ml). Then Me₃SiCl (2.5 ml) is added and the solution stirred for 1 h at 50° C. The mixture is concentrated in vacuo, the residue diluted with EtOAc and water/sat. NaHCO₃ solution 2:1, the aq. phase separated off and extracted twice with EtOAc. The organic layers are washed with H₂O and brine, dried (Na₂SO₄) and concentrated. Reversed phase chromatography gives the title compound. ES-MS: [M+1]⁺=442/444; HPLC: _{B}t_{Ret}=1.34 min.

### Intermediate 410.2

### 2-(3-Chloro-2-fluoro-phenyl)-3-(3-chloro-phenyl)-1-phenyl-propenone

A solution of Intermediate 410.3 (770 mg, 3.1 mmol), freshly distilled 3-chloro-benzaldehyde (436 mg, 3.1 mmol), piperidine (109 μl, 1.10 mmol) and AcOH (186 μl, 3.25 mmol) in benzene (10 ml) is heated on a water separation equipment for 4 h under reflux conditions. Then 5 ml benzene are distilled off and heating of the residue is continued for 2 h. The mixture is cooled to rt and diluted with EtOAc and water. The aq. phase is separated off and extracted twice with EtOAc. The organic layers are washed with H₂O and brine, dried (Na₂SO₄) and concentrated, yielding the crude title compound as an E/Z mixture. ¹⁹F-NMR (DMSO-d₆): δ ppm −114.9, −117.2; ES-MS: [M+1]⁺=371/373; TLC(hexane/EtOAc 19:1): R_{f}=0.14/0.20; HPLC: _{B}t_{Ret}=1.44 min.

### Intermediate 410.3

### 2-(3-Chloro-2-fluoro-phenyl)-1-phenyl-ethanone

A few drops of a solution of 3-chloro-2-fluoro-benzylbromide (3.27 g, 14.64 mmol) in Et₂O (12 ml) are added to dry magnesium (382 mg, 15.7 mmol) in Et₂O (24 ml). Then the mixture is heated for a short period to start the reaction. The rest of the 3-chloro-2-fluoro-benzylbromide solution is added dropwise. Then the mixture is heated for 2 h under reflux conditions, giving the Grignard solution.

In a second vessel a solution of N-methoxy-N-methyl-benzamide (2022 mg, 12.2 mmol) in THF (15 ml) is cooled in an ice-bath. Above Grignard solution is added dropwise at a temperature of 0-10° C. The resulting suspension is stirred for 1 h in the ice-bath. Then 30 ml of HCl 2 N are added. After 10 min the mixture is diluted with Et₂O and water, the aq. layer separated off and extracted with 2 portions of Et₂O. The organic layers are washed with H₂O and brine, dried (Na₂SO₄) and concentrated. Combi Flash chromatography (hexane→hexane/EtOAc 4:1) gives the title compound. ES-MS: [M+1]⁺=249/251; TLC(hexane/EtOAc 9:1): R_{f}=0.30; HPLC: _{B}t_{Ret}=1.22 min.

### Example 427

### 4-(3-Chloro-2-fluoro-phenyl)-3-(3-chloro-phenyl)-5-phenyl-1H-pyrrole-2-carboxylic acid

A solution of Example 410 (64 mg, 0.145 mmol) in dioxane (5 ml) and 0.1 M ^{aq.}LIOH (5.5 ml) is stirred for 45 h at 45° C. Concentration and reversed phase chromatography gives the title compound. ES-MS: [M+1]⁺=426/428; HPLC: _{B}t_{Ret}=1.30 min.

### Example 435

### 1-[5-(3-Chloro-4-fluoro-phenyl)-4-(3-chloro-2-fluoro-phenyl)-3-m-tolyl-pyrazol-1-yl]-2-hydroxy-ethanone

A solution of Intermediate 435.1-B (27 mg, 0.048 mmol) in DCM (2 ml) is cooled in an ice-bath. Then MeSO₃H (½ ml) is added and the solution stirred for 30 min in the ice-bath and 45 min at rt. This solution is poured into a mixture of ice (30 g) and sat. NaHCO₃ solution (30 ml) and extracted with 3 portions of EtOAc. The organic layers are washed with brine, dried (Na₂SO₄) and concentrated. Reversed phase chromatography gives the title compound. HPLC: _{B}t_{Ret}=1.49 min; IR: 1745 cm⁻¹ (s); ¹H NMR (DMSO d₆; rotamer signals) δ 7.63 (m, 1H), 7.57 (t, 1H), 7.44 (t, 1H), 7.30 (m, 1H), 7.3-7.1 (m, 5H), 7.06 and 701 (2d, 1H), 5.45 (t, HO), 4.98 (d, 2H), 2.24 (s, H₃C).

### Intermediate 435.1

### 2-Benzyloxy-1-[3-(3-chloro-4-fluoro-phenyl)-4-(3-chloro-2-fluoro-phenyl)-5-m-tolyl-pyrazol-1-yl]-ethanone A and 2-benzyloxy-1-[5-(3-chloro-4-fluoro-phenyl)-4-(3-chloro-2-fluoro-phenyl)-3-m-tolyl-pyrazol-1-yl]-ethanone B

A solution of Intermediate 435.2 (196 mg, 0.47 mmol) in DCM (4 ml) and pyridine (2.5 ml) is cooled in an ice-bath. Then a solution of benzyloxy-acetyl chloride (129 mg, 0.70 mmol) in DCM (1 ml) is added and the solution stirred for 1 h in the ice-bath and 16 h at rt. The mixture is diluted with EtOAc and water, the aq. layer separated off and extracted twice with EtOAc. The organic layers are washed with water and brine, dried (Na₂SO₄) and concentrated. Combi Flash chromatography (hexane/toluene 19:1→1:1 toluene) gives A followed by B; A: ¹H-NMR (DMSO-d₆): δ ppm 5.12 [s, H₂C—CO; NOE to H—C(2) tolyl], 4.64 (s, H₂C); HPLC: _{B}t_{Ret}=1.60. B: ¹H-NMR (DMSO-d₆): δ ppm 5.15 [s, H₂C—CO; NOE to H—C(2) 3-chloro-4-fluoro-phenyl], 4.62 (s, H₂C); HPLC: _{B}t_{Ret}=1.61.

### Intermediate 435.2

### 5-(3-Chloro-4-fluoro-phenyl)-4-(3-chloro-2-fluoro-phenyl)-3-m-tolyl-1H-pyrazole

Degased dioxane (13 ml) is added to Intermediate 435.3 (519 mg, 1.28 mmol), m-tolyl-boronic acid (522 mg, 3.84 mmol), K₃PO₄ (815 mg, 3.84 mmol and Pd(dppf)Cl₂CH₂Cl₂ ([95464-05-4]; 105 mg, 0.128 mmol). The mixture is heated for 40 min at 160° C. in a micro wave oven. Then it is diluted with EtOAc and water, the aq. layer separated off and extracted twice with EtOAc. The organic layers are washed with water and brine, dried (Na₂SO₄) and concentrated. Combi Flash chromatography (hexane/EtOAc 19:1→7:3) gives the title compound. ES-MS: [M+1]⁺=415/417; TLC(hexane/EtOAc 3:1): R_{f}=0.24.

### Intermediate 435.3

### 3-Bromo-5-(3-chloro-4-fluoro-phenyl)-4-(3-chloro-2-fluoro-phenyl)-1H-pyrazole

A solution of Intermediate 435.4 (512 mg, 1.57 mmol) and NBS (559 mg, 3.14 mmol) in acetonitrile (100 ml) is stirred at rt. On days 3, 6 and 8, other portions of NBS (559 mg each) are added. After 10 d, SiO₂ is added to the solution and the mixture is concentrated in vacuo. Combi Flash chromatography (hexane/EtOAc 9:1→3:2) gives the title compound. HPLC: _{B}t_{Ref}=1.31 min; TLC(hexane/EtOAc 2:1): R_{f}=0.50.

### Intermediate 435.4

### 5-(3-chloro-4-fluoro-phenyl)-4-(3-chloro-2-fluoro-phenyl)-1H-pyrazole

A solution of intermediate 435.5 (748 mg, 1.83 mmol) and hydrazine hydrate (134 μl, 2.75 mmol) in ⁱpropanol (20 ml) is stirred for 1 h at it and 1 h at 40° C. Concentration and Combi Flash chromatography (hexane/EtOAc 9:1→1:1) gives the title compound. HPLC: _{B}t_{Ret}=1.25 min; TLC(hexane/EtOAc 2:1): R_{f}=0.30.

### Intermediate 435.5

### 2-(3-Chloro-2-fluoro-phenyl)-1-(3-chloro-4-fluoro-phenyl)-3-dimethylamino-propenone

To a solution of Intermediate 435.6 (903 mg, 3.0 mmol) in DMF (5 ml), dimethoxymethyl-dimethyl-amine (503 μl, 3.6 mmol) is added. This mixture is heated up to 75° C. for totally 2½ h. After 1 h heating, another portion of dimethoxymethyl-dimethyl-amine (250 μl) is added. Finally the solution is diluted with EtOAc and water, the aq. layer separated off and extracted twice with EtOAc. The organic layers are washed with water and brine, dried (Na₂SO₄) and concentrated. Combi Flash chromatography (hexane/EtOAc 50:1→3:7) gives the title compound. ES-ES-MS: [M+1]⁺=356/358; HPLC: _{B}t_{Ref}=1.19/1.21 min.

### Intermediate 435.6

### 2-(3-Chloro-2-fluoro-phenyl)-1-(3-chloro-4-fluoro-phenyl)-ethanone

A solution of Intermediate 435.7 (4.00 g, 18.4 mmol) in THF (23 ml) is cooled in an ice-bath. Then a solution of 3-chloro-2-fluoro-benzyl-magnesium bromide (22.1 mmol in 50 ml Et₂O; prepared as described for Intermediate 410.3) is added dropwise at a temperature of 0-10° C. during 60 min. The resulting suspension is stirred for 2½ h in the ice-bath. Then 45 ml of HCl 2 N are added. After 10 min the mixture is diluted with Et₂O and water, the aq. layer separated off and extracted with 2 portions of Et₂O. The organic layers are washed with H₂O and brine, dried (Na₂SO₄) and concentrated. Crystallization from hexane gives the title compound. mp: 109-111° C.; HPLC: _{B}t_{Ret}=1.32 min.

### Intermediate 435.7

### 3-Chloro-4-fluoro-N-methoxy-N-methyl-benzamide

3-Chloro-4-fluoro-benzoic acid (3.73 g, 21.4 mmol) and O,N-dimethyl-hydroxylamine hydrochloride (3.12 g, 32 mmol) in DMF (30 ml) are cooled in an ice-bath. Et₃N (30 ml, 215 mmol) and 2,4,6-tripropyl-1,3,5,2,4,6-trioxatriphosphorinane-2,4,6-trioxide ([68957-94-8] 50% in DMF; 25 ml, 42.8 mmol) are added. The mixture is stirred for 2 h and then poured into EtOAc and water. The aq. layer is separated off and extracted twice with EtOAc. The organic phases are washed with water and brine, dried (Na₂SO₄) and concentrated. Combi Flash chromatography (hexane/EtOAc 49:1→1:1) gives the title compound. ES-MS: [M+1]⁺=218/220; TLC(hexane/EtOAc 1:1): R_{f}=0.50.

### Example 436

### 2-[5-(3-Chloro-4-fluoro-phenyl) 1-(3-chloro-2-fluoro-phenyl)-2-phenyl-1H-imidazol-4-yl]-4-methyl-oxazole

Example 486 (93 mg, 0.21 mmol) is dissolved in EtOH (2 mL) and treated with chloroacetone (194 mg, 2.1 mmol). The reaction vessel is sealed and stirred at 140° C. with microwave irradiation for 6 h. The reaction mixture is allowed to cool to rt and submitted to aqueous work up. The remaining crude product is purified by flash chromatography (SiO₂; DCM/MeOH, gradient: 0-5% MeOH) to give the title compound as a white powder. ES-MS: [M+1]=484.1. HPLC: _{A}t_{Ret}=5.56 min.

### Example 437

### 5-(3-Chloro-4-fluoro-phenyl) 1-(3-chloro-2-fluoro-phenyl)-2-phenyl-1H-imidazole-4-caroxylic acid (2-oxo-propyl)amide

Obtained as a side product from the preparation of Example 436. ES-MS: [M+1]=502.7. HPLC: _{A}t_{Ret}=5.20 min.

### Example 438

### 1-(5-Chloro-2-methyl-phenyl)-5-(3-chloro-phenyl)-2-phenyl-1H-imidazol-4-ylamine

Example 439 (99 mg, 0.20 mmol) is dissolved in a solution of HCl in dioxane (4M, 3 ml) and stirred for 1.5 h at rt. The volatiles are removed under reduced pressure and the residue is taken up in EtOAc and washed with NaHCO₃ and brine, dried over Na₂SO₄ and concentrated. The remaining crude product is purified by flash chromatography (SiO₂, DCM/MeOH, gradient 0-4% MeOH) to give the title compound as a yellow solid. ES-MS: M+=395.9; HPLC: _{A}t_{Ret}=4.56 min.

### Example 439

### [1-(5-Chloro-2-methyl-phenyl)-5-(3-chloro-phenyl)-2-phenyl-1H-imidazol-4-yl]-carbamic acid tert-butyl ester

Example 37 (100 mg, 0.23 mmol) is dissolved in ^{t}BuOH (3 ml) at rt. DPPA (102 μl, 0.46 mmol) and TEA (66 μl, 0.46 mmol) are added and the reaction mixture is stirred at reflux for 3 h. It is allowed to cool to ambient temperature and concentrated under reduced pressure. The residue is taken up in EtOAc and the organic layer is washed with 5% aq citric acid, sat. aq NaHCO₃ and brine, dried over Na₂SO₄ and concentrated to give the title compound as a yellow oil. ES-MS: M+=496.0; HPLC: _{A}t_{Ret}=5.34 min.

### Example 440

### N-[1-(5-Chloro-2-methyl-phenyl)-5-(3-chloro-phenyl)-2-phenyl-1H-imidazol-4-yl]-acetamide

Example 438 (29 mg, 0.073 mmol) is dissolved in DCM (2 ml) at rt. TEA (20 μl, 0.15 mmol) and acetyl chloride (16 μl, 0.080 mmol) are added and the reaction mixture is stirred 20 min at ambient temperature. All volatiles are removed under reduced pressure and the residual material is taken up in EtOAc. The organic layer is washed with brine and H₂O, dried over Na₂SO₄ and concentrated. The remaining crude product is purified by flash chromatography (SiO₂, DCM/MeOH, gradient 0-3% MeOH) to give the title compound as a yellow foam. ES-MS: M+=437.9; HPLC: _{A}t_{Ret}=4.60 min.

### Example 441

### [5-(3-Chloro-2-fluoro-phenyl)-1-(3-chloro-2-methyl-phenyl)-2-phenyl-1-H-imidazol-4-yl]methanol

Example 106 (74 mg, 0.11 mmol) is dissolved in THF (2 ml) and cooled to 5° C. LAH (2M solution in THF; 110 μl, 0.22 mmol) is added dropwise. After addition the reaction mixture is stirred for 5 min. at 5° C. and then at rt for 30 min. It is diluted with EtOAc and the organic layer is washed with brine (2×), dried over Na₂SO₄ and concentrated. The remaining crude product is purified by MPLC (RP18, 70 ml/min; TFA/water (0.1/100, v/v)/TFA/acetonitrile (0.1/100, v/v),

gradient: linear gradient from 2% to 60% acetonitrile in 15 min then 5 min 60% acetonitrile) to give the title compound as a white solid. ES-MS: M+=414.9; HPLC: _{A}t_{Ret}=4.20 min.

### Example 443

### 5-[1-(3-Chloro-2-fluoro-phenyl)-5-(3-chloro-4-fluoro-phenyl)-2-phenyl-1-H-imidazol-4-yl]-acetonitrile

Intermediate 443.1 (290 mg, 0.64 mmol) is dissolved in acetonitrile (8 ml) and treated with tetrabutylammonium cyanide (572 mg, 2.1 mmol) at rt. The reaction mixture is then allowed to stir at 40° C. for 30 min, cooled to rt again and diluted with EtOAc. The organic layer is washed with H₂O and brine, dried over Na₂SO₄ and concentrated. The remaining crude product is purified by flash chromatography (SiO₂, DCM/MeOH, gradient 0-5% MeOH) to give the title compound as a yellow solid. ES-MS: M+=405.8 (M+—Cl); HPLC: _{A}t_{Ret}=5.29 min.

### Intermediate 443.1

### 1-(3-Chloro-2-fluoro-phenyl)-5-(3-chloro-4-fluoro-phenyl)-4-chloromethyl-2-phenyl-1-H-imidazole

The product from Example 442 (280 mg, 0.64 mmol) is dissolved in THF (4 ml) and treated with thionyl chloride (236 μL, 3.2 mmol) at rt. After 1 h of stirring at rt the reaction mixture is concentrated under reduced pressure and the residual crude product dried under high vacuum to give the title compound as a yellow solid. ES-MS: M+=416.6 (M+—Cl); HPLC: _{A}t_{Ret}=5.47 min.

### Example 444

### 5-[1-(3-Chloro-2-fluoro-phenyl)-5-(3-chloro-4-fluoro-phenyl)-2-phenyl-1-H-imidazol-4-ylmethyl]-2H-tetrazole

Sodium azide (202 mg, 3.1 mmol) is suspended in toluene (0.5 ml) and cooled to 0° C. Diethylaluminumchloride (1.7 ml, 3.1 mmol; 1.8 M solution in toluene) is added dropwise and the reaction mixture is then allowed to stir at rt for 2 h. The resulting suspension is then added to a solution of Example 443 (105 mg, 0.23 mmol) in toluene (0.5 ml) at rt and stirring is continued for 12 h. The reaction mixture is then diluted with EtOAc and washed with citric acid (5% aq. sol.), H₂O and brine. It is dried over Na₂SO₄ and concentrated. The remaining crude product is purified by MPLC (RP18, H₂O/acetonitrile/0.1% TFA, gradient 2-80% acetonitrile) to give the title compound as a yellow solid. ES-MS: M+=451.0 (M+—Cl); HPLC: _{A}t_{Ret}=4.59 min.

### Example 447

### [1-(3-Chloro-2-fluoro-phenyl)-5-(5-chloro-2-methoxy-phenyl)-2-phenyl-1-H-imidazol-4-yl]-acetic acid

Example 448 (83 mg, 0.18 mmol) is dissolved in a 4M solution of HCl in dioxane (4 ml) and stirred at 40° C. for 2 h. The reaction mixture is concentrated under reduced pressure and the remaining crude material purified by MPLC (RP18, H₂O/acetonitrile/0.1% TFA, gradient 2-70 acetonitrile) to give the title compound as a yellow solid. ES-MS: M==469.9 (M+—Cl); HPLC: _{A}t_{Ret}=3.93 min.

### Example 448

### 1-[3-(3-Chloro-4-fluoro-phenyl)-4-(3-chloro-2-fluoro-phenyl)-5-m-tolyl-pyrazol-1-yl]-2-hydroxy-ethanone

A solution of Intermediate 435.1-A (20 mg, 0.036 mmol) in DCM (2 ml) is debenzylated as described in Example 435 to the title compound. HPLC: _{B}t_{Ret}=1.48 min; TLC(hexane/EtOAc 3:1): R_{f}=0.34.

### Example 449

### 1-(5-Chloro-2-oxo-1,2,-dihydro-pyridin-3-yl)-5-(3-chloro-phenyl)-2-phenyl-2-phenyl-1-H-imidazole carboxylic acid

Example 391 (50 mg, 0.11 mmol) is dissolved in acetonitrile (3 ml) and treated with iodo trimethyl silane (62 ml, 0.44 mmol) at rt for 1.5 h. The reaction mixture is then concentrated taken up in MeOH and concentrated. The remaining material is taken up in EtOAc, washed with H₂O and brine, dried and concentrated again. The remaining crude product is titurated with DCM, filtered and dried at high vacuum to give the title compound as an off white solid. ES-MS: M−=423.9; HPLC: _{A}t_{Ret}=3.71 min.

### Example 450

### 5-Chloro-3-[5-(3-chloro-phenyl)-2-phenyl-4-(1H-tetrazol-5-yl)-imidazol-1-yl]-1H-pyridin-2-one

The title compound is synthesized by demethylation of Example 394 analogously to the preparation of Example 449; ES-MS: M+=451.9; HPLC: _{A}t_{Ret}=2.03 min.

### Example 451

### 1-(3-Chloro-2-fluoro-phenyl)-5-(3-chloro-phenyl)-2-phenyl-1-H-imidazole-4-sulfonic acid amide

Example 405 (104 mg, 0.27 mmol) is dissolved in chlorosulfonic acid (0.3 ml) and allowed to stir at 60° C. for 1.5 h. The reaction mixture is then allowed to cool to rt again and thionylchloride (20 μL, 0.27 mmol) is added. The reaction mixture is then stirred in a sealed tube at 60° C. for 45 min. It is allowed to cool to rt and partitioned between DCM and brine. The organic layer is dried over Na₂SO₄ and filtered. A solution of NH₃ in dioxane (0.5 M; 22 ml) is added to the filtrate and stirring is continued for 48 h. The reaction mixture is then poured into H₂O and the aq. phase repeatedly extracted with DCM. Combined extracts are dried over Na₂SO₄ and concentrated. The remaining crude product is purified by flash chromatography (SiO₂; hexanes/EtOAc; gradient 0-6% EtOAc) ES-MS: M−=461.9; HPLC: _{A}t_{Ret}=3.73.

### Example 452

### 1-(3-Chloro-2-fluoro-phenyl)-5-(3-chloro-phenyl)-2-phenyl-1-H-imidazole-4-sulfonic acid (2-methoxy-ethyl)-amide

Intermediate 452.1 (122 mg, 0.25 mmol) is dissolved in THF (5 ml) at rt and treated with 2-methoxy ethylamine (44 ml, 0.51 mmol) at rt. The reaction mixture is allowed to stir for 20, diluted with EtOAc and the organic layer washed with aq. citric acid (5% wt) The organic layer is dried over Na₂SO₄ and concentrated. The remaining crude product is purified by flash chromatography (SiO₂; DCM/MeOH; gradient 0-5% MeOH) ES-MS: M+=521.9; HPLC: _{A}t_{Ret}=4.16

### Intermediate 452.1

### 1-(3-Chloro-2-fluoro-phenyl)-5-(3-chloro-phenyl)-2-phenyl-1-H-imidazole-4-sulfonyl chloride

Example 405 (310 mg, 0.8 mmol) is dissolved in chlorosulfonic acid (1 ml) and allowed to stir at 60° C. for 1.5 h. The reaction mixture is then allowed to cool to rt again and thionylchloride (66 μl, 0.9 mmol) is added. The reaction mixture is then stirred in a sealed tube at 60° C. for 45 min. It is allowed to cool to rt and partitioned between DCM and brine. The organic layer is over Na₂SO₄, concentrated to give the title compound, which is submitted directly to the next step. ES-MS: M+=482.7; HPLC: _{A}t_{Ret}=5.09.

### Example 454

### 1-(3-Chloro-2-fluoro-phenyl)-5-(3-chloro-4-fluoro-phenyl)-2-phenyl-4-(1H-pyrrol-2-yl)-1-H-imidazole

Intermediate 454.1 (100 mg, 0.21 mmol) is dissolved in dioxane (5 ml). 1-N-Boc-pyrrole-2-boronic acid (57 mg, 0.27 mmol), Pd(PPh₃)₄ (24 mg, 0.02 mmol), K₃PO₄ (177 mg, 0.83 mmol) and water (2 ml) are added to this solution at rt. The reaction mixture is then stirred at 100° C. in a sealed tube for 1 h. It is allowed to cool to rt again, diluted with EtOAc and the organic layer is washed with water and brine, dried over Na₂SO₄ and concentrated. The remaining crude product is purified by flash chromatography (SiO₂, DCM/MeOH, gradient 0-5% MeOH) to give the title compound as a yellow solid. ES-MS: M−=465.9; HPLC: _{A}t_{Ret}=5.21 min.

### Intermediate 454.1

### 4-Bromo-1-(3-chloro-2-fluoro-phenyl)-5-(3-chloro-4-fluoro-phenyl)-2-phenyl-1H-imidazole

Example 404 (215 mg, 0.53 mmol) is dissolved in acetonitrile (5 ml) and treated with NBS at rt. The reaction mixture is then stirred at 40° C. for 2 h. It is allowed to cool to rt and diluted with EtOAc. The organic layer is washed with water and brine, dried over Na₂SO₄ and concentrated. The remaining crude product is purified by flash chromatography (SiO₂, DCM/MeOH, gradient 0-2% MeOH) to give the title compound as a yellow solid. ES-MS: M+=482.5; HPLC: _{A}t_{Ret}=5.99 min.

### Example 457

### {2-[4-Carbamoyl-5-(3-chloro-4-fluoro-phenyl)-2-cyclohexyl-imidazol-1-yl]-4-chloro-phenyl}-acetic acid

A solution of Example 456 (30 mg, 0.055 mmol) in 2 N HCl in dioxane (2 ml) is stirred for 3 d at 45° C. Lyophilization gives the title compound. ES-MS: [M+1]⁺=490/492; HPLC: _{B}t_{Ret}=1.14 min.

### Example 460 and 461

### {4-Chloro-2-[5-(3-chloro-4-fluoro-phenyl)-2-cyclohexyl-4-(2H-tetrazol-5-yl)-imidazol-1-yl]-phenyl}-acetic acid tert-butyl ester A and {4-chloro-2-[5-(3-chloro-4-fluoro-phenyl)-2-cyclohexyl-4-(2H-tetrazol-5-yl)-imidazol-1-yl]-phenyl}-acetic acid B

A suspension of NaN₃ (74.4 mg, 1.145 mmol) in toluene (0.1 ml) is cooled in an ice bath. Then Et₂AlCl (1.8 M in toluene; 0.636 ml, 1.145 mmol) is added and the mixture is stirred for 2 h at rt. The mixture is cooled to 0° C. again and Example 459 (47 mg, 0.088 mmol) in toluene (0.5 ml) is added. The mixture is stirred for 17 h at it and then poured into 5% citric acid and EtOAc. The aq. layer is separated off and extracted twice with EtOAc. The organic phases are washed with H₂O and brine, dried (Na₂SO₄) and concentrated.

Reversed phase chromatography gives B followed by A. A: ES-MS: [M+1]⁺=571/573; HPLC: _{B}t_{Ret}=1.50 min. B: ES-MS: [M+1]⁺=515/517; HPLC: _{B}t_{Ret}=1.20 min.

### Example 462

### 2-{4-Chloro-2-[5-(3-chloro-4-fluoro-phenyl)-2-cyclohexyl-4-(2H-tetrazol-5-yl)-imidazol-1-yl]phenyl}-N-(4-methoxy-benzyl)-acetamide

Example 461 (50 mg, 0.087 mmol) dissolved in DMF (1 ml), 4-methoxy-benzylamine (16 μl, 123 μmol), Et₃N (0.16 ml, 1.15 mmol), DMAP (4.6 mg, 38 μmol) and 2,4,6-tripropyl-1,3,5,2,4,6-trioxatriphosphorinane-2,4,6-trioxide ([50% in DMF; 85 μl, 145 μmol) are converted to the title compound as described in Example 375. ES-MS: [M+1]⁺=634/636; HPLC: _{B}t_{Ret}=1.33 min.

### Example 463

### 5-(3-Chloro-4-fluoro-phenyl)-1-(4-chloro-pyridin-2-yl)-2-m-tolyl-1H-imidazole-4-carboxylic acid ethyl ester A and 5-(3-chloro-4-fluoro-phenyl)-1-(4-bromo-pyridin-2-yl)-2-m-tolyl-1H-imidazole-4-carboxylic acid ethyl ester as by-product

To a mixture of 2-bromo-5-(3-chloro-4-fluoro-phenyl)-1-(4-chloro-pyridin-2-yl)-1H-imidazole-4-carboxylic acid ethyl ester and 2-bromo-5-(3-chloro-4-fluoro-phenyl)-1-(4-bromo-pyridin-2-yl)-1H-imidazole-4-carboxylic acid ethyl ester (0.093 mmol) in 2.1 ml of a degassed 2:1-mixture of dioxane and H₂O, K₃PO₄ (113 mg, 0.532 mmol), m-tolyl-boronic acid (15.2 mg, 0.112 mmol) and Pd(PPh₃)₄ (15 mg, 0.013 mmol) are added. The mixture is stirred for ½ h at 85° C., cooled to rt and diluted with EtOAc and water. The aq. layer is separated off and extracted twice with EtOAc. The organic phases are washed with H₂O and brine, dried (Na₂SO₄) and concentrated. Reversed phase chromatography gives the TFA-salts of A, followed by B. A: ES-MS: [M+1]⁺=470/472; HPLC: _{B}t_{Ret}=1.37 min. B: ES-MS: [M+1]⁺=514/516; HPLC: _{B}t_{Ret}=1.45 min.

### Intermediate 463.1

### 2-Bromo-5-(3-chloro-4-fluoro-phenyl)-1-(4-chloro-pyridin-2-yl)-1H-imidazole-4-carboxylic acid ethyl ester A and 2-bromo-5-(3-chloro-4-fluoro-phenyl)-1-(4-bromo-pyridin-2-yl)-1H-imidazole-4-carboxylic acid ethyl ester B

To a suspension of Intermediate 463.2 (185 mg, 0.467 mmol) in toluene (8.9 ml), OPBr₃ (268 mg, 0.934 mmol) is added. After 20 h at 110° C., the reaction mixture is poured into sat. NaHCO₃ and ice and extracted with 3 portions of EtOAc. The organic phases are washed with H₂O and brine, dried (Na₂SO₄) and concentrated. Combi Flash chromatography (DCM/EtOAc 99:1→7:3) gives a ≈1:4 mixture of A and B. A: ES-MS: [M+1]⁺=458/460. B: ES-MS: [M+1]⁺=502/504/506.

### Intermediate 463.2

### 5-(3-Chloro-4-fluoro-phenyl)-1-(4-chloro-pyridin-2-yl)-2-oxo-2,3-dihydro-1H-imidazole-4-carboxylic acid ethyl ester

Intermediate 463.3 (448 mg, 1.082 mmol) is added to polyphosphoric acid (CAS: 8017-16-1; 1.9 g) and 1,2-dichlorethane (6.4 ml) in a sealed vessel. The mixture is heated for 8½ h at 100° C. After cooling to rt, it is diluted with sat. NaHCO₃, water and EtOAc. The aq. layer is extracted twice with EtOAc. The organic phases are washed with H₂O and brine, dried (Na₂SO₄) and concentrated. Combi Flash chromatography (DCM/EtOAc 99:1→1:4) gives the title compound. ES-MS: [M+1]⁺=396/398; HPLC: _{B}t_{Ret}=1.15 min.

### Intermediate 463.3

### 3-(3-Chloro-4-fluoro-phenyl)-2-[3-(4-chloro-pyridin-2-yl)-ureido]-3-oxo-propionic acid ethyl ester

Solvent mixture: A 1:1-mixture of 1,2-dichloro-ethane and dioxane is degassed by repeated evacuation and flushing with N₂. Intermediate 463.4 (400 mg, 2.33 mmol) is suspended in 24 ml of this solvent mixture, Rh₂Oct₄ ([Cas: 73482-96-9]; 46 mg, 0.059 mmol) is added and the suspension is warmed up to 80° C. A solution of Intermediate 7.4 (1893 mg, 6.99 mmol) in 36 ml of the solvent mixture is added during 3 h. After 1 h and 2 h at 80° C., two additional portions of 46 mg Rh₂Oct₄ each are added. After totally 3½ h, the resulting solution is cooled to it and then diluted with EtOAc and water/sat. NaHCO₃ 4:1, the aq. layer is separated off and extracted twice with EtOAc. The organic phases are washed with H₂O and brine, dried (Na₂SO₄) and concentrated. Reversed phase chromatography gives the title compound. ES-MS: [M+1]⁺=414/416; HPLC: _{B}t_{Ret}=1.23 min.

### Intermediate 463.4

### (4-Chloro-pyridin-2-yl)-urea

A mixture of Pd(OAc)₂ (144 mg; 0.64 mmol) and Xantphos ([CAS: 161265-03-8]; 752 mg, 1.30 mmol) in dioxane (54 ml) is degassed by repeated evacuation and flushing with N₂. Then 2,4-dichlorpyridine (3.22 g, 21.8 mmol), urea (2.619 g, 43.6 mmol), NaO^{t}Bu (3.02 g, 31.4 mmol) and degassed H₂O (560 μl, 31 mmol) are added. The mixture is stirred for 2 h at 100° C. and then cooled to it. Filtration and combi flash chromatography [(DCM→DCM/MeOH 1:1 (poor solubility)] or reversed phase chromatography gives the title compound. ES-MS: [M+1]⁺=172/174.

### Example 464

### 4-[5-(3-Chloro-4-fluoro-phenyl) 1-(3-chloro-2-fluoro-phenyl)-2-phenyl-1H-imidazol-4-yl]-isoxazole

Intermediate 454.1 (100 mg, 0.21 mmol) is dissolved in toluene (5 ml) and water (2.5 mL). Isoxazole boronic acid (81 mg, 0.42 mmol), K₃PO₄ (133 mg, 0.63 mmol) and Pd(PPh₃)₄ are added and the reaction mixture is stirred at 90° C. for 16 h. It is allowed to cool to rt and submitted to aqueous workup. The remaining crude material is purified by flash chromatography (SiO₂; hexanes/EtOAc, gradient: 0-40% EtOAc). ES-MS: [M+1]=469.8. HPLC: _{A}t_{Ret}=5.43 min.

### Example 466

### 3-[5-(3-Chloro-4-fluoro-phenyl)-1-(3-chloro-2-fluoro-phenyl)-2-phenyl-1H-imidazol-4-yl]-5-methyl-[1,2,4]oxadiazole

The title compound is prepared from Intermediate 466.1 and phenylboronic acid by Suzuki coupling as described for Example 7. ES-MS: [M+1]=484.7. HPLC: _{A}t_{Ret}=5.44 min.

### Intermediate 466.1

### 3-[2-Bromo-5-(3-chloro-4-fluoro-phenyl)-1-(3-chloro-2-fluoro-phenyl)-1H-imidazol-4-yl]-5-methyl-[1,2,4]oxadiazole

The title compound is prepared from Intermediate 466.2 in analogy to the method described for Intermediate 6.1. ES-MS: [M+1]=488.7. HPLC: _{A}t_{Ret}=5.28 min.

### Intermediate 466.2

### 3-[5-(3-Chloro-4-fluoro-phenyl)-1-(3-chloro-2-fluoro-phenyl)-1H-imidazol-4-yl]-5-methyl-[1,2,4]oxadiazole

Dimethylacetamide dimethyl acetal (5.9 ml, 40.5 mmol) is added to Intermediate 466.3 (0.85 g, 1.6 mmol) and heated to 120° C. for 2 hr. The reaction is cooled to rt and all volatiles are evaporated. The remaining crude material is purified by flash chromatography (SiO₂; DCM/MeOH, gradient: 0-10% MeOH) to give the title compound as a white powder. ES-MS: [M+1]=408.9. HPLC: _{A}t_{Ret}=4.85 min.

### Intermediate 466.3

### 5-(3-Chloro-4-fluoro-phenyl)-1-(3-chloro-2-fluoro-phenyl)-N-hydroxy-1H-imidazol-4-carboxamide

Hydroxylamine hydrochloride (327 mg, 4.7 mmol) is added to a solution of Intermediate 227.3 (750 mg, 2.1 mmol) and Et₃N (0.98 ml, 7.1 mmol) in THF (5 ml) and stirred for 16 hr at 60° C. The reaction mixture is diluted with EtOAc and filtered (precipitate: Et₃N.HCl). After removal of all volatiles under reduced pressure the crude material is directly submitted to the next step. ES-MS: [M+1]=383.9. HPLC: _{A}t_{Ret}=3.76 min.

### Example 467

### {2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-acetic acid tert-butyl ester

A mixture of Example 405 (1.1 g, 1.90 mmol) and NaHCO₃ (184 mg, 2.19 mmol) in dioxane (25 ml) and water (12.5 ml) is stirred for 5 min at rt. Then BrCN (222 mg, 2.095 mmol) is added. After 6 h at rt, the suspension is diluted with EtOAc and water, the aq. layer separated off and extracted twice with EtOAc. The organic phases are washed with H₂O and brine, dried (Na₂SO₄) and concentrated. Column chromatography (SiO₂; hexane/EtOAc 99:1→EtOAc) gives the title compound; ES-MS: [M+1]⁺=604/606; ¹H NMR (DMSO d₆) δ 7.78 (t, 1H), 7.46 (d, 1H), 7.35 (t, 1H), 7.31 (d, 1H), 7.17 (m, 1H7.13 (s, H₂N), 3.35 (d, 1H), 3.20 (d, 1H), 2.21 (m, 1H), 1.75-1.55, 1.43 and 1.3-1.0 (3m, 10H), 1.34 (s, 9H).

### Example 468

### (2-[4-(5-Amino-[1,3,4]oxadiazol-2-yl)-5-(3-chloro-4-fluoro-phenyl)-2-(cyclohexyl)-imidazol-1-yl]-3-fluoro-4-chloro-phenyl) acetic acid

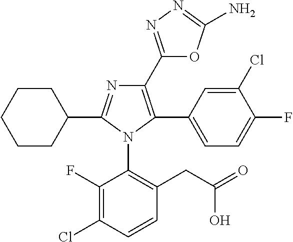

Example 467 (720 mg, 1.19 mmol) is dissolved in dioxane (22 ml) at rt and treated with a 4M solution of HCl in dioxane (22 ml). The reaction mixture is allowed to stir at rt for 40 h. The resulting precipitate is filtered off and washed with dioxane and Et₂O, yielding the title compound; ES-MS: [M+1]⁺=548/550; HPLC: _{B}t_{Ret}=1.11 min; ¹H NMR (DMSO d₆) δ 7.77 (t, 1H), 7.67 (sb, 2H), 7.46 (d, 1H), 7.34 (m, 2H), 7.17 (m, 1H), 3.36 (d, 1H), 3.18 (d, 1H), 2.21 (m, 1H), 1.8-1.0 (4m, 10H).

### Example 469

### 2-{2-[4-(5-Amino-[1,3,4]oxadiazol-2-yl)-5-(3-chloro-4-fluoro-phenyl)-2-cyclohexyl-imidazol-1-yl]-4-chloro-3-fluoro-phenyl}-N-methyl-acetamide

To a mixture of Example 468 (54.8 mg, 0.100 mmol) dissolved in DMF (1 ml), methylamine hydrochloride (20.3 mg, 0.30 mmol), Et₃N (181 μl, 1.3 mmol), DMAP (5.3 mg, 0.043 mmol) and 2,4,6-tripropyl-1,3,5,2,4,6-trioxatriphosphorinane-2,4,6-trioxide ([68957-94-8] 50% in DMF; 117 μl, 0.20 mmol) are added. The solution is stirred for 5 h at rt and then poured into EtOAc and water. The aq. layer is separated off and extracted twice with EtOAc. The organic phases are washed with water and brine, dried (Na₂SO₄) and concentrated. Reversed phase chromatography gives the title compound; ES-MS: [M+1]⁺=561/563; ¹H NMR (DMSO d₆) δ 7.91 (q, HN), 7.74 (t, 1H), 7.49 (d, 1H), 7.35 (t, 1H), 7.30 (d, 1H), 7.15 (m, 1H), 7.13 (s, H₂N), 3.15 (d, 1H), 3.10 (d, 1H), 2.51 (d, 3H), 2.17 (m, 1H), 1.8-1.55, 1.39 and 1.25-1.0 (3m, 10H).

### Example 476

### 2-{4-Chloro-2-[5-(3-chloro-4-fluoro-phenyl)-2-cyclohexyl-4-(5-methyl-[1,3,4]oxadiazol-2-yl)-imidazol-1-yl]-phenyl}-1-piperidin-1-yl-ethanone

To Example 475 (15.9 mg, 0.030 mmol) dissolved in DMF (0.31 ml), piperidine (3.2 μl, 0.033 mmol), Et₃N (46 μl, 0.33 mmol), DMAP (1.7 mg, 0.014 mmol) and 2,4,6-tripropyl-1,3,5,2,4,6-trioxatriphosphorinane-2,4,6-trioxide ([68957-94-8] 50% in DMF; 37 μl, 0.064 mmol) are added. The solution is stirred for ½ h at it and then worked up as described in Example 469; ES-MS: [M+1]⁺=596/598; HPLC: _{B}t_{Ret}=1.35 min.

### Example 478

### {4-Chloro-2-[5-(3-chloro-4-fluoro-phenyl)-2-cyclohexyl]-4-hydrazinocarbonyl-imidazol-1-yl]-phenyl}-acetic acid tert-butyl ester

The title compound is prepared according to the procedure described for Example 405 from Example 455. ES-MS: [M+1]=563.1; HPLC: _{A}t_{Ret}=5.25 min.

### Example 479

### {2-[4-(5-Amino-[1,3,4]oxadiazol-2-yl)-5-(3-chloro-4-fluoro-phenyl)-2-(cyclohexyl)-imidazol-1-yl]-4-chloro-phenyl}-acetic acid tert butylester

The title compound is prepared according to the procedure described for Example 467 from Example 478. The crude product is purified by flash chromatography (SiO₂; DCM/MeOH; 0-10% MeOH). ES-MS: [M+1]=588.1; HPLC: _{A}t_{Ret}=5.55 min. ¹HNMR (CDCl₃) 7.44 (dd, 1H), 7.36 (d, 1H), 7.29 (d, 1H), 7.25-7.12 (m, 2H), 7.02 (dd, 1H), 5.21 (bs, 2H), 2.81 (s, 2H), 2.36-2.23 (m, 1H), 1.86-1.63 (m, 6H), 1.41 (s, 9H), 1.31-1.10 (m, 4H).

### Example 480

### {2-[4-(5-Amino-[1,3,4]oxadiazol-2-yl)-5-(3-chloro-4-fluoro-phenyl)-2-(cyclohexyl)-imidazol-1-yl]-4-chloro-phenyl}acetic acid

Example 479 (700 mg, 1.19 mmol) is dissolved in dioxane (12 mL) at it and treated with a 4M solution of HCl in dioxane (12 mL). The reaction mixture is allowed to stir at it for 20 h. The volatiles are removed under reduced pressure and the remaining crude product is purified by flash chromatography (SiO₂; DCM/MeOH; 0-10% MeOH). HPLC: _{A}t_{Ret}=4.57 min. ¹HNMR (CDCl₃) 7.46 (dd, 1H), 7.37-7.28 (m, 3H), 7.21-7.14 (m, 1H), 6.98 (dd, 1H), 3.17 (d, 2H), 2.34-2.23 (m, 1H), 1.89-1.58 (m, 7H), 1.35-1.04 (m, 3H).

### Example 482

### 2-{2-[4-(5-Amino-[1,3,4]oxadiazol-2-yl)-5-(3-chloro-4-fluoro-phenyl)-2-cyclohexyl-imidazol-1-yl]-4-chloro-phenyl}-N-methyl-acetamide

To Example 480 (39.2 mg, 0.074 mmol) dissolved in DMF (0.72 ml), methylamine hydrochloride (15 mg, 0.223 mmol), Et₃N (135 μl, 0.967 mmol), DMAP (4 mg, 0.032 mmol) and 2,4,6-tripropyl-1,3,5,2,4,6-trioxatriphosphorinane-2,4,6-trioxide ([68957-94-8] 50% in DMF; 87 μl, 0.149 mmol) are added. The mixture is stirred as described in Example 469; ES-MS: [M+1]⁺=543/545; HPLC: _{B}t_{Ret}=1.07 min; ¹H NMR (DMSO d₆) δ 7.83 (s, 1H), 7.80 (m, HN), 7.60 (d, 1H), 7.51 (d, 1H), 7.35 (d, 1H), 7.32 (t, 1H), 7.28 (m, 1H), 7.09 (s, H₂N), 3.3 (H₃C), 2.99 (d, 1H), 2.93 (d, 1H), 2.16 (m, 1H), 1.81 (m, 1H), 1.73-1.55, 1.41, 1.17 and 1.06 (4m, 9H).

### Example 500

### N-{3-[5-(3-Chloro-4-fluoro-phenyl)-1-(3-chloro-2-fluoro-phenyl)-5-methyl-[1,2,4]oxadiazol-3-yl)-1H-imidazol-2-yl]-phenyl}-acetamide

The title compound is prepared from Intermediate 466.1 and 3-acetamido-phenylboronic acid by Suzuki coupling as described for Example 7. ES-MS: [M+1]=541.8. HPLC: _{A}t_{Ret}=4.59 min.

### Example 501

### 1-(6-Carboxymethyl-3-chloro-2-fluoro-phenyl)-5-(3-chloro-4-fluoro-phenyl)-2-cyclohexyl-1H-imidazole-4-carboxylic acid benzyl ester

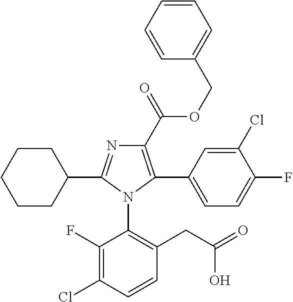

The title compound is synthesized by Suzuki Coupling of Intermediate 501.1 with 3-chloro-4-fluoro-phenyl boronic acid at 100° C. during 4¼ h as described in Example. 518; ES-MS: [M+1]⁺=599/601; HPLC: _{B}t_{Ret}=1.42 min; ¹H NMR (DMSO d₆) δ 12.7 (sb, HOOC), 7.72 (t, 1H), 7.43 (d, 1H), 7.27 (m, 5H), 7.15 (m, 2H), 7.12 (m 1H), 5.12 and 5.09 (2d, 2H), 3.33 (d, 1H), 3.16 (d, 1H), 2.18 (m, 1H), 1.6 (m, 6H), 1.38 (m, 1H), 1.12 (m, 3H).

### Intermediate 501.1

### 5-Bromo-1-(6-carboxymethyl-3-chloro-2-fluoro-phenyl)-2-cyclohexyl-1H-imidazole-4-carboxylic acid benzyl ester

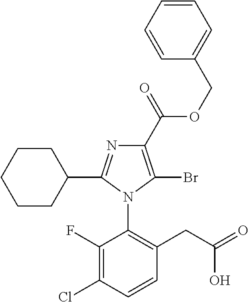

The title compound is synthesized by bromination of Intermediate 501.2 analogously to

### Intermediate 501.2

### 1-(6-Carboxymethyl-3-chloro-2-fluoro-phenyl)-2-cyclohexyl-1H-imidazole-4-carboxylic acid benzyl ester

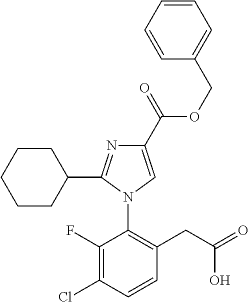

Hydroboration and oxidative work-up of Intermediate 501.3 analogously to the preparation of Intermediate 371.2 and purification by column chromatography [hexane/EtOAc 7:3→hexane/(EtOAc+1% HOAc) 7:3→1:19] gives the title compound; ES-MS: [M+1]⁺=471/473; HPLC: _{B}t_{Ret}=1.24 min.

### Intermediate 501.3

### 1-(3-Chloro-2-fluoro-6-trimethylsilanylethynyl-phenyl)-2-cyclohexyl-1H-imidazole-4-carboxylic acid benzyl ester

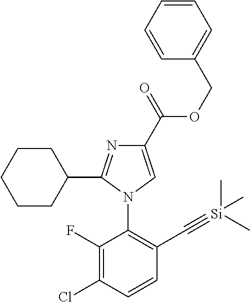

A solution of Intermediate 501.4 (22.4 g, 41.7 mmol) in Et₃N (434 ml) is degassed by repeated evacuation and flushing with N₂. Then Pd(OAc)₂ (721 mg, 3.21 mmol), CuI (238 mg, 1.25 mmol), PPh₃ (1.64 g, 6.26 mmol) and ethynyl-trimethyl-silane (15.6 ml, 113 mmol) are added. After 20 h stirring at rt, the suspension is worked up as described for Intermediate 395.3; ES-MS: [M+1]⁺=509/511; HPLC: _{B}t_{Ret}=1.59 min; ¹H NMR (DMSO d₆) δ 8.04 (s, 1H), 7.82 (t, 1H), 7.53 (d, 1H), 7.38 (m, 5H), 5.26 (s, 2H), 2.27 (m, 1H), 1.8-1.4 (m, 7H), 1.25-1.0 (m, 3H), 0.01 (s, 9H).

### Intermediate 501.4

### 1-(3-Chloro-2-fluoro-6-iodo-phenyl)-2-cyclohexyl-1H-imidazole-4-carboxylic acid benzyl ester

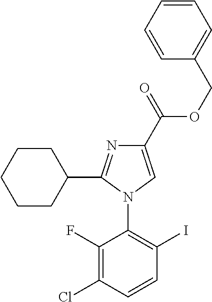

1-(3-Chloro-2-fluoro-6-iodo-phenyl)-2-cyclohexyl-1H-imidazole-4-carboxylic acid ethyl ester (Intermediate 395.4; 20 g, 41.7 mmol) is dissolved in toluene (400 ml). Then benzylalcohol (86 ml, 833 mmol) and titanium(IV)-isopropoxide (19.6 ml, 66 mmol) are added. The mixture is heated up and during 5 h. Toluene is partly distilled off via a Vigreux column. The residue is cooled to RT and diluted with 2 N aq. HCl and EtOAc. The aq. layer is separated off and extracted twice with EtOAc. The organic phases are washed with H₂O and brine, dried (Na₂SO₄) and concentrated. Kugelrohr destillation (200° C., HV) and crystallization from hexane gives the title compound; ES-MS: [M+1]⁺=539/541; HPLC: _{B}t_{Ret}=1.45 min; ¹H NMR (DMSO d₆) δ 8.07 (s, 1H), 7.92 (d, 1H), 7.65 (t, 1H), 7.45 (m, 2H), 7.40 (m, 2H), 7.35 (m, 1H), 5.30 and 5.27 (2d, 2H), 2.18 (m, 1H), 1.83 (m, 1H), 1.8-1.5 (m, 5H), 1.43 (m, 1H), 1.15 (m, 3H).

### Example 502

### 1-(6-tert-Butoxycarbonylmethyl-3-chloro-2-fluoro-phenyl)-5-(3-chloro-4-fluoro-phenyl)-2-cyclohexyl-1H-imidazole-4-carboxylic acid benzyl ester

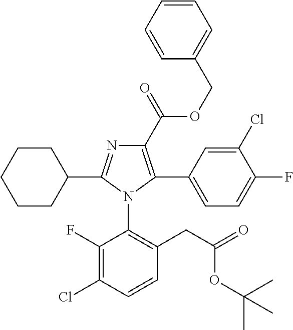

The title compound is synthesized from Example 501 as described in Example, 372; ES-MS: [M+1]⁺=655/657; HPLC: _{B}t_{Ret}=1.65 min; ¹H NMR (DMSO d₆) δ 7.73 (t, 1H), 7.44 (d, 1H), 7.27 (m, 5H), 7.13 (m, 3H), 5.12 and 5.09 (2d, 2H), 3.33 (d, 1H), 3.17 (d, 1H), 2.18 (m, 1H), 1.7-1.0 (4m, 10H), 1.34 (s, Me₃C).

### Example 514

### N-(2-Amino-1,1-dimethyl-ethyl)-2-{2-[4-(5-amino-[1,3,4]oxadiazol-2-yl)-5-(3-chloro-4-fluoro-phenyl)-2-cyclohexyl-imidazol-1-yl]-4-chloro-3-fluoro-phenyl}-acetamide

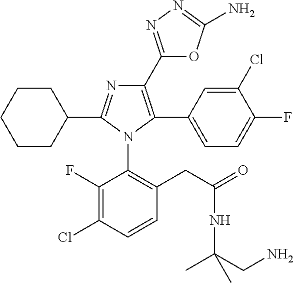

A mixture of [2-(2-{2-[4-(5-amino-[1,3,4]oxadiazol-2-yl)-5-(3-chloro-4-fluoro-phenyl)-2-cyclohexyl-imidazol-1-yl]-4-chloro-3-fluoro-phenyl}-acetylamino)-2-methyl-propyl]-carbamic acid tert-butyl ester (Ex. 513; 20 mg, 0.028 mmol), dioxane (0.5 ml) and HCl (0.5 ml; 4 N in dioxane) is stirred at RT for 30 min. Lyophylization and reversed phase chromatography gives the title compound; ES-MS: [M+1]⁺=618/620; ¹H NMR (DMSO d₆) δ 7.74 (1, 1H), 7.49 (m, 2H), 7.36 (m, 2H), 7.21 (m, 1H), 7.13 (s, H₂N), 3.17 (d, 1H), 3.13 (d, 1H), 2.6 (m, 2H), 2.23 (m, 1H), 1.9-1.0 (5m, 10H), 1.13 and 1.12 (2s, 2H₃C).

### Example 518

### 1-(6-Carboxymethyl-3-chloro-2-fluoro-phenyl)-5-(3-chloro-4-fluoro-phenyl)-2-cycloheptyl-1H-imidazole-4-carboxylic acid benzyl ester

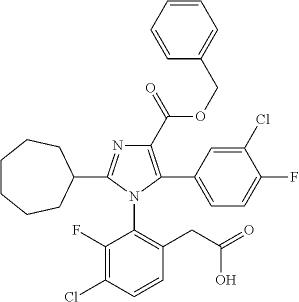

A mixture of Intermediate 518.1 (4.56 g, 8.09 mmol), degased dioxane (67 ml), degased H₂0 (33 ml), K₃PO₄ (6.7 g, 31.5 mmol), 3-chloro-4-fluoro-phenyl boronic acid (2.5 g, 14.3 mmol) and Pd(PPh₃)₄ (925 mg, 0.80 mmol) is stirred for 3½ h at 100° C. After cooling the mixture to ambient temperature, it is diluted with EtOAc, water and citric acid, the aq. layer is separated off and extracted twice with EtOAc. The organic phases are washed with H₂O and brine, dried (Na₂SO₄) and concentrated. Combi Flash chromatography [hexane/EtOAc 9:1→hexane/(EtOAc+1% HOAc) 9:1→EtOAc+1% HOAc)] gives the title compound. ES-MS: [M+1]⁺=613/615; HPLC: _{B}t_{Ret}=1.45 min; ¹H NMR (DMSO d₆) δ 12.8 (sb, HOOC), 7.74 (t, 1H), 7.46 (m, 1H), 7.31 (d, 1H), 7.29 (m, 3H), 7.25 (t, 1H), 7.15 (m 3H), 5.14 and 5.10 (2d, 2H), 3.36 (d, 1H), 3.17 (d, 1H), 2.38 (m, 1H), 1.91, 1.80, 1.63, 1.46, 1.33 and 1.12 (5m, 12H).

### Intermediate 518.1

### 5-Bromo-1-(6-carboxymethyl-3-chloro-2-fluoro-phenyl)-2-cycloheptyl-1H-imidazole-4-carboxylic acid benzyl ester

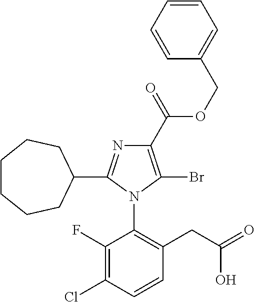

The title compound is synthesized by bromination of Intermediate 518.2 analogously to the preparation of Intermediate 6.1; ES-MS: [M+1]⁺=563/565; HPLC: _{B}t_{Ret}=1.40 min.

### Intermediate 518.2

### 1-(6-Carboxymethyl-3-chloro-2-fluoro-phenyl)-2-cycloheptyl-1H-imidazole-4-carboxylic acid benzyl ester

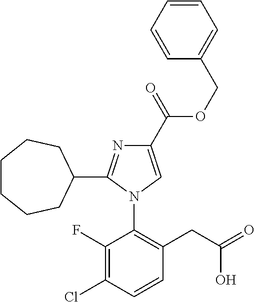

Hydroboration and oxidative work-up of Intermediate 518.3 analogously to the preparation of Intermediate 371.2 and purification by column chromatography [hexane/EtOAc 7:3→hexane/(EtOAc+1% HOAc) 3:2→1:19] gives the title compound; ES-MS: [M+1]⁺=485/487; HPLC: _{B}t_{Ret}=1.28 min.

### Intermediate 518.3

### 1-(3-Chloro-2-fluoro-6-trimethylsilanylethynyl-phenyl)-2-cycloheptyl-1H-imidazole-4-carboxylic acid benzyl ester

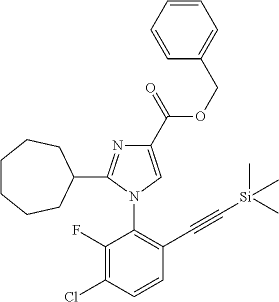

A solution of Intermediate 518.4 (7.1 g, 12.8 mmol) in Et₃N (134 ml) is degassed by repeated evacuation and flushing with N₂. Then Pd(OAc)₂ (222 mg, 0.99 mmol), CuI (74 mg, 0.385 mmol), PPh₃ (505 mg, 1.927 mmol) and ethynyl-trimethyl-silane (4.9 ml, 34.7 mmol) are added. After 22 h stirring at rt, the suspension is worked up as described for Intermediate 395.3; ES-MS: [M+1]⁺=523/525; HPLC: _{B}t_{Ret}=1.64 min; ¹H NMR (DMSO d₆) δ 8.05 (s, 1H), 7.82 (t, 1H), 7.53 (d, 1H), 7.38 (m, 5H), 5.26 (s, 2H), 2.46 (m, 1H), 1.78, 1.63, 1.45 and 1.23 (4m, 12H), −0.01 (s, 9H).

### Intermediate 518.4

### 1-(3-Chloro-2-fluoro-6-iodo-phenyl)-2-cycloheptyl-1H-imidazole-4-carboxylic acid benzyl ester

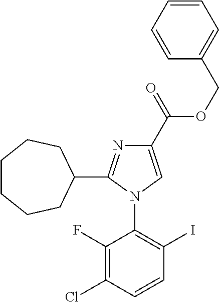

A solution of Intermediate 518.5 (7.2 g, 14.7 mmol) in toluene (141 ml), benzylalcohol (30.2 ml, 293 mmol) and titanium(IV)-isopropoxide (6.91 ml, 23.3 mmol) is heated up to boiling temperature. During 4 h, toluene is partly distilled off via a Vigreux column. The residue is worked up as described for Intermediate 501.4, giving the title compound; ES-MS: [M+1]⁺=553/555; HPLC: _{B}t_{Ret}=1.49 min; ¹H NMR (DMSO d₆) δ 8.05 (s, 1H), 7.91 (d, 1H), 7.62 (t, 1H), 7.43 (m, 2H), 7.4-7.3 (m, 3H), 5.28 and 5.25 (2d, 2H), 2.36 (m, 1H), 1.86 (m, 2H), 1.75-1.55 (m, 4H), 1.46 (m, 4H), 1.30 (m, 1H), 1.19 (m, 1H).

### Intermediate 518.5

### 1-(3-Chloro-2-fluoro-6-iodo-phenyl)-2-cycloheptyl-1H-imidazole-4-carboxylic acid ethyl ester

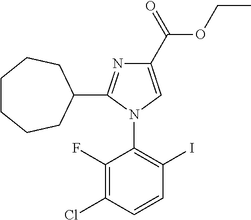

The title compound is synthesized by dehydration of Intermediate 518.6 analogously to the preparation of Intermediate 6.2; ES-MS: [M+1]⁺=491/493; HPLC: _{B}t_{Ret}=1.38 min.

### Intermediate 518.6

### rac. 1-(3-Chloro-2-fluoro-6-iodo-phenyl)-2-cycloheptyl-4-hydroxy-4,5-dihydro-1H-imidazole-4-carboxylic acid ethyl ester

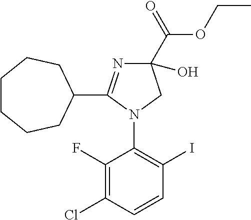

The title compound is synthesized by reaction of ethyl bromopyruvate and Intermediate 518.7 analogously to the preparation of Intermediate 6.3; ES-MS: [M+1]⁺=509/511.

### Intermediate 518.7

### N-(3-Chloro-2-fluoro-6-iodo-phenyl)-cycloheptanecarboxamidine

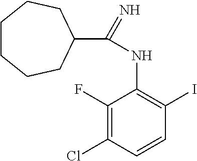

The reaction mixture of the addition of Intermediate 395.7 to cycloheptylcarbonitrile as described for Intermediate 395.6 is poured into a mixture of MeOH/DCM 1:2 and stirred for 1 h at rt. Then SiO₂ is added, the mixture concentrated and the resulting pouder applied to a chromatography column (SiO₂). Eluation with (CH₂Cl₂/hexane 1:1)/MeOH 99:1→19:1 gives the title compound; ES-MS: [M+1]⁺=395/397; HPLC: _{B}t_{Ret}=0.94.

### Example 519

### 1-(6-tert-Butoxycarbonylmethyl-3-chloro-2-fluoro-phenyl)-5-(3-chloro-4-fluoro-phenyl)-2-cycloheptyl-1H-imidazole-4-carboxylic acid benzyl ester

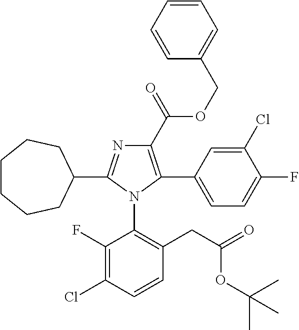

To Example 518 (2.98 g, 4.86 mmol) in DCM (23 ml), a solution of 2,2,2-trichloro-acetimidic acid tert-butyl ester (3.5 ml, 19.4 mmol) in cyclohexane (18 ml) is added, followed by BF₃Et₂O (98 μl, 0.78 mmol). After 28 h at RT, the reaction mixture is worked up as described in Example. 372. ES-MS: [M+1]⁺=6691671; HPLC: _{B}t_{Ret}=1.68 min; ¹H NMR (DMSO d₆) δ 7.76 (t, 1H), 7.48 (m, 1H), 7.29 (m, 4H), 7.25 (t, 1H), 7.14 (m, 3H), 5.14 and 5.10 (2d, 2H), 3.35 (d, 1H), 3.19 (d, 1H), 2.40 (m, 1H), 1.91 (m, 1H), 1.78 (m, 1H), 1.7-1.1 (4m, 10H), 1.36 (s, Me₃C).

### Example 520

### 1-(6-tert-Butoxycarbonylmethyl-3-chloro-2-fluoro-phenyl)-5-(3-chloro-4-fluoro-phenyl)-2-cycloheptyl-1H-imidazole-4-carboxylic acid

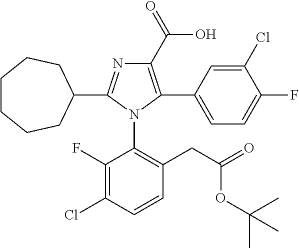

A mixture of 1-(6-tert-butoxycarbonylmethyl-3-chloro-2-fluoro-phenyl)-5-(3-chloro-4-fluoro-phenyl)-2-cycloheptyl-1H-imidazole-4-carboxylic acid benzyl ester (1.0 g, 1.49 mmol), THF (127 ml), 1,2-dichlorbenzene (127 μl) and Pd/C (5% Engelhard 4522; 127 mg) is hydrogenated under normal pressure at RT for 30 min. The catalyst is filtered off and the filtrate concentrated. Reversed phase chromatography gives the title compound; ES-MS: [M+1]⁺=579/581; ¹H NMR (DMSO d₆) δ 12.4 (s, HOOC), 7.76 (t, 1H), 7.44 (m, 1H), 7.30 (m, 2H), 7.13 (m, 1H), 3.32 (d, 1H), 3.16 (d, 1H), 2.39 (m, 1H), 1.90 (m, 1H), 1.77 (m, 1H), 1.7-1.1 (4m, 10H), 1.37 (s, Me₃C).

TABLE 2
Mdm2 and Mdm4 inhibitory activity of representative compounds
of the present invention

| | IC₅₀ (□M) | | |
|---|---|---|---|
| | of p53-Hdm2 | IC₅₀ (□M) of | IC₅₀ (□M) of |
| | inhibition | p53-Hdm2 | p53-Hdm4 |
| | Fluorescence | inhibition | inhibition |
| Example | polarisation assay | (TR-FRET) Assay | (TR-FRET) Assay |
| 1 | 2.24 | | |
| 2 | 0.35 | | |
| 4 | 0.96 | | |
| 5 | 0.097 | | |
| 13 | 3.27 | | |
| 14 | 2.33 | | |
| 17 | | 0.0016 | 7.3 |
| 18 | | 0.0074 | 39.5 |
| 19 | | 0.0026 | 10.0 |
| 20 | 12.7 | | |
| 21 | 13.5 | | |
| 25 | | 0.060 | 62.6 |
| 28 | 52.0 | | |
| 30 | | 0.048 | 65.4 |
| 31 | | 0.032 | 49.7 |
| 47 | 3.57 | | |
| 48 | 0.048 | | |
| 50 | 0.85 | | |
| 52 | 0.96 | | |
| 53 | 0.82 | | |
| 54 | 0.28 | | |
| 66 | 0.20 | | |
| 68 | 3.07 | | |
| 69 | 0.16 | | |
| 93 | 1.74 | | |
| 110 | 36.0 | | |
| 111 | 2.79 | | |
| 112 | 14.8 | | |
| 113 | 21.7 | | |
| 122 | 28.5 | | |
| 123 | 56.26 | | |
| 137 | 0.33 | | |
| 138 | 3.00 | | |
| 156 | 0.119 | | |
| 182 | 1.80 | | |
| 184 | 0.64 | | |
| 193 | 0.45 | | |
| 195 | | 0.025 | 49.1 |
| 197 | 0.085 | | |
| 202 | 0.32 | | |
| 212 | | 0.0051 | 47.0 |
| 214 | 0.096 | | |
| 218 | 0.122 | | |
| 226 | | 0.0027 | 71.26 |
| 237 | 1.33 | | |
| 241 | 0.45 | | |
| 246 | 6.33 | | |
| 248 | | 1.229 | 52.8 |
| 252 | | 0.056 | 56.77 |
| 254 | | 0.0115 | 48.3 |
| 257 | 0.164 | | |
| 259 | | 0.0038 | 28.44 |
| 263 | 0.160 | | |
| 266 | | 0.015 | 56.29 |
| 268 | | 0.0073 | 20.32 |
| 306 | | 0.0067 | 31.45 |
| 334 | | 0.0033 | 69.70 |
| 344 | | 0.0089 | 27.2 |
| 356 | 0.67 | | |
| 362 | | 0.091 | 56.9 |
| 364 | 0.15 | | |
| 387 | 0.32 | | |
| 397 | | 0.0015 | 24.11 |
| 409 | 0.15 | | |
| 413 | 0.098 | | |
| 415 | 0.56 | | |
| 423 | 0.17 | | |
| 425 | | 0.012 | 33.96 |
| 472 | | 0.0031 | 31.6 |
| 479 | | 0.005 | 39 |
| 482 | | 0.003 | 25 |
| 483 | | 0.002 | 22 |
| 491 | 0.26 | | |
| 497 | | 0.008 | 44.98 |
| 501 | | 0.062 | 14.7 |
| 502 | | 1.080 | |
| 503 | | 0.007 | 50 |
| 504 | | 0.005 | 49 |
| 505 | | 0.002 | 22 |
| 506 | | 0.007 | 21 |
| 507 | | 0.003 | 14 |
| 508 | | 0.002 | 12 |
| 509 | | 0.003 | 21 |
| 510 | | 0.009 | 18 |
| 511 | | 0.002 | 45 |
| 512 | | 0.007 | 36 |
| 513 | | 0.002 | 29 |
| 514 | | 0.020 | |
| 515 | | 0.006 | 41 |
| 516 | | 0.004 | |
| 517 | | 0.005 | 41 |
| 518 | | 0.081 | 12 |
| 520 | | 0.001 | 14 |

1. A compound of the formula (I), or a tautomer or a N-oxide or a pharmaceutically acceptable salt thereof, 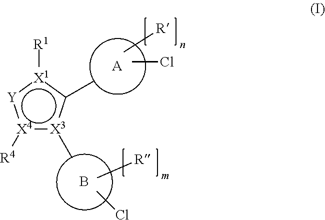
wherein
X¹, X³ and X⁴ are independently C or N,
Y is C—H, N—H or N,
wherein the total number of nitrogen atoms represented by X¹, X³, X⁴ and Y is 1 or 2;
rings A and B are independently selected from phenyl or pyridyl, wherein the Chlorine substituents are independently in the 3 or 4 position;
R¹ is selected from the group consisting of
cyano-
cyano-methyl-
carboxy-C₁-C₂-alkyl-
carboxyl-
C₁-C₇-alkoxy-carbonyl-
amino-carbonyl-
N—C₁-C₇-alkyl-amino-carbonyl-
N,N-di-C₁-C₇-alkyl-amino-carbonyl-
N-hydroxyl-amino-carbonyl-
N-hydroxyl-N—C₁-C₇-alkyl-amino-carbonyl-
N—C₁-C₇-alkoxy-amino-carbonyl-
N—C₁-C₇-alkoxy-N—C₁-C₇-alkyl-amino-carbonyl-
C₁-C₇-alkyl-carbonyl-amino-C₁-C₂-alkyl-
heterocyclyl-
C₁-C₇-alkyl-carbonyl-
formyl-
hydroxy-C₁-C₂-alkyl-
heterocyclyl-carbonyl-
S—C₁-C₇-alkyl-sulfonimidoyl-
S—C₁-C₇-alkyl-N—C₁-C₇-alkyl-sulfonimidoyl-
S—C₁-C₇-alkyl-N—C₁-C₇-alkyl-sulfonimidoyl-
C₁-C₇-alkyl-sulfonyl-
amino-
S—C₁-C₇-alkyl-sulfoximino-
N—C₁-C₇-alkyl-amino-
N,N-di-C₁-C₇-alkyl-amino-
C₁-C₇-alkoxy-carbonyl-amino-
N—(C₁-C₇-alkoxy-carbonyl)-N—C₁-C₇-alkyl-amino-
C₁-C₇-alkyl-carbonyl-amino-
N—(C₁-C₇-alkyl-carbonyl)-N—C₁-C₇-alkyl-amino-
amino-sulfonyl-
N—C₁-C₇-alkyl-amino-sulfonyl-
N,N-di-C₁-C₇-alkyl-amino-sulfonyl-
hydrazinocarbonyl-
N—C₁-C₇-alkyl-hydrazino-carbonyl-
N,N-di-C₁-C₇-alkyl-hydrazino-carbonyl-
N—C₁-C₇-alkyl-hydrazino-carbonyl-
N,N-di-C₁-C₇-alkyl-hydrazino-carbonyl-
N,N-di-C₁-C₇-alkyl-N′—C₁-C₇-alkyl-hydrazino-carbonyl-
C₁-C₇-alkyl-carbonyl-hydrazino-carbonyl-
C₁-C₇-alkyl-carbonyl-N—C₁-C₇-alkyl-N′—C₁-C₇-alkyl-hydrazino-carbonyl-
phosphonyl-
C₁-C₇-alkyl-phosphonyl-
di-C₁-C₇-alkyl-phosphonyl-,
wherein C₁-C₇-alkyl or C₁-C₇-alkoxy groups are unsubstituted or substituted by 1-4 substituents selected from:
amino-
N—C₁-C₇-alkyl-amino-
N,N-di-C₁-C₇-alkyl-amino-
N-aryl-amino-
N-aryl-N—C₁-C₇-alkyl-amino-
heterocyclyl-
heterocyclyl-carbonyl-
C₃-C₁₀-cycloalkyl-
hydroxy-
cyano-
halogen-
halo-C₁-C₇-alkyl-
C₁-C₇-alkoxy-
C₁-C₇-alkyl-carbonyl-amino-
N—(C₁-C₇-alkyl-carbonyl)-N—C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
amino-carbonyl-amino-
N—C₁-C₇-alkyl-amino-carbonyl-amino-
N,N-di-C₁-C₇-alkyl-amino-carbonyl-amino-
amino-carbonyl-N′—(C₁-C₇-alkyl)-amino-
N—C₁-C₇-alkyl-amino-carbonyl-N′—(C₁-C₇-alkyl)-amino-
N,N-di-C₁-C₇-alkyl-amino-carbonyl-N′—(C₁-C₇-alkyl)-amino-
carboxyl-
C₁-C₇-alkoxy-carbonyl-
aryl- and
and wherein C₁-C₇-alkyl and C₁-C₇-alkoxy groups as part of these substituents can be further substituted as described above for C₁-C₇-alkyl and C₁-C₇-alkoxy;
and wherein heterocyclic groups are unsubstituted or substituted by 1-4 substituents selected from:
amino-
N—C₁-C₇-alkyl-amino-
N,N-di-C₁-C₇-alkyl-amino-
heterocyclyl-
C₃-C₁₀-cycloalkyl-
cyano-
halogen-
halo-C₁-C₇-alkyl-
C₁-C₇-alkoxy-
C₁-C₇-alkoxy-C₁-C₇-alkyl-
protected hydroxy-C₁-C₇-alkyl-
C₁-C₇-alkyl-carbonyl-amino-
N—(C₁-C₇-alkyl-carbonyl)-N—C₁-C₇-alkyl-amino-
N,N-di-C₁-C₇-alkyl-amino-C₁-C₇-alkyl-amino-
C₁-C₇-alkoxy-C₁-C₇-alkyl-amino-
aryl-C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
carboxyl-
C₁-C₇-alkoxy-carbonyl-
C₁-C₇-alkyl-
oxo (O═)
thiono (S═)
wherein C₁-C₇-alkyl and C₁-C₇-alkoxy as part of these substituents can be further substituted as described above for C₁-C₇-alkyl and C₁-C₇-alkoxy.
R⁴ is selected from the group consisting of
substituted C₁-alkyl-
C₂-C₇-alkyl-
aryl-
heteroaryl-
heterocyclyl-
C₃-C₁₀-cycloalkyl-
aryl-C₁-C₇-alkyl-
heteroaryl-C₁-C₇-alkyl-
heterocyclyl-C₁-C₇-alkyl-
C₃-C₁₀-cycloalkyl-C₁-C₇-alkyl-
wherein substituents R⁴ are unsubstituted or substituted by 1-3 substituents selected from
hydroxy-
C₁-C₇-alkoxy-
C₁-C₇-alkoxy-carbonyl-
halogen-
halo-C₁-C₇-alkyl-
nitro-
C₁-C₇-alkyl-carbonyl-
formyl-
C₁-C₇-alkyl-
amino-
N—C₁-C₇-alkyl-amino-
N,N-di-C₁-C₇-alkyl-amino-
C₁-C₇-alkyl-carbonyl-amino-
N—(C₁-C₇-alkyl-carbonyl)-N—C₁-C₇-alkyl-amino-
hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N—C₁-C₇-alkyl-hydrazino-carbonyl-C₁-C₇-alkyl-amino-
N,N-di-C₁-C₇-alkyl-hydrazino-carbonyl-C₁-C₇-alkyl-amino-
N,N-di-C₁-C₇-alkyl-N′—C₁-C₇-alkyl-hydrazino-carbonyl-C₁-C₇-alkyl-amino-
C₁-C₇-alkyl-carbonyl-hydrazino-carbonyl-C₁-C₇-alkyl-amino-
C₁-C₇-alkyl-carbonyl-N—C₁-C₇-alkyl-N′—C₁-C₇-alkyl-hydrazino-carbonyl-C₁-C₇-alkyl-amino-
hydrazinocarbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N—C₁-C₇-alkyl-hydrazino-carbonyl-C₁-C₇-alkyl-N—(C₁-C₇-alkyl)-amino-
N,N-di-C₁-C₇-alkyl-hydrazino-carbonyl-C₁-C₇-alkyl-N—(C₁-C₇-alkyl)-amino-
N,N-di-C₁-C₇-alkyl-N′—C₁-C₇-alkyl-hydrazino-carbonyl-C₁-C₇-alkyl-N—(C₁-C₇-alkyl)-amino-
C₁-C₇-alkyl-carbonyl-hydrazino-carbonyl-C₁-C₇-alkyl-N—(C₁-C₇-alkyl)-amino-
C₁-C₇-alkyl-carbonyl-N—C₁-C₇-alkyl-N′—C₁-C₇-alkyl-hydrazino-carbonyl-C₁-C₇-alkyl-
N—(C₁-C₇-alkyl)-amino-
tert-butyl-diphenyl-silanyloxy-
heterocyclyl-
protected hydroxy-
wherein C₁-C₇-alkyl or C₁-C₇-alkoxy groups as part of substituents for R⁴ as defined above are unsubstituted or substituted by 1-4 groups, independently selected from:
amino-
N—C₁-C₇-alkyl-amino-
N,N-di-C₁-C₇-alkyl-amino-
N-aryl-amino-
N-aryl-N—C₁-C₇-alkyl-amino-
heterocyclyl-
heterocyclyl-carbonyl-
C₃-C₁₀-cycloalkyl-
hydroxy-
cyano-
halogen-
halo-C₁-C₇-alkyl-
C₁-C₇-alkoxy-
C₁-C₇-alkyl-carbonyl-amino-
N—(C₁-C₇-alkyl-carbonyl)-N—C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
amino-carbonyl-amino-
N—C₁-C₇-alkyl-amino-carbonyl-amino-
N,N-di-C₁-C₇-alkyl-amino-carbonyl-amino-
amino-carbonyl-N′—(C₁-C₇-alkyl)-amino-
N—C₁-C₇-alkyl-amino-carbonyl-N′—(C₁-C₇-alkyl)-amino-
N,N-di-C₁-C₇-alkyl-amino-carbonyl-N′—(C₁-C₇-alkyl)-amino-
carboxyl-
C₁-C₇-alkoxy-carbonyl-
aryl- and
and wherein C₁-C₇-alkyl and C₁-C₇-alkoxy groups as part of these substituents can be further substituted as described above for C₁-C₇-alkyl and C₁-C₇-alkox_{Y};
and wherein heterocyclyl as part of substituents for R⁴ as defined above is unsubstituted or substituted by 1-4 groups, independently selected from:
amino-
N—C₁-C₇-alkyl-amino-
N,N-di-C₁-C₇-alkyl-amino-
heterocyclyl-
C₃-C₁₀-cycloalkyl-
cyano-
halogen-
halo-C₁-C₇-alkyl-
C₁-C₇-alkoxy-
C₁-C₇-alkoxy-C₁-C₇-alkyl-
protected hydroxy-C₁-C₇-alkyl-
C₁-C₇-alkyl-carbonyl-amino-
N—(C₁-C₇-alkyl-carbonyl)-N—C₁-C₇-alkyl-amino-
N,N-di-C₁-C₇-alkyl-amino-C₁-C₇-alkyl-amino-
C₁-C₇-alkoxy-C₁-C₇-alkyl-amino-
aryl-C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
carboxyl-
C₁-C₇-alkoxy-carbonyl-
C₁-C₇-alkyl-
oxo (O═)
thiono (S═)
and wherein C₁-C₇-alkyl and C₁-C₇-alkoxy groups as part of these substituents can be further substituted as described above for C₁-C₇-alkyl and C₁-C₇-alkox_{Y};
R′ and R″ are independently selected from the group consisting of:
hydroxy-
C₁-C₇-alkoxy-
halogen-
halo-C₁-C₇-alkyl-
cyano-
C₁-C₇-alkyl-carbonyl-
formyl-
C₁-C₇-alkyl-
amino-carbonyl-
N—C₁-C₇-alkyl-amino-carbonyl-
N,N-di-C₁-C₇-alkyl-amino-carbonyl-
heterocyclyl-
N-(hydroxy-C₁-C₇-alkyl)-amino-carbonyl-C₁-C₇-alkyl-
N—(C₁-C₇-alkoxy-C₁-C₇-alkyl)-amino-carbonyl-C₁-C₇-alkyl-
N-(hydroxy-C₁-C₇-alkyl)-N—C₁-C₇-alkyl-amino-carbonyl-C₁-C₇-alkyl-
N—(C₁-C₇-alkoxy-C₁-C₇-alkyl)-N—C₁-C₇-alkyl-amino-carbonyl-C₁-C₇-alkyl-
C₁-C₇-alkoxy-carbonyl-
C₁-C₇-alkyl-carbonyl-amino-
carboxyl- and
and where A and B, or A or B are pyridyl, R′ and R″ may also be independently selected from ═O, to form the group 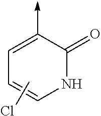
which may be further substituted with R′ and R″ as described above;
wherein C₁-C₇-alkyl or C₁-C₇-alkoxy groups as part of substituents on R′ or R″ as defined above are unsubstituted or substituted by 1-4 groups, independently selected from:
amino-
N—C₁-C₇-alkyl-amino-
N,N-di-C₁-C₇-alkyl-amino-
N-aryl-amino-
N-aryl-N—C₁-C₇-alkyl-amino-
heterocyclyl-
heterocyclyl-carbonyl-
C₃-C₁₀-cycloalkyl-
hydroxy-
cyano-
halogen-
halo-C₁-C₇-alkyl-
C₁-C₇-alkoxy-
C₁-C₇-alkyl-carbonyl-amino-
N—(C₁-C₇-alkyl-carbonyl)-N—C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
amino-carbonyl-amino-
N—C₁-C₇-alkyl-amino-carbonyl-amino-
N,N-di-C₁-C₇-alkyl-amino-carbonyl-amino-
amino-carbonyl-N′—(C₁-C₇-alkyl)-amino-
N—C₁-C₇-alkyl-amino-carbonyl-N′—(C₁-C₇-alkyl)-amino-
N,N-di-C₁-C₇-alkyl-amino-carbonyl-N′—(C₁-C₇-alkyl)-amino-
carboxyl-
C₁-C₇-alkoxy-carbonyl-
C₁-C₇-alkoxy-carbonyl-amino-
aryl-
aryl-amino-carbonyl-, wherein said aryl is optionally substituted as described herein,
C₃-C₁₀-cycloalkyl-amino-carbonyl-
heterocyclyl-amino-carbonyl- and
and wherein C₁-C₇-alkyl and C₁-C₇-alkoxy groups as part of these substituents can be further substituted as described above for C₁-C₇-alkyl and C₁-C₇-alkox_{Y};
and wherein heterocyclyl as part of substituents on R′ or R″ as defined above is unsubstituted or substituted by 1-4 groups, independently selected from:
amino-
N—C₁-C₇-alkyl-amino-
N,N-di-C₁-C₇-alkyl-amino-
heterocyclyl-
C₃-C₁₀-cycloalkyl-
cyano-
halogen-
halo-C₁-C₇-alkyl-
C₁-C₇-alkoxy-C₁-C₇-alkyl-
protected hydroxy-C₁-C₇-alkyl-
C₁-C₇-alkoxy-
C₁-C₇-alkyl-carbonyl-amino-
N—(C₁-C₇-alkyl-carbonyl)-N—C₁-C₇-alkyl-amino-
N,N-di-C₁-C₇-alkyl-amino-C₁-C₇-alkyl-amino-
C₁-C₇-alkoxy-C₁-C₇-alkyl-amino-
aryl-C₁-C₇-alkyl-amino-
C₁-C₇-alkyl-carbonyl-
formyl-
amino-carbonyl-
N—C₁-C₇-alkyl-amino-carbonyl-
N,N-di-C₁-C₇-alkyl-amino-carbonyl-
carboxyl-
C₁-C₇-alkoxy-carbonyl-
C₁-C₇-alkyl-
oxo (O═)
thiono (S═)
and wherein C₁-C₇-alkyl and C₁-C₇-alkoxy groups as part of these substituents can be further substituted as described above for C₁-C₇-alkyl and C₁-C₇-alkox_{Y};
and
n and m are independently 0 to 2,
wherein “aryl” means an aromatic hydrocarbon group having 6-20 carbon atoms in the ring portion;
“heterocyclyl” or “heterocyclic” means an unsaturated, saturated or partially saturated ring or ring system containing at least one heteroatom selected from N, O and S, where the N and S can also optionally be oxidized to various oxidation states, and which include fused, bridged rings and spirocyclic rings;
“cycloalkyl” means saturated or partially unsaturated monocyclic, bicyclic or tricyclic hydrocarbon groups of 3-12 carbon atoms;
“protected hydroxy” refers to a hydroxy functionality bearing a hydroxy protecting group;
“heteroaryl” means an unsaturated heterocyclyl ring or ring system carrying the highest possible number of conjugated double bonds in the ring(s);
aryl is unsubstituted or substituted by 1-4 substituents, independently selected from
C₁-C₇-alkyl-
halo-C₁-C₇-alkyl-
hydroxy-C₁-C₇-alkyl-
C₃-C₁₀-cycloalkyl-
halogen-
hydroxy-
protected hydroxy-
C₁-C₇-alkoxy-
C₁-C₇-alkoxy-carbonyl-
C₁-C₇-alkyl-carbonyl-oxy-
aryl-carbonyl-oxy-
aryl-oxy-
heterocyclyl-oxy-
amino-
N—C₁-C₇-alkyl-amino-
N—C₁-C₇-alkyl-amino-N—C₁-C₇-alkyl-amino-
N—C₁-C₇-alkyl-amino-N,N-di-C₁-C₇-alkyl-amino-
N,N-di-C₁-C₇-alkyl-amino
N,N-di-C₁-C₇-alkyl-amino-N—C₁-C₇-alkyl-amino
N,N-di-C₁-C₇-alkyl-amino-N,N-di-C₁-C₇-alkyl-amino
C₁-C₇-alkoxy-N—C₁-C₇-alkyl-amino-
C₁-C₇-alkoxy-N,N-di-C₁-C₇-alkyl-amino-
aryl-C₁-C₇-alkyl-amino-
thio-
C₁-C₇-alkyl-thio-
aryl-thio-
aryl-C₁-C₇-alkyl-
nitro-
cyano-
carboxy-
C₁-C₇-alkoxy-carbonyl-
C₁-C₇-alkyl-carbonyl-
formyl-
amino-carbonyl-
C₁-C₇-alkyl-carbonyl-amino-
N—(C₁-C₇-alkyl-carbonyl)-N—C₁-C₇-alkyl-amino-
N—C₁-C₇-alkyl-amino-carbonyl-
N,N-di-C₁-C₇-alkyl-amino-carbonyl-
C₁-C₇-alkyl-sulfinyl-
C₁-C₇-alkyl-sulfonyl-
amino-sulfonyl-
N—C₁-C₇-alkyl-amino-sulfonyl-
N,N-di-C₁-C₇-alkyl-amino-sulfonyl-
aryl-
trimethylsilanyl-ethoxymethyl
heterocyclyl-;
heterocyclyl, heterocyclic and heteroaryl are each independently unsubstituted or substituted by 1-4 substituents selected from the group of substituents described for aryl, and can also optionally be substituted by substituents independently selected from oxo (O═) and thiono (S═).

2. The compound of claim 1, or a tautomer or a N-oxide or a pharmaceutically acceptable salt thereof, wherein the compound of formula (I) is a compound of formula

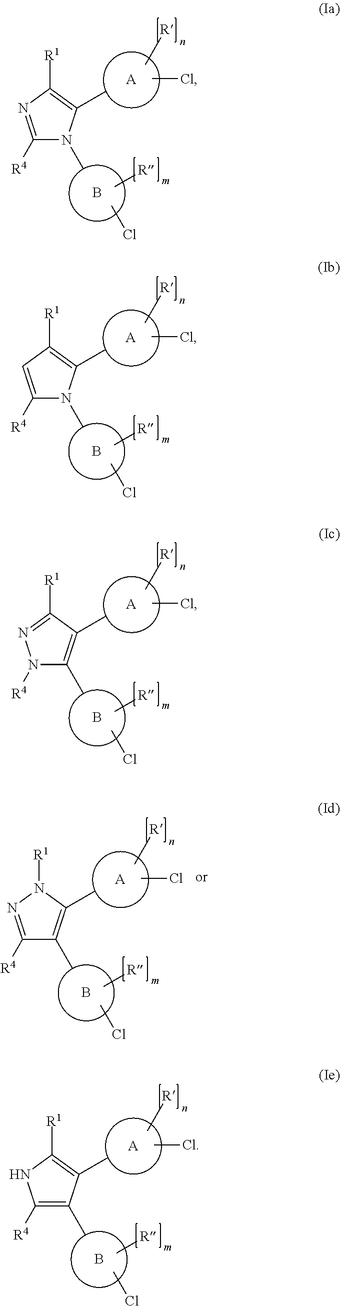

3. The compound according to claim 1, or a tautomer or a N-oxide or a pharmaceutically acceptable salt thereof, wherein ring A is phenyl and the chlorine substituent is in the 3 position, and wherein ring B is phenyl and the chlorine substituent is in the 3 position.

4. The compound according to claim 1 or a tautomer or a N-oxide or a pharmaceutically acceptable salt thereof, wherein the compound of formula (I) is a compound of formula (Ia):

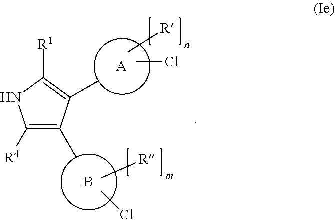

5. The compound according to claim 1, or a tautomer or a N-oxide or a pharmaceutically acceptable salt thereof, wherein R¹ is selected from the group consisting of:
cyano-
carboxyl-
C₁-C₇-alkoxy-carbonyl-
amino-carbonyl-
N—C₁-C₇-alkyl-amino-carbonyl-
N,N-di-C₁-C₇-alkyl-amino-carbonyl-
N,N-di-C₁-C₇-alkyl-amino-C₁-C₇-alkyl-amino-carbonyl-
N,N-di-C₁-C₇-alkyl-amino-C₁-C₇-alkyl-N′—C₁-C₇-alkyl-amino-carbonyl-
N-(heterocyclyl-C₁-C₇-alkyl)-amino-carbonyl-
N-(cycloalkyl-C₁-C₇-alkyl)-amino-carbonyl-
N-hydroxyl-amino-carbonyl-
N—C₁-C₇-alkoxy-amino-carbonyl-
N-benzyloxy-amino-carbonyl-
benzyloxycarbonyl
heterocyclyl-
heterocyclyl-C₁-C₇-alkyl-
hydroxy-C₁-C₇-alkyl-
hydroxy-C₁-C₇-alkyl-carbonyl-
C₁-C₇-alkyl-carbonyl-
cyano-C₁-C₇-alkyl-
carboxyl-C₁-C₇-alkyl-
C₁-C₇-alkoxy-C₁-C₇-alkyl-
heterocyclyl-carbonyl-
C₁-C₇-alkyl-carbonyl-amino-C₁-C₇-alkyl-
hydroxy-C₁-C₇-alkyl-amino-carbonyl-
C₁-C₇-alkyl-carbonyl-C₁-C₇-alkyl-amino-carbonyl-
N—(C₁-C₇-alkoxy-C₁-C₇-alkyl)-amino-sulfonyl-
S—C₁-C₇-alkyl-sulfonimidoyl-
N,N-di-C₁-C₇-alkyl-amino-C₁-C₇-alkyl-amino-carbonyl-C₁-C₇-alkyl-amino-carbonyl-
C₁-C₇-alkoxy-carbonyl-C₁-C₇-alkyl-amino-carbonyl-
carboxyl-C₁-C₇-alkyl-amino-carbonyl-
C₁-C₇-alkyl-sulfonyl-
amino-
N,N-di-C₁-C₇-alkyl-amino-
C₁-C₇-alkoxy-carbonyl-amino-
C₁-C₇-alkyl-carbonyl-amino-
amino-sulfonyl-
N—C₁-C₇-alkyl-amino-sulfonyl-
C₁-C₇-alkoxy-C₁-C₇-alkyl-amino-sulfonyl-
hydroxy-C₁-C₇-alkyl-amino-sulfonyl-
hydrazino-carbonyl-
C₁-C₇-alkyl-carbonyl-hydrazino-carbonyl-
phosphonyl-
C₁-C₇-alkyl-phosphonyl- and
di-C₁-C₇-alkyl-phosphonyl-.

6. The compound according to claim 1, or a tautomer or a N-oxide or a pharmaceutically acceptable salt thereof, wherein R⁴ is selected from the group consisting of:
substituted C₁-alkyl-
C₂-C₇-alkyl-
aryl-
heteroaryl-
heterocyclyl-
C₃-C₁₀-cycloalkyl-
aryl-C₁-C₇-alkyl-
heterocyclyl-C₁-C₇-alkyl-
C₃-C₁₀-cycloalkyl-C₁-C₇-alkyl-
unsubstituted or substituted by 1-2 substituents selected from
hydroxy-
C₁-C₇-alkoxy-
halogen-
hydroxy-C₁-C₇-alkyl-
N,N-di-C₁-C₇-alkyl-aminocarbonyl C₁-C₇-alkyl-amino-
amino-heterocyclyl-C₁-C₇-alkyl-amino-phenyl-
formyl-
carboxy-C₁-C₇-alkyl-amino
halo-C₁-C₇-alkyl-
nitro-
C₁-C₇-alkyl-carbonyl-
C₁-C₇-alkyl-
amino-
N,N-di-C₁-C₇-alkyl-amino-
amino-C₁-C₇-alkyl-amino-
amino-carbonyl-C₁-C₇-alkyl-amino-
hydroxy-alkyl-
C₁-C₇-alkyl-carbonyl-amino-
hydrazino-carbonyl-C₁-C₇-alkyl-amino-
heterocyclyl-C₁-C₇-alkyl-N—C₁-C₇-alkyl-amino-carbonyl-C₁-C₇-alkyl-amino-
heterocyclyl-C₁-C₇-alkyl-amino-
C₃-C₁₀-cycloalkyl-C₁-C₇-alkyl-N—C₁-C₇-alkyl-amino-carbonyl-C₁-C₇-alkyl-amino-
di-C₁-C₇-alkoxy-C₁-C₇-alkyl-amino-carbonyl-C₁-C₇-alkyl-amino-
C₁-C₇-alkoxy-carbonyl-C₁-C₇-alkyl-amino-
hydroxy-carbonyl-C₁-C₇-alkyl-amino-
C₁-C₇-alkyl-carbonyl-C₁-C₇-alkyl-amino-
C₁-C₇-alkyl-carbonyl-amino-C₁-C₇-alkyl-amino-
C₁-C₇-alkyl-amino carbonyl-amino-C₁-C₇-alkyl-amino-
benzyloxy-carbonyl-
C₁-C₇-alkyl-carbonyl-C₁-C₇-alkyl-amino-carbonyl-
heterocyclyl- and
protected hydroxy-.

7. The compound according to claim 1, or a tautomer or a N-oxide or a pharmaceutically acceptable salt thereof, wherein R′ and R″ are independently selected from the group consisting of
heterocyclyl-heterocyclyl-carbonyl-C₁-C₇-alkyl-
hydroxy-
C₁-C₇-alkoxy-
halogen-
halo-C₁-C₇-alkyl-
cyano-
C₁-C₇-alkyl-carbonyl-
formyl-
C₁-C₇-alkyl-
amino-carbonyl-
N—C₁-C₇-alkyl-amino-carbonyl-
N,N-di-C₁-C₇-alkyl-amino-carbonyl-
amino-C₁-C₇-alkyl-
heterocyclyl-C₁-C₇-alkyl-
N—C₁-C₇-alkyl-amino-C₁-C₇-alkyl-
N,N-di-C₁-C₇-alkyl-amino-C₁-C₇-alkyl-
heterocyclyl-carbonyl-C₁-C₇-alkyl-
heterocyclyl-heterocyclyl-carbonyl-C₁-C₇-alkyl-heterocyclyl-C₁-C₇-alkyl-amino-carbonyl-
heterocyclyl-C₁-C₇-alkyl-aminocarbonyl
heterocyclyl-C₁-C₇-alkyl-N—C₁-C₇-alkyl-amino-carbonyl-
amino-carbonyl-C₁-C₇-alkyl-
N—C₁-C₇-alkyl-amino-carbonyl-C₁-C₇-alkyl-
N,N-di-C₁-C₇-alkyl-amino-carbonyl-C₁-C₇-alkyl-
amino-C₁-C₇-alkyl-amino-C₁-C₇-alkyl-
amino-C₁-C₇-alkyl-N′—C₁-C₇-alkyl-amino-C₁-C₇-alkyl-
N—C₁-C₇-alkyl-amino-C₁-C₇-alkyl-amino-C₁-C₇-alkyl-
N—C₁-C₇-alkyl-amino-C₁-C₇-alkyl-N′—C₁-C₇-alkyl-amino-C₁-C₇-alkyl-
N,N-di-C₁-C₇-alkyl-amino-C₁-C₇-alkyl-amino-C₁-C₇-alkyl-
N,N-di-C₁-C₇-alkyl-amino-C₁-C₇-alkyl-N′—C₁-C₇-alkyl-amino-C₁-C₇-alkyl-
C₁-C₇-alkoxy-C₁-C₇-alkyl-amino-carbonyl-C₁-C₇-alkyl-
C₁-C₇-alkoxy-C₁-C₇-alkyl-N—C₁-C₇-alkyl-amino-carbonyl-C₁-C₇-alkyl-
C₁-C₇-alkoxy-carbonyl-C₁-C₇-alkyl-
C₃-C₁₀-cycloalkyl-C₁-C₇-alkyl-amino-carbonyl-C₁-C₇-alkyl-
C₃-C₁₀-cycloalkyl-C₁-C₇-alkyl-N—C₁-C₇-alkyl-amino-carbonyl-C₁-C₇-alkyl-
C₃-C₁₀-cycloalkyl-amino-carbonyl-C₁-C₇-alkyl-
heterocyclyl-C₁-C₇-alkyl-amino-carbonyl-C₁-C₇-alkyl-
heterocyclyl-amino-carbonyl-C₁-C₇-alkyl-
heterocyclyl-C₁-C₇-alkyl-N—C₁-C₇-alkyl-amino-carbonyl-C₁-C₇-alkyl-
aryl-C₁-C₇-alkyl-amino-carbonyl-C₁-C₇-alkyl-
aryl-C₁-C₇-alkyl-N—C₁-C₇-alkyl-amino-carbonyl-C₁-C₇-alkyl-
aryl-amino-carbonyl-C₁-C₇-alkyl-
aryl-amino-C₁-C₇-alkyl-amino-carbonyl-C₁-C₇-alkyl-
aryl-amino-C₁-C₇-alkyl-N—C₁-C₇-alkyl-amino-carbonyl-C₁-C₇-alkyl-
aryl-N—C₁-C₇-alkyl-amino-C₁-C₇-alkyl-amino-carbonyl-C₁-C₇-alkyl-
aryl-N—C₁-C₇-alkyl-amino-C₁-C₇-alkyl-N′—C₁-C₇-alkyl-amino-carbonyl-C₁-C₇-alkyl-
amino-C₁-C₇-alkyl-amino-carbonyl-
amino-C₁-C₇-alkyl-N′—C₁-C₇-alkyl-carbonyl-
N—C₁-C₇-alkyl-amino-C₁-C₇-alkyl-amino-carbonyl-
N—C₁-C₇-alkyl-amino-C₁-C₇-alkyl-N′—C₁-C₇-alkyl-amino-carbonyl-
N,N-di-C₁-C₇-alkyl-amino-C₁-C₇-alkyl-amino-carbonyl-
N,N-di-C₁-C₇-alkyl-amino-C₁-C₇-alkyl-N′—C₁-C₇-alkyl-amino-carbonyl-
amino-C₁-C₇-alkyl-amino-carbonyl-C₁-C₇-alkyl-
amino-C₁-C₇-alkyl-N′—C₁-C₇-alkyl-amino-carbonyl-C₁-C₇-alkyl-
N—C₁-C₇-alkyl-amino-C₁-C₇-alkyl-amino-carbonyl-C₁-C₇-alkyl-
N—C₁-C₇-alkyl-amino-C₁-C₇-alkyl-N′—C₁-C₇-alkyl-amino-carbonyl-C₁-C₇-alkyl-
N,N-di-C₁-C₇-alkyl-amino-C₁-C₇-alkyl-amino-carbonyl-C₁-C₇-alkyl-
N,N-di-C₁-C₇-alkyl-amino-C₁-C₇-alkyl-N′—C₁-C₇-alkyl-amino-carbonyl-C₁-C₇-alkyl-
di-C₁-C₇-alkoxy-C₁-C₇-alkyl-amino-carbonyl-C₁-C₇-alkyl-
carboxyl-C₁-C₇-alkyl-
hydroxy-C₁-C₇-alkyl-
heterocyclyl-
N-(hydroxy-C₁-C₇-alkyl)-amino-carbonyl-C₁-C₇-alkyl-
N—(C₁-C₇-alkoxy-C₁-C₇-alkyl)-amino-carbonyl-C₁-C₇-alkyl-
N-(hydroxy-C₁-C₇-alkyl)-N—C₁-C₇-alkyl-amino-carbonyl-C₁-C₇-alkyl-
N—(C₁-C₇-alkoxy-C₁-C₇-alkyl)-N—C₁-C₇-alkyl-amino-carbonyl-C₁-C₇-alkyl-
C₁-C₇-alkyl-carbonyl-amino-C₁-C₇-alkyl-
C₁-C₇-alkyl-carbonyl-N—C₁-C₇-alkyl-amino-C₁-C₇-alkyl-
C₁-C₇-alkoxy-carbonyl-
C₁-C₇-alkyl-carbonyl-amino-
carboxyl-
hydroxy-C₁-C₇-alkyl-cyclopropyl-amino-carbonyl-methyl-, and
C₁-C₇-alkoxy-carbonyl-amino-C₁-C₇-alkyl-aminocarbonyl-alkyl-.

8. The compound according to claim 7, or a tautomer or a N-oxide or a pharmaceutically acceptable salt thereof, wherein R′ and/or R″ are selected from the group consisting of hydrogen,
chloro,
fluoro,
methoxy
hydroxy
amino-carbonyl-C₁-C₇-alkyl-
N—C₁-C₇-alkyl-amino-carbonyl-C₁-C₇-alkyl-, or
N,N-di-C₁-C₇-alkyl-amino-carbonyl-C₁-C₇-alkyl-
C₁-C₇-alkoxy-C₁-C₇-alkyl-amino-carbonyl-C₁-C₇-alkyl-
C₁-C₇-alkoxy-C₁-C₇-alkyl-N—C₁-C₇-alkyl-amino-carbonyl-C₁-C₇-alkyl-
C₃-C₁₀-cycloalkyl-C₁-C₇-alkyl-amino-carbonyl-C₁-C₇-alkyl-
C₃-C₁₀-cycloalkyl-C₁-C₇-alkyl-N—C₁-C₇-alkyl-amino-carbonyl-C₁-C₇-alkyl-
C₃-C₁₀-cycloalkyl-amino-carbonyl-C₁-C₇-alkyl-
heterocyclyl-C₁-C₇-alkyl-amino-carbonyl-C₁-C₇-alkyl-
heterocyclyl-amino-carbonyl-C₁-C₇-alkyl-
heterocyclyl-C₁-C₇-alkyl-N—C₁-C₇-alkyl-amino-carbonyl-C₁-C₇-alkyl-
aryl-C₁-C₇-alkyl-amino-carbonyl-C₁-C₇-alkyl-
aryl-C₁-C₇-alkyl-N—C₁-C₇-alkyl-amino-carbonyl-C₁-C₇-alkyl-
aryl-amino-carbonyl-C₁-C₇-alkyl-
aryl-amino-C₁-C₇-alkyl-amino-carbonyl-C₁-C₇-alkyl-
aryl-amino-C₁-C₇-alkyl-N—C₁-C₇-alkyl-amino-carbonyl-C₁-C₇-alkyl-
aryl-N—C₁-C₇-alkyl-amino-C₁-C₇-alkyl-amino-carbonyl-C₁-C₇-alkyl-
aryl-N—C₁-C₇-alkyl-amino-C₁-C₇-alkyl-N′—C₁-C₇-alkyl-amino-carbonyl-C₁-C₇-alkyl-
amino-C₁-C₇-alkyl-amino-carbonyl-C₁-C₇-alkyl-
amino-C₁-C₇-alkyl-N′—C₁-C₇-alkyl-amino-carbonyl-C₁-C₇-alkyl-
N—C₁-C₇-alkyl-amino-C₁-C₇-alkyl-amino-carbonyl-C₁-C₇-alkyl-
N—C₁-C₇-alkyl-amino-C₁-C₇-alkyl-N′—C₁-C₇-alkyl-amino-carbonyl-C₁-C₇-alkyl-
N,N-di-C₁-C₇-alkyl-amino-C₁-C₇-alkyl-amino-carbonyl-C₁-C₇-alkyl-
N,N-di-C₁-C₇-alkyl-amino-C₁-C₇-alkyl-N′—C₁-C₇-alkyl-amino-carbonyl-C₁-C₇-alkyl-
di-C₁-C₇-alkoxy-C₁-C₇-alkyl-amino-carbonyl-C₁-C₇-alkyl-
N-(hydroxy-C₁-C₇-alkyl)-amino-carbonyl-C₁-C₇-alkyl-
N—(C₁-C₇-alkoxy-C₁-C₇-alkyl)-amino-carbonyl-C₁-C₇-alkyl-
N-(hydroxy-C₁-C₇-alkyl)-N—C₁-C₇-alkyl-amino-carbonyl-C₁-C₇-alkyl-
N—(C₁-C₇-alkoxy-C₁-C₇-alkyl)-N—C₁-C₇-alkyl-amino-carbonyl-C₁-C₇-alkyl-
hydroxy-C₁-C₇-alkyl-cyclopropyl-amino-carbonyl-methyl-, and
C₁-C₇-alkoxy-carbonyl-amino-C₁-C₇-alkyl-aminocarbonyl-alkyl-.

9. A pharmaceutical composition comprising a compound of claim 1, or a tautomer, or a N-oxide or a pharmaceutically acceptable salt thereof, and at least one pharmaceutically acceptable carrier material.

10. A method of treating a disorder or a disease in a subject mediated by the activity of MDM2 or MDM4, or variants thereof comprising administering to the subject a therapeutically effective amount of a compound of anyone of formulae (I), (Ia), (Ib), (Ic), (Id) or (Ie), as defined in claim 2, or a tautomer, or a N-oxide, or a pharmaceutically acceptable salt thereof.

11-12. (canceled)

13. The method of claim 10 wherein the disease is a proliferative disease.

14. A combination of a compound of claim 1, or a tautomer, or a N-oxide, or a pharmaceutically acceptable salt thereof, and another pharmacologically active agent.
